SEQUENCE LISTING (52 sequences)

SEQ ID NO: 1 - protein, 1038 aa, Homo sapiens
MFPTESAWLGKRGANPGPEAAVRETVMLLLCLGVPTGRPYNVDTESALLYQGPHNTLFGYSVVLHSHGANRWLLVGAPTANWLANASVINPGAIYRCRIGKNPGQTCEQLQLGSPNGEPCGKTCLEERDNQWLGVTLSRQPGENGSIVTCGHRWKNIFYIKNENKLPTGGCYGVPPDLRTELSKRIAPCYQDYVKKFGENFASCQAGISSFYTKDLIVMGAPGSSYWTGSLFVYNITTNKYKAFLDKQNQVKFGSYLGYSVGAGHFRSQHTTEVVGGAPQHEQIGKAYIFSIDEKELNILHEMKGKKLGSYFGASVCAVDLNADGFSDLLVGAPMQSTIREEGRVFVYINSGSGAVMNAMETNLVGSDKYAARFGESIVNLGDIDNDGFEDVAIGAPQEDDLQGAIYIYNGRADGISSTFSQRIEGLQISKSLSMFGQSISGQIDADNNGYVDVAVGAFRSDSAVLLRTRPVVIVDASLSHPESVNRTKFDCVENGWPSVCIDLTLCFSYKGKEVPGYIVLFYNMSLDVNRKAESPPRFYFSSNGTSDVITGSIQVSSREANCRTHQAFMRKDVRDILTPIQIEAAYHLGPHVISKRSTEEFPPLQPILQQKKEKDIMKKTINFARFCAHENCSADLQVSAKIGFLKPHENKTYLAVGSMKTLMLNVSLFNAGDDAYETTLHVKLPVGLYFIKILELEEKQINCEVTDNSGVVQLDCSIGYIYVDHLSRIDISFLLDVSSLSRAEEDLSITVHATCENEEEMDNLKHSRVTVAIPLKYEVKLTVHGFVNPTSFVYGSNDENEPETCMVEKMNLTFHVINTGNSMAPNVSVEIMVPNSFSPQTDKLFNILDVQTTTGECHFENYQRVCALEQQKSAMQTLKGIVRFLSKTDKRLLYCIKADPHCLNFLCNFGKMESGKEASVHIQLEGRPSILEMDETSALKFEIRATGFPEPNPRVIELNKDENVAHVLLEGLHHQRPKRYFTIVIISSSLLLGLIVLLLISYVMWKAGFFKRQYKSILQEENRRDSWSYINSKSNDD

SEQ ID NO: 2 - protein, 798 aa, Homo sapiens [sequence omitted]

SEQ ID NO: 3 - protein, 798 aa, Homo sapiens
MVALPMVLVLLLVLSRGESELDAKIPSTGDATEWRNPHLSMLGSCQPAPSCQKCILSHPSCAWCKQLNFTASGEAEARRCARREELLARGCPLEELEEPRGQQEVLQDQPLSQGARGEGATQLAPQRVRVTLRPGEPQQLQVRFLRAEGYPVDLYYLMDLSYSMKDDLERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGHCHLDSNGLYSRSTEFDYPSVGQVAQALSAANIQPIFAVTSAALPVYQELSKLIPKSAVGELSEDSSNVVQLIMDAYNSLSSTVTLEHSSLPPGVHISYESQCEGPEKREGKAEDRGQCNHVRINQTVTFWVSLQATHCLPEPHLLRLRALGFSEELIVELHTLCDCNCSDTQPQAPHCSDGQGHLQCGVCSCAPGRLGRLCECSVAELSSPDLESGCRAPNGTGPLCSGKGHCQCGRCSCSGQSSGHLCECDDASCERHEGILCGGFGRCQCGVCHCHANRTGRACECSGDMDSCISPEGGLCSGHGRCKCNRCQCLDGYYGALCDQCPGCKTPCERHRDCAECGAFRTGPLATNCSTACAHTNVTLALAPILDDGWCKERTLDNQLFFFLVEDDARGTVVLRVRPQEKGADHTQAIVLGCVGGIVAVGLGLVLAYRLSVEIYDRREYSRFEKEQQQLNWKQDSNPLYKSAITTTINPRFQEADSPTL

SEQ ID NO: 4 - protein, 1170 aa, Homo sapiens
MKDSCITVMAMALLSGFFFFAPASSYNLDVRGARSFSPPRAGRHFGYRVLQVGNGVIVGAPGEGNSTGSLYQCQSGTGHCLPVTLRGSNYTSKYLGMTLATDPTDGSILACDPGLSRTCDQNTYLSGLCYLFRQNLQGPMLQGRPGFQECIKGNVDLVFLFDGSMSLQPDEFQKILDFMKDVMKKLSNTSYQFAAVQFSTSYKTEFDFSDYVKRKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVIEGTSKQDLTSFNMELSSSGISADLSRGHAVVGAVGAKDWAGGFLDLKADLQDDTFIGNEPLTPEVRAGYLGYTVTWLPSRQKTSLLASGAPRYQHMGRVLLFQEPQGGGHWSQVQTIHGTQIGSYFGGELCGVDVDQDGETELLLIGAPLFYGEQRGGRVFIYQRRQLGFEEVSELQGDPGYPLGRFGEAITALTDINGDGLVDVAVGAPLEEQGAVYIFNGRHGGLSPQPSQRIEGTQVLSGIQWFGRSIHGVKDLEGDGLADVAVGAESQMIVLSSRPVVDMVTLMSFSPAEIPVHEVECSYSTSNKMKEGVNITICFQIKSLYPQFQGRLVANLTYTLQLDGHRTRRRGLFPGGRHELRRNIAVTTSMSCTDFSFHFPVCVQDLISPINVSLNFSLWEEEGTPRDQRAQGKDIPPILRPSLHSETWEIPFEKNCGEDKKCEANLRVSFSPARSRALRLTAFASLSVELSLSNLEEDAYWVQLDLHFPPGLSFRKVEMLKPHSQIPVSCEELPEESRLLSRALSCNVSSPIFKAGHSVALQMMFNTLVNSSWGDSVELHANVTCNNEDSDLLEDNSATTIIPILYPINILIQDQEDSTLYVSFTPKGPKIHQVKHMYQVRIQPSIHDHNIPTLEAVVGVPQPPSEGPITHQWSVQMEPPVPCHYEDLERLPDAAEPCLPGALFRCPVVFRQEILVQVIGTLELVGEIEASSMFSLCSSLSISFNSSKHFHLYGSNASLAQVVMKVDVVYEKQMLYLYVLSGIGGLLLLLLIFIVLYKVGFFKRNLKEKMEAGRGVPNGIPAEDSEQLASGQEAGDPGCLKPLHEKDSESGGGKD

SEQ ID NO: 5 - protein, 769 aa, Homo sapiens
MLGLRPPLLALVGLLSLGCVLSQECTKFKVSSCRECIESGPGCTWCQKLNFTGPGDPDSIRCDTRPQLLMRGCAADDIMDPTSLAETQEDHNGGQKQLSPQKVTLYLRPGQAAAFNVTFRRAKGYPIDLYYLMDLSYSMLDDLRNVKKLGGDLLRALNEITESGRIGFGSFVDKTVLPFVNTHPDKLRNPCPNKEKECQPPFAFRHVLKLTNNSNQFQTEVGKQLISGNLDAPEGGLDAMMQVAACPEEIGWRNVTRLLVFATDDGFHFAGDGKLGAILTPNDGRCHLEDNLYKRSNEFDYPSVGQLAHKLAENNIQPIFAVTSRMVKTYEKLTEIIPKSAVGELSEDSSNVVHLIKNAYNKLSSRVFLDHNALPDTLKVTYDSFCSNGVTHRNQPRGDCDGVQINVPITFQVKVTATECIQEQSFVIRALGFTDIVTVQVLPQCECRCRDQSRDRSLCHGKGFLECGICRCDTGYIGKNCECQTQGRSSQELEGSCRKDNNSIICSGLGDCVCGQCLCHTSDVPGKLIYGQYCECDTINCERYNGQVCGGPGRGLCFCGKCRCHPGFEGSACQCERTTEGCLNPRRVECSGRGRCRCNVCECHSGYQLPLCQECPGCPSPCGKYISCAECLKFEKGPFGKNCSAACPGLQLSNNPVKGRTCKERDSEGCWVAYTLEQQDGMDRYLIYVDESRECVAGPNIAAIVGGTVAGIVLIGILLLVIWKALIHLSDLREYRRFEKEKLKSQWNNDNPLFKSATTTVMNPKFAES

SEQ ID NO: 6 - protein, 739 aa, Homo sapiens
MPGKMVVILGASNILWIMFAASQAFKIETTPESRYLAQIGDSVSLTCSTTGCESPFFSWRTQIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPKNTVISVNPSTKLQEGGSVTMTCSSEGLPAPEIFWSKKLDNGNLQHLSGNATLTLIAMRMEDSGIYVCEGVNLIGKNRKEVELIVQEKPFTVEISPGPRIAAQIGDSVMLTCSVMGCESPSFSWRTQIDSPLSGKVRSEGTNSTLTLSPVSFENEHSYLCTVTCGHKKLEKGIQVELYSFPRDPEIEMSGGLVNGSSVTVSCKVPSVYPLDRLEIELLKGETILENIEFLEDTDMKSLENKSLEMTFIPTIEDTGKALVCQAKLHIDDMEFEPKQRQSTQTLYVNVAPRDTTVLVSPSSILEEGSSVNMTCLSQGFPAPKILWSRQLPNGELQPLSENATLTLISTKMEDSGVYLCEGINQAGRSRKEVELIIQVTPKDIKLTAFPSESVKEGDTVIISCTCGNVPETWIILKKKAETGDTVLKSIDGAYTIRKAQLKDAGVYECESKNKVGSQLRSLTLDVQGRENNKDYFSPELLVLYFASSLIIPAIGMIIYFARKANMKGSYSLVEAQKSKV

SEQ ID NO: 7 - protein, 406 aa, Homo sapiens
MDFGLALLLAGLLGLLLGQSLQVKPLQVEPPEPVVAVALGASRQLTCRLACADRGASVQWRGLDTSLGAVQSDTGRSVLTVRNASLSAAGTRVCVGSCGGRTFQHTVQLLVYAFPDQLTVSPAALVPGDPEVACTAHKVTPVDPNALSFSLLVGGQELEGAQALGPEVQEEEEEPQGDEDVLFRVTERWRLPPLGTPVPPALYCQATMRLPGLELSHRQAIPVLHSPTSPEPPDTTSPESPDTTSPESPDTTSPESPDTTSQEPPDTTSQEPPDTTSQEPPDTTSPEPPDKTSPEPAPQQGSTHTPRSPGSTRTRRPEISQAGPTQGEVIPTGSSKPAGDQLPAALWTSSAVLGLLLLALPTYHLWKRCRHLAEDDTHPPASLRLLPQVSAWAGLRGTGQVGISPS

SEQ ID NO: 8 - protein, 106 aa, Artificial sequence [sequence omitted]

SEQ ID NO: 9 - protein, 106 aa, Artificial sequence
  sequence is synthesized
DIQMTQSPSSLSASVGDRVTITCKTSQDINKYMAWYQQTPGKAPRLLIYYTSALQPGIPSRFSGSGSGRDYTFTISSLQPEDIATYYCLQYDNLWTFGQGTKVEIK

SEQ ID NO: 10 - protein, 124 aa, Artificial sequence
  sequence is synthesized
QVQLVQSGAEVKKPGASSVKVSCKASGFNIKDTYIHWVRQAPGQRLEWMBRIDPANGYTKYDPKFQGRVTITADTSASTAYMELSSLRSEDTAVYYCAREGYYGNYGVYAMDYWGQGTLVTVSS

SEQ ID NO: 11 - protein, 124 aa, Artificial sequence
  sequence is synthesized
QVQLVQSGAEVKKPGASSVKVSCKASGFNIKDTYIHWVRQAPGQGLEWMBRIDPANGYTKYDPKFQGRVTITADTSASTAYMELSSLRSEDTAVYYCAREGYYGNYGVYAMDYWGQGTLVTVSS

SEQ ID NO: 12 - protein, 124 aa, Artificial sequence
  sequence is synthesized
QVQLVQSGAEVKKPGASSVKVSCKASGFNIKDTYIHWVRQAPGQRLEWMBRIDPANGYTKYDPKFQGRVTITADTSASTAYMELSSLRSEDTAVYYCAREGYFGNYGVYAMDYWGQGTLVTVSS

SEQ ID NO: 13 - protein, 107 aa, Artificial sequence
  sequence is synthesized
SIVMTQSPSSLSASVGDRVTITCKASQSVTNDVAWYQQKPGKAPKLLIYYASNRYTGVPDRFSGSGYGTDFTFTISSLQPEDIATYYCQQDYSSPYTFGQGTKVEIK

SEQ ID NO: 14 - protein, 107 aa, Artificial sequence
  sequence is synthesized
DIQMTQSPSSLSASVGDRVTITCKASQSVTNDVAWYQQKPGKAPKLLIYYASNRYTGVPDRFSGSGSGTDFTFTISSLQPEDIATYYCQQDYSSPYTFGQGTYVEIK

SEQ ID NO: 15 - protein, 107 aa, Artificial sequence
  sequence is synthesized
SIVMTQSPDSLAVSLGERVTINCKASQSVTNDVAWYQQKPGQSPKLLIYYASNRYTGVPDRFSGSGYGTDFTFTISSVQAEDVAVYYCQQDYSSPYTFGGGTKLEIK

SEQ ID NO: 16 - protein, 121 aa, Artificial sequence
  sequence is synthesized
QVQLQESGPGLVRPSQTLSLTCTVSGFNIKDTYMHWVRQPPGRGLEWIGRIDPASGDTKYDPKFQVRVTMLVDTSSNTAWLRLSSVTAADTAVYYCADGMWVSTGYALDFWGQGTTVTVSS

SEQ ID NO: 17 - protein, 120 aa, Artificial sequence
  sequence is synthesized
QVQLQESPGLVRPSQTLSLTCTVSGFNIKDTYMHWVRQPPGRGLEWIGRIDPASGDTKYDPKFQVKATITADTSSNQFSLRLSSVTAADTAVYYCADGMWVSTGYALDFWGQGTTVTVSS

SEQ ID NO: 18 - protein, 121 aa, Artificial sequence
  sequence is synthesized
QVQLQESGPGLVRPSQTLSLTCTVSGFNIKDTYMHWVRQPPGRGLEWIGRIDPASGDTKYDPKFQVRVTMLVDTSSNQFSLRLSSVTSEDTAVYYCADGMWVSTGYALDFWGQGTTVTVSS

SEQ ID NO: 19 - protein, 121 aa, Artificial sequence
  sequence is synthesized
QVQLQESGPGLVRPSQTLSLTCTVSGFNIKDTYMHWVKQRPGRGLEWIGRIDPASGDTKYDPKFQVRVTMLVDTSSNQFSLRLSSVTAADTAVYYCADGMWVSTGYALDFWGQGTTVTVSS

SEQ ID NO: 20 - protein, 121 aa, Artificial sequence
  sequence is synthesized
QVQLQESGPGLVRPSQTLSLTCTASGFNIKDTYMHWVRQPPGRGLEWIGRIDPASGDTKYDPKFQVRVTMLVDTSSNQFSLRLSSVTAADTAVYYCADGMWVSTGYALDFWGQGTTVTVSS

SEQ ID NO: 21 - protein, 121 aa, Artificial sequence
  sequence is synthesized
QVQLQESGAEVVKPGSSVKLSCKASGFNIKDTYMHWVKQRPGQGLEWIGRIDPASGDTKYDPKFQVKATITADESTSTAYLELSSLRSEDTAVYYCADGMWVSTGYALDFWGQGTTVTVSS

SEQ ID NO: 22 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTREVTVNVLSPRYEIVIITVVAAAVIMGTAGLSTYLYNRQRKIKKYRLQQAQKGTPMKPNTQATPP

SEQ ID NO: 23 - protein, 275 aa, Homo sapiens
MSSFGYRTLTVALFTLICCPGSDEKVFEVHVRPKKLAVEPKGSLEVNCSTTCNQPEVGGLETSLDKILLDEQAQWKHYLVSNISHDTVLQCHFTCSGKQESMNSNVSVYQPPRQVILTLQPTLVAVGKSFTIECRVPTVEPLDSLTLFLFRGNETLHYETFGKAAPAPQEATATFNSTADREDGHRNFSCLAVLDLMSRGGNIFHKHSAPKMLEIYEPVSDSQMVIIVTVVSVLLSLFVTSVLLCFIFGQHLRQQRMGTYGVRAAWRRLPQAFRP

SEQ ID NO: 24 - protein, 547 aa, Homo sapiens
MATMVPSVLWPRACWTLLVCCLLTPGVQGQEFLLRVEPQNPVLSAGGSLFVNCSTDCPSSEKIALETSLSKELVASGMGWAAFNLSNVTGNSRILCSVYCNGSQITGSSNITVYGLPERVELAPLPPWQPVGQNFTLRCQVEGGSPRTSLTVVLLRWEEELSRQPAVEEPAEVTATVLASRDDHGAPFSCRTELDMQPQGLGLFVNTSAPRQLRTFVLPVTPPRLVAPRFLEVETSWPVDCTLDGLFPASEAQVYLALGDQMLNATVMNHGDTLTATATATARADQEGAREIVCNVTLGGERREARENLTVFSFLGPIVNLSEPTAHEGSTVTVSCMAGARVQVTLDGVPAAAPGQPAQLQLNATESDDGRSFFCSATLEVDGEFLHRNSSVQLRVLYGPKIDRATCPQHLKWKDKTRHVLQCQARGNPYPELRCLKEGSSREVPVGIPFFVNVTHNGTYQCQASSSRGKYTLVVVMDIEAGSSHFVPVFVAVLLTLGVVTIVLALMYVFREHQRSGSYHVREESTYLPLTSMQPTEAMGEEPSRAE

SEQ ID NO: 25 - protein, 271 aa, Homo sapiens
MGSLFPLSLLFFLAAAYPGVGSALGRRTKRAQSPKGSPLAPSGTSVPFWVRMSPEFVAVQPGKSVQLNCSNSCPQPQNSSLRTPLRQGKTLRGPGWVSYQLLDVRAWSSLAHCLVTCAGKTRWATSRITAYKPPHSVILEPPVLKGRKYTLRCHVTQVFPVGYLVVTLRHGSRVIYSESLERFTGLDLANVTLTYEFAAGPRDFWQPVICHARLNLDGLVVRNSSAPITLMLAWSPAPTALASGSIAALVGILLTVGAAYLCKCLAMKSQA

SEQ ID NO: 26 - protein, 924 aa, Homo sapiens
MPGPSPGLRRALLGLWAALGLGLFGLSAVSQEPFWADLQPRVAFVERGGSLWLNCSTNCPRPERGGLETSLRRNGTQRGLRWLARQLVDIREPETQPVCFFRCARRTLQARGLIRTFQRPDRVELMPLPPWQPVGENFTLSCRVPGAGPRASLTLTLLRGAQELIRRSFAGEPPRARGAVLTATVLARREDHGANFSCRAELDLRPHGLGLFENSSAPRELRTFSLSPDAPRLAAPRLLEVGSERPVSCTLDGLFPASEARVYLALGDQNLSPDVTLEGDAFVATATATASAEQEGARQLVCNVTLGGENRETRENVTIYSFPAPLLTLSEPSVSEGQMVTVTCAAGAQALVTLEGVPAAVPGQPAQLQLNATENDDRRSFFCDATLDVDGETLIKNRSAELRVLYAPRLDDSDCPRSWTWPEGPEQTLRCEARGNPEPSVHCARSDGGAVLALGLLGPVTRALSGTYRCKAANDQGEAVKDVTLTVEYAPALDSVGCPERITWLEGTEASLSCVAHGVPPPDVICVRSGELGAVIEGLLRVAREHAGTYRCEATNPRGSAAKNVAVTVEYGPRFEEPSCPSNWTWVEGSGRLFSCEVDGKPQPSVKCVGSGGTTEGVLLPLAPPDPSPRAPRIPRVLAPGIYVCNATNRHGSVAKTVVVSAESPPEMDESTCPSHQTWLEGAEASALACAARGRPSPGVRCSREGIPWPEQQRVSREDAGTYHCVATNAHGTDSRTVTVGVEYRPVVAELAASPPGGVRPGGNFTLTCRAEAWPPAQISWRAPPRALNIGLSSNNSTLSVAGAMGSHGGEYECARTNAHGRHARRITVRVAGPWLWVAVGGAAGGAALLAAGAGLAFYVQSTACKKGEYNVQEAESSGEAVCLNGAGGGAGGAAGAEGGPEAAGGAAESPAEGEVFAIQLTSA

SEQ ID NO: 27 - protein, 121 aa, Mus musculus
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVS

SEQ ID NO: 28 - protein, 106 aa, Mus musculus [sequence omitted]

SEQ ID NO: 29 - protein, 107 aa, Artificial sequence
  sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 30 - protein, 122 aa, Artificial sequence [sequence omitted]

SEQ ID NO: 31 - protein, 213 aa, Artificial sequence
  sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 106 aa, Artificial sequence
  sequence is synthesized
DIQLTQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIK

SEQ ID NO: 35 - protein, 121 aa, Artificial sequence
  sequence is synthesized
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFDVWGQGTTVTVSS

SEQ ID NO: 36 - protein, 106 aa, Artificial sequence
  sequence is synthesized
DIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIK

SEQ ID NO: 37 - protein, 121 aa, Artificial sequence
  sequence is synthesized
QVQLQQSGAEVKKPGSSVKVSCKASGYTFTSYNMHWVKQAPGQGLEWIGAIYPGNGDTSYNQKFKGKATLTADESTNTAYMELSSLRSEDTAFYYCARSTYYGGDWYFDVWGQGTTVTVSS

SEQ ID NO: 38 - protein, 121 aa, Artificial sequence
  sequence is synthesized
QVQLQQSGAEVKKPGSSVKVSCKASGYTFSSYNMHWVRQAPGQGLEWMGAIYPGNGDTSYNQKFKGRATITADESTNTAYMELSSLRSEDTAFYFCARSTYYGGDWYFDVWGQGTTVTVSS

SEQ ID NO: 39 - protein, 122 aa, Artificial sequence
  sequence is synthesized
QVQLVASGAEVNKPGASVKVSCKASGYTFTSYNMHWVRQPPGRGLEWIGAIYPGNGDTSYNQKFKGKATLTADKSSSTAYMQLSSLTSEDSAVYYCARSHYGSNYVDYFDYWGQGTTVTVSS

SEQ ID NO: 40 - protein, 107 aa, Artificial sequence
  sequence is synthesized
DIQLTQSPSSLSASVGDRVTITCRASSSLSFMHWYQQKPGSSPKPWIYATSNLASGVPSRFSGSGSGTEFTLTISSLQPEDFATYFCHQWSSNPLTFGAGTKLTVLR

SEQ ID NO: 41 - protein, 122 aa, Artificial sequence
  sequence is synthesized
QVQLVASGAEVNKPGASVKVSCKASGYTFTSYNMHWVRQPPGRGLEWIGAIYPGNGDTSYNQKFKGRVTITADKSTSTAYMELSSLRSEDTAVYYCARSHYGSNYVDYFDYWGQGTTVTVSS

SEQ ID NO: 42 - protein, 107 aa, Artificial sequence
  sequence is synthesized
DIQLTQSPSSLSASVGDRVTITCRASSSLSFMHWYQQKPGQAPVPVIYATSNLASGVPSRFSGSGSGTEFTLTISSLQPEDFATYFCHQWSSNPLTFGAGTKLTVLR

SEQ ID NO: 43 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 213 aa, Artificial sequence
  sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 47 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 107 aa, Artificial sequence
  sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKR

SEQ ID NO: 49 - protein, 107 aa, Artificial sequence [sequence omitted]

SEQ ID NO: 50 - protein, 121 aa, Artificial sequence [sequence omitted]

SEQ ID NO: 51 - protein, 107 aa, Mus musculus
DVQITQSPSYLAASPGETISINCRASKTISKYLAWYQEKPGKTNKLLIYSGSTLQSGIPSRFSGSGSGTDFTLTISSLEPEDFAMYYCQQHNEYPLTFGTGTKLELK

SEQ ID NO: 52 - protein, 121 aa, Mus musculus
EVQLQQPGAELMRPGASVKLSCKASGYSFTGHWMNWVRQRPGQGLEWIGMIHPSDSETRLNQKFKDKATLTVDKSSSSAYMQLSSPTSEDSAVYYCARGIYFYGTTYFDYWGQGTTLTVSS

(19) United States
(12) Patent Application Publication
(10) Pub. No.: US 2005/0276803 A1
(43) Pub. Date: Dec. 15, 2005

(54) Method for augmenting B cell depletion

(75) Inventors: Andrew C. Chan, Menlo Park, CA (US); Qian Gong, Foster City, CA (US); Flavius Martin, Hayward, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 11/107,028

(22) Filed: Apr. 15, 2005

Related U.S. Application Data
(60) Provisional application No. 60/563,263, filed on Apr. 16, 2004.

(51) Int. Cl. A61K 39/395

(52) U.S. Cl. 424/141.1; 424/144.1

(57) ABSTRACT

The present invention provides methods of augmenting B cell depletion by promoting intravascular access of B cell subsets sequestered in lymphoid tissues rendering the B cells sensitive to killing mediated by the B cell depleting agent. One method of promoting intravascular access is by the use of integrin antagonists. Methods of treating B cell disorders by this approach is also provided.

### RELATED APPLICATIONS

This application claims the benefit under U.S.C. § 1.19(e)(1) to U.S. provisional application 60/563,263 filed on 16 Apr. 2004, the entire disclosure of which is incorporated herein by reference.

### FIELD OF THE INVENTION

The invention relates to methods of killing B cells.

### BACKGROUND OF THE INVENTION

Lymphocytes are one of several populations of white blood cells; they specifically recognize and respond to foreign antigen. The three major classes of lymphocytes are B lymphocytes (B cells), T lymphocytes (T cells) and natural killer (NK) cells. B lymphocytes are the cells responsible for antibody production and provide humoral immunity. B cells mature within the bone marrow and leave the marrow expressing an antigen-binding antibody on their cell surface. When a naive B cell first encounters the antigen for which its membrane-bound antibody is specific, the cell begins to divide rapidly and its progeny differentiate into memory B cells and effector cells called “plasma cells.” Memory B cells have a longer life span and continue to express membrane-bound antibody with the same specificity as the original parent cell. Plasma cells do not produce membrane-bound antibody but instead produce secreted form of the antibody. Secreted antibodies are the major effector molecules of humoral immunity.

Antibody therapeutics directed against B cell targets that rely on the ability of passively infused antibodies to deplete antigen-bearing cells have been developed to treat B cell diseases. For example, antibodies targeting the CD20, CD22, and CD52 surface molecules (Treon et al., 2000, Seminars in Oncology 27(6 suppl 12):79-85; Juweid, 2003, Current Opinion in Molecular Therapeutics 5(2):192-198; Cersosimo, 2003, Monoclonal antibodies in the treatment of cancer, Part 1, American Journal of Health-System Pharmacy 60(15):1531-1548; part II in 60(16) 1631-1641) have been developed.

The CD20 antigen (also called human B-lymphocyte-restricted differentiation antigen, Bp35) is a transmembrane phosphoprotein with a molecular weight of approximately 35 kD that is expressed exclusively on normal and malignant B cells. Its expression is regulated during B cell development emerging in late pre-B cells and is present on immature B and mature B lymphocytes (Valentine et al,. 1989, J. Biol. Chem. 264:11282-11287; and Einfeld et al., 1988, EMBO J. 7:711-717). The antigen is also expressed on greater than 90% of B cell non-Hodgkin's lymphomas (NHL) (Anderson et al., 1984, Blood 63:1424-1433), but is not found on hematopoietic stem cells, pro-B cells, normal plasma cells or other normal tissues (Tedder et al., 1985, J. Immunol. 135:973-979). CD20 is thought to regulate an early step(s) in the activation process for cell cycle initiation and differentiation (Tedder et al., supra) and possibly functions as a calcium ion channel (Tedder et al., 1990, J. Cell. Biochem. 14D: 195).

Integrins are a family of heterodimeric, transmembrane, cell adhesion receptors that can mediate cell-cell and cell-extracellular matrix interactions (Humphries, et al., 1990, TIBS 28:313-320). Integrins comprise two unrelated, type I membrane glycoproteins, known as alpha and beta subunits that non-covalently associate with each other (Humphries, supra). All alpha and beta subunits have large extracellular domains (700-1100 residues), one transmembrane helix and small cytoplasmic domains (30-50 residues) per subunit (Humphries, 2000, supra).

Mammals have at least nineteen different alpha subunits and eight beta subunits that assemble to form at least 25 different receptors (Humphries, 2000, Biochem. Soc. Trans. 28:311-339). Alpha subunits include alphaE, alphas 1-11, alphaV, alphaIIB, alphaL, alphaM, alphaX and alphaD (Arnaout et al., 2002, Immunological Reviews 186:125-140). Beta subunits include betas 1-8 (Arnaout, supra). The integrin subunits are expressed in different combinations and in different cell types. Alpha1, alpha2, alphaE, alphaL, alphaM, alphaX, alphaD, and beta2 share a distal N-terminal extracellular domain called the “I domain” or “A domain,” so called because the domain has been inserted into the integrin or because of its homology to the A motif in von Willebrand factor (Harris et al., 2000, JBC 275:23409-23412). The I domain is approximately 200 residues and has been reported to be critical for ligand binding (Harris, supra).

Alpha4, also known as CD49d or the alpha subunit of VLA-4, has been shown to associate with beta1 (CD29) and beta7 (Arnaout, supra; Barclay et al., Eds., 1997, The Leukocyte Antigen Facts Book, 2nd Ed, p. 262-263). (See also the listed subunits and cited references disclosed on “the Integrin Page,” located at http://integrins.hypermart.net.) Alpha4beta1 integrins are also known as very late antigen-4 integrin (VLA-4) (Mousa, 2002, Cur. Opin. Chem. Biol. 6:534-541). The VLA-4 integrin is expressed on most leukocytes, with the exception of neutrophils and platelets (Barclay, supra). It binds to ligands VCAM-1, fibronectin, thrombospondin, collagens, and invasin (Plow et al., 2000, JBC 275:21785-21788). The alpha4beta7 is also known as lymphocyte Peyer's patch adhesion molecule-1 (LPAM-1). Alpha4beta7 is expressed on most lymph node T and B cells, NK cells, and eosinophils (Barclay, supra), and binds to vascular cell adhesion molecule-1 (VCAM-1), mucocosal addressin cell adhesion molecule-1 (MAdCAM-1), and fibronectin (Plow, supra).

AlphaL, also known as CD11a or the alpha subunit of the integrin leukocyte function-associated antigen-1 (LFA-1), has been shown to associate with beta2 (CD18) to form LFA-1 (Arnaout, supra; Barclay, supra, p. 156-157). (See also, “the Integrin Page” and references cited therein, supra.) Unlike alpha4, alphaL contains an “I domain” (Harris, supra). The alphaLbeta2 (LFA-1) integrin is expressed on all leukocytes in humans. It binds to at least five ligands CD54 (ICAM-1), CD102 (ICAM-2), CD50 (ICAM-3), ICAM-4, and ICAM-5 (Plow, supra).

VCAM-1, also called INCAM-110 or CD106, is expressed predominantly on vascular endothelium but has also been identified on follicular and interfollicular dendritic cells, some macrophages, bone marrow stromal cells and non-vascular cell populations within joints, kidney, muscle, heart, placenta, and brain (The Leukocyte Antigen Facts Book, 2nd edition, eds., Barclay et al. Academic Press, Harcourt Brace & Company, San Diego, Calif., 1977).

The therapeutic use of several anti-integrins in treating various diseases, including various inflammation and autoimmune diseases has been explored due to activity of integrins in leukocyte trafficking (Mousa, supra; Yusuf-Makagiansar et al., 2002, Medicinal Research Reviews 22:146-167; Vincenti, 2002, American Journal of Transplantation 2:898-903). Recently, it has been reported that alphaLbeta2 (LFA-1) and alpha4beta1 (VLA-4) make substantial and mostly overlapping contributions to B cell retention within the marginal zone (MZ) in mice (Lu et al., 2002, Science 297:409-412). Lu reported that MZ B cells express elevated levels of alphaLbeta2 (LFA-I) and alpha4beta I (VLA4) and they bind to the ligands ICAM-1 (CD54) and VCAM-1 (CD106) that are expressed in the MZ. MZ is rich in IgM+ memory cells and cells that react with autoantigens and bacterial antigens. Mice treated with anti-alpha4 and anti-alphaL blocking antibodies were reported to have lost marginal zone B cells from the spleen and the blood (Lu, supra, p. 410-411). It was speculated that displacing B cells from an adhesive, LT alpha1beta2-mediated niche in the spleen by blocking integrin function might be a way to purge the compartment of autoreactive or malignant cells (Lu, supra, p. 412). The clinical relevance of removing these B cells from the compartment in the spleen is unclear; these cells may be moved out of one compartment only to move to another compartment. For a B cell malignancy, it is possible purging these pathogenic B cells may actually result in or exacerbate metastasis, thus worsening the disease.

Rituximab (Rituxan™, Genentech, Inc, South San Francisco, Calif. and Biogen-IDEC, Cambridge, Mass.; Mabthera®, F. Hoffman-LaRoche, Ltd., Basel, Switzerland) is a chimeric monoclonal antibody directed against the CD20 molecule. Rituximab is currently used for the treatment of patients with relapsed or refractory low-grade or follicular, CD20 positive, B cell non-Hodgkin's lymphoma. It is observed that in some patients treated with Rituximab, a small number of residual B cells are present in the blood. The mechanism of B cell depletion through anti-CD20 therapy is not completely clear. It has been speculated, for example, that Rituxan induces apoptosis of the B cells or that the B cells are killed by NK cells entering the spleen. It is generally thought that all B cells expressing CD20 are equally sensitive to killing by the anti-CD20 antibody.

It would be advantageous to develop improved therapies for treating diseases mediated by B cells because current therapies do not deplete all B cells. The present invention solves these problems and provides other advantages, as described in detail below.

### SUMMARY OF THE INVENTION

The present invention is based in part on the identification herein of in vivo mechanisms by which anti-hCD20 antibodies eliminate B cells. It was discovered surprisingly that certain B lymphocytes residing in tissues and organs, in particular those in the marginal zone (MZ) of the spleen, were resistant to killing with anti-human CD20 antibody, even though these cells expressed sufficient levels of CD20 on their surface and were found to be saturated with the administered anti-CD20 antibody. Interestingly, promoting the egress of these B cells from the tissues in which they are resident into the vascular system and/or prolonging their presence in circulation rendered them sensitive to killing by the anti-CD20 antibody. In view of this observation, one approach to improving intravascular access of these sequestered B cells is to mobilize them into the circulation with antagonists of integrins that tether these B cells to certain zones in the lymphoid tissues.

The present invention provides a method of augmenting B cell depletion in a mammal suffering from a B cell disorder, comprising administering to the mammal, one or more B cell mobilizing agent such as an alpha1 integrin antagonist and/or an alpha4 integrin antagonist, and a therapeutically effective amount of one or more B cell depleting agent such as an anti-CD20 antibody. B cell depletion can be augmented by administering a combination of alpha4 and alphaL integrin antagonists and a B cell depleting agent. In the preferred embodiment, the mammal or patient is a human.

The invention also provides a method of enhancing the efficacy of B cell depletion by a depletion agent such as a CD20 binding antibody, comprising administering to a patient suffering from a B cell disorder, at least one B cell mobilizing agent. An αL integrin antagonist and an α4 integrin antagonist act synergistically to enhance B cell depletion.

The invention further provides a method of treating a B cell neoplasm or malignancy characterized by B cells expressing a specific marker such as CD20, comprising administering to a patient suffering from the neoplasm or malignancy, a therapeutically effective amount of an antibody that binds the specific marker, such as a CD20 binding antibody and at least one B cell mobilizing agent, such as an alphaL integrin antagonist and/or an alpha4 integrin antagonist. In one embodiment, the B cell neoplasm is selected from the group consisting of non-Hodgkin's lymphoma (NHL), small lymphocytic (SL) NHL, lymphocyte predominant Hodgkin's disease (LPHD), follicular center cell (FCC) lymphomas, acute lymphocytic leukemia (ALL), chronic lymphocytic leukemia (CLL), and Hairy cell leukemia. For treating these cancers, in one embodiment, the antibody is administered via intravenous infusion. The dosage administered is in the range of about 100 mg/m² to 375 mg/m² per dose.

Yet another aspect of the invention is a method of alleviating a B-cell regulated autoimmune disorder comprising administering to a patient suffering from the autoimmune disorder, a therapeutically effective amount of a B cell depletion agent, such as a CD20 binding antibody, and at least one B cell mobilizing agent, such as an alphaL integrin antagonist and/or an alpha4 integrin antagonist. In specific embodiments, the autoimmune disease is selected from the group consisting of rheumatoid arthritis and juvenile rheumatoid arthritis, systemic lupus erythematosus (SLE) including lupus nephritis, Wegener's disease, inflammatory bowel disease, ulcerative colitis, idiopathic thrombocytopenic purpura (ITP), thrombotic throbocytopenic purpura (TTP), autoimmune thrombocytopenia, multiple sclerosis, psoriasis, IgA nephropathy, IgM polyneuropathies, myasthenia gravis, ANCA associated vasculitis, diabetes mellitus, Reynaud's syndrome, Sjorgen's syndrome, Neuromyelitis Optica (NMO) and glomerulonephritis. In preferred embodiments the CD20 binding antibody is administered intravenously or subcutaneously. In preferred embodiments, the antibody is administered intravenously at a dosage in the range of 10 mg to 500 mg per dose and in a specific embodiment, the dosage is 100 mg/dose.

Additionally, the invention provides a method of depleting B cells of the marginal zone B cells in the spleen and/or in germinal centers of lymphoid tissues of a patient suffering from a B cell disorder such as a B cell neoplasm or a B-cell regulated autoimmune disorder, comprising administering to the patient a therapeutically effective amount of a depletion agent such as a CD20 binding antibody and at least one B cell mobilizing agent, such as an alphaL integrin antagonist and/or an alpha4 integrin antagonist.

In any of the methods of the invention, the B cell mobilizing agent can be an alphaL integrin antagonist or alpha4 integrin antagonist, or a combination of these. In one embodiment, the alpha4 integrin antagonist is an antagonist of alpha4beta1. In an alternative embodiment, the antagonist is an antagonist of alpha4beta7. In yet another embodiment, the antagonist is an antagonist of alphaLbeta2.

In any of the methods of the present invention, in different embodiments, the alphaL or alpha4 integrin antagonist can be an antibody that binds the integrin, or the alpha or beta subunit of the integrin, or a ligand of the integrin. Thus, antibodies that bind ICAM-1 (CD-54) or VCAM-1 (CD-106) are encompassed. Similarly, biologically active fragments of antibodies that function essentially the same as a full-length antibody to bind and block biological activity of the alpha4 or alphaL integrin, such as the anti-CD18 Fab′₂ fragment H52 (Genentech, South San Francisco, Calif.), are encompassed. Where the mobilizing agent is an alphaL antagonist, in one embodiment the alphaL integrin antagonist antibody is an antibody that binds the alphaL subunit, CD11a, preferably the antibody efalizumab (Raptiva™, Genentech, Inc.), or a CD11a binding antibody that comprises the VL and VH sequence of SEQ ID NO. 49 and 50, respectively, of efalizumab, or a biologically active fragment of these antibodies. Where the mobilizing agent is an alpha4 integrin antagonist, in one embodiment the antagonist is the antibody natalizumab (Tysabri™, Biogen-IDEC), or a biologically active fragment thereof, that binds the alpha4 subunit. In preferred embodiments, the antibody is a humanized, human, or chimeric antibody, or a fragment of these.

In another embodiment, the alphaL or alpha4 integrin antagonist is a small molecule. Many such integrin antagonist small molecules are known. Any one or more of the compounds having the formula XI and particularly the compounds of Table 4 is an embodiment of an alphaL integrin antagonist small molecule. Any one or more of the compounds having the formula I, II, or III, any compound of formula X and having any one of the substituents shown in Tables 1 and 2, and particularly any compound of Table 3 is an embodiment of an alpha4 integrin antagonist small molecule.

In a further embodiment, the alphaL or alpha4 integrin antagonist can be an immunoadhesin comprising the soluble, integrin-binding portion or extracellular domain of the respective ligand. In one embodiment, the immunoadhesin is a soluble, alphaL ligand-binding portion of ICAM-1 (CD-54) fused to the hinge and Fc of a human IgG1. In a separate embodiment, the immunoadhesin is a soluble, alpha4 ligand-binding portion of VCAM-1 (CD-106) fused to the hinge and Fc of a human IgG1.

In any of the methods of the invention, the B cell depleting agent is an antagonist of a B cell surface marker, such as CD20, CD22, CD54, and the like. In a preferred embodiment, the B cell surface marker is CD20. In another embodiment, the B cell surface marker is CD22. In one embodiment, the B cell depleting agent is an antibody or antibody fragment that binds a B cell surface marker such as CD20, preferably human CD20 (hCD20). Many such anti-CD20 antibodies are known, including human, chimeric, and humanized anti-CD20 antibodies disclosed herein. In preferred embodiments, the anti-hCD20 antibody is Rituximab (Rituxan™); a humanized antibody comprising the VL and VH amino acid sequence of SEQ ID No. 29 and SEQ ID NO. 30, respectively; humanized antibody 2H7 v31, v114, v138, v477, v588, or v511 comprising the sequences provided herein, or a biologically active fragment thereof, or fucose deficient variants thereof. In one embodiment, humanized 2H7.v511 is provided in a liquid formulation comprising antibody at 20 mg/mL, 10 mM histidine sulfate at pH5.8, 60 mg/ml sucrose, 0.2 mg/ml polysorbate 20.

In any of the methods of the invention, any combination of antibody, small molecule, and/or immunoadhesin as B cell mobilizing agent and/or any combination of B cell depleting agent can be administered. For example, the B cell depleting agent can be an antibody that binds CD20 and the B cell mobilizing agent can be one or more small molecule antagonist of alpha4 and/or alphaL integrin.

In any of the methods of the invention, the B cell mobilizing agent or agents and the B cell depleting agent can be administered concurrently, sequentially, or alternating between concurrently and sequentially, in any order. Where two or more mobilizing agents are used, for example, an alphaL integrin antagonist in combination with an alpha4 integrin antagonist, the two agents can be administered concurrently, sequentially, or alternating between concurrently and sequentially, in any order. In one embodiment, an anti-CD20 antibody is administered to first deplete circulating B cells, followed by administration of an alphaL integrin antagonist or by a combination of alphaL integrin antagonist and alpha4 integrin antagonist to mobilize B cells residing in organs such as the spleen, lymph node, germinal centers, peritoneal cavity, and the like, further followed by repeat treatment with an anti-CD20 binding antibody to deplete residual mobilized B cells.

In a further embodiment, the invention comprises compositions that contain two or more mobilizing agents, for example, a combination of an alphaL integrin antagonist and an alpha4 integrin antagonist. Compositions of the invention further include a combination of one or more B cell mobilization agents with one or more B cell depleting agents. A particular embodiment is a composition that contains an alphaL integrin antagonist, an alpha4 antagonist, and an anti-CD20 antibody.

### BRIEF DESCRIPTION OF THE FIGURES

FIG. 1 graphically shows expression of hCD20 in populations of circulating lymphocytes of hCD20 transgenic (hCD20 Tg⁺) mice, the lymphocyte population characterized by surface expression of B220 and CD3.

FIG. 2 shows surface expression of hCD20 during B cell ontogeny and in lymphoid tissues. B cell progenitors and subsets in the bone marrow (top panel), spleen (middle panel), and other lymphoid organs (bottom panel) were analyzed for hCD20 expression.

FIG. 3 demonstrates depletion of B cell populations characterized by B220 and CD43 expression, from bone marrow of hCD20 Tg⁺ mice treated with control or with anti-hCD20 mAb (2H7) (left panel). Quantitation of hCD20 detected on populations of B cells is also shown (right panel).

FIG. 4 shows depletion of B cells by anti-hCD20 mAbs from the peripheral blood of hCD20 Tg⁺ mice treated with anti-CD20 antibodies.

FIG. 5 shows depletion and repletion of B cells following anti-hCD20 mAb treatment.

FIG. 6 shows distinct kinetics of B cell depletion in blood, lymph node, and peritoneal cavity of hCD20 Tg⁺ mice treated with anti-hCD20 antibody.

FIG. 7 shows sensitivity of splenic B cells from transgenic mice treated with 0.5 mg of anti-hCD20 mAb (bottom) or control IgG₂ₐ mAb (top).

FIG. 8 shows enumeration of FO and MZ B cell depletion in the spleen of mice described in FIG. 7.

FIG. 9 shows saturation of CD20 with anti-hCD20 mAbs on resistant splenic B cells.

FIG. 10 shows resistance of Peyer's Patch GC B cells to anti-hCD20 mAb depletion. Peyer's Patch B cells were isolated from control IgG₂ₐ (top panel) or anti-hCD20 mAb (bottom panel) treated mice and characterized by B220 and CD38 staining. Mature and GC B cells from control (open bars) and anti-hCD20 MAb treated (filled bars) mice were quantified (right panel).

FIG. 11 shows resistance of splenic GC B cells to depletion by anti-hCD20 mAb.

FIG. 12 shows depletion of marginal zone B cells after treatment with control or anti-hCD20 mAbs over 15 weeks (0.1 mg per 2 weeks, 1P).

FIG. 13 shows depletion of B cells by administering high doses of anti-α-hCD20 mAb. Doses as shown.

FIG. 14 shows B cell immune responses following hCD20 mAb treatment, specifically secondary immune responses as described in Example 3.

FIG. 15 shows T-independent immune response to a bacterial antigen as assessed by FACS analysis (left panel) of antigen (Ag)-specific plasmablasts isolated from B-cell depleted mice 4 days following administration of heat-inactivated Streptococcus Pneumoniae.

FIG. 16 shows FACS plots demonstrating mobilization of marginal zone B cells into the vasculature enhances sensitivity of MZ B cells to anti-hCD20 mAb depletion.

FIG. 17 shows results of quantization of MZ B cells (CD21^{hi}CD23^{lo}) in blood of mice treated with mobilization agents.

FIG. 18 is a graph showing quantization of total B220+ cells in the spleen of mice treated with anti-hCD20 mAb alone and in combination with mobilization agents.

FIG. 19 shows FACS plots of cells from mice treated with 25 μg lipopolysaccharide (LPS) and anti-hCD20 mAb.

FIG. 20 graphically shows quantization of lymphocytes from hCD20 Tg⁺ mice treated with vehicle control or Compound A. Lymphocytes isolated from lymph nodes (panels 1 and 2) and blood (panels 3 and 4) at 20 hours, were quantified and expressed as mean±standard error (n=4).

FIG. 21 demonstrates that the liver is required for B cell depletion, as described more fully in Example 5. Mice underwent sham (left panel) or clamping of the portal vein and hepatic artery (right panel) followed by immediate IV injection of control or anti-hCD20 (0.2 mg) mAb.

FIG. 22 shows quantization of B cells in blood from the sham or clamp treated mice of Example 5. All cells isolated from anti-hCD20 mAb treated mice were saturated with the in vivo administered mAb (data not shown).

FIG. 23 shows that the spleen is not required for B cell depletion, as described more fully in Example 5. Mice underwent either sham splenectomy (top row) or splenectomy (bottom row) and were analyzed for B cell depletion.

FIG. 24 shows the percentage of B cells in peripheral blood of the sham or splenectomy treated mice of Example 5, quantified and expressed as mean±standard error.

FIG. 25 shows that Kupfer cells engulf B220⁺ B cells, as described more fully in Example 5. Mice were treated with 0.1 mg control IgG (top left) or anti-hCD20 mAb.

### DETAILED DESCRIPTION OF THE EMBODIMENTS

A. Definitions

The following terms, as used herein, are intended to have the following definitions:

The term “antagonist” or “inhibitor” of an integrin, as used herein, means a compound that reduces or prevents binding of an integrin, such as alpha4beta1, alpha1 beta7, or alphaLbeta2 integrin, to a ligand, such as a VCAM-1, MAdCAM-1, ICAM 1-5, and the like, or reduces or prevents retention of B cells in lymphoid tissues, including Germinal Centers and/or marginal zone of the spleen. An “effective amount” is an amount is an amount sufficient to at least partially inhibit the binding and and may be an inhibitory amount.

The term “antibody” is used in the broadest sense and specifically includes monoclonal antibodies (including full length monoclonal antibodies), multispecific antibodies (e.g., bispecific antibodies), and antibody fragments that exhibit a desired biological activity or function. The antibodies comprising a polypeptide of this invention can be chimeric, humanized, or human. The antibodies comprising a polypeptide of this invention can be an antibody fragment. Such antibodies and methods of generating them are described in more detail below. Alternatively, an antibody of this invention can be produced by immunizing an animal with a polypeptide of this invention. Thus, an antibody directed against a polypeptide of this invention is contemplated.

“Antibody fragments” comprise a portion of a full length antibody, generally the antigen binding or variable region thereof. Examples of antibody fragments include Fab, Fab′, F(ab′)₂, and Fv fragments; diabodies; linear antibodies; single-chain antibody molecules; and multispecific antibodies formed from antibody fragments. “Functional fragments” substantially retain binding to an antigen of the full length antibody, and retain a biological activity.

“CD20 binding antibody” and “anti-CD20 antibody” are used interchangeably herein and encompass all antibodies that bind CD20 with sufficient affinity such that the antibody is useful as a therapeutic agent in targeting a cell expressing the antigen, and do not significantly cross-react with other proteins such as a negative control protein in the assays described below. Bispecific antibodies wherein one arm of the antibody binds CD20 are also contemplated. Also encompassed by this definition of CD20 binding antibody are functional fragments of the preceding antibodies. The CD20 binding antibody can bind CD20 with a Kd, for example, of <10 nM. In preferred embodiments, the binding is at a Kd of <7.5 nM, more preferably <5 nM, even more preferably at between 1-5 nM, most preferably, <1 nM.

In a specific embodiment, the anti-CD20 antibodies bind human and primate CD20. In specific embodiments, the antibodies that bind CD20 are humanized or chimeric. CD20 binding antibodies include, for example, rituximab (RITUXAN®), m2H7 (murine 2H7), hu2H7 (humanized 2H7) and all its functional variants, including without limitation, hu2H7.v16 (v stands for version), v31, v114, v138, v477, v588, or v511 or a biologically active fragment thereof, as well as fucose deficient variants thereof that have improved ADCC function.

Patents and patent publications concerning CD20 antibodies include U.S. Pat. Nos. 5,776,456, 5,736,137, 6,399,061, and 5,843,439, as well as U.S. Patent Application Nos. U.S. 2002/0197255A1 and 2003/0021781A1 (Anderson et al.); U.S. Pat. No. 6,455,043B1 and WO00/09160 (Grillo-Lopez, A.); WO00/27428 (Grillo-Lopez and White); WO00/27433 (Grillo-Lopez and Leonard); WO00/44788 (Braslawsky et al.); WO01/10462 (Rastetter, W.); WO01/10461 (Rastetter and White); WO01/10460 (White and Grillo-Lopez); US Application No. U.S. 2002/0006404 and WO02/04021 (Hanna and Hariharan); U.S. Application No. U.S. 2002/0012665 A1 and WO01/74388 (Hanna, N.); U.S. Application No. U.S. 2002/0009444A1, and WO01/80884 (Grillo-Lopez, A.); WO01/97858 (White, C.); U.S. Application No. U.S. 2002/0128488A1 and WO02/34790 (Reff, M.);WO02/060955 (Braslawsky et al.);WO2/096948 (Braslawsky et al.);WO02/079255 (Reff and Davies); U.S. Pat. No. 6,171,586B 1, and WO98/56418 (Lam et al.); WO98/58964 (Raju, S.); WO99/22764 (Raju, S.);WO99/51642, U.S. Pat. No. 6,194,551B1, U.S. Pat. No. 6,242,195B1, U.S. Pat. No. 6,528,624B1 and U.S. Pat. No. 6,538,124 (Idusogie et al.); WO00/42072 (Presta, L.); WO00/67796 (Curd et al.); WO01/03734 (Grillo-Lopez et al.); U.S. Application No. U.S. 2002/0004587A1 and WO01/77342 (Miller and Presta); U.S. application no. U.S. 2002/0197256 (Grewal, I.); U.S. Pat. Nos. 6,090,365B1, 6,287,537B1, 6,015,542, 5,843,398, and 5,595,721, (Kaminski et al.); U.S. Pat. Nos. 5,500,362, 5,677,180, 5,721,108, and 6,120,767 (Robinson et al.); U.S. Pat. No. 6,410,391B1 (Raubitschek et al.); U.S. Pat. No. 6,224,866B1 and WO00/20864 (Barbera-Guillem, E.); WO01/13945 (Barbera-Guillem, E.); WO00/67795 (Goldenberg); WO00/74718 (Goldenberg and Hansen); WO00/76542 (Golay et al.);WO01/72333 (Wolin and Rosenblatt); U.S. Pat. No. 6,368,596B1 (Ghetie et al.); U.S. Application No. U.S. 2002/0041847A1, (Goldenberg, D.); U.S. Application No. U.S. 2003/0026801A1 (Weiner and Hartmann); WO02/102312 (Engleman, E.), each of which is expressly incorporated herein by reference. See, also, U.S. Pat. No. 5,849,898 and EP appln no. 330,191 (Seed et al.); U.S. Pat. No. 4,861,579 and EP332,865A2 (Meyer and Weiss); and WO95/03770 (Bhat et al.).

The CD20 antibodies can be naked antibody or conjugated to a cytotoxic compound such as a radioisotope, or a toxin. Such antibodies include the antibody ZEVALIN®, which is linked to the radioisotope, Yttrium-90 (IDEC Pharmaceuticals, San Diego, Calif.), and BEXXAR®, which is conjugated to I-131 (Corixa, Wash.).

Humanized 2H7 variants include those that have amino acid substitutions in the FR and affinity maturation variants with changes in the grafted CDRs. The substituted amino acids in the CDR or FR are not limited to those present in the donor or acceptor antibody. In other embodiments, the anti-CD20 antibodies of the invention further comprise changes in amino acid residues in the Fc region that lead to improved effector function including enhanced CDC and/or ADCC function and B-cell killing (also referred to herein as B-cell depletion). In particular, three mutations have been identified for improving CDC and ADCC activity: S298A/E333A/K334A, also referred to herein as a triple Ala mutant or variant; numbering in the Fc region is according to the EU numbering system; Kabat et al., supra, as described in Idusogie et al., 2001, supra; Shields et al., supra).

Other anti-CD20 antibodies suitable for use with the present invention include those having specific changes that improve stability. In some embodiments, the chimeric anti-CD20 antibody has murine V regions and human C region. One such specific chimeric anti-CD20 antibody is RITUXAN® (RITUXIMAB®; Genentech, Inc.). Rituximab and hu2H7 can mediate lysis of B-cells through both complement-dependent cytotoxicity (CDC) and antibody-dependent cellular cytotoxicity (ADCC). Antibody variants with altered Fc region amino acid sequences and increased or decreased C1q binding capability are described in U.S. Pat. No. 6,194,551 B1 and WO99/51642. The contents of those patent publications are specifically incorporated herein by reference. See, also, Idusogie et al. 2000, J. Immunol. 164: 4178-4184.

WO00/42072 (Presta) describes polypeptide variants with improved or diminished binding to FcRs. The content of that patent publication is specifically incorporated herein by reference. See, also, Shields et al., 2001, J. Biol. Chem. 9(2): 6591-6604.

“Autoimmune disease” is used herein in a broad, general sense to refer to disorders or conditions in mammals in which destruction of normal or healthy tissue arises from humoral or cellular immune responses of the individual mammal to his or her own tissue constituents, or a manifestation thereof or resulting condition thereof.

The terms “cancer”, “cancerous”, and “malignant” refer to or describe the physiological condition in mammals that is typically characterized by unregulated cell growth. Examples of cancer include but are not limited to, carcinoma including adenocarcinoma, lymphoma, blastoma, melanoma, sarcoma, and leukemia. More particular examples of such cancers include squamous cell cancer, small-cell lung cancer, non-small cell lung cancer, gastrointestinal cancer, Hodgkin's and non-Hodgkin's lymphoma, pancreatic cancer, glioblastoma, cervical cancer, ovarian cancer, liver cancer such as hepatic carcinoma and hepatoma, bladder cancer, breast cancer, colon cancer, colorectal cancer, endometrial carcinoma, salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer. Optionally, the cancer will express, or have associated with the cancer cell, BLyS. In some embodiments, the cancers for treatment herein include lymphoma, leukemia and myeloma, and subtypes thereof, such as Burkitt's lymphoma, multiple myeloma, acute lymphoblastic or lymphocytic leukemia, non-Hodgkin's and Hodgkin's lymphoma, and acute myeloid leukemia.

An “extracellular domain” or “ECD” refers to a form of a polypeptide that is essentially free of the transmembrane and cytoplasmic domains.

The term “immune related disease” means a disease in which a component of the immune system of a mammal causes, mediates, or otherwise contributes to morbidity in the mammal. Also included are diseases in which stimulation or intervention of the immune response has an ameliorative effect on progression of the disease. Included within this term are autoimmune diseases, immune-mediated inflammatory diseases, non-immune-mediated inflammatory diseases, infectious diseases, and immunodeficiency diseases. Examples of immune-related and inflammatory diseases, some of which are immune or T cell mediated, which can be treated according to the invention include 1, rheumatoid arthritis, juvenile chronic arthritis, spondyloarthropathies, systemic sclerosis (scleroderma), idiopathic inflammatory myopathies (dermatomyositis, polymyositis), Sjogren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barrd syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory and fibrotic lung diseases such as inflammatory bowel disease (ulcerative colitis: Crohn's disease), gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease. Infectious diseases include AIDS (HIV infection), hepatitis A, B, C, D, and E, bacterial infections, fungal infections, protozoal infections and parasitic infections.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., 1975, Nature 256:495, or may be made by recombinant DNA methods (see, e.g., U.S. Pat. No. 4,816,567). The “monoclonal antibodies” may also be isolated from phage antibody libraries using the techniques described in Clackson et al. 1991, Nature 352:624-628 and Marks et al., 1991, J. Mol. Biol. 222:581-597, for example.

“Chimeric” antibodies (immunoglobulins) have a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., 1984, Proc. Natl. Acad. Sci. USA 81:6851-6855). Humanized antibody as used herein is a subset of chimeric antibodies.

“Carriers” as used herein include physiologically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONIC®.

A “composition” of this invention can comprise one or more B cell depleting agent and/or one or more B cell mobilizing agent, optionally in combination with a physiologically acceptable carrier. The composition can further comprise an additional therapeutic agent to treat the indication intended. In some embodiments, the composition comprises a second therapeutic agent selected from a drug for treating an immune-related disease and a drug for treating a cancer. In some embodiments, the drug for treating a cancer is selected from the group consisting of a cytotoxic agent, a chemotherapeutic agent, a growth inhibiting agent and a chemotherapeutic agent.

“Humanized” forms of non-human (e.g., murine) antibodies are chimeric antibodies that contain minimal sequence derived from non-human immunoglobulin. For the most part, humanized antibodies are human immunoglobulins (recipient or acceptor antibody) in which hypervariable region residues of the recipient are replaced by hypervariable region residues from a non-human species (donor antibody), such as mouse, rat, rabbit, or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance such as binding affinity. Generally, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence although the FR regions may include one or more amino acid substitutions that improve binding affinity. The number of these amino acid substitutions in the FR are typically no more than 6 in the H chain, and in the L chain, no more than 3. The humanized antibody optionally also will comprise at least a portion of an immunoglobulin constant region (Fc), typically that of a human immunoglobulin. For further details, see Jones et al., 1986, Nature 321:522-525; Reichmann et al., 1988, Nature 332:323-329; and Presta, 1992, Curr. Op. Struct. Biol. 2:593-596.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g. B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch et al., 1991, Annu. Rev. Immunol 9:457-92. To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al., 1998, PNAS (USA) 95:652-656.

“Mammal” for purposes of treatment or therapy refers to any animal classified as a mammal, including humans, domestic and farm animals, and zoo, sports, or pet animals, such as dogs, horses, cats, cows, and the like. Preferably, the mammal is human.

As used herein, “B cell depletion” refers to a reduction in B cell levels in an animal or human after drug or antibody treatment, as compared to the level before treatment. B cell levels are measurable using well known assays such as by getting a complete blood count, by FACS analysis staining for known B cell markers, and by methods such as described in the Experimental Examples. B cell depletion can be partial or complete. In one embodiment, the depletion of CD20 expressing B cells is 25% or more. In a patient receiving a B cell depleting drug, B cells are generally depleted for the duration of time when the drug is circulating in the patient's body and the time for recovery of B cells.

B cell depletion is augmented if the level or percentage of B cells depleted after treatment with the B cell depleting agent combined with B cell mobilizing agent is greater than the level obtained with B cell killing (depleting) agent alone. The levels of B cell depletion can be measured by methods familiar to the skilled medical practitioner. B cell depletion can be measured by the number of B cells in the blood without and with treatment with B cell mobilizing agent. As another exemplary method of quantifying B cells, a lymph node biopsy of a cancer patient can be performed after treatment with the B cell depleting agent such as an anti-CD20 antibody, to obtain a baseline level of B cells before treatment with B cell mobilizing agent(s). The patient is then administered one or more B cell mobilization agents together with or followed by B cell depleting agent again. Post this second round of B cell depletion treatment regimen, a second lymph node biopsy is performed to quantify the B cells remaining.

A “B cell depleting agent” as used herein is any antagonist that binds to or otherwise targets a B cell through a B-cell surface marker resulting directly or indirectly in the death of the targeted B cell. As used herein, the B cell is eliminated in the circulation, such as by ADCC, CDC or other mechanism. The B cell depleting agent can be a protein such as an antibody or ligand of the cell surface marker, or a small molecule. The B cell depleting agent can be conjugated to a cytotoxic agent or growth inhibitory agent. In one embodiment, the B cell depleting agent is a monoclonal antibody (mAb) that binds CD20, CD22, or CD54. CD20 binding antibodies are disclosed below. In preferred embodiments, the CD20 binding antibody is rituximab, or humanized 2H7v 16, or a variant of h2H7v16.

A “B cell mobilizing agent” as used herein is any molecule that promotes the circulation of B cells in mammals in the blood by, e.g., inhibiting the adhesion and retention of B cells in lymphoid organs and other B cell laden tissues or otherwise promoting egress of B cells from these sites, or by inhibiting homing of B cells to lymphoid and other organs and tissues. In one specific embodiment, the B cell mobilizing agent inhibits B cell retention in at least the marginal zone of the spleen, and preferably the MZ and germinal center of the spleen and lymphoid tissues. In another embodiment, the B cell mobilizing agent inhibits homing of the B cell to the spleen. In yet another embodiment, the agent inhibits homing of the B cell to the gut. An increase in B cells in the peripheral blood with administration of the B cell mobilizing agent can be quantified by known methods such as described in the examples.

A “B cell disorder” includes a B cell neoplasm (e.g., CD20 positive B cell neoplasm) or a B-cell regulated autoimmune disease or autoimmune related condition, both disclosed in detail below.

“Complement dependent cytotoxicity” or “CDC” refers to the lysis of a target cell in the presence of complement. Activation of the classical complement pathway is initiated by the binding of the first component of the complement system (C I q) to antibodies (of the appropriate subclass) that are bound to their cognate antigen. To assess complement activation, a CDC assay, e.g. as described in Gazzano-Santoro et al., 1996, J. Immunol Methods 202:163, may be performed.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The term “therapeutically effective amount” refers to an amount of a composition of this invention effective to “alleviate” or “treat” a disease or disorder in a subject or mammal. Generally, alleviation or treatment of a disease or disorder involves the lessening of one or more symptoms or medical problems associated with the disease or disorder. In some embodiments, it is an amount that results in the reduction in the number of B cells in the mammal. In the case of cancer, the therapeutically effective amount of the drug can reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. In some embodiments, a composition of this invention can be used to prevent the onset or reoccurrence of the disease or disorder in a subject or mammal. For example, in a subject with autoimmune disease, a composition of this invention can be used to prevent or alleviate flare-ups.

“Treating” or “treatment” or “alleviation” refers to both therapeutic treatment and prophylactic or preventative measures, wherein the object is to prevent or slow down (lessen) the targeted pathologic condition or disorder. A subject is successfully “treated” for a CD20 positive cancer or an autoimmune disease if, after receiving a therapeutic amount of a CD20 binding antibody of the invention according to the methods of the present invention, the subject shows observable and/or measurable reduction in or absence of one or more signs and symptoms of the particular disease. For example, for cancer, reduction in the number of cancer cells or absence of the cancer cells; reduction in the tumor size; inhibition (i.e., slow to some extent and preferably stop) of tumor metastasis; inhibition, to some extent, of tumor growth; increase in length of remission, and/or relief to some extent, one or more of the symptoms associated with the specific cancer; reduced morbidity and mortality, and improvement in quality of life issues. Reduction of the signs or symptoms of a disease may also be felt by the patient. Treatment can achieve a complete response, defined as disappearance of all signs of cancer, or a partial response, wherein the size of the tumor is decreased, preferably by more than 50 percent, more preferably by 75%. A patient is also considered treated if the patient experiences stable disease. In a preferred embodiment, the cancer patients are still progression-free in the cancer after one year, preferably after 15 months. These parameters for assessing successful treatment and improvement in the disease are readily measurable by routine procedures familiar to a physician of appropriate skill in the art.

“Chronic” administration refers to administration of the agent(s) in a continuous mode as opposed to an acute mode, so as to maintain the initial therapeutic effect (activity) for an extended period of time.

“Intermittent” administration is treatment that is not consecutively done without interruption, but rather is cyclic in nature.

The term “cytotoxic agent” as used herein refers to a substance that inhibits or prevents the function of cells and/or causes destruction of cells. The term is intended to include radioactive isotopes (e.g., I¹³¹, I¹²⁵, Y⁹⁰ and Re¹⁸⁶), chemotherapeutic agents, and toxins such as enzymatically active toxins of bacterial, fungal, plant or animal origin, or fragments thereof.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin I and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omega1I (see, e.g., Agnew, 1994. Chem Intl. Ed. Engl. 33:183-186); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhône-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluorometlhylornithine (DMFO); retinoids such as retinoic acid; capecitabine; and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in aberrant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rlL-2; LURTOTECAN® topoisomerase I inhibitor; ABARELIX® rmRH; and pharmaceutically acceptable salts, acids or derivatives of any of the above.

A “growth inhibitory agent” when used herein refers to a compound or composition which inhibits growth of a cell in vitro and/or in vivo. Thus, the growth inhibitory agent may be one that significantly reduces the percentage of cells in S phase. Examples of growth inhibitory agents include agents that block cell cycle progression (at a place other than S phase), such as agents that induce GI arrest and M-phase arrest. Classical M-phase blockers include the vincas (vincristine and vinblastine), TAXOL® paclitaxel, and topo II inhibitors such as doxorubicin, epirubicin, daunorubicin, etoposide, and bleomycin. Those agents that arrest GI also spill over into S-phase arrest, for example, DNA alkylating agents such as tanoxifen, prednisone, dacarbazine, mechlorethamine, cisplatin, methotrexate, 5-fluorouracil, and ara-C. Further information can be found in Murakaini et al., 1995, In: The Molecular Basis of Cancer, Mendelsohn and Israel, eds., Chapter 1, “Cell cycle regulation, oncogenes, and antieioplastic drugs,” (W B Saunders: Philadelphia), see p. 13.

A “Germinal Center” is a microenviroment within a lymphoid secondary follicle where B-cell proliferation, somatic hypermutation, and antigen binding selection occur.

The “marginal zone” is a region of the spleen containing a population of B cells that produce low-affinity, polyreactive antibodies. Due to this anatomical location, marginal zone B cells frequently come into contact with antigen, including self-antigen. Marginal zone B cells have low activation thresholds, are particularly reactive to self-antigens (Viau et al., 2005, Clin. Immunol., 114: 17-26), and reactive to blood-borne antigens. Autoreactive B cells are sequestered in the marginal zone to prevent high-affinity autoreactivity.

A “soluble” portion of a polypeptide, as used herein, refers to a portion that is soluble in water and lacks appreciable affinity for lipids (e.g., missing the transmembrane domain or the transmembrane and the cytoplasmic domains).

B. Intregrin Subunits

1. Alpha4

The terms “alpha 4” or “alpha4 polypeptide” or “alpha4 protein” (also referred to as CD49d, integrin alpha4 subunit or VLA-4 alpha subunit) when used herein encompass “native sequence alpha4 polypeptides” that have a biological activity of a native sequence alpha 4. In one embodiment, the biological activity of an alpha4 polypeptide promotes the adhesion and retention of B lymphocytes in an organ or an area of a lymphoid tissue, e.g., through association with a beta subunit such as beta1 (CD29) or beta7 to form an integrin that binds to an extracellular matrix or ligand on at least an immobilized marginal zone spleen cell in the germinal centers of lymphoid tissues, thus limiting intravascular access of the B lymphocyte. A “native sequence” alpha4 polypeptide comprises a polypeptide having the same amino acid sequence as a corresponding alpha4 polypeptide derived from nature. Such native sequence alpha4 polypeptides can be isolated from nature or can be produced by recombinant and/or synthetic means. The term “native sequence alpha4 polypeptide” includes naturally-occurring truncated forms, naturally-occurring variant forms (e.g., alternatively spliced forms), naturally-occurring isoforms, and naturally-occurring allelic variants of the polypeptide. An example of a human alpha4 polypeptide sequence is shown below (Genbank Accession No. S06046):

| [SEQ ID NO: 1] | |
|---|---|
| 1 | mfptesawlg krganpgpea avretvmlll clgvptgrpy nvdtesally qgphntlfgy |
| 61 | svvlhshgan rwllvgapta nwlanasvin pgaiyrcrig knpgqtceql qlgspngepc |
| 121 | gktcleerdrn qwlgvtlsrq pgengsivtc ghrwknifyi knenklptgg cygvppdlrt |
| 181 | elskriapcy qdyvkkfgen fascqagiss fytkdlivmg apgssywtgs lfvynittnk |
| 241 | ykafldkqnq vkfgsylgys vgaghfrsqh ttevvggapq heqigkayif sidekelnil |
| 301 | hemkgkklgs yfgasvcavd lnadgfsdll vgapmqstir eegrvfvyin sgsgavmnam |
| 361 | etnlvgsdky aarfgesivn lgdidndgfe dvaigapqed dlqgaiyiyn gradgisstf |
| 421 | sqrieglqis kslsmfgqsi sgqidadnng yvdvavgafr sdsavllrtr pvvivdasls |
| 481 | hpesvnrtkf dcvengwpsv cidltlcfsy kgkevpgyiv lfynmsldvn rkaespprfy |
| 541 | fssngtsdvi tgsiqvssre ancrthqafm rkdvrdiltp iqieaayhlg phviskrste |
| 601 | efpplqpilq qkkekdimkk tinfarfcah encsadlqvs akigflkphe nktylavgsm |
| 661 | ktlmlnvslf nagddayett lhvklpvgly fikileleek qincevtdns gvvqldcsig |
| 721 | yiyvdhlsri disflldvss lsraeedlsi tvhatcenee emdnlkhsrv tvaiplkyev |
| 781 | kltvhgfvnp tsfvygsnde nepetcmvek mnltfhvint gnsmapnvsv eimvpnsfsp |
| 841 | qtdklfnild vqtttgechf enyqrvcale qqksamqtlk givrflsktd krllycikad |
| 901 | phclnflcnf gkmesgkeas vhiqlegrps ilemdetsal kfeiratgfp epnprvieln |
| 961 | kdenvahvll eglhhqrpkr yftiviisss lllglivlll isyvmwkagf fkrqyksilq |
| 1021 | eenrrdswsy insksndd |

(Residues 1-39 are amino acids of the signal sequence. Residues 40 to 1048 are amino acids of the product α4 integrin).

Alpha4 combines with β1 to form the integrin α4β1 (VLA-4, CD49d/CD29), or with the β7 subunit to form the integrin α4β7.

2. Beta1

The terms “beta1I” (CD29) or “beta1 polypeptide” or “beta1 protein” when used herein encompass “native sequence beta1 polypeptides” which have a biological activity of the native sequence beta 1. In one preferred embodiment, the biological activity of a beta1 polypeptide according to this invention is to promote the adhesion and retention of B lymphocytes in an organ or an area of a lymphoid tissue, e.g., through association with an alpha subunit such as alpha4 or alpha2 to form an integrin that binds to an extracellular matrix or ligand on at least an immobilized marginal zone spleen cell or germinal center cell, thus limiting intravascular access of the B lymphocyte. A “native sequence” beta1 polypeptide comprises a polypeptide having the same amino acid sequence as a corresponding beta I polypeptide derived from nature. Such native sequence beta1 polypeptides can be isolated from nature or can be produced by recombinant and/or synthetic means. The term “native sequence beta1 polypeptide” include naturally-occurring truncated forms, naturally-occurring variant forms (e.g., alternatively spliced forms), naturally-occurring isoforms (such as A-D), and naturally-occurring allelic variants of the polypeptide. An example of a human beta1 polypeptide sequence is shown below (Genbank Accession No. P05556):

| [SEQ ID NO: 2] | |
|---|---|
| 1 | mnlqpifwig lissvccvfa qtdenrclka nakscgeciq agpncgwctn stflqegmpt |
| 61 | sarcddleal kkkgcppddi enprgskdik knknvtnrsk gtaeklkped ihqiqpqqlv |
| 121 | lrlrsgepqt ftlkfkraed ypidlyylmd lsysmkddle nvkslgtdlm nemrritsdf |
| 181 | rigfgsfvek tvmpyisttp aklrnpctse qncttpfsyk nvlsltnkge vfnelvgkqr |
| 241 | isgnldspeg gfdaimqvav cgsligwrnv trllvfstda gfhfagdgkl ggivlpndgq |
| 301 | chlennmytm shyydypsia hlvqklsenn iqtifavtee fqpvykelkn lipksavgtl |
| 361 | sanssnviql iidaynslss evilengkls egvtisyksy ckngvngtge ngrkcsnisi |
| 421 | gdevqfeisi tsnkcpkkds dsfkirplgf teevevilqy icececqseg ipespkcheg |
| 481 | ngtfecgacr cnegrvgrhc ecstdevnse dmdaycrken sseicsnnge cvcgqcvcrk |
| 541 | rdntneiysg kfcecdnfnc drsnglicgg ngvckcrvce cnpnytgsac dcsldtstce |
| 601 | asngqicngr gicecgvckc tdpkfqgqtc emcqtclgvc aehkecvqcr afnkgekkdt |
| 661 | ctqecsyfni tkvesrdklp qpvqpdpvsh ckekdvddcw fyftysvngn nevmvhvven |
| 721 | pecptgpdii pivagvvagi vliglallli wkllmiihdr refakfekek mnakwdtgen |
| 781 | piyksavttv vnpkyegk |

3. Beta7

The terms “beta7” or “beta7 polypeptide” or “beta7 protein” when used herein encompass “native sequence beta7 polypeptides” which have a biological activity of the native sequence beta7. In one embodiment, the biological activity of a beta7 polypeptide according to this invention is to promote the homing of alpha4beta7+ lymphocytes to the gut thus limiting intravascular access of the B lymphocytes. In another embodiment, the biological activity of a beta7 polypeptide is to promote the adhesion and retention of B lymphocytes in an organ or an area of a lymphoid tissue such as the MZ of the spleen e.g., through association with an alpha subunit such as alpha4. A “native sequence” beta7 polypeptide comprises a polypeptide having the same amino acid sequence as a corresponding beta7 polypeptide derived from nature. Such native sequence beta7 polypeptides can be isolated from nature or can be produced by recombinant and/or synthetic means. The term “native sequence beta7 polypeptide” include naturally-occurring truncated forms, naturally-occurring variant forms (e.g., alternatively spliced forms), naturally-occurring isoforms (such as A-D), and naturally-occurring allelic variants of the polypeptide. An example of a human beta7 polypeptide sequence is shown below (Genbank Accession No. P26010):

| [SEQ ID NO: 3] | |
|---|---|
| 1 | mvalpmvlvl llvlsrgese ldakipstgd atewrnphls mlgscqpaps cqkcilshps |
| 61 | cawckqlnft asgeaearrc arreellarg cpleeleepr gqqevlqdqp lsqgargega |
| 121 | tqlapqrvrv tlrpgepqql qvrflraegy pvdlyylmdl sysmkddler vrqlghallv |
| 181 | rlqevthsvr iqfgsfvdkt vlpfvstvps klrhpcptrl ercqspfsfh hvlsltgdaq |
| 241 | aferevgrqs vsgnldspeg gfdailqaal cqeqigwrnv srllvftsdd tfhtagdgkl |
| 301 | ggifmpsdgh chldsnglys rstefdypsv gqvaqalsaa niqpifavts aalpvyqels |
| 361 | klipksavge lsedssnvvq limdaynsls stvtlehssl ppgvhisyes qcegpekreg |
| 421 | kaedrgqcnh vrinqtvtfw vslqathclp ephllrlral gfseelivel htlcdcncsd |
| 481 | tqpqaphcsd gqghlqcgvc scapgrlgrl cecsvaelss pdlesgcrap ngtgplcsgk |
| 541 | ghcqcgrcsc sgqssghlce cddascerhe gilcggfgrc qcgvchchan rtgracecsg |
| 601 | dmdscispeg glcsghgrck cnrcqcldgy ygalcdqcpg cktpcerhrd caecgafrtg |
| 661 | platncstac ahtnvtlala pilddgwcke rtldnqlfff lveddargtv vlrvrpqekg |
| 721 | adhtqaivlg cvggivavgl glvlayrlsv eiydrreysr fekeqqqlnw kqdsnplyks |
| 781 | aitttinprf qeadsptl |

4. AlphaL

The terms “alpha L” or “alphaL polypeptide” or “alphaL protein” or “CD11a” when used herein encompass “native sequence alphaL polypeptides” that have a biological activity of the native sequence alphaL (CD11a). In one embodiment, the biological activity of an alphaL polypeptide is to promote the adhesion and retention of B lymphocytes in an organ or an area of a lymphoid tissue, e.g., through association with a beta subunit such as beta2 (CD18), to form an integrin that binds to an extracellular matrix or ligand on at least an immobilized marginal zone spleen cell or a germinal center cell. Another biological activity of alphaLbeta2 (CD11a/CD18) (LFA-1) is in promoting homing of B lymphocytes from the blood to the spleen and lymph node. Both these biological activities result in limiting intravascular access of these B lymphocytes.

AlphaLbeta2 (LFA-1) binds to at least CD54 (ICAM-1), CD102 (ICAM2), and CD50 (ICAM-3). A “native sequence” alphaL polypeptide comprises a polypeptide having the same amino acid sequence as a corresponding alphaL polypeptide derived from nature. Such native sequence alphaL polypeptides can be isolated from nature or can be produced by recombinant and/or synthetic means. The term “native sequence alphaL polypeptide” include naturally-occurring truncated forms, naturally-occurring variant forms (e.g., alternatively spliced forms), naturally-occurring isoforms, and naturally-occurring allelic variants of the polypeptide. An example of a human alphaL polypeptide sequence is shown below (SWISSPROT Accession No. P207017; EMBL/GENBANK Accession No. Y00796):

| [SEQ ID NO: 4] | |
|---|---|
| 1 | mkdscitvma mallsgffff apassynldv rgarsfsppr agrhfgyrvl qvgngvivga |
| 61 | pgegnstgsl yqcqsgtghc lpvtlrgsny tskylgmtla tdptdgsila cdpglsrtcd |
| 121 | qntylsglcy lfrqnlqgpm lqgrpgfqec ikgnvdlvfl fdgsmslqpd efqkildfmk |
| 181 | dvmkklsnts yqfaavqfst syktefdfsd yvkrkdpdal lkhvkhmlll tntfgainyv |
| 241 | atevfreelg arpdatkvli iitdgeatds gnidaakdii ryiigigkhf qtkesqetlh |
| 301 | kfaskpasef vkildtfekl kdlftelqkk iyviegtskq dltsfnmels ssgisadlsr |
| 361 | ghavvgavga kdwaggfldl kadlqddtfi gnepltpevr agylgytvtw lpsrqktsll |
| 421 | asgapryqhm grvllfqepq ggghwsqvqt ihgtqigsyf ggelcgvdvd qdgetellli |
| 481 | gaplfygeqr ggrvfiyqrr qlgfeevsel qgdpgyplgr fgeaitaltd ingdglvdva |
| 541 | vgapleeqga vyifngrhgg lspqpsqrie gtqvlsgiqw fgrsihgvkd legdgladva |
| 601 | vgaesqmivl ssrpvvdmvt lmsfspaeip vhevecsyst snkmkegvni ticfqiksly |
| 661 | pqfqgrlvan ltytlqldgh rtrrrglfpg grhelrrnia vttsmsctdf sfhfpvcvqd |
| 721 | lispinvsln fslweeegtp rdqraqgkdi ppilrpslhs etweipfekn cgedkkcean |
| 781 | lrvsfspars ralrltafas lsvelslsnl eedaywvqld lhfppglsfr kvemlkphsq |
| 841 | ipvsceelpe esrllsrals cnvsspifka ghsvalqmmf ntlvnsswgd svelhanvtc |
| 901 | nnedsdlled nsattiipil ypiniliqdq edstlyvsft pkgpkihqvk hmyqvriqps |
| 961 | ihdhniptle avvgvpqpps egpithqwsv qmeppvpchy edlerlpdaa epclpgalfr |
| 1021 | cpvvfrqeil vqvigtlelv geieassmfs lcsslsisfn sskhfhlygs naslaqvvmk |
| 1081 | vdvvyekqml ylyvlsgigg llllllifiv lykvgffkrn lkekmeagrg vpngipaeds |
| 1141 | eqlasgqeag dpgclkplhe kdsesgggkd |

5. Beta2

The terms “beta2” (CD18) or “beta2 polypeptide” or “beta2 protein” when used herein encompass “native sequence beta2 polypeptides” that have a biological activity of a native sequence beta2. A “native sequence” beta2 polypeptide comprises a polypeptide having the same amino acid sequence as a corresponding beta2 polypeptide derived from nature. Such native sequence beta2 polypeptides can be isolated from nature or can be produced by recombinant and/or synthetic means. The term “native sequence beta2 polypeptide” include naturally-occurring truncated forms, naturally-occurring variant forms (e.g., alternatively spliced forms), naturally-occurring isoforms, and naturally-occurring allelic variants of the polypeptide. An example of a human beta2 polypeptide sequence is shown below (Genbank Accession No. P05107):

| [SEQ ID NO: 5] | |
|---|---|
| 1 | mlglrpplla lvgllslgcv lsqectkfkv sscreciesg pgctwcqkln ftgpgdpdsi |
| 61 | rcdtrpqllm rgcaaddimd ptslaetqed hnggqkqlsp qkvtlylrpg qaaafnvtfr |
| 121 | rakgypidly ylmdlsysml ddlrnvkklg gdllralnei tesgrigfgs fvdktvlpfv |
| 181 | nthpdklrnp cpnkekecqp pfafrhvlkl tnnsnqfqte vgkqlisgnl dapeggldam |
| 241 | mqvaacpeei gwrnvtrllv fatddgfhfa gdgklgailt pndgrchled nlykrsnefd |
| 301 | ypsvgqlahk laenniqpif avtsrmvkty eklteiipks avgelsedss nvvhliknay |
| 361 | nklssrvfld hnalpdtlkv tydsfcsngv thrnqprgdc dgvqinvpit fqvkvtatec |
| 421 | iqeqsfvira lgftdivtvq vlpqcecrcr dqsrdrslch gkgflecgic rcdtgyigkn |
| 481 | cecqtqgrss qelegscrkd nnsiicsglg dcvcgqclch tsdvpgkliy gqycecdtin |
| 541 | ceryngqvcg gpgrglcfcg kcrchpgfeg sacqcertte gclnprrvec sgrgrcrcnv |
| 601 | cechsgyqlp lcqecpgcps pcgkyiscae clkfekgpfg kncsaacpgl qlsnnpvkgr |
| 661 | tckerdsegc wvaytleqqd gmdryliyvd esrecvagpn iaaivggtva givligilll |
| 721 | viwkalihls dlreyrrfek eklksqwnnd nplfksattt vmnpkfaes |

C. Alpha4 Integrin

The term “alpha4 integrin” when used herein refers to a heterodimer comprising an alpha4 subunit and a beta subunit. Examples of alpha4 integrins include alpha4beta1 (VLA4 or VLA4 integrin) or alpha4beta7 (LPAM-1 or LPAM-1 integrin). Alpha4beta1 (α4β1) is expressed on most leukocytes with the possible exception of neutrophils and platelets; it is also expressed in non-lymphoid tissue. Alpha4beta7 (alpha4beta7) is expressed on most lymph node T and B cells, NK cells and eosinophils. alpha4beta1 is involved in the migration of leukocytes from blood to tissues at sites of inflammation. alpha4beta7 is involved in the homing of α4β7+ lymphocytes to the gut through recognition of MAdCAM-I on mucosal high endothelial venules.

Examples of the biological activity of an alpha 4 integrin can include any one or a combination of the following activities: (1) binding to a ligand of alpha4beta1 (e.g., any one of the ligands selected from the group consisting of VCAM-1, fibronectin, thrombospondin, collagens and invasin), (2) binding to a ligand of alpha4beta7 (e.g., any one of the ligands selected from the group consisting of vascular cell adhesion molecule-1 (VCAM-1), mucosal addressin cell adhesion molecule-1 (MAdCAM-1), and fibronectin, and (3) promoting the adhesion and retention and/or homing of B lymphocytes to an organ or an area of a lymphoid tissue such as the marginal zone in the spleen.

1. Ligands of Alpha4 Integrin

The alpha4 integrin ligand, VCAM-1 (CD106), contains seven IgSF C2 domains in its extracellular portion (Barclay et al., 1997, supra, page 386-387). VCAM-1 contains two independent binding sites for alpha4beta I (VLA4) in domains 1 and 4, respectively (see, for example, Vonderheide, et al., 1994, J. Cell Biol. 125:215-222; Jones, et al., 1995, Nature 373: 539-544 for integrin binding sites). The full length amino acid sequence of human VCAM-1 (CD106) is provided on page 387 of Barclay et al, supra, and through GenBank Accession No. M73255 or SWISSPROT P19320.

An example of a human VCAM-1 polypeptide sequence is shown below (SWISSPROT Accession No. P19320):

| (SEQ ID NO: 6) | |
|---|---|
| 1 | mpgkinvvilg asnilwimfa asqafkiett pesrylaqig dsvsltcstt gcespffswr |
| 61 | tqidsplngk vtnegttstl tmnpvsfgne hsylctatce srklekgiqv eiysfpkdpe |
| 121 | ihlsgpleag kpitvkcsva dvypfdrlei dllkgdhlmk sqefledadr ksletkslev |
| 181 | tftpviedig kvlvcraklh idemdsvptv rqavkelqvy ispkntvisv npstklqegg |
| 241 | svtmtcsseg lpapeifwsk kldngnlqhl sgnatltlia mrmedsgiyv cegvnligkn |
| 301 | rkevelivqe kpftveispg priaaqigds vmltcsvmgc espsfswrtq idsplsgkvr |
| 361 | segtnstltl spvsfenehs ylctvtcghk klekgiqvel ysfprdpeie msgglvngss |
| 421 | vtvsckvpsv ypldrleiel lkgetileni efledtdmks lenkslemtf iptiedtgka |
| 481 | lvcqaklhid dmefepkqrq stqtlyvnva prdttvlvsp ssileegssv nmtclsqgfp |
| 541 | apkilwsrql pngelqplse natltlistk medsgvylce ginqagrsrk eveliiqvtp |
| 601 | kdikltafps esvkegdtvi isctcgnvpe twiilkkkae tgdtvlksid gaytirkaql |
| 661 | kdagvyeces knkvgsqlrs ltldvqgren nkdyfspell vlyfasslii paigmiiyfa |
| 721 | rkanmkgsys lveaqkskv |

(Signal sequence at residues 1 to 24; Extracellular domain at residues 25 to 698; Transmembrane domain at residues 699 to 720; and Cytoplasmic domain at residues 721 to 739).

The alpha 4 integrin ligand, MAdCAM contains two IgSF C2 domains in its extracellular portion (Tan et al. 1998, Structure 6: 793-801). MAdCAM is a receptor for alpha4beta7 and L-selectin (Elangbam et al., 1997, Vet. Pathol., 34: 61-73). An example of a full-length amino acid sequence of human MAdCAM is provided through SWISSPROT Accession No: Q13477.

| (SEQ ID NO: 7) | |
|---|---|
| 1 | mdfglallla gllglllgqs lqvkplqvep pepvvavalg asrqltcrla cadrgasvqw |
| 61 | rgldtslgav qsdtgrsvlt vrnaslsaag trvcvgscgg rtfqhtvqll vyafpdqltv |
| 121 | spaalvpgdp evactahkvt pvdpnalsfs llvggqeleg aqalgpevqe eeeepqgded |
| 181 | vlfrvterwr lpplgtpvpp alycqatmrl pglelshrqa ipvlhsptsp eppdttspes |
| 241 | pdttspespd ttspespdtt sqeppdttsq eppdttsqep pdttspeppd ktspepapqq |
| 301 | gsthtprspg strtrrpeis qagptqgevi ptgsskpagd qlpaalwtss avlgllllal |
| 361 | ptyhlwkrcr hlaeddthpp aslrllpqvs awaglrgtgq vgisps |

(Signal sequence at residues 1 to 18; Extracellular domain at residues 19 to 341; Transmembrane domain at residues 342 to 362; and Cytoplasmic domain at residues 363 to 406).
2. Alpha4 Integrin Antagonist

The term “alpha4 integrin antagonist” as used herein is used in the broadest sense, and includes any molecule that partially or fully blocks a biological activity of an alpha4 integrin. According to one embodiment, alpha4 integrin antagonist partially or fully blocks the interaction between an alpha4 integrin and its ligand, and performs any one or a combination of the following events: (1) promotes lymphocyte egress from lymphoid organs or tissues and/or otherwise promotes the circulation of B lymphocytes in mammals and (2) partially or fully blocks, inhibits, or neutralizes native sequence alpha4 integrin signaling. In one embodiment, the alpha4 integrin antagonist inhibits B cell adhesion and retention in the spleen and gut. In a more specific embodiment, the alpha4beta1 antagonist inhibits B cell adhesion and retention in at least the marginal zone of the spleen or germinal center of lymphoid tissue. Useful antagonists of alpha4 integrin include antagonists of the alpha subunit, antagonists of the beta subunit, and antagonists of both the alpha and the beta subunits.

According to one preferred embodiment, the alpha4 integrin antagonist is an alpha4beta1 (VLA-4) antagonist, for example, those described in WO 99/06432. According to another preferred embodiment, the alpha4 integrin antagonist is an alpha4beta7 (LPAM-1) antagonist, for example, the humanized MAb MLN-02/LDP-02, described in U.S. application Ser. No. 08/700,737 or the pyroglutamic acid derivatives and related compounds described in U.S. Pat. No. 6,407,066. According to one embodiment, the alpha4 integrin antagonist is a dual alpha4beta1/alpha4beta7 antagonist, for example, R-41 I (Hijazi et al., 2004, J. Clin. Pharmacol., 44:1368-1378), or the antagonists described in U.S. Pat. No. 6,482,849, or in Egger et al., 2002 Jul., J. Pharmacol. Exp. Ther., 302(1):53-62.

According to one embodiment, the antagonist binds to the alpha4 subunit. According to another embodiment, the antagonist binds a ligand of the alpha4 integrin, for example the ligands, VCAM-1, or MAdCAM-1 ligand. Antagonists of alpha4 integrins, for example alpha4beta1 and alpha4beta7, can be used together, simultaneously or sequentially, to promote circulation of B lymphocytes in mammals. Multiple different antagonists of alpha4beta1 (VLA-4) and/or alpha4beta7 (LPAM-1) can be used together, simultaneously or sequentially, to promote the circulation of B lymphocytes in mammals.

The alpha4 integrin antagonist can be an antibody, a small molecule, or an immunoadhesin.

3. Antibody Antagonists of Alpha4 Integrin

In one embodiment, the alpha4 integrin antagonist is an antibody. The term “antibody” is broadly used, and includes polyclonal and monoclonal, full length and fragments, humanized, chimeric, bi-specific, and the like antibodies. In a preferred embodiment, the alpha4 integrin antagonist is an antibody that binds alpha4beta1 (VLA4), alpha4beta7 (LPAM-1), or an antibody that binds the alpha subunit alone, such as the anti-CD49d antibody disclosed in the Examples below.

Examples of antibodies that are alpha4 integrin antagonists include Biogen-Idec's TYSABRI® (natalizumab), previously called Antegren (U.S. Pat. Nos. 6,602,503, 5,840,299, and 5,730,978, which are hereby incorporated by reference), and the like.

According to another embodiment, the alpha4 integrin antagonist is an antibody that binds a ligand of an alpha4 integrin, for example, any of the ligands listed above, and particularly an anti-VCAM-1 antibody or an anti-MAdCAM-1 antibody. For example, a humanized VCAM-1 antibody, 2A2, is available from Alexion Pharmaceuticals Inc. (New Haven, Conn.).

Examples of humanized Abs that specifically bind alpha4beta (VLA-4) include those comprising one or more the VL and VH chains shown below:

1) a light chain variable region comprising the sequence

| (SEQ ID NO: 8) | | |
|---|---|---|
| a) | 1 | DIQMTQSPSS LSASVGDRVT ITCKTSQDIN KYMAWYQQTP GKAPRLLIHY TSALQPGIPS |
| | 61 | RFSGSGSGRD YTFTISSLQP EDIATYYCLQ YDNLWTFGQG TEVEIK; |
| | | or |
| (SEQ ID NO: 9) | | |
| b) | 1 | DIQMTQSPSS LSASVGDRVT ITCKTSQDIN KYMAWYQQTP GKAPRLLIYY TSALQPGIPS |
| | 61 | RFSGSGSGRD YTFTISSLQP EDIATYYCLQ YDNLWTFGQG TEVEIK; |
| | | and |

2) a heavy chain variable region comprising the sequence

| (SEQ ID NO: 10) | | |
|---|---|---|
| c) | 1 | QVQLVQSGAE VKKPGASSVK VSCKASGFNI KDTYIHWVRQ APGQRLEWMB RIDPANGYTK |
| | 61 | YDPKEQGRVT ITADTSASTA YMELSSLRSE DTAVYYCARE GYYGNYGVYA MDYWGQGTLV |
| | 121 | TVSS, |
| (SEQ ID NO: 11) | | |
| d) | 1 | QVQLVQSGAE VKKPGASSVK VSCKASGFNI KDTYIHWVRQ APGQGLEWMB RIDPANGYTK |
| | 61 | YDPKFQGRVT ITADTSASTA YMELSSLRSE DTAVYYCARE GYYGNYGVYA MDYWGQGTLV |
| | 121 | TVSS, |
| | | or |
| (SEQ ID NO: 12) | | |
| e) | 1 | QVQLVQSGAE VKKPGASSVK VSCKASGFNI KDTYIHWVRQ APGQRLEWMB RIDPANGYTK |
| | 61 | YDPKFQGRVT ITADTSASTA YMELSSLRSE DTAVYYCARE GYFGNYGVYA MDYWGQGTLV |
| | 121 | TVSS. |

An example of a humanized antibody that specifically binds VLA4 comprises:

1) a light chain variable region comprising the sequence

| (SEQ ID NO: 13) | | |
|---|---|---|
| a) | 1 | SIVMTQSPSSL SASVGDRVTI TCKASQSVTN DVAWYQQKPG KAPKLLIYYA SNRYTGVPDR |
| | 61 | FSGSGYGTDFT FTISSLQPED IATYYCQQDY SSPYTFGQGT KVEIK, |
| (SEQ ID NO: 14) | | |
| b) | 1 | DIQMTQSPSSL SASVGDRVTI TCKASQSVTN DVAWYQQKPG KAPKLLIYYA SNRYTGVPDR |
| | 61 | FSGSGSGTDFT FTISSLQPED IATYYCQQDY SSPYTFGQGT YVEIK, |
| | | or |
| (SEQ ID NO: 15) | | |
| c) | 1 | SIVMTQSPDSL AVSLGERVTI NCKASQSVTN DVAWYQQKPG QSPKLLIYYA SNRYTGVPDR |
| | 61 | FSGSGYGTDFT FTISSVQAED VAVYYCQQDY SSPYTFGGGT KLEIK |
| | | and |

2) a heavy chain variable region comprising the sequence

| (SEQ ID NO: 16) | | |
|---|---|---|
| d) | 1 | QVQLQESGPGL VRPSQTLSLT CTVSGFNIKD TYMHWVRQPP GRGLEWIGRI DPASGDTKYD |
| | 61 | PKFQVRVTMLV DTSSNTAWLR LSSVTAADTA VYYCADGMWV STGYALDFWG QGTTVTVSS, |
| (SEQ ID NO: 17) | | |
| e) | 1 | QVQLQESPGL VRPSQTLSLTC TVSGFNIKDT YMHWVRQPPG RGLEWIGRID PASGDTKYDP |
| | 61 | KFQVKATITA DTSSNQFSLRL SSVTAADTAV YYCADGMWVS TGYALDFWGQ GTTVTVSS, |
| (SEQ ID NO: 18) | | |
| f) | 1 | QVQLQESGPG LVRPSQTLSLT CTVSGFNIKD TYMHWVRQPP GRGLEWIGRI DPASGDTKYD |
| | 61 | PKFQVRVTML VDTSSNQFSLR LSSVTSEDTA VYYCADGMWV STGYALDFWG QGTTVTVSS, |
| (SEQ ID NO: 19) | | |
| g) | 1 | QVQLQESGPG LVRPSQTLSLT CTVSGFNIKD TYMHWVKQRP GRGLEWIGRI DPASGDTKYD |
| | 61 | PKFQVRVTML VDTSSNQFSLR LSSVTAADTA VYYCADGMWV STGYALDFWG QGTTVTVSS, |
| (SEQ ID NO: 20) | | |
| h) | 1 | QVQLQESGPG LVRPSQTLSLT CTASGFNIKD TYMHWVRQPP GRGLEWIGRI DPASGDTKYD |
| | 61 | PKFQVRVTML VDTSSNQFSLR LSSVTAADTA VYYCADGMWV STGYALDFWG QGTTVTVSS, |
| | | or |
| (SEQ ID NO: 21) | | |
| i) | 1 | QVQLQESGAE VVKPGSSVKLS CKASGFNIKD TYMHWVKQRP GQGLEWIGRI DPASGDTKYD |
| | 61 | PKFQVKATIT ADESTSTAYLE LSSLRSEDTA VYYCADGMWV STGYALDFWG QGTTVTVSS. |

4. Immunoadhesin Antagonists of Alpha4 Integrin

According to yet another embodiment, the integrin antagonist is an immunoadhesin. An example of such an immunoadhesin is one that comprises a soluble portion of a ligand of alpha4 integrin that binds to alpha4, for example, the ligand binding domain or the extracellular domain of a ligand of the alpha4 integrin, such as VCAM-1 (CD106) and/or MAdCAM-1. In one embodiment, the immunoadhesin antagonist is a soluble ligand-binding domain fused to an Fc region of an IgG such as human IgG1.

The binding domains of VCAM-1 and MAdCAM-I are known in the art. VCAM-1 binds to alpha4beta1 primarily via several residues (residues 39, 40, and 43) within Domain I (residues 25-105 according to UniProt) with a contribution from several residues from Domain 2 (residues 109-212 according to UniProt); VCAM-1 binds to alpha4beta7 primarily via residues within Domain 2 with a contribution from residues within Domain 1. (Newham et al., 1997, J. Biol. Chem., 272: 19429-19440). MAdCAM-1 binds to alpha4beta7 via both Domain 1 (residues 23-112 according to UniProt) and Domain 2 (residues 113-231 according to UniProt); MAdCAM-1 residues 40, 41, 42, and 44 were required for full binding, and removal of residues 143-150 abolished binding. MAdCAM-1 poorly binds to α4β1, and removal of residues 143-150 also abolished binding to α4β1. (Newham et al., 1997, supra). Although both alpha4beta1 and alpha4beta7 can bind both VCAM-1 and MAdCAM-1, there is a ligand preference. alpha4beta1 is primarily a receptor for VCAM-1, and alpha4beta7 is primarily a receptor for MAdCAM-1. (Newham et al., 1997, supra).

5. Small Molecule Antagonists of Alpha4 Integrin

In another embodiment, the alpha4 integrin antagonist is a small molecule. Examples of small molecules that are alpha4 integrin antagonists include those disclosed in U.S. Pat. Nos. 6,239,108, 6,469,047, 6;482,849, and 6,706,753, published PCT Application Nos. WO 01/21584 and WO 02/16313, and in U.S. Provisional Patent Application No. 60/472,072, filed May 20, 2003. According to one embodiment, the antagonist is any one of the small molecules recited as alpha4 integrin antagonists in WO 01/21584 and as described more completely below. According to another embodiment, the antagonist is any one of the small molecules recited in WO 01/21584 or any of those shown in the Tables below.

a. Chemical Definitions

As used to define the small molecules disclosed herein, the following chemical terms have the indicated definitions:

The term “alkyl”, used alone or as part of another term, for example alkylamino, alkylsulfonyl, alkylthio, etc., means a branched or unbranched, saturated or unsaturated aliphatic hydrocarbon group, having the number of carbon atoms specified, or if no number is specified, having up to and including 12 carbon atoms. “Alkyl” when used alone or as part of another term preferably means a saturated hydrocarbon chain, however also includes unsaturated hydrocarbon carbon chains such as “alkenyl” and “alkynyl”. Examples of alkyl groups include methyl, ethyl, n-propyl, isopropyl, n-butyl, iso-butyl, sec-butyl, tert-butyl, n-pentyl, 2-methylbutyl, 2,2-dimethylpropyl, n-hexyl, 2-methylpentyl, 2,2-dimethylbutyl, n-heptyl, 3-heptyl, 2-methylhexyl, and the like. The terms “lower alkyl” “C₁-C₆ alkyl” and “alkyl of 1 to 6 carbon atoms” are synonymous and used interchangeably. Preferred “C₁-C₆ alkyl” groups are methyl, ethyl, 1-propyl, isopropyl, 1-butyl or sec-butyl.

The terms “substituted alkyl” or “substituted Cₙ-Cₘ alkyl” where m and n are integers identifying the range of carbon atoms contained in the alkyl group, denotes the above alkyl groups that are substituted by one, two, three or four halogen, trifluoromethyl, hydroxy, unsubstituted and substituted C₁-C₇ alkoxy, protected hydroxy, amino (including alkyl and dialkyl amino), protected amino, unsubstituted and substituted C₁-C₇ acyloxy, unsubstituted and substituted C₃-C₇ heterocyclyl, unsubstituted and substituted phenoxy, nitro, carboxy, protected carboxy, unsubstituted and substituted carboalkoxy, unsubstituted and substituted acyl, carbamoyl, carbamoyloxy, cyano, methylsulfonylamino, unsubstituted and substituted benzyloxy, unsubstituted and substituted C₃-C₆ carbocyclyl or C₁-C₄ alkoxy groups. The substituted alkyl groups may be substituted once (preferably), twice or three times with the same or with different substituents.

Examples of the above substituted alkyl groups include, but are not limited to; cyanomethyl, nitromethyl, hydroxymethyl, trityloxymethyl, propionyloxymethyl, aminomethyl, carboxymethyl, carboxyethyl, carboxypropyl, alkyloxycarbonylmethyl, allyloxycarbonylaminomethyl, carbamoyloxymethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichloro(n-butyl), 2-amino(iso-propyl), 2-carbamoyloxyethyl and the like. The alkyl group may also be substituted with a carbocyclyl group. Examples include cyclopropylmethyl, cyclobutylmethyl, cyclopentylmethyl, and cyclohexylmethyl groups, as well as the corresponding -ethyl, -propyl, -butyl, -pentyl, -hexyl groups, etc. A preferred group of examples within the above group includes the substituted methyl group, e.g. a methyl group substituted by the same substituents as the “substituted Cₙ-Cₘ alkyl” group. Examples of the substituted methyl group include groups such as hydroxymethyl, protected hydroxymethyl (e.g. tetrahydropyranyloxymethyl), acetoxymethyl, carbamoyloxymethyl, trifluoromethyl, chloromethyl, carboxymethyl, bromomethyl and iodomethyl.

The term “non-aromatic” refers to carbocycle or heterocycle rings that do not have the properties which define aromaticity. For aromaticity, a ring must be planar, have p-orbitals that are perpendicular to the plane of the ring at each ring atom and satisfy the Huckel rule where the number of pi electrons in the ring is (4n+2) wherein n is an integer (i.e. the number of pi electrons is 2, 6, 10 or 14). Non-aromatic rings provided herein do not satisfy one or all of these criteria for aromaticity.

The term “alkoxy” as used herein includes saturated, i.e. O-alkyl, and unsaturated, i.e. O-alkenyl and O-alkynyl groups. Exemplary alkoxy groups have the number of carbon atoms specified such as methoxy, ethoxy, n-propoxy, isopropoxy, n-butoxy, t-butoxy and like groups. The term “substituted alkoxy” means these alkoxy groups substituted by the same substituents as the “substituted alkyl” group.

The term “acyloxy” denotes carboacyloxy groups having the specified number of carbon atoms such as formyloxy, acetoxy, propionyloxy, butyryloxy, pentanoyloxy, hexanoyloxy, heptanoyloxy, and the like. The term “substituted acyloxy” means these acyloxy groups substituted by the same substituents as the “substituted alkyl” group.

The term “alkylcarbonyl”, “alkanoyl” and “acyl” are used interchangeably herein encompass groups having the specified number of carbon atoms such as formyl, acetyl, propionyl, butyryl, pentanoyl, hexanoyl, heptanoyl, benzoyl and the like.

The term “alkylsulfonyl” denotes the groups —NH—SO₂-alkyl, —SO₂—NH-alkyl, —N—(SO₂-alkyl)₂ and —SO₂—N(alkyl)₂. Preferred alkylsulfonyl groups are —NH—SO₂—Me, —NH—SO₂-Et, —NH—SO₂—Pr, —NH—SO₂—Pr, —N—(SO₂—Me)₂ and —N—(SO₂—Bu)₂.

The term “amino” denotes primary (i.e. —NH₂), secondary (i.e. —NRH) and tertiary (i.e. —NRR) amines. Preferred secondary and tertiary amines are alkylamine and dialkyl amines such as methylamine, ethylamine, propylamine, isopropylamine, dimethylamine, diethylamine, dipropylamine and disopropylamine.

By “carboxyl” is meant herein to be a free acid —COOH as well as esters thereof such as alkyl, aryl and aralkyl esters. Preferred esters are methyl, ethyl, propyl, butyl, i-butyl, s-butyl and t-butyl esters.

The terms “carbocyclyl”, “carbocyclylic” and “carbocyclo” alone and when used as a moiety in a complex group such as a carbocycloalkyl group, refers to a mono-, bi-, or tricyclic aliphatic ring having 3 to 14 carbon atoms and preferably 3 to 7 carbon atoms. Preferred carbocyclic groups include cyclopropyl, cyclobutyl, cyclopentyl and cyclohexyl groups. The terms “substituted carbocyclyl” and “carbocyclo” mean these groups substituted by the same substituents as the “substituted alkyl” group.

A “carbocycloalkyl” group is a carbocyclo group as defined above covalently bonded to an alkyl group as defined above.

The term “heterocycle” refers to a mono-, bi- or tri-cyclic ring system having 5-16 members wherein at least one ring atom is a heteroatom (i.e. N, O and S as well as SO, or SO₂). The ring system is saturated, unsaturated or partially unsaturated and may be aromatic (unless specified as non-aromatic). Exemplary heterocycles include piperidine, piperazine, pyridine, pyrazine, pyrimidine, pyridazine, morpholine, pyran, pyrole, furan, thiophene (thienyl), imidazole, pyrazole, thiazole, isothiazole, dithiazole, oxazole, isoxazole, dioxazole, thiadiazole, oxadiazole, tetrazole, triazole, thiatriazole, oxatriazole, thiadiazole, oxadiazole, purine and benzofused derivatives thereof.

The phrase “optionally substituted with” is understood to mean, unless otherwise stated, that one or more of the specified substituents is covalently attached to the substituted moiety. When more than one, the substituents may be the same or different group.

The term “alkenyl” means a branched or unbranched hydrocarbon group having the number of carbon atoms designated containing one or more carbon-carbon double bonds, each double bond being independently cis, trans, or a nongeometric isomer. The term “substituted alkenyl” means these alkenyl groups substituted by the same substituents as the “substituted alkyl” group.

The term “alkynyl” means a branched or unbranched hydrocarbon group having the number of carbon atoms designated containing one or more carbon-carbon triple bonds. The term “substituted alkynyl” means these alkynyl groups substituted by the same substituents as the “substituted alkyl” group.

The terms “alkylthio” and “C₁-C₁₂ substituted alkylthio” denote C₁-C₁₂ alkyl and C₁-C₁₂ substituted alkyl groups, respectively, attached to a sulfur which is in turn the point of attachment for the alkylthio or substituted alkylthio group to the group or substituent designated.

An “alkylenedioxy” group is a —O-alkyl-O— group, where alkyl is as defined above. Preferred alkylenedioxy groups are methylenedioxy and ethylenedioxy.

The term “aryl” when used alone or as part of another term means a homocyclic aromatic group whether or not fused having the number of carbon atoms designated or if no number is designated, up to 14 carbon atoms. Preferred aryl groups include phenyl, naphthyl, biphenyl, phenanthrenyl, naphthacenyl, and the like (see e.g. Lang's Handbook of Chemistry (Dean, J. A., ed), 1985, 13^{th} ed. Table 7-2).

The term “aroyl” means an aryl group bonded to a carbonyl, such as benzoyl, etc.

The term “substituted phenyl” or “substituted aryl” denotes a phenyl group or aryl group substituted with one, two, three, four or five, preferably 1-2,1-3 or 14 substituents chosen from halogen (F, Cl, Br, I), hydroxy, protected hydroxy, cyano, nitro, alkyl (preferably C₁-C₆ alkyl), alkoxy (preferably C₁-C₆ alkoxy), benzyloxy, carboxy, protected carboxy, carboxymethyl, protected carboxymethyl, hydroxymethyl, protected hydroxymethyl, aminomethyl, protected aminomethyl, trifluoromethyl, alkylsulfonylamino, arylsulfonylamino, heterocyclylsulfonylamino, heterocyclyl, aryl, or other groups specified. One or methyne (CH) and/or methylene (CH₂) groups in these substituents may in tern be substituted with a similar group as those denoted above. Examples of the term “substituted phenyl” includes but is not limited to a mono- or di(halo)phenyl group such as 2-chlorophenyl, 2-bromophenyl, 4-chlorophenyl, 2,6-dichlorophenyl, 2,5-dichlorophenyl, 3,4-dichlorophenyl, 3-chlorophenyl, 3-bromophenyl, 4-bromophenyl, 3,4-dibromophenyl, 3-chloro-4-fluorophenyl, 2-fluorophenyl and the like; a mono- or di(hydroxy)phenyl group such as 4-hydroxyphenyl, 3-hydroxyphenyl, 2,4-dihydroxyphenyl, the protected-hydroxy derivatives thereof and the like; a nitrophenyl group such as 3- or 4-nitrophenyl; a cyanophenyl group, for example, 4-cyanophenyl; a mono- or di(lower alkyl)phenyl group such as 4-methylphenyl, 2,4-dimethylphenyl, 2-methylphenyl, 4-(isopropyl)phenyl, 4-ethylphenyl, 3-(n-propyl)phenyl and the like; a mono or di(alkoxy)phenyl group, for example, 3,4-dimethoxyphenyl, 3-methoxy-4-benzyloxyphenyl, 3-methoxy-4-(1-chloromethyl)benzyloxyphenyl, 3-ethoxyphenyl, 4-(isopropoxy)phenyl, 4-(t-butoxy)phenyl, 3-ethoxy-4-methoxyphenyl and the like; 3- or 4-trifluoromethylphenyl; a mono- or dicarboxyphenyl or (protected carboxy)phenyl group such 4-carboxyphenyl, a mono- or di(hydroxymethyl)phenyl or (protected hydroxymethyl)phenyl such as 3-(protected hydroxymethyl)phenyl or 3,4-di(hydroxymethyl)phenyl; a mono- or di(aminomethyl)phenyl or (protected aminomethyl)phenyl such as 2-(aminomethyl)phenyl or 2,4-(protected aminomethyl)phenyl; or a mono- or di(N-(methylsulfonylamino))phenyl such as 3-(N-methylsulfonylamino))phenyl. Also, the term “substituted phenyl” represents disubstituted phenyl groups where the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl, and the like, as well as trisubstituted phenyl groups where the substituents are different, for example 3-methoxy-4-benzyloxy-6-methyl sulfonylamino, 3-methoxy-4-benzyloxy-6-phenyl sulfonylamino, and tetrasubstituted phenyl groups where the substituents are different such as 3-methoxy-4-benzyloxy-5-methyl-6-phenyl sulfonylamino. Preferred substituted phenyl groups include the 2-chlorophenyl, 2-aminophenyl, 2-bromophenyl, 3-methoxyphenyl, 3-ethoxy-phenyl, 4-benzyloxyphenyl, 4-methoxyphenyl, 3-ethoxy-4-benzyloxyphenyl, 3,4-diethoxyphenyl, 3-methoxy-4-benzyloxyphenyl, 3-methoxy-4-(1-chloromethyl)benzyloxy-phenyl, 3-methoxy-4-(1-chloromethyl)benzyloxy-6-methyl sulfonyl aminophenyl groups. Also, the term “substituted phenyl” represents phenyl groups having an aryl, phenyl or heteroaryl group fused thereto. The fused ring may also be substituted with any, preferably 1, 2 or 3, of the substituents identified above for “substituted alkyl” groups.

The term “arylalkyl” means one, two, or three aryl groups having the number of carbon atoms designated, appended to an alkyl group having the number of carbon atoms designated including but not limited to; benzyl, napthylmethyl, phenethyl, benzhydryl (diphenylmethyl), trityl, and the like. A preferred arylalkyl group is the benzyl group.

The term “substituted arylalkyl” denotes an alkyl group, preferably a C₁-C₈alkyl group, substituted at any carbon with an aryl group, preferably a C₆-C₁₀aryl group, bonded to the alkyl group through any aryl ring position and substituted on the alkyl portion with one, two or three groups chosen from halogen (F, Cl, Br, I), hydroxy, protected hydroxy, amino, protected amino, C₁-C₇acyloxy, nitro, carboxy, protected carboxy, carbamoyl, carbamoyloxy, cyano, C₁-C₆alkylthio, N-(methylsulfonylamino) or C₁-C₄alkoxy. Optionally the aryl group may be substituted with one, two, three, four or five groups chosen from halogen, hydroxy, protected hydroxy, nitro, C₁-C₆alkyl, C₁-C₆alkoxy, carboxy, protected carboxy, carboxymethyl, protected carboxymethyl, hydroxymethyl, protected hydroxymethyl, aminomethyl, protected aminomethyl, or an N-(methylsulfonylamino) group. As before, when either the C₁-C₈ alkyl portion or the aryl portion or both are disubstituted, the substituents can be the same or different. This group may also appear as the substituted aralkyl moiety of a substituted aralkoxy group.

Examples of the term “substituted aralkyl” and this group when it occurs in a “substituted aralkoxy” group include groups such as 2-phenyl-1-chloroethyl, 1-phenyl-1-chloromethyl, 1-phenyl-1-bromomethyl, 2-(4-methoxyphenyl)ethyl, 2,6-dihydroxy-4-phenyl(n-hexyl), 5-cyano-3-methoxy-2-phenyl(n-pentyl), 3-(2,6-dimethylphenyl)n-propyl, 4-chloro-3-aminobenzyl, 6-(4-methoxyphenyl)-3-carboxy(n-hexyl), 5-(4-aminomethyl phenyl)-3-(aminomethyl)(n-pentyl), and the like.

The term “carboxy-protecting group” as used herein refers to one of the ester derivatives of the carboxylic acid group commonly employed to block or protect the carboxylic acid group while reactions are carried out on other functional groups on the compound. Examples of such carboxylic acid protecting groups include 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxybenzhydryl, 2,21,4,4′-tetramethoxybenzhydryl, alkyl such as t-butyl or t-amyl, trityl, 4-methoxytrityl, 4,4′-dimethoxytrityl, 4,4′,4″-trimethoxytrityl, 2-phenylprop-2-yl, trimethylsilyl, t-butyldimethylsilyl, phenacyl, 2,2,2-trichloroethyl, beta-(trimethylsilyl)ethyl, beta-(di(n-butyl)methylsilyl)ethyl, p-toluenesulfonylethyl, 4-nitrobenzylsulfonylethyl, allyl, cinnamyl, 1-(trimethylsilylmethyl)prop-1-en-3-yl, and like moieties. The species of carboxy-protecting group employed is not critical so long as the derivatized carboxylic acid is stable to the condition of subsequent reaction(s) on other positions of the molecule and can be removed at the appropriate point without disrupting the remainder of the molecule. In particular, it is important not to subject a carboxy-protected molecule to strong nucleophilic bases or reductive conditions employing highly activated metal catalysts such as Raney nickel. (Such harsh removal conditions are also to be avoided when removing amino-protecting groups and hydroxy-protecting groups, discussed below.) Preferred carboxylic acid protecting groups are the allyl and p-nitrobenzyl groups. Similar carboxy-protecting groups used in the cephalosporin, penicillin and peptide arts can also be used to protect a carboxy group substituents. Further examples of these groups are found in T. W. Greene and P. G. M. Wuts, “Protective Groups in Organic Synthesis”, 2nd ed., John Wiley & Sons, Inc., New York, N.Y., 1991, chapter 5; E. Haslam, “Protective Groups in Organic Chemistry”, J. G. W. McOmie, Ed., Plenum Press, New York, N.Y., 1973, Chapter 5, and T. W. Greene, “Protective Groups in Organic Synthesis”, John Wiley and Sons, New York, N.Y., 1981, Chapter 5. The term “protected carboxy” refers to a carboxy group substituted with one of the above carboxy-protecting groups.

The term “hydroxy-protecting group” as used herein refers to a derivative of the hydroxy group commonly employed to block or protect the hydroxy group while reactions are carried out on other functional groups on the compound. Examples of such protecting groups include tetrahydropyranyloxy, acetoxy, carbamoyloxy, trifluoro, chloro, carboxy, bromo and iodo groups. Further examples of these groups are found in T. W. Greene and P. G. M. Wuts, “Protective Groups in Organic Synthesis”, 2nd ed., John Wiley & Sons, Inc., New York, N.Y., 1991, chapters 2-3; E. Haslam, “Protective Groups in Organic Chemistry”, J. G. W. McOmie, Ed., Plenum Press, New York, N.Y., 1973, Chapter 5, and T. W. Greene, “Protective Groups in Organic Synthesis”, John Wiley and Sons, New York, N.Y., 1981. The term “protected hydroxy” refers to a hydroxy group substituted with one of the above hydroxy-protecting groups.

The term “amino-protecting group” as used herein refers to a derivative of the groups commonly employed to block or protect an amino group while reactions are carried out on other functional groups on the compound. Examples of such protecting groups include carbamates, amides, alkyl and aryl groups, imines, as well as many N-heteroatom derivatives which can be removed to regenerate the desired amine group. Further examples of these groups are found in T. W. Greene and P. G. M. Wuts, “Protective Groups in Organic Synthesis”, 2nd ed., John Wiley & Sons, Inc., New York, N.Y., 1991, chapter 7; E. Haslam, “Protective Groups in Organic Chemistry”, J. G. W. McOmie, Ed., Plenum Press, New York, N.Y., 1973, Chapter 5, and T. W. Greene, “Protective Groups in Organic Synthesis”, John Wiley and Sons, New York, N.Y., 1981. The term “protected amino” refers to an amino group substituted with one of the above amino-protecting groups.

The terms “heterocyclic group”, “heterocyclic”, “heterocyclyl”, or “heterocyclo” alone and when used as a moiety in a complex group such as a heterocycloalkyl group, are used interchangeably and refer to any mono-, bi-, or tricyclic saturated or non-aromatically unsaturated ring having the number of atoms designated, generally from 3 to about 10 ring atoms, where the ring atoms are carbon and 1,2, 3 or 4 nitrogen, sulfur or oxygen atoms. Typically, a 5-membered ring has 0 to 2 double bonds and 6- or 7-membered ring has 0 to 3 double bonds and the nitrogen or sulfur heteroatoms may optionally be oxidized, and any nitrogen heteroatom may optionally be quaternized. Examples include morpholinyl, pyrrolidinyl, oxiranyl, oxetanyl, tetrahydrofttranyl, 2,3-dihydrofuranyl, 2H-pyranyl, tetrahydropyranyl, thiiranyl, thietanyl, tetrahydrothietanyl, aziridinyl, azetidinyl, 1-methyl-2-pyrrolyl, piperidinyl, and 3,4,5,6-tetrahydropiperidinyl. A preferred group is the morpholinyl group.

A “heterocycloalkyl” or a “heterocycloalkenyl” group is a heterocyclo group as defined above covalently bonded to an alkyl or alkenyl group as defined above.

Unless otherwise specified, “heteroaryl” alone and when used as a moiety in a complex group such as a heteroaralkyl group, refers to any mono-, bi-, or tricyclic aromatic ring system having the number of atoms designated where at least one ring is a 5-, 6- or 7-membered ring containing from one to four heteroatoms selected from the group nitrogen, oxygen, and sulfur, and preferably at least one heteroatom is nitrogen (Lang's Handbook of Chemistry, supra). Included in the definition are any bicyclic groups where any of the above heteroaryl rings are fused to a benzene ring. Heteroaryls in which nitrogen or oxygen is the heteroatom are preferred.

The following ring systems are examples of the heteroaryl (whether substituted or unsubstituted) groups denoted by the term “heteroaryl”: thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, thiazinyl, oxazinyl, triazinyl, thiadiazinyl, oxadiazinyl, dithiazinyl, dioxazinyl, oxathiazinyl, tetrazinyl, thiatriazinyl, oxatriazinyl, dithiadiazinyl, imidazolinyl, dihydropyrimidyl, tetrahydropyrimidyl, tetrazolo[1,5-b]pyridazinyl and purinyl, as well as benzo-fused derivatives, for example benzoxazolyl, benzofuryl, benzothiazolyl, benzothiadiazolyl, benzotriazolyl, benzoimidazolyl and indolyl.

Heterocyclic 5-membered ring systems containing a sulfur or oxygen atom and one to three nitrogen atoms are also suitable for use in the instant invention. Examples of such preferred groups include thiazolyl, in particular thiazol-2-yl and thiazol-2-yl N-oxide, thiadiazolyl, in particular 1,3,4-thiadiazol-5-yl and 1,2,4-thiadiazol-5-yl, oxazolyl, preferably oxazol-2-yl, and oxadiazolyl, such as 1,3,4-oxadiazol-5-yl, and 1,2,4-oxadiazol-5-yl. A group of further preferred examples of 5-membered ring systems with 2 to 4 nitrogen atoms include imidazolyl, preferably imidazol-2-yl; triazolyl, preferably 1,3,4-triazol-5-yl; 1,2,3-triazol-5-yl, 1,2,4-triazol-5-yl, and tetrazolyl, preferably I H-tetrazol-5-yl. A preferred group of examples of benzo-fused derivatives are benzoxazol-2-yl, benzthiazol-2-yl and benzimidazol-2-yl.

Further suitable specific examples of the above heterocylic ring systems are 6-membered ring systems containing one to three nitrogen atoms and optionally a sulfur or oxygen atom. Such examples include pyridyl, such as pyrid-2-yl, pyrid-3-yl, and pyrid-4-yl; pyrimidyl, preferably pyrimid-2-yl and pyrimid-4-yl; triazinyl, preferably 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are a preferred group. The substituents for the optionally substituted heterocyclic ring systems, and further examples of the 5- and 6-membered ring systems discussed above can be found in W. Druckheimer et al., U.S. Pat. No. 4,278,793.

A particularly preferred group of “heteroaryl” include; 1,3-thiazol-2-yl, 4-(carboxymethyl)-5-methyl-1,3-thiazol-2-yl, 4-(carboxymethyl)-5-methyl-1,3-thiazol-2-yl sodium salt, 1,2,4-thiadiazol-5-yl, 3-methyl-1,2,4-thiadiazol-5-yl, 1,3,4-triazol-5-yl, 2-methyl-1,3,4-triazol-5-yl, 2-hydroxy-1,3,4-triazol-5-yl, 2-carboxy-4-methyl-1,3,4-triazol-5-yl sodium salt, 2-carboxy-4-methyl-1,3,4-triazol-5-yl, 1,3-oxazol-2-yl, 1,3,4-oxadiazol-5-yl, 2-methyl-1,3,4-oxadiazol-5-yl, 2-(hydroxymethyl)-1,3,4-oxadiazol-5-yl, 1,2,4-oxadiazol-5-yl, 1,3,4-thiadiazol-5-yl, 2-thiol-1,3,4-thiadiazol-5-yl, 2-(methylthio)-1,3,4-thiadiazol-5-yl, 2-amino-1,3,4-thiadiazol-5-yl, 1H-tetrazol-5-yl, 1-methyl-1H-tetrazol-5-yl, 1-(1-(dimethylamino)eth-2-yl)-1H-tetrazol-5-yl, 1-(carboxymethyl)-1H-tetrazol-5-yl, 1-(carboxymethyl)-1H-tetrazol-5-yl sodium salt, 1-(methylsulfonic acid)-1H-tetrazol-5-yl, 1-(methylsulfonic acid)-1H-tetrazol-5-yl sodium salt, 2-methyl-1H-tetrazol-5-yl, 1,2,3-triazol-5-yl, 1-methyl-1,2,3-triazol-5-yl, 2-methyl-1,2,3-triazol-5-yl, 4-methyl-1,2,3-triazol-5-yl, pyrid-2-yl N-oxide, 6-methoxy-2-(n-oxide)-pyridaz-3-yl, 6-hydroxypyridaz-3-yl, 1-methylpyrid-2-yl, 1-methylpyrid-4-yl, 2-hydroxypyrimid-4-yl, 1,4,5,6-tetrahydro-5,6-dioxo-4-methyl-as-triazin-3-yl, 1,4,5,6-tetrahydro-4-(formylmethyl)-5,6-dioxo-as-triazin-3-yl, 2,5-dihydro-5-oxo-6-hydroxy-astriazin-3-yl, 2,5-dihydro-5-oxo-6-hydroxy-as-triazin-3-yl sodium salt, 2,5-dihydro-5-oxo-6-hydroxy-2-methyl-astriazin-3-yl sodium salt, 2,5-dihydro-5-oxo-6-hydroxy-2-methyl-as-triazin-3-yl, 2,5-dihydro-5-oxo-6-methoxy-2-methyl-as-triazin-3-yl, 2,5-dihydro-5-oxo-as-triazin-3-yl, 2,5-dihydro-5-oxo-2-methyl-as-triazin-3-yl, 2,5-dihydro-5-oxo-2,6-dimethyl-as-triazin-3-yl, tetrazolo[1,5-b]pyridazin-6-yl and 8-aminotetrazolo[1,5-b]-pyridazin-6-yl.

An alternative group of “heteroaryl” includes; 4-(carboxymethyl)-5-methyl-1,3-thiazol-2-yl, 4-(carboxymethyl)-5-methyl-1,3-thiazol-2-yl sodium salt, 1,3,4-triazol-5-yl, 2-methyl-1,3,4-triazol-5-yl, 1H-tetrazol-5-yl, 1-methyl-1H-tetrazol-5-yl, 1-(1-(dimethylamino)eth-2-yl)-1H-tetrazol-5-yl, 1-(carboxymethyl)-1H-tetrazol-5-yl, 1-(carboxymethyl)-1H-tetrazol-5-yl sodium salt, 1-(methylsulfonic acid)-1H-tetrazol-5-yl, 1-(methylsulfonic acid)-1H-tetrazol-5-yl sodium salt, 1,2,3-triazol-5-yl, 1,4,5,6-tetrahydro-5,6-dioxo-4-methyl-as-triazin-3-yl, 1,4,5,6-tetrahydro-4-(2-formylmethyl)-5,6-dioxo-as-triazin-3-yl, 2,5-dihydro-5-oxo-6-hydroxy-2-methyl-as-triazin-3-yl sodium salt, 2,5-dihydro-5-oxo-6-hydroxy-2-methyl-as-triazin-3-yl, tetrazolo[1,5-b]pyridazin-6-yl, and 8-aminotetrazolo[1,5-b]pyridazin-6-yl.

The term “lower” when used with a term such as alkyl to form “lower alkyl”, for example, means containing from 1 to 6 carbon atoms.

“Pharmaceutically acceptable salts” include both acid and base addition salts. “Pharmaceutically acceptable acid addition salt” refers to those salts which retain the biological effectiveness and properties of the free bases and which are not biologically or otherwise undesirable, formed with inorganic acids such as hydrochloric acid, hydrobromic acid, sulfuric acid, nitric acid, carbonic acid, phosphoric acid and the like, and organic acids may be selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, salicyclic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium and magnesium salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, polyamine resins and the like. Particularly preferred organic non-toxic bases are isopropylamine, diethylamine, ethanolamine, trimethamine, dicyclohexylamine, choline, and caffeine.

b. Alpha4 Integrin Antagonist—Formula I, II, and III

Small molecule antagonists of alpha4 integrins useful in the methods of the invention include compounds of formula I, II, or III and as described in WO 01/21584: 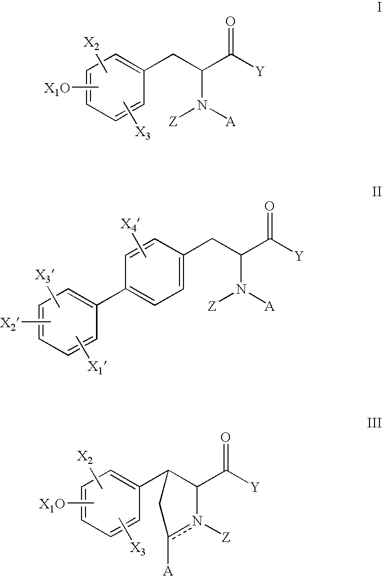
where
Z is H or lower alkyl;
A can have the structure: 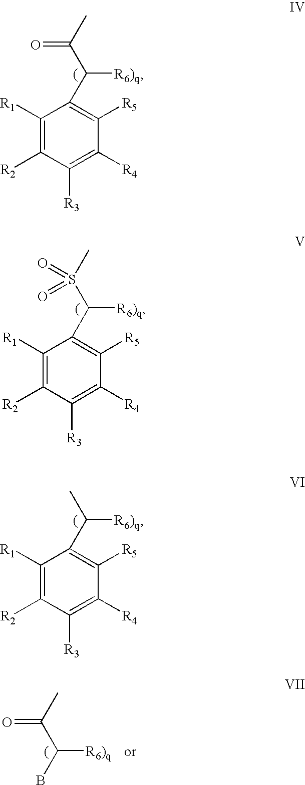
in which
B is cyanoalkyl, a carbocycle or a heterocycle optionally substituted with one or more R₁ substituents; q is 0-3;
R₁, R₂, R₃, R₄, R₅ and R₆ independently are hydrogen, alkyl, amino, alkylamino, dialkylamino, nitro, urea, cyano, thio, alkylthio, hydroxy, alkoxy, alkoxyalkyl, alkoxycarbonyl, alkoxycarbonylamino, aryloxycarbonylamino, alkylsulfinyl, sulfonyl, alkylsulfonyl, aralkylsulfonyl, arylsulfonyl, heteroarylsulfonyl, alkanoyl, alkanoylamino, cycloalkanoylamino, aryl, arylalkyl, halogen, or alkylphosphonyl, and R₁, R₂, R₃, R₄ and R₅ are substituted with 0-3 substituents selected from the group consisting of hydroxy, carboxyl, lower alkoxycarbonyl, lower alkyl, nitro, oxo, cyano, carbocyclyl, heterocyclyl, heteroaryl, lower alkylthio, lower alkoxy, lower alkylamino, lower alkanoylamino, lower alkylsulfinyl, lower sulfonyl, lower alkylsulfonyl, lower alkanoyl, aryl, aroyl, heterocyclylcarbonyl, halogen and lower alkylphosphonyl; or two of R₁ to R₅ together form a carbocycle or heterocyclic ring;
   Y is H, alkoxy, alkoxyalkoxy, aryloxy, alkylaminoalkoxy, dialkylaminoalkoxy, alkylamino, arylamino, heterocyclyl or heteroarylalkyl, where each of the forgoing may be substituted or unsubstituted;
   X₁ is H, C(O)OR, C(O)NRaRb, C(O)R, or C(O)SR, wherein R, Ra and Rb, individually, is hydrogen or alkyl, alkoxy, aryl, heterocyclyl, heteroaryl, substituted with 0-4 substituents selected from the group consisting of halogen, hydroxy, amino, carboxyl, nitro, cyano, heterocylyl, heteroaryl, aryl, aroyl, aryloxy, aralkyl, aralkyloxy, aryloxycarbonyl, aralkyloxycarbonyl, alkylenedioxy, lower alkoxycarbonyl, lower alkyl, lower alkenyl, lower alkynyl, lower alkylthio, lower alkoxy, lower alkylamino, lower alkylsulfinyl, lower sulfonyl, lower alkylsulfonyl, lower alkanoyl, lower alkylphosphonyl, aminosulfonyl lower alkyl, hydroxy lower alkyl, alkylsulfinyl lower alkyl, alkylsulfonyl lower alkyl, alkylthio lower alkyl, heteroarylthio lower alkyl, heteroaryloxy lower alkyl, heteroarylamino lower alkyl, halo lower alkyl, and alkoxy lower alkyl; wherein said heterocyclyl, heteroaryl, aryl, aroyl, aryloxy, aralkyl, aralkyloxy, aryloxycarbonyl and aralkyloxycarbonyl is optionally substituted with halogen, hydroxyl, amino, carboxyl, nitro, cyano, alkyl and alkoxy; and wherein Ra and Rb together with the nitrogen to which they are attached may form a heterocyclyl or heteroaryl group substituted with 0-5 substituents R or Rd; wherein Rd has the structure: 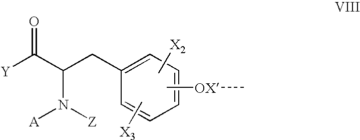
      where X′ is a divalent linker selected from the group consisting of C(O)NRa, C(O) or a bond;
   X₂ and X₃ are each independently hydrogen, halogen, hydroxy, amino, carboxyl, nitro, cyano, or substituted or unsubstituted alkyl, aryl, heterocylyl, heteroaryl, aryl, aroyl, aryloxy, alkylenedioxy, lower alkyl carbonylamino, lower alkenyl carbonylamino, aryl carbonylamino, arylalkyl carbonylamino, lower alkoxy carbonylamino, lower alkylamino carbonylamino, arylamino carbonylamino, lower alkoxycarbonyl, lower alkyl, lower alkenyl, lower alkynyl, lower alkylthio, lower alkoxy, lower alkylamino, lower alkylsulfinyl, lower sulfonyl, lower alkylsulfonyl, lower alkanoyl, lower alkylphosphonyl, aminosulfonyl lower alkyl, hydroxy lower alkyl, alkylsulfinyl lower alkyl, alkylsulfonyl lower alkyl, alkylthio lower alkyl, heteroarylthio lower alkyl, heteroaryloxy lower alkyl, heteroarylamino lower alkyl, halo lower alkyl, alkoxy lower alkyl; and wherein X₁ and X₂ or X₃ may be bonded together to form a heterocylic or heteroaryl ring(s); or X₃ and Z together form a heterobicyclic ring;
   X_{1′}, X_{2′}, X_{3′} and X_{4′} are each independently hydrogen, halogen, hydroxy, amino, carboxyl, nitro, cyano, or substituted or unsubstituted alkyl, alkenyl, alkynyl, arylalkyl, heterocylyl, heteroaryl, aryl, aroyl, aryloxy, alkylenedioxy, lower alkyl carbonylamino, lower alkenyl carbonylamino, aryl carbonylamino, arylalkyl carbonylamino, lower alkoxy carbonylamino, lower alkylamino carbonylamino, arylamino carbonylamino, lower alkoxycarbonyl, lower alkyl, lower alkenyl, lower alkynyl, lower alkylthio, lower alkoxy, lower alkylamino, lower alkylsulfinyl, lower sulfonyl; lower alkylsulfonyl, lower alkanoyl, lower alkylphosphonyl, aminosulfonyl lower alkyl, hydroxy lower alkyl, alkylsulfinyl lower alkyl, alkylsulfonyl lower alkyl, alkylthio lower alkyl, heteroarylthio lower alkyl, heteroaryloxy lower alkyl, heteroarylamino lower alkyl, halo lower alkyl, alkoxy lower alkyl; or a pharmaceutically acceptable salt thereof.

The compounds of the invention contain one or more asymmetric carbon atoms. Accordingly, the compounds may exist as diasteriomers, enantiomers or mixtures thereof. The syntheses described above may employ racemates, diasteriomers or enantiomers as starting materials or as intermediates. Diasteriomeric compounds may be separated by chromatographic or crystallization methods. Similarly, enantiomeric mixtures may be separated using the same techniques or others known in the art. Each of the asymmetric carbon atoms may be in the R or S configuration and both of these configurations are within the scope of the invention. Compounds having the S configuration are preferred.

In one preferred embodiment, XI in structure I is C(O)OR, C(O)R, or C(O)SR, more preferably C(O)NRaRb, with the remaining variables A, Z, Y, X₂, X₃ and X₄ having any of the definitions given above. The X₁ group is preferably in the para position relative to the point of ring attachment, but may also be preferably in the meta position. Ra and Rb together with the nitrogen to which they are attached may preferably form a 5-membered or 6-membered heterocyclyl or heteroaryl group substituted with 0-5 substituents R. The heterocyclyl or heteroaryl ring system will preferably contain one nitrogen atom, but may also preferably contain another nitrogen or an oxygen atom in the ring system. The hetero ring systems may contain fused heterocyclyl or heteroaryl rings or a combination of both and the rings may be substituted or unsubstituted.

Representative examples of suitable specific heterocyclyl and heteroaryl groups are:

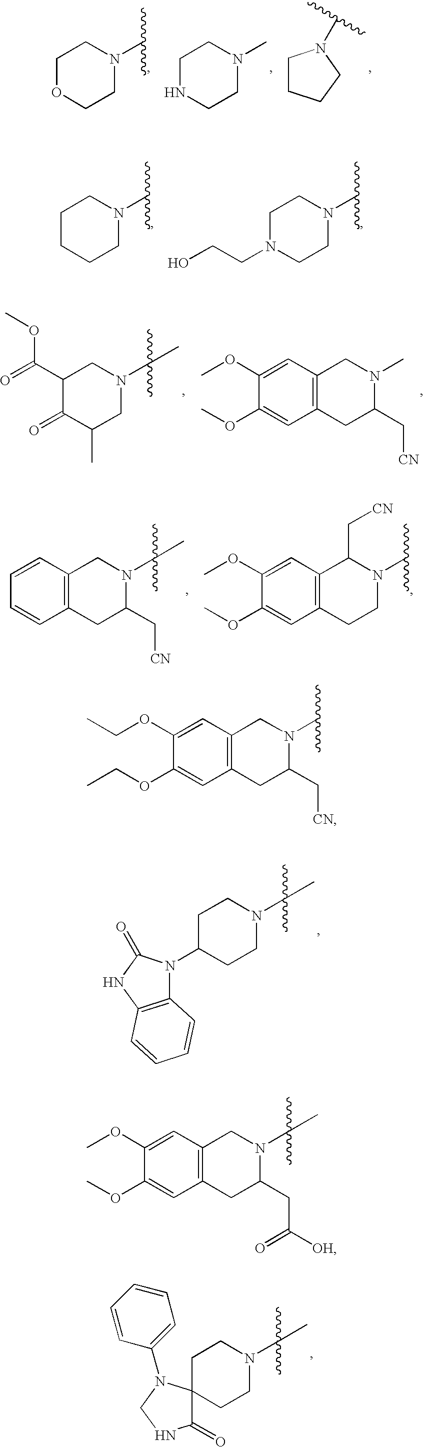

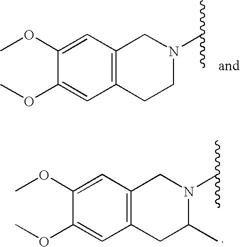

R, Ra and Rb may also be non-cyclic, for example an hydrogen or alkyl, aryl, heterocyclyl, heteroaryl, substituted with 0-4 substituents selected from the group consisting of halogen, hydroxy, amino, carboxyl, nitro, cyano, heterocylyl, heteroaryl, aryl, aroyl, aryloxy, alkylenedioxy, lower alkoxycarbonyl, lower alkyl, lower alkenyl, lower alkynyl, lower alkylthio, lower alkoxy, lower alkylamino, lower alkylsulfinyl, lower sulfonyl, lower alkylsulfonyl, lower alkanoyl, lower alkylphosphonyl, aminosulfonyl lower alkyl, hydroxy lower alkyl, alkylsulfinyl lower alkyl, alkylsulfonyl lower alkyl, alkylthio lower alkyl, heteroarylthio lower alkyl, heteroaryloxy lower alkyl, heteroarylamino lower alkyl, halo lower alkyl, alkoxy lower alkyl; optionally substituted as described above. Preferred groups are substituted and unsubstituted lower alkyl, lower alkenyl, aryl, and aryl lower alkyl.

Some representative examples of such R, Ra and Rb groups are shown below:

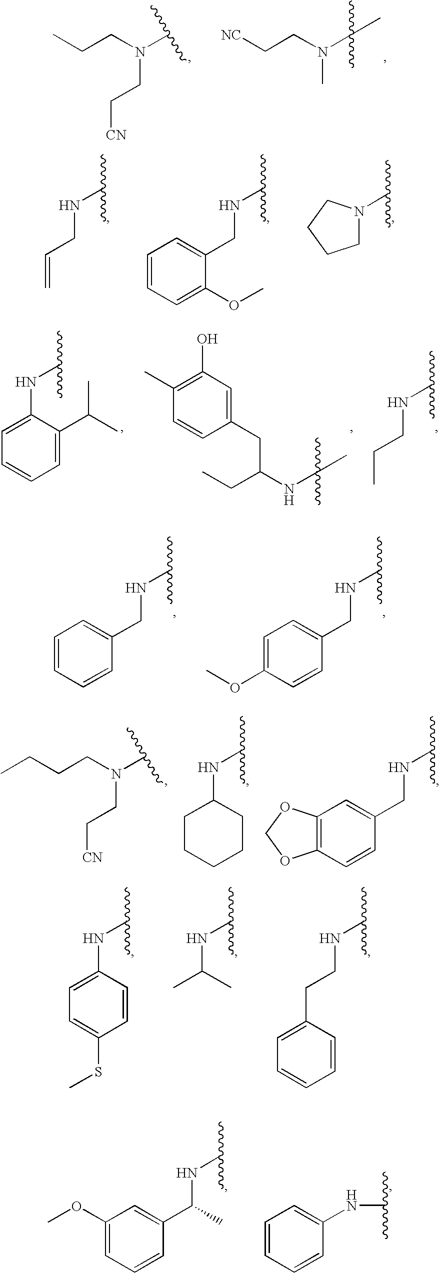

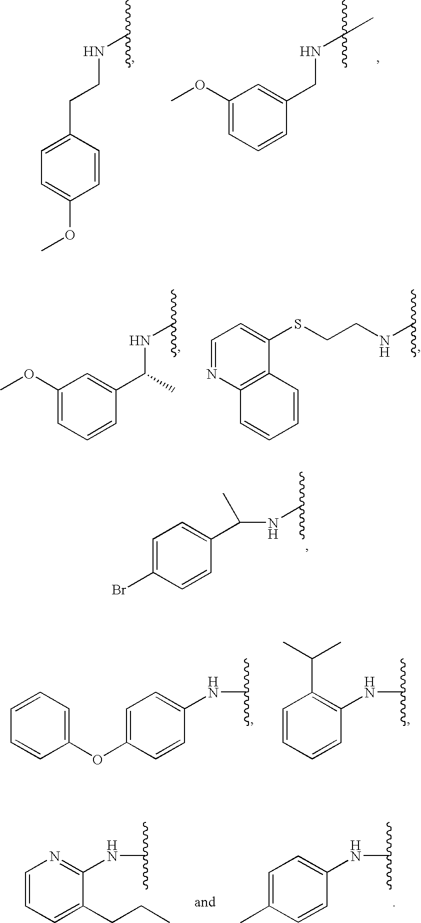

In a preferred embodiment, A can have the structure (IX) 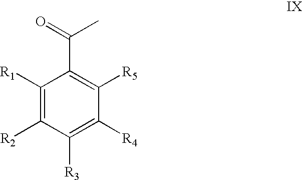
where preferably R₁, R₅, or both R₁ and R₅ are not hydrogen. That is, preferred A groups are ortho-substituted benzoyl groups. Particularly preferred ortho substituents are chloro, bromo, amino and hydroxy. In addition to R₁ and/or R₅, the phenyl ring of the benzoyl may preferably have one or two additional substituents at R₂, R₃ or R₄. Preferred R₁, R₂, R₃ R₄, and R₅ include nitro, halogen (Cl, Br, F, I), amino, aryl, lower alkyl, lower alkylthio, lower alkoxy, lower alkylamino, lower alkyl sulfinyl, lower alkylsulfonyl, lower alkanoyl, and lower alkylphosphonyl, which may each be substituted or unsubstituted.

Some representative examples of the structure A (IX) are include:

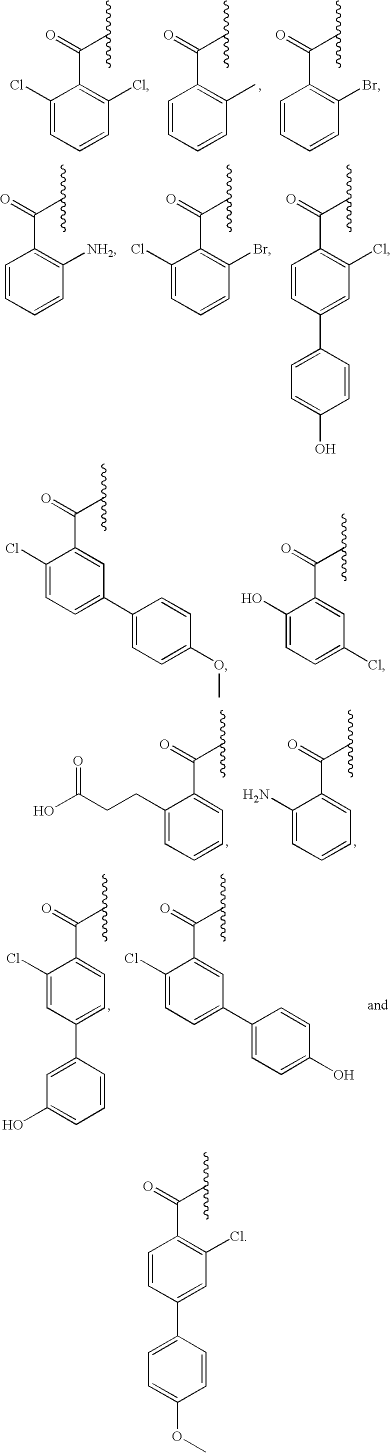

Y is preferably OH or an ester or pharmaceutically acceptable carboxylic acid salt thereof. Preferred esters are substituted or unsubstituted alkyl, alkenyl, aryl, and aryl alkyl esters.

Z is preferably hydrogen.

Preferred X₂, X₃ and X₄ include halogen, alkyl, amino, alkylamino, and alkyl carbonylamino, the alkyl group of which may be substituted or unsubstituted. For compounds having structure I, X₂ and X₃ are more preferably hydrogen. For compounds having structure II, X₂, X₃ and X₄ are more preferably hydrogen.

In a particular embodiment, X₁ of Formulas I, II, or III, can be any one of the groups shown in Table 1 below, which is designated as substituent R when combined with the carbonyl from which it depends. In a particular embodiment, A is any of the groups shown in Table 1 which is designated as substituent R′.

c. Preferred Compounds of Formula X

Specific alpha4 integrin antagonists include those of formula X below, having the R and R′ substituents shown in Tables 1 and 2, as well as the specific compounds shown in Table 3.

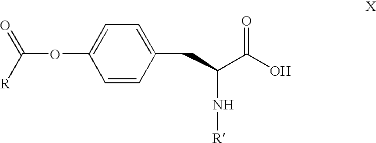

TABLE 1
R and R′ Substituents of Formula X

| R | R′-compound no. |
|---|---|
| | 129 |
| 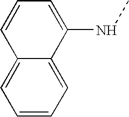 | 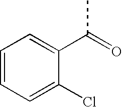 |
| | 130 |
| 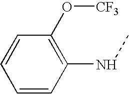 | 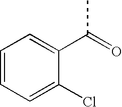 |
| | 131 |
| 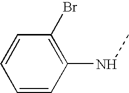 | 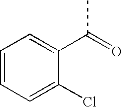 |
| | 132 |
| 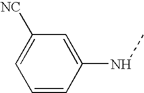 | 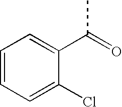 |
| | 133 |
| 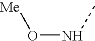 | 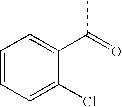 |
| | 134 |
| 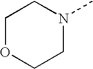 | 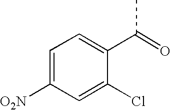 |
| | 135 |
| 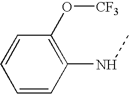 | 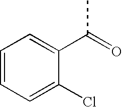 |
| | 136 |
| 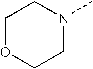 | 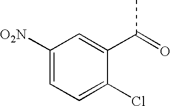 |
| | 137 |
| 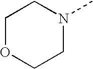 | 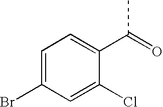 |
| | 138 |
| 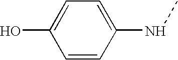 | 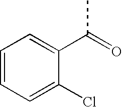 |
| | 139 |
| 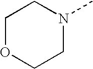 | 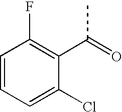 |
| | 140 |
| 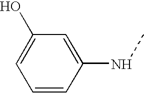 | 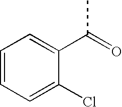 |
| | 141 |
| 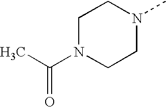 | 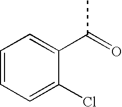 |
| | 142 |
| 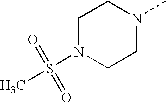 | 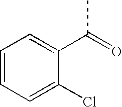 |
| | 143 |
| 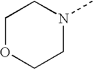 | 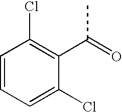 |
| | 144 |
| 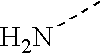 | 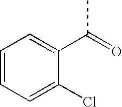 |
| | 145 |
| 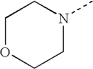 | 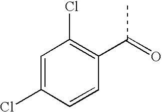 |
| | 146 |
| 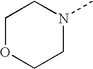 | 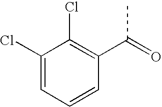 |
| | 147 |
| 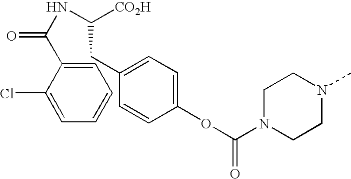 | 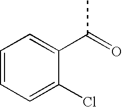 |
| | 148 |
| 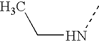 | 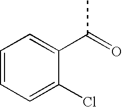 |
| | 149 |
| 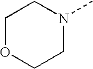 | 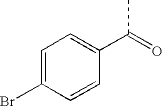 |
| | 150 |
| 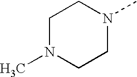 | 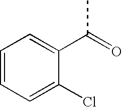 |
| | 151 |
| 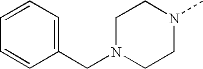 | 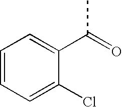 |
| | 152 |
| 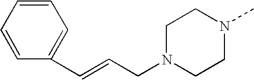 | 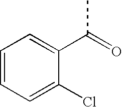 |
| | 153 |
| 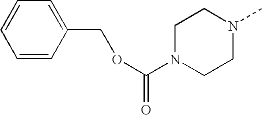 | 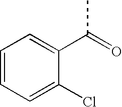 |
| | 154 |
| 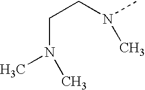 | 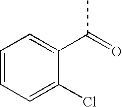 |
| | 155 |
| 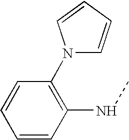 | 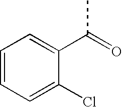 |
| | 156 |
| 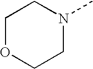 | 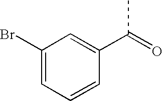 |
| | 157 |
| 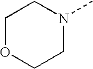 | 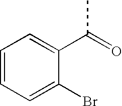 |
| | 158 |
| 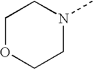 | 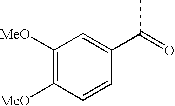 |
| | 159 |
| 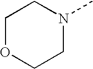 | 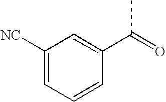 |
| | 160 |
| 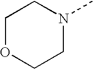 | 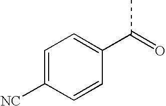 |
| | 161 |
| 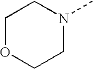 | 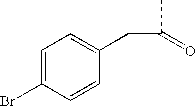 |
| | 162 |
| 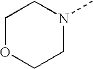 | 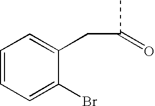 |
| | 163 |
| 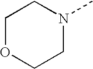 | 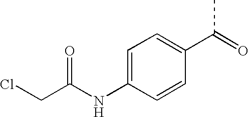 |
| | 164 |
| 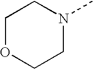 | 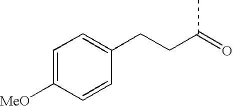 |
| | 165 |
| 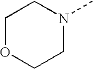 | 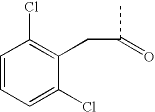 |
| | 166 |
| 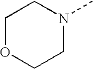 | 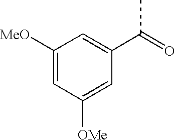 |
| | 167 |
| 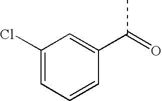 | 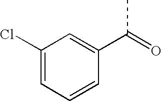 |
| | 168 |
| 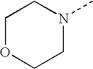 | 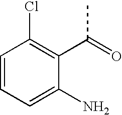 |
| | 169 |
| 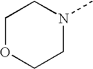 | 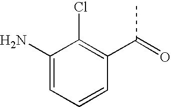 |
| | 170 |
| 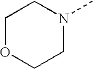 | 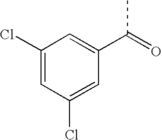 |
| | 171 |
| 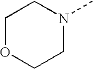 | 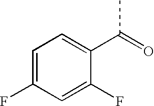 |
| | 172 |
| 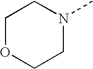 | 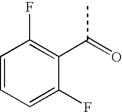 |
| | 173 |
| 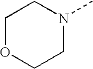 | 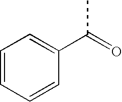 |
| | 174 |
| 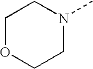 | 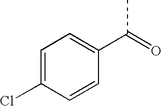 |
| | 175 |
| 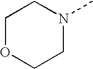 | 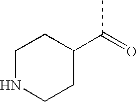 |
| | 176 |
| 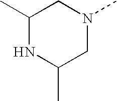 | 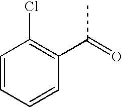 |
| | 177 |
| 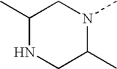 | 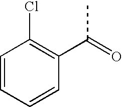 |
| | 178 |
| 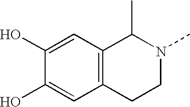 | 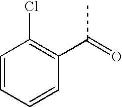 |
| | 179 |
| 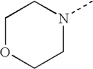 | 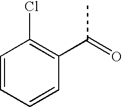 |
| | 180 |
| 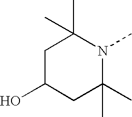 | 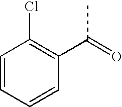 |
| | 181 |
| 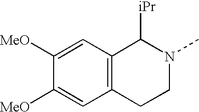 | 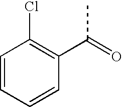 |
| | 182 |
| 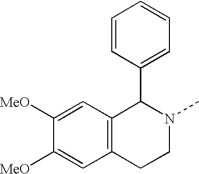 | 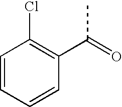 |
| | 183 |
| 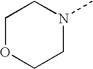 | 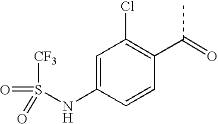 |
| | 184 |
| 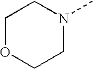 | 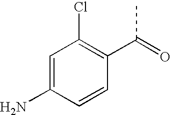 |
| | 185 |
| 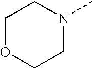 | 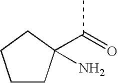 |
| | 186 |
| 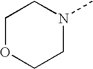 | 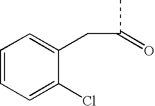 |
| | 187 |
| 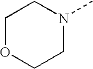 | 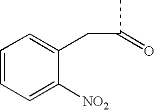 |
| | 188 |
| 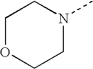 | 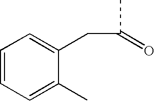 |
| | 189 |
| 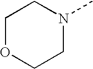 | 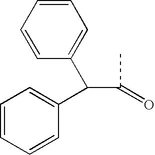 |
| | 190 |
| 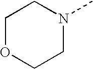 | 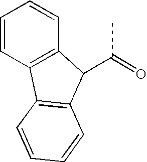 |
| | 191 |
| 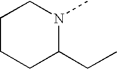 | 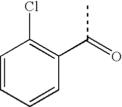 |
| | 192 |
| 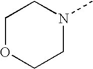 | 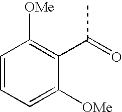 |
| | 193 |
| 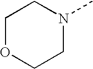 | 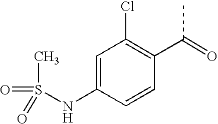 |
| | 194 |
| 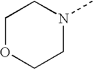 | 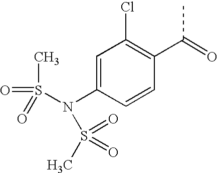 |
| | 195 |
| 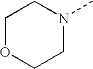 | 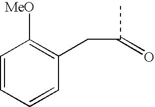 |
| | 196 |
| 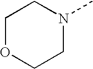 | 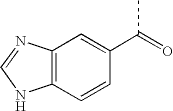 |
| | 197 |
| 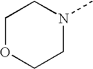 | 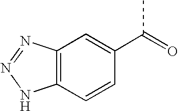 |
| | 198 |
| 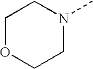 | 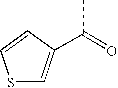 |
| | 199 |
| 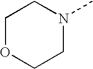 | 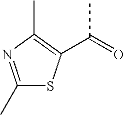 |
| | 200 |
| 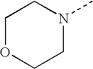 | 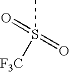 |
| | 201 |
| 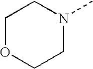 | 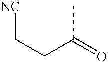 |
| | 202 |
| 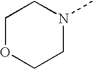 | 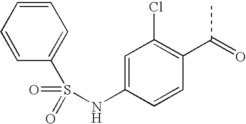 |
| | 203 |
| 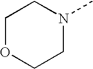 | 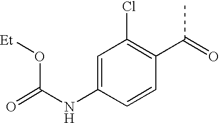 |
| | 204 |
| 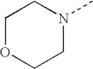 | 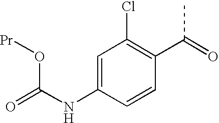 |
| | 205 |
| 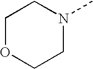 | 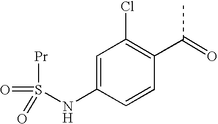 |
| | 206 |
| 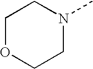 | 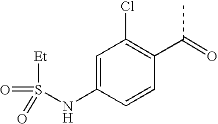 |
| | 207 |
| 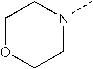 | 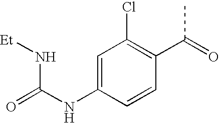 |
| | 208 |
| 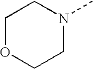 | 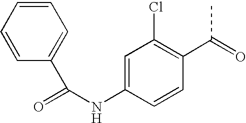 |
| | 209 |
| 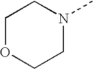 | 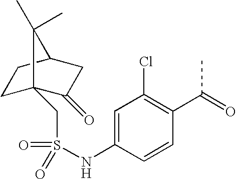 |
| | 210 |
| 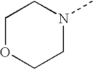 | 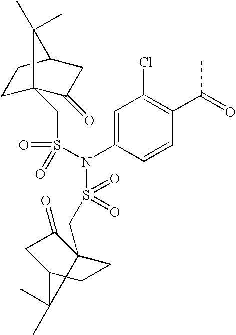 |
| | 211 |
| 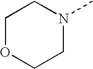 | 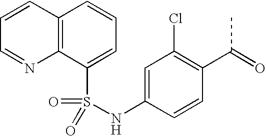 |
| | 212 |
| 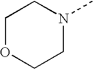 | 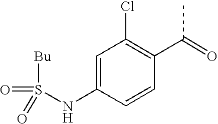 |
| | 213 |
| 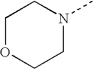 | 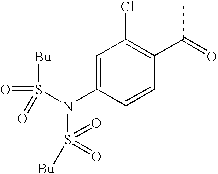 |
| | 214 |
| 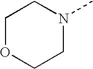 | 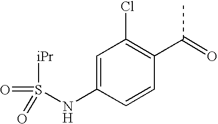 |
| | 215 |
| 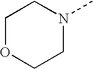 | 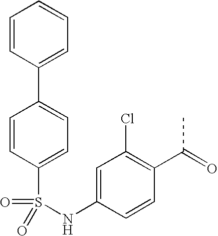 |
| | 216 |
| 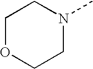 | 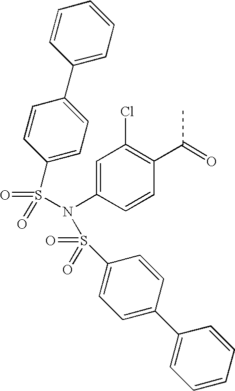 |
| | 217 |
| 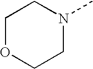 | 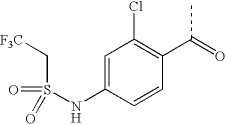 |
| | 218 |
| 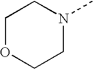 | 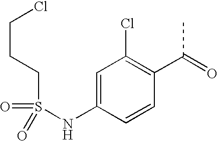 |
| | 219 |
| 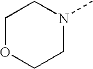 | 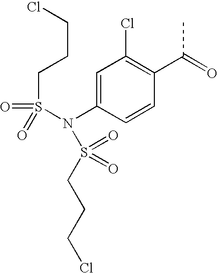 |
| | 220 |
| 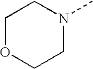 | 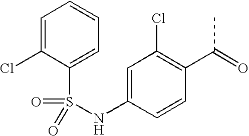 |
| | 221 |
| 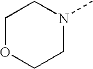 | 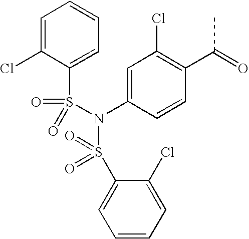 |
| | 222 |
| 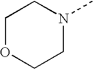 | 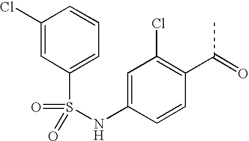 |
| | 223 |
| 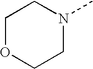 | 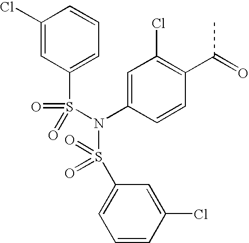 |
| | 224 |
| 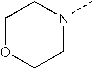 | 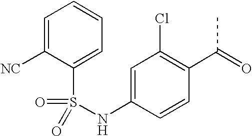 |
| | 225 |
| 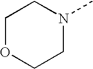 | 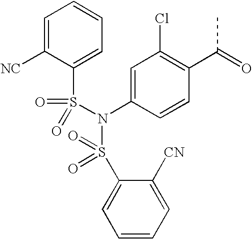 |
| | 226 |
| 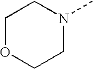 | 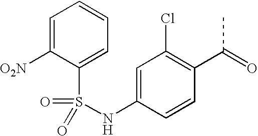 |
| | 227 |
| 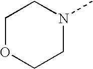 | 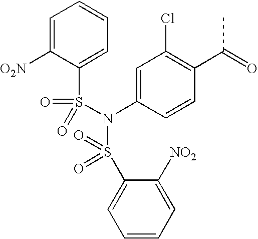 |
| | 228 |
| 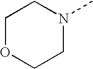 | 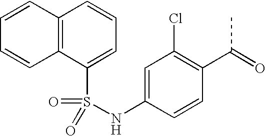 |
| | 229 |
| 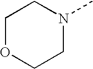 | 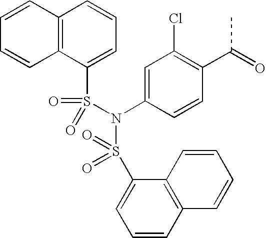 |
| | 230 |
| 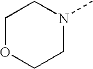 | 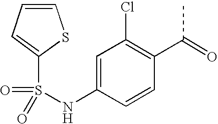 |
| | 231 |
| 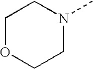 | 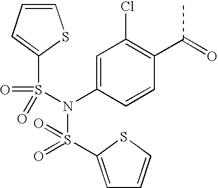 |
| | 232 |
| 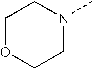 | 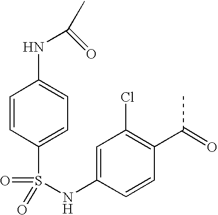 |
| | 233 |
| 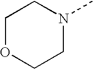 | 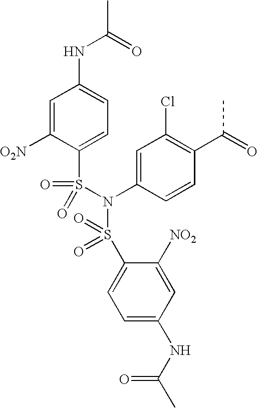 |
| | 234 |
| 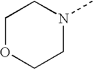 | 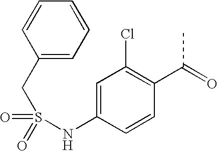 |
| | 235 |
| 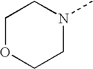 | 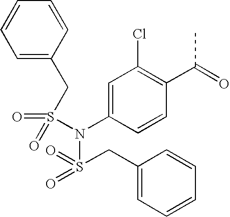 |
| | 236 |
| 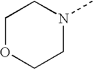 | 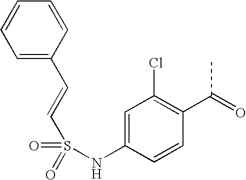 |
| | 237 |
| 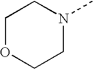 | 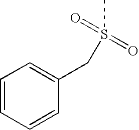 |
| | 238 |
| 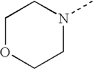 | 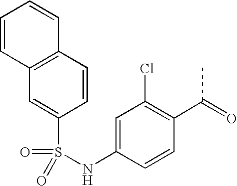 |
| | 239 |
| 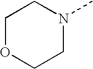 | 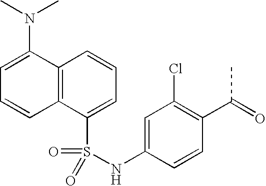 |
| | 240 |
| 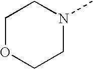 | 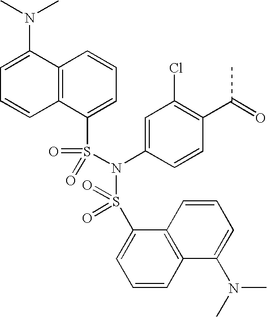 |
| | 241 |
| 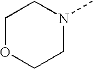 | 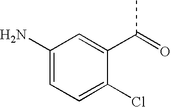 |
| | 242 |
| 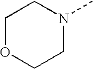 | 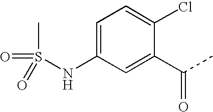 |
| | 243 |
| 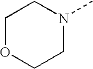 | 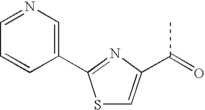 |
| | 244 |
| 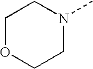 | 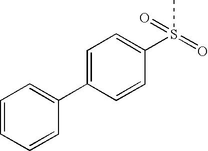 |
| | 245 |
| 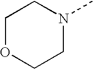 | 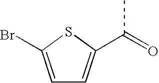 |
| | 246 |
| 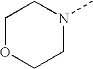 | 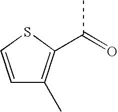 |
| | 247 |
| 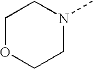 | 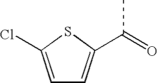 |
| | 248 |
| 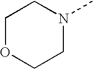 | 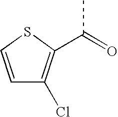 |
| | 249 |
| 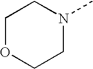 | 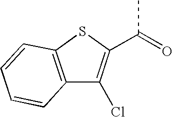 |
| | 250 |
| 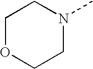 | 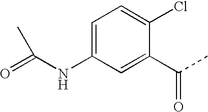 |
| | 251 |
| 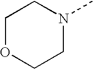 | 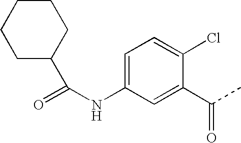 |
| | 252 |
| 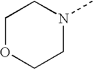 | 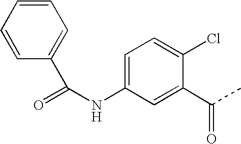 |
| | 253 |
| 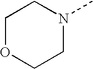 | 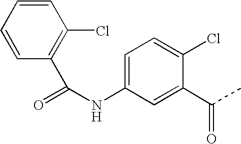 |
| | 254 |
| 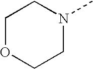 | 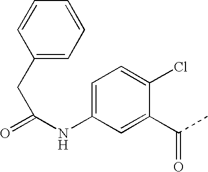 |
| | 255 |
| 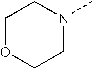 | 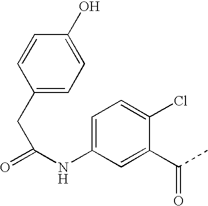 |
| | 256 |
| 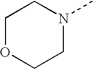 | 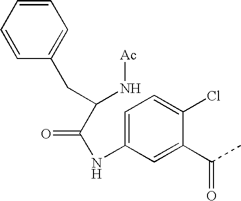 |
| | 257 |
| 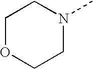 | 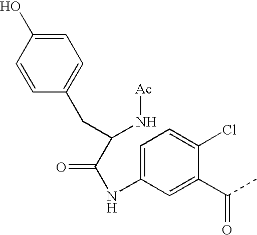 |
| | 258 |
| 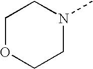 | 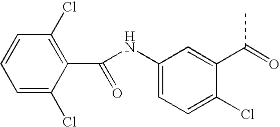 |
| | 259 |
| 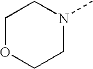 | 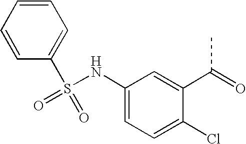 |
| | 260 |
| 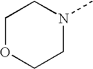 | 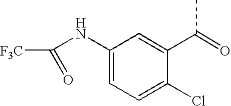 |
| | 261 |
| 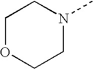 | 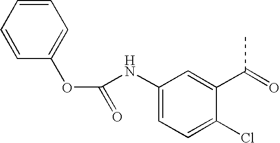 |
| | 262 |
| 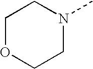 | 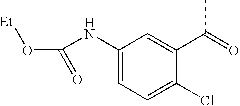 |
| | 263 |
| 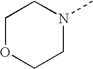 | 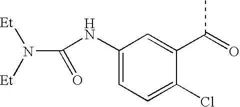 |
| | 264 |
| 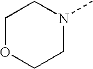 | 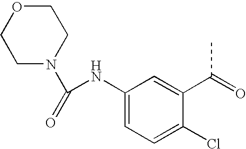 |
| | 265 |
| 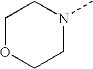 | 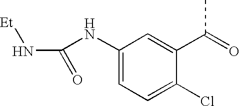 |
| | 266 |
| 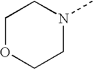 | 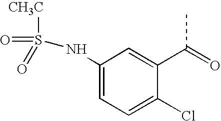 |
| | 267 |
| 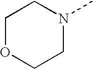 | 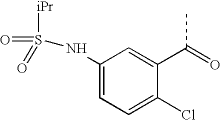 |
| | 268 |
| 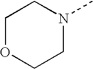 | 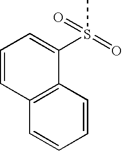 |
| | 269 |
| 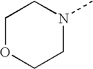 | 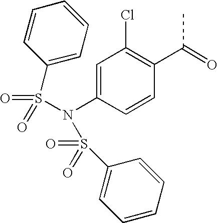 |
| | 270 |
| 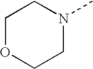 | 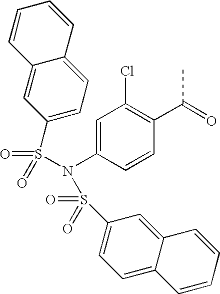 |
| | 271 |
| 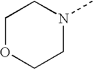 | 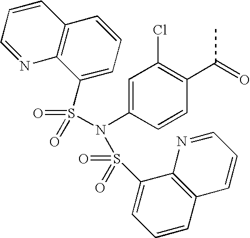 |
| | 272 |
| 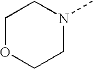 | 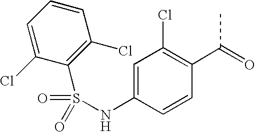 |
| | 273 |
| 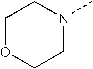 | 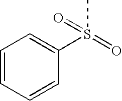 |
| | 274 |
| 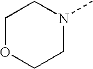 | 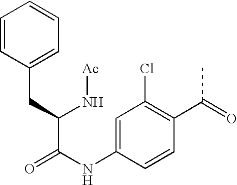 |
| | 275 |
| 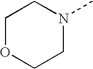 | 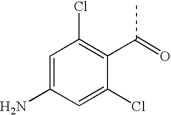 |
| | 276 |
| 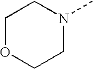 | 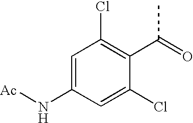 |
| | 277 |
| 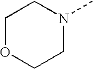 | 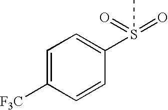 |
| | 278 |
| 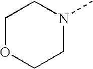 | 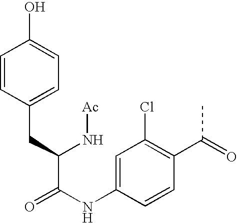 |
| | 279 |
| 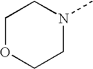 | 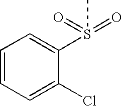 |
| | 280 |
| 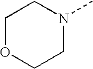 | 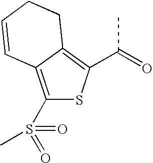 |
| | 281 |
| 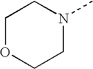 | 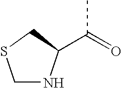 |
| | 282 |
| 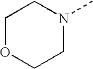 | 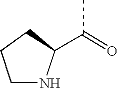 |
| | 283 |
| 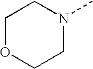 | 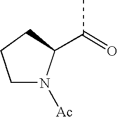 |
| | 284 |
| 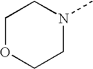 | 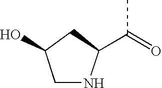 |
| | 285 |
| 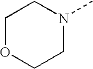 | 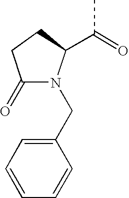 |
| | 286 |
| 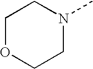 | 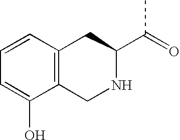 |
| | 287 |
| 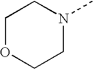 | 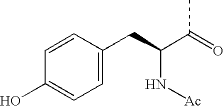 |
| | 288 |
| 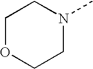 | 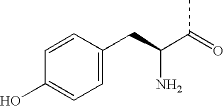 |
| | 289 |
| 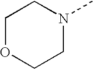 | 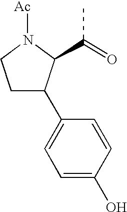 |
| | 290 |
| 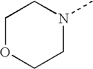 | 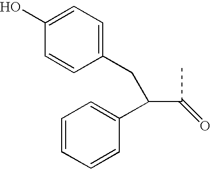 |
| | 291 |
| 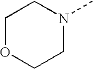 | 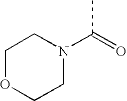 |
| | 292 |
| 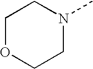 | 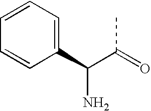 |
| | 293 |
| 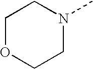 | 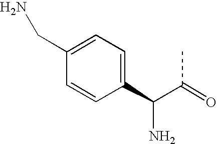 |
| | 294 |
| 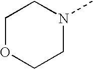 | 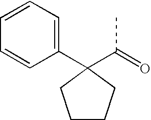 |
| | 295 |
| 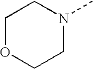 | 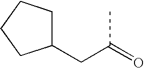 |
| | 296 |
| 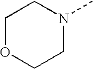 | 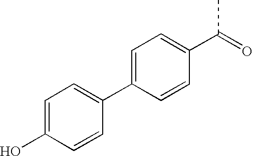 |
| | 297 |
| 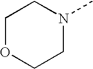 | 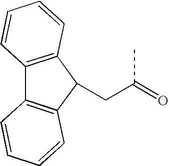 |
| | 298 |
| 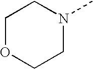 | 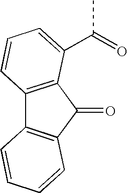 |
| | 299 |
| 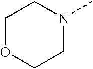 | 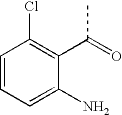 |
| | 300 |
| 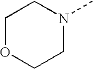 | 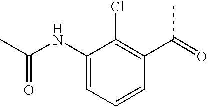 |
| | 301 |
| 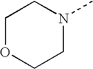 | 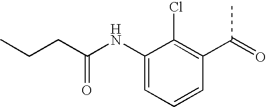 |
| | 302 |
| 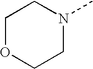 | 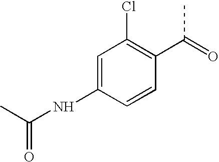 |
| | 303 |
| 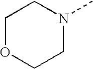 | 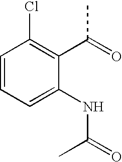 |
| | 304 |
| 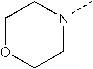 | 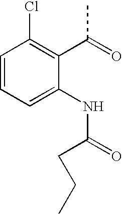 |
| | 305 |
| 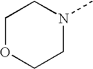 | 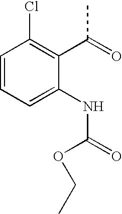 |
| | 306 |
| 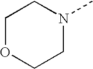 | 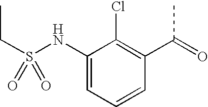 |
| | 307 |
| 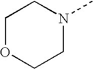 | 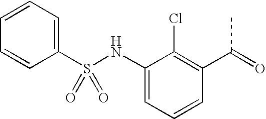 |
| | 308 |
| 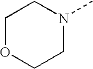 | 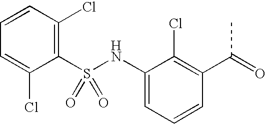 |
| | 309 |
| 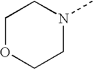 | 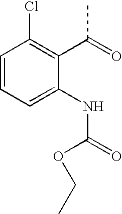 |
| | 310 |
| 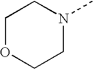 | 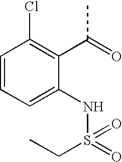 |
| | 311 |
| 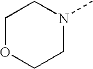 | 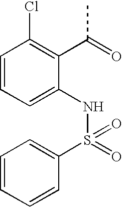 |
| | 312 |
| 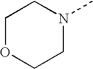 | 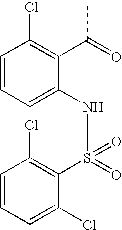 |
| | 313 |
| 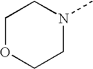 | 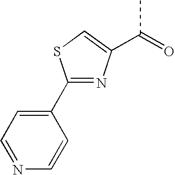 |
| | 314 |
| 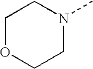 | 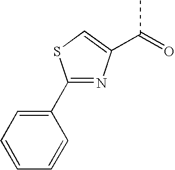 |
| | 315 |
| 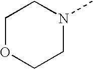 | 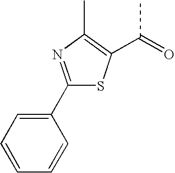 |
| | 316 |
| 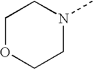 | 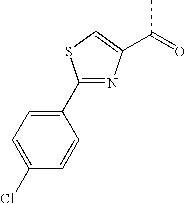 |
| | 317 |
| 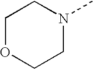 | 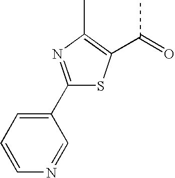 |
| | 318 |
| 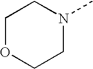 | 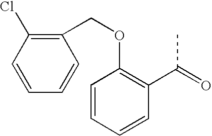 |
| | 319 |
| 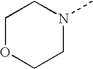 | 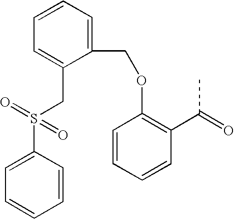 |
| | 320 |
| 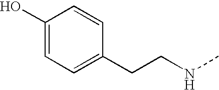 | 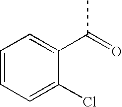 |
| | 321 |
| 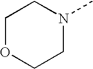 | 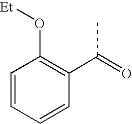 |
| | 322 |
| 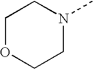 | 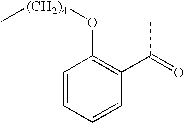 |
| | 323 |
| 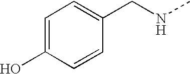 | 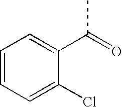 |
| | 324 |
| 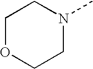 | 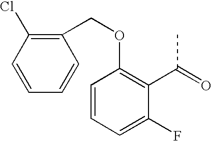 |
| | 325 |
| 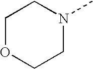 | 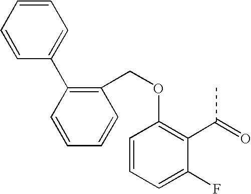 |
| | 326 |
| 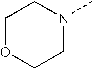 | 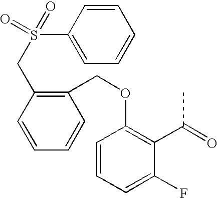 |
| | 327 |
| 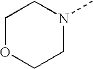 | 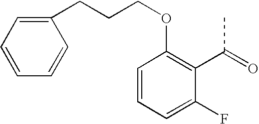 |
| | 328 |
| 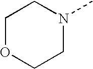 | 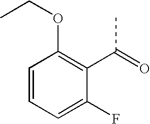 |
| | 329 |
| 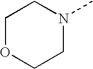 | 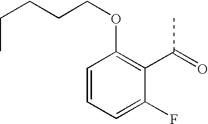 |
| | 330 |
| 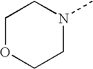 | 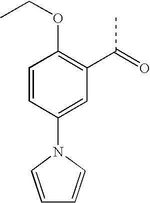 |
| | 331 |
| 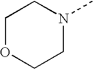 | 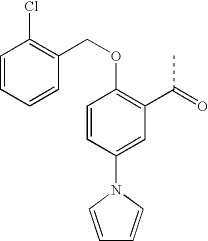 |
| | 332 |
| 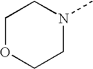 | 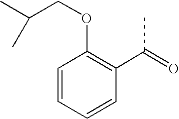 |
| | 333 |
| 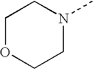 | 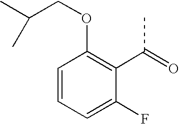 |
| | 334 |
| 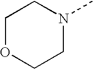 | 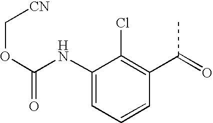 |
| | 335 |
| 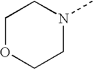 | 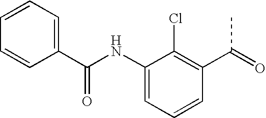 |
| | 336 |
| 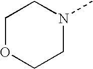 | 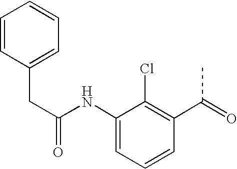 |
| | 337 |
| 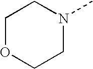 | 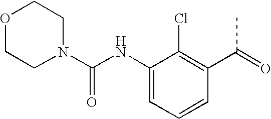 |
| | 338 |
| 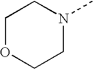 | 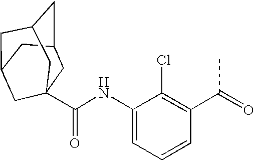 |
| | 339 |
| 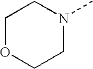 | 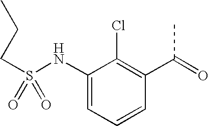 |
| | 340 |
| 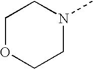 | 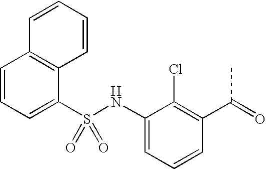 |
| | 341 |
| 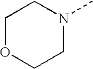 | 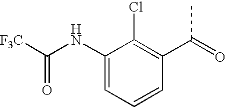 |
| | 342 |
| 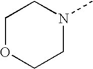 | 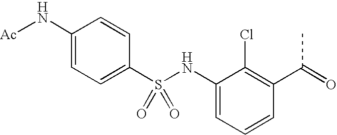 |
| | 343 |
| 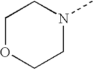 | 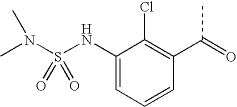 |
| | 344 |
| 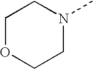 | 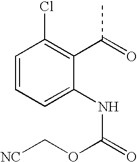 |
| | 345 |
| 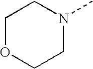 | 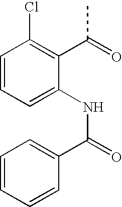 |
| | 346 |
| 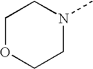 | 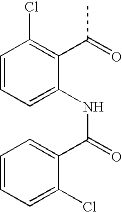 |
| | 347 |
| 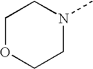 | 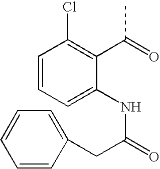 |
| | 348 |
| 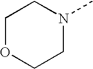 | 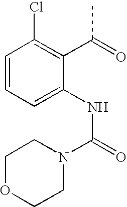 |
| | 349 |
| 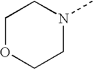 | 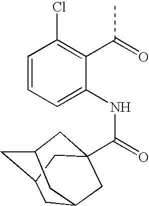 |
| | 350 |
| 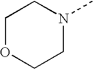 | 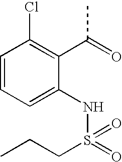 |
| | 351 |
| 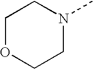 | 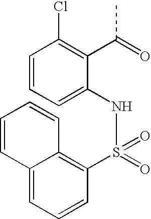 |
| | 352 |
| 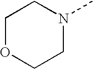 | 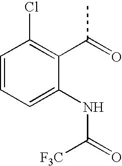 |
| | 353 |
| 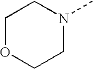 | 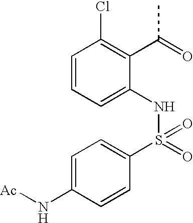 |
| | 354 |
| 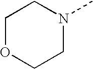 | 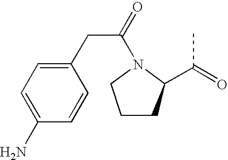 |
| | 355 |
| 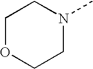 | 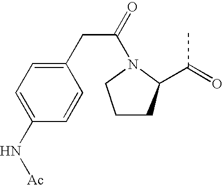 |
| | 356 |
| 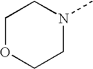 | 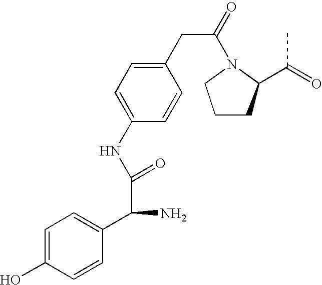 |
| | 357 |
| 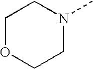 | 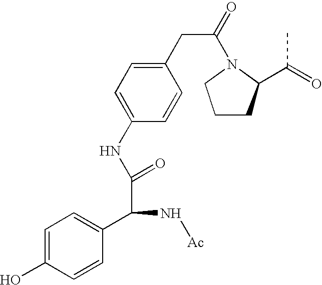 |
| | 358 |
| 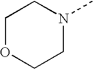 | 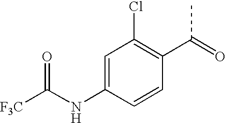 |
| | 359 |
| 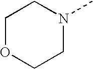 | 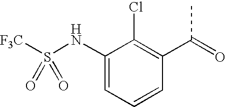 |
| | 360 |
| 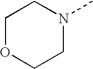 | 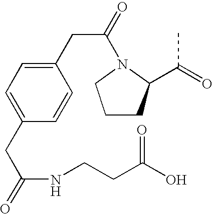 |
| | 361 |
| 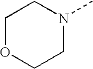 | 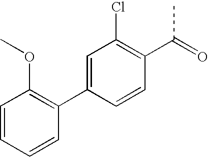 |
| | 362 |
| 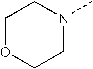 | 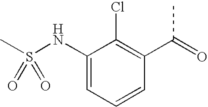 |
| | 363 |
| 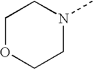 | 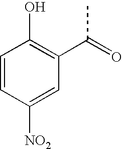 |
| | 364 |
| 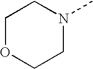 | 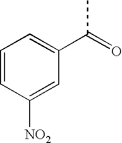 |
| | 365 |
| 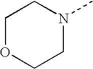 | 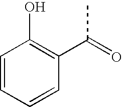 |
| | 366 |
| 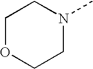 | 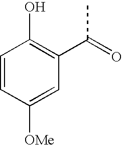 |
| | 367 |
| 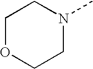 | 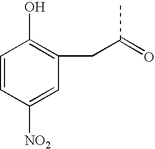 |
| | 368 |
| 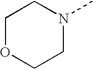 | 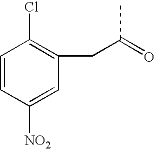 |
| | 369 |
| 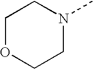 | 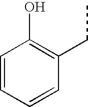 |
| | 370 |
| 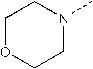 | 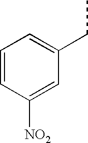 |
| | 371 |
| 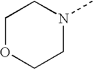 | 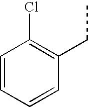 |
| | 372 |
| 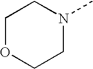 | 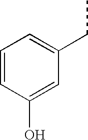 |
| | 373 |
| 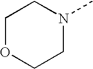 | 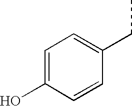 |
| | 374 |
| 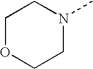 | 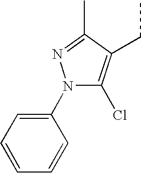 |
| | 375 |
| 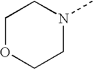 | 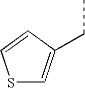 |
| | 376 |
| 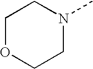 | 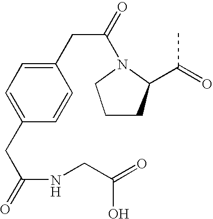 |
| | 377 |
| 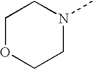 | 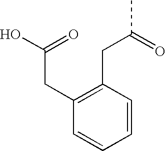 |
| | 378 |
| 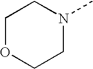 | 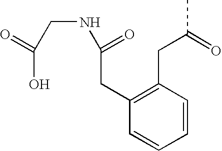 |
| | 379 |
| 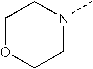 | 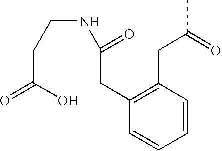 |
| | 380 |
| 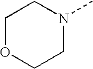 | 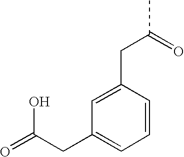 |
| | 381 |
| 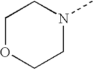 | 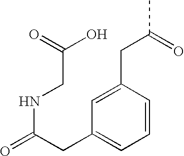 |
| | 382 |
| 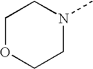 | 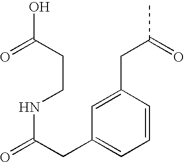 |
| | 383 |
| 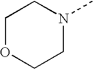 | 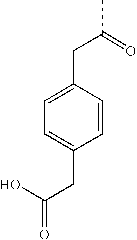 |
| | 384 |
| 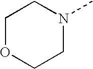 | 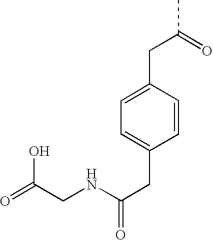 |
| | 385 |
| 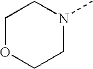 | 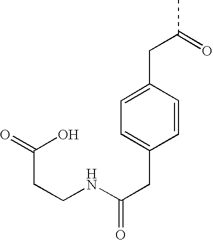 |
| | 386 |
| 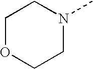 | 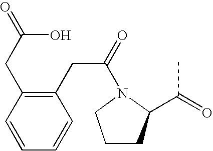 |
| | 387 |
| 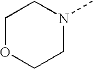 | 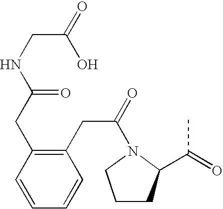 |
| | 388 |
| 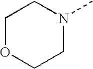 | 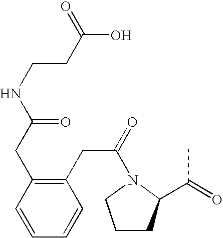 |
| | 389 |
| 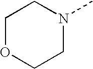 | 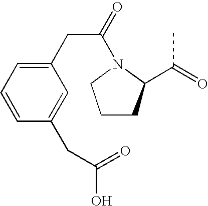 |
| | 390 |
| 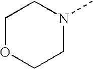 | 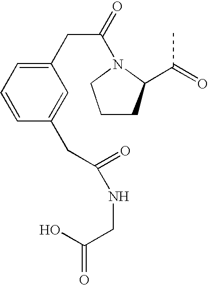 |
| | 391 |
| 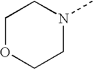 | 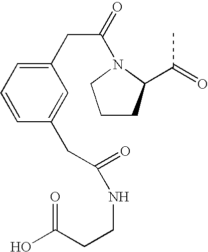 |
| | 392 |
| 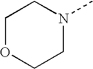 | 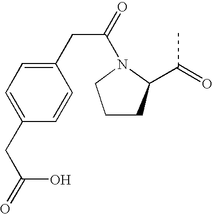 |

Other alpha4 integrin small molecule antagonists include those listed in the following table.

TABLE 2

| R | R′-compound no. |
|---|---|
| | 393 |
| 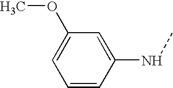 | 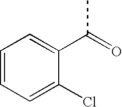 |
| | 394 |
| 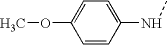 | 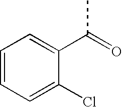 |
| | 395 |
| 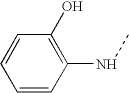 | 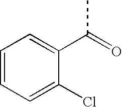 |
| | 396 |
| 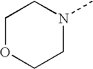 | 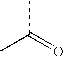 |
| | 397 |
| 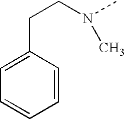 | 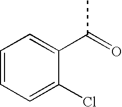 |
| | 398 |
| 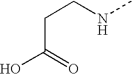 | 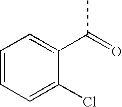 |
| | 399 |
| 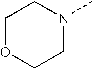 | 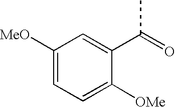 |
| | 400 |
| 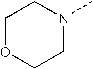 | 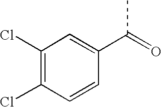 |
| | 401 |
| 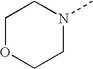 | 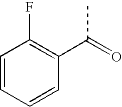 |
| | 402 |
| 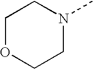 | 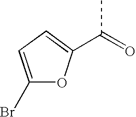 |
| | 403 |
| 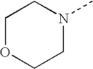 | 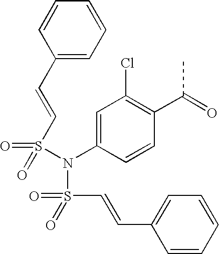 |
| | 404 |
| 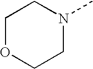 | 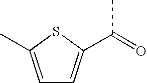 |
| | 405 |
| 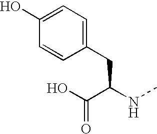 | 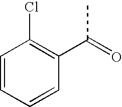 |
| | 406 |
| 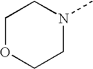 | 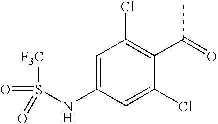 |
| | 407 |
| 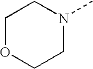 | 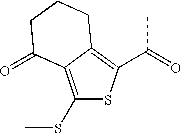 |
| | 408 |
| 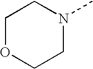 | 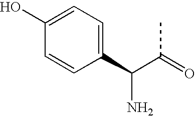 |
| | 409 |
| 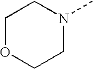 | 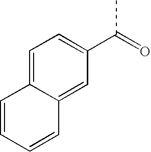 |
| | 410 |
| 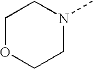 | 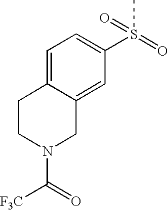 |
| | 411 |
| 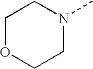 | 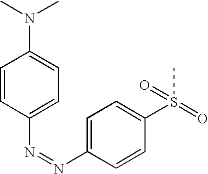 |
| | 412 |
| 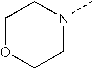 | 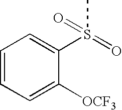 |
| | 413 |
| 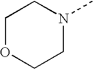 | 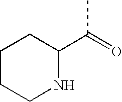 |
| | 414 |
| 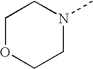 | 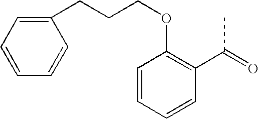 |
| | 415 |
| 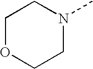 | 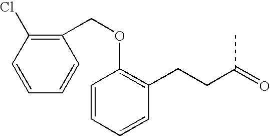 |
| | 416 |
| 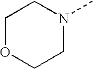 | 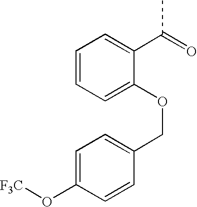 |

d. Specific Alpha4 Integrin Antagonist Small Molecules

Particular and representative compounds for alpha4 integrin small molecule antagonists are listed in the following Table 3:

TABLE 3

| Structure | Compound number |
|---|---|
| 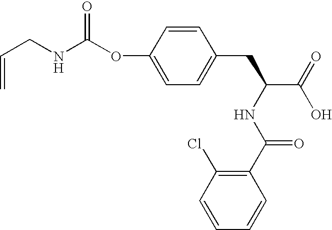 | 001 |
| 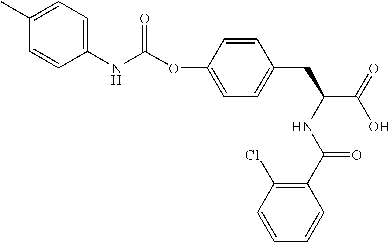 | 002 |
| 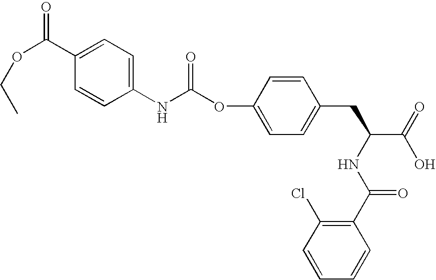 | 003 |
| 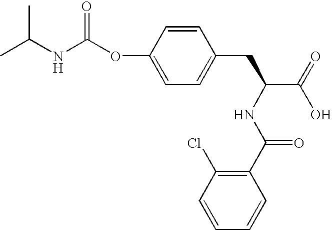 | 004 |
| 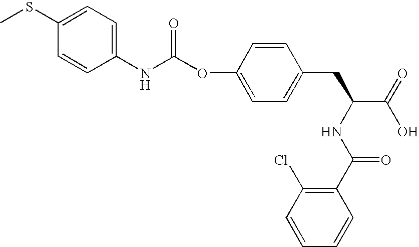 | 005 |
| 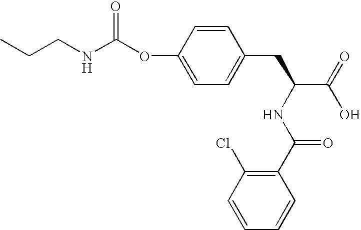 | 006 |
| 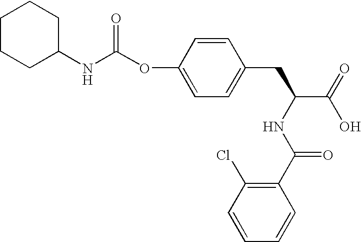 | 007 |
| 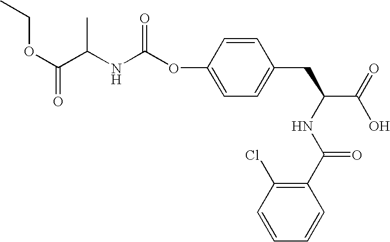 | 008 |
| 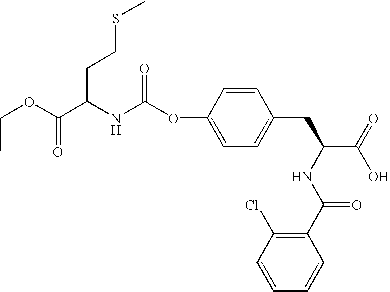 | 009 |
| 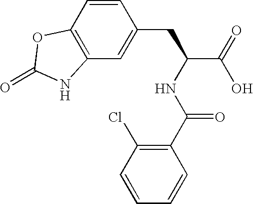 | 010 |
| 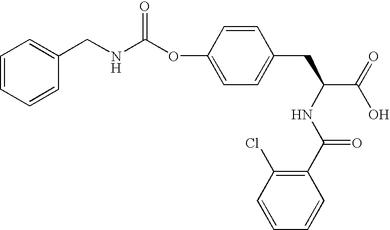 | 011 |
| 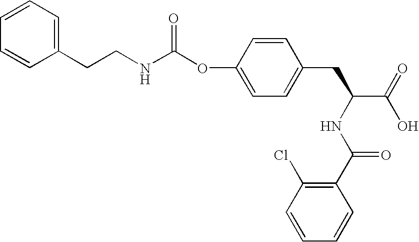 | 012 |
| 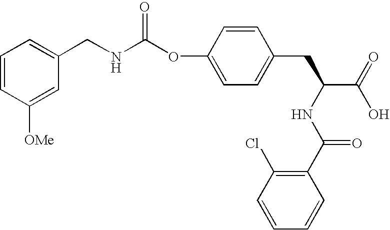 | 013 |
| 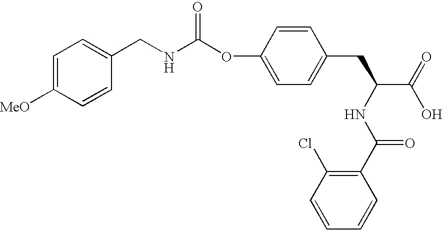 | 014 |
| 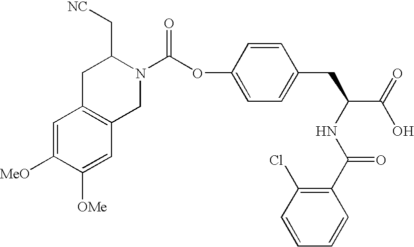 | 015 |
| 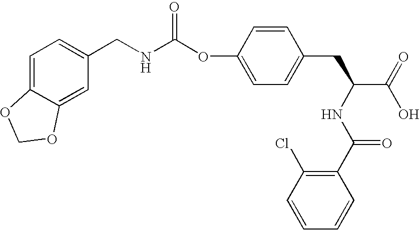 | 016 |
| 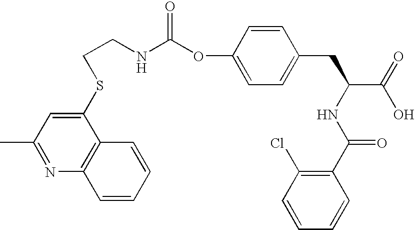 | 017 |
| 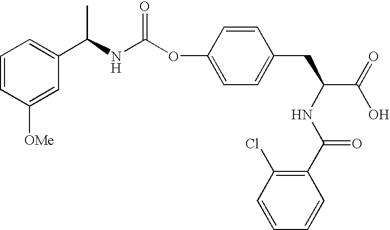 | 018 |
| 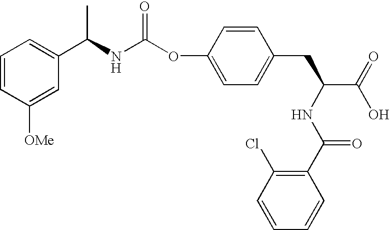 | 019 |
| 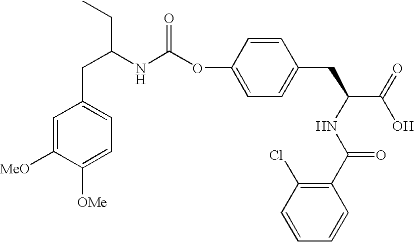 | 020 |
| 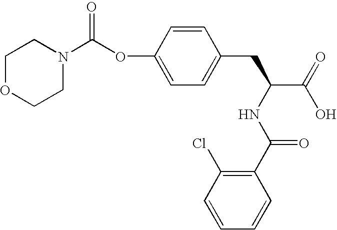 | 021 |
| 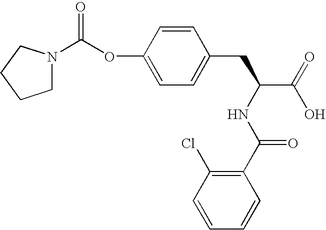 | 022 |
| 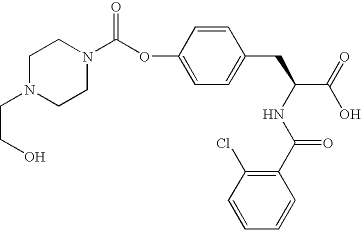 | 023 |
| 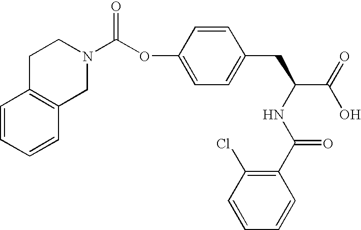 | 024 |
| 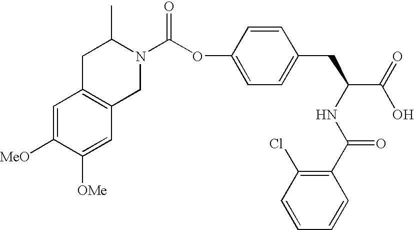 | 025 |
| 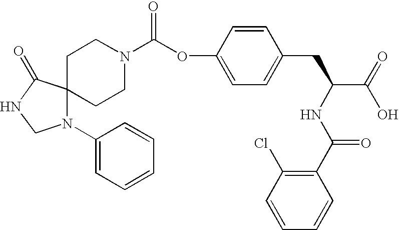 | 026 |
| 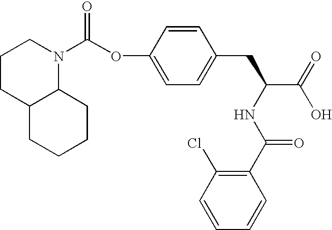 | 027 |
| 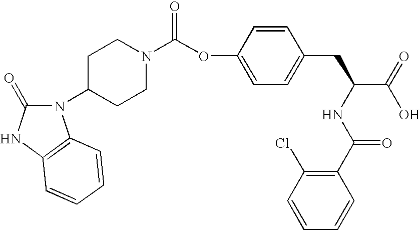 | 028 |
| 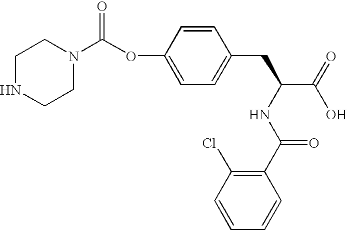 | 029 |
| 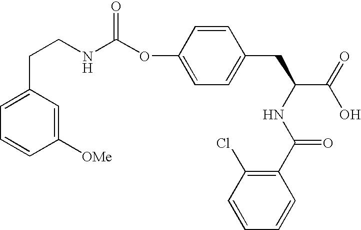 | 030 |
| 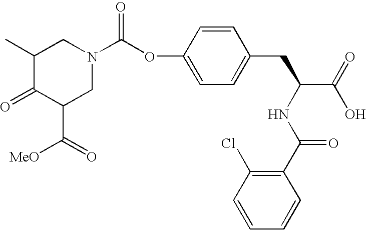 | 031 |
| 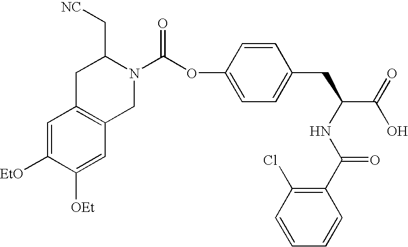 | 032 |
| 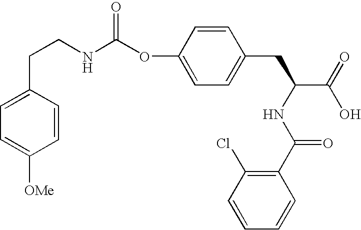 | 033 |
| 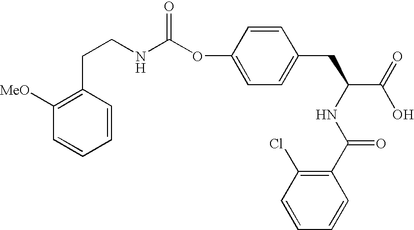 | 034 |
| 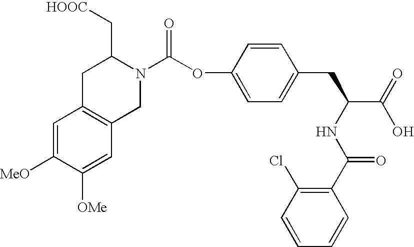 | 035 |
| 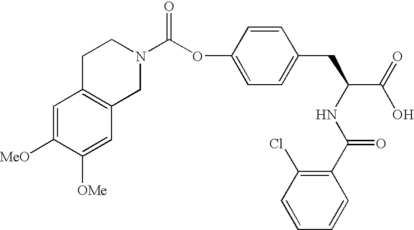 | 036 |
| 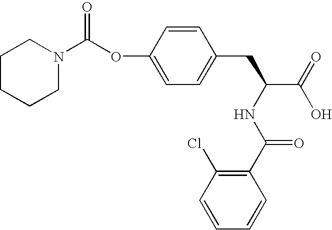 | 037 |
| 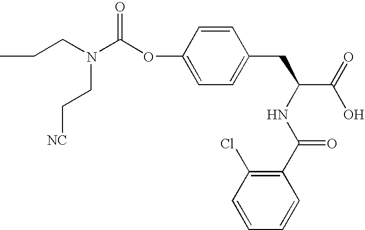 | 038 |
| 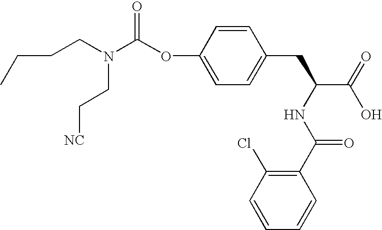 | 039 |
| 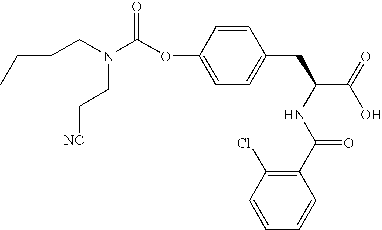 | 040 |
| 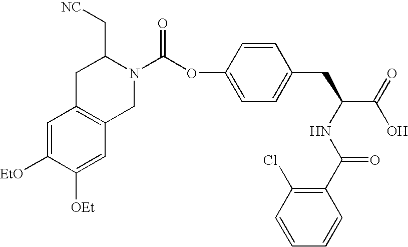 | 041 |
| 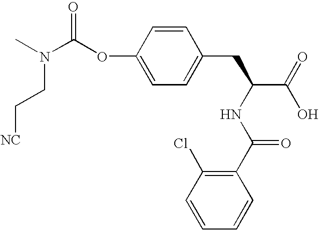 | 042 |
| 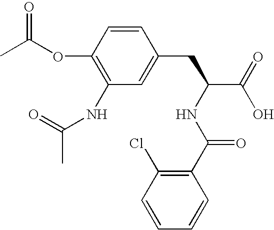 | 043 |
| 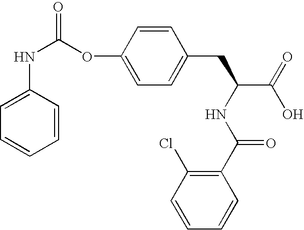 | 044 |
| 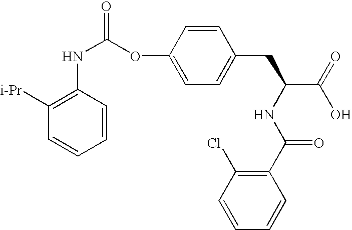 | 045 |
| 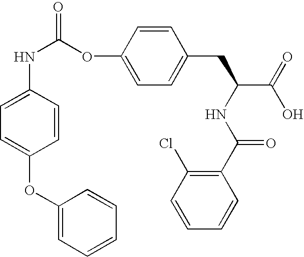 | 046 |
| 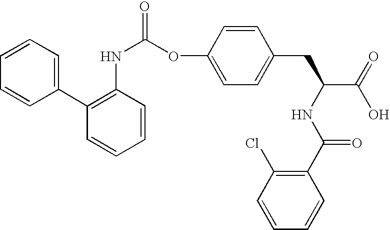 | 047 |
| 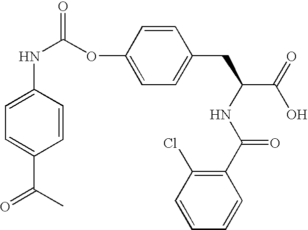 | 048 |
| 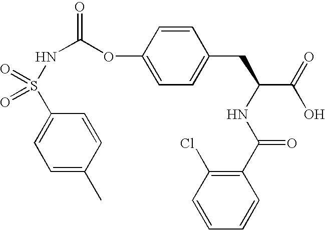 | 049 |
| 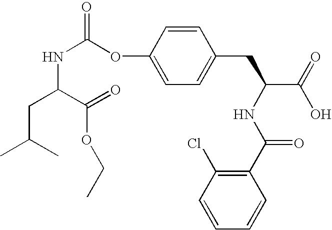 | 050 |
| 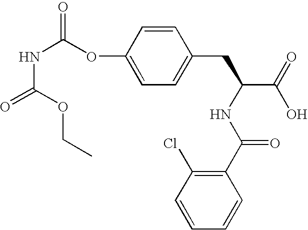 | 051 |
| 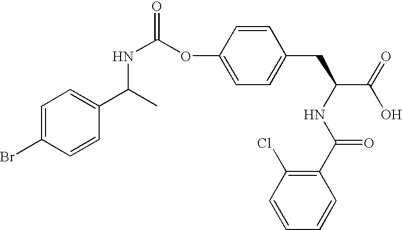 | 052 |
| 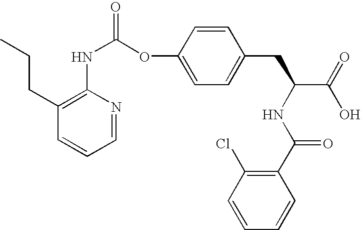 | 053 |
| 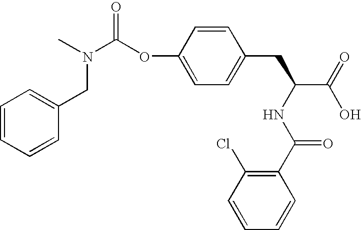 | 054 |
| 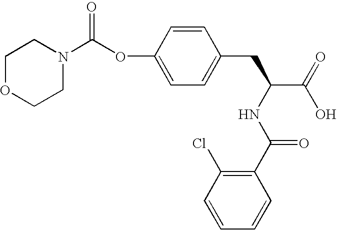 | 055 |
| 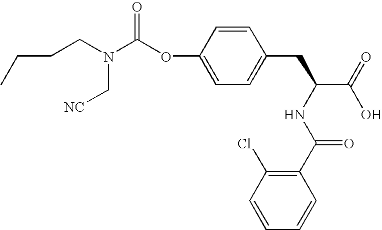 | 056 |
| 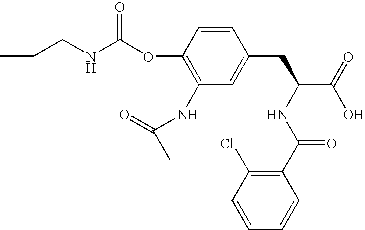 | 057 |
| 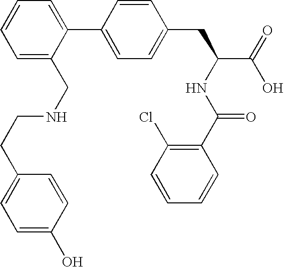 | 058 |
| 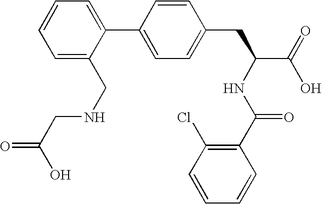 | 059 |
| 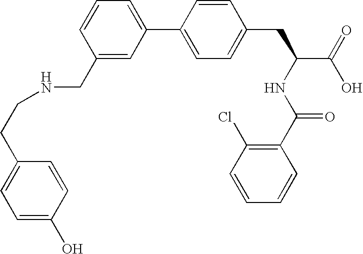 | 060 |
| 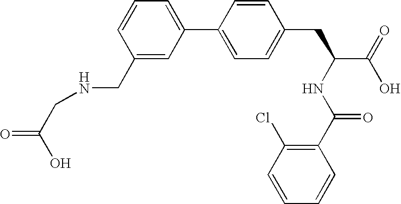 | 061 |
| 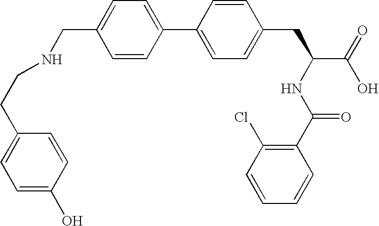 | 062 |
| 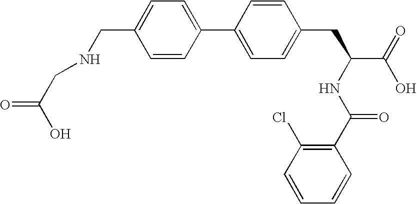 | 063 |
| 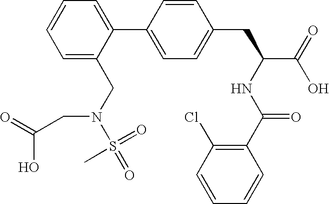 | 064 |
| 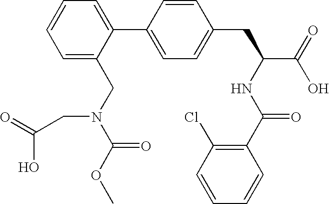 | 065 |
| 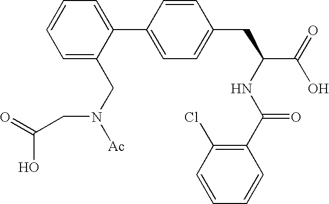 | 066 |
| 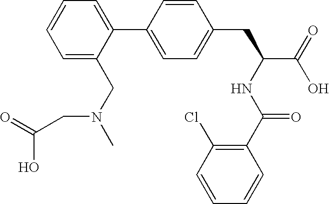 | 067 |
| 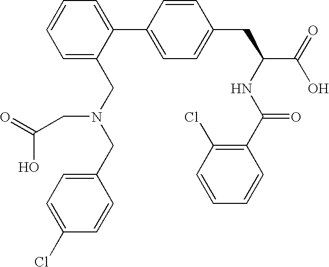 | 068 |
| 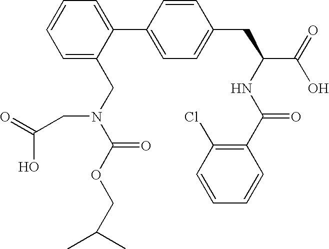 | 069 |
| 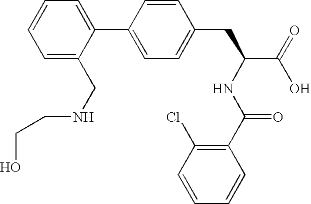 | 070 |
| 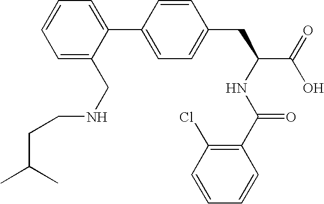 | 071 |
| 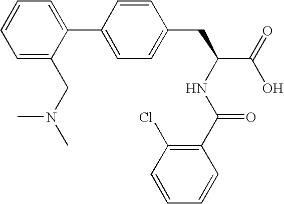 | 072 |
| 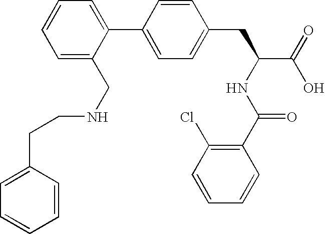 | 073 |
| 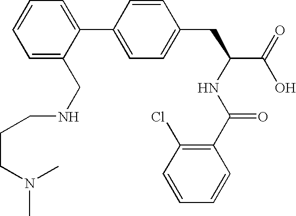 | 074 |
| 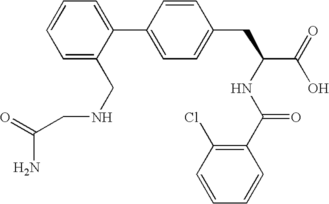 | 075 |
| 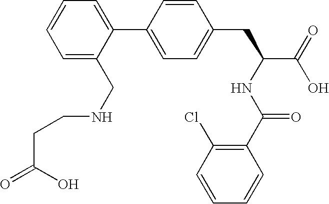 | 076 |
| 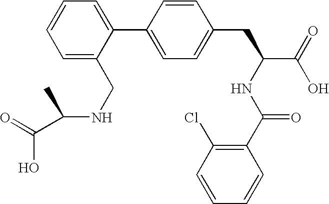 | 077 |
| 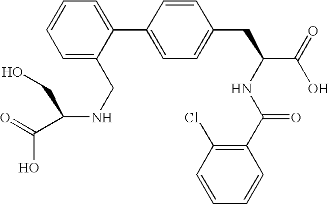 | 078 |
| 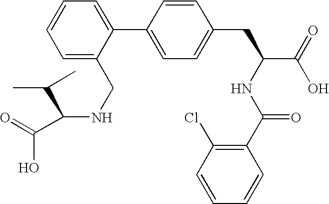 | 079 |
| 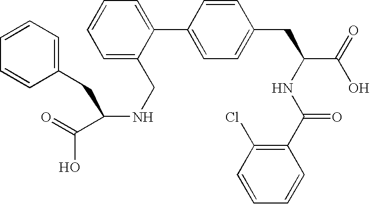 | 080 |
| 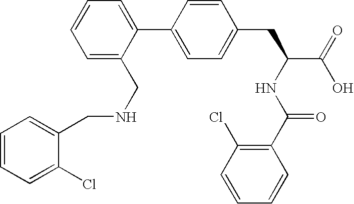 | 081 |
| 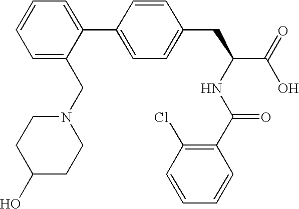 | 082 |
| 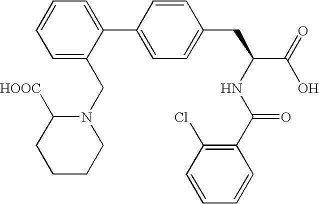 | 083 |
| 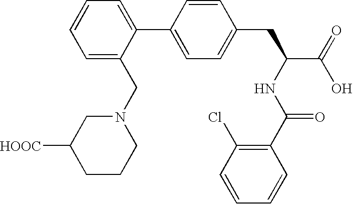 | 084 |
| 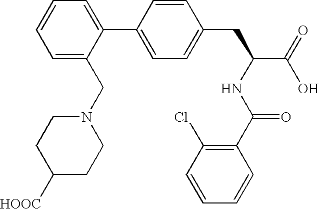 | 085 |
| 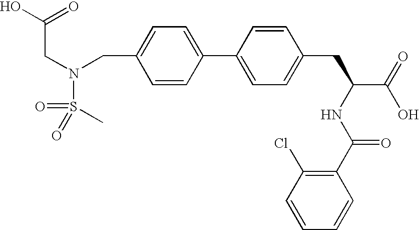 | 086 |
| 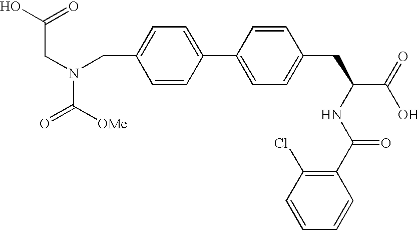 | 087 |
| 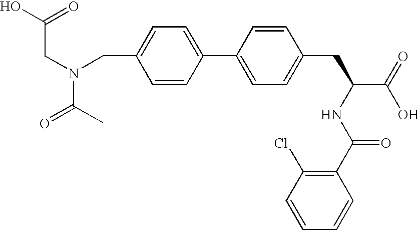 | 088 |
| 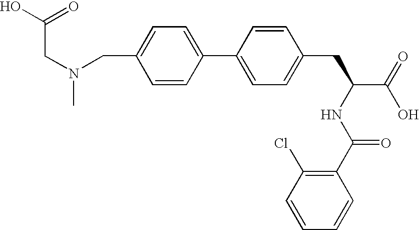 | 089 |
| 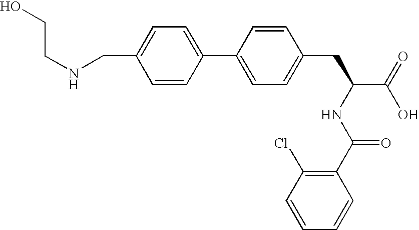 | 090 |
| 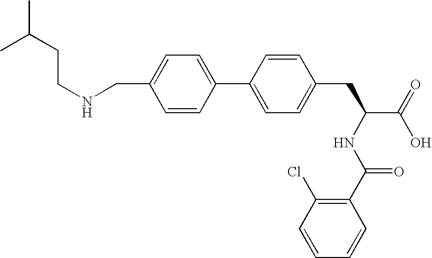 | 091 |
| 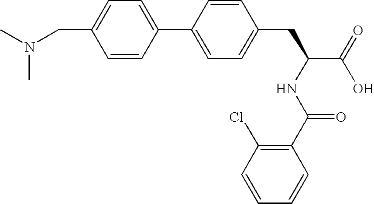 | 092 |
| 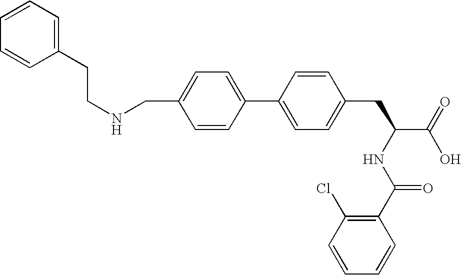 | 093 |
| 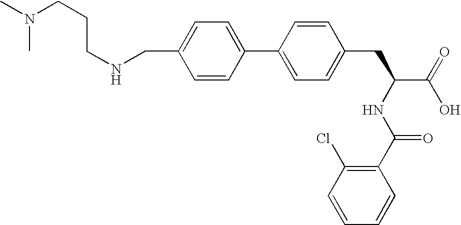 | 094 |
| 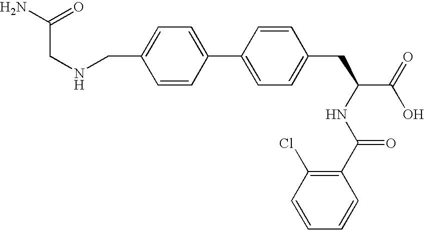 | 095 |
| 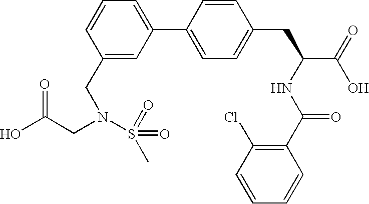 | 096 |
| 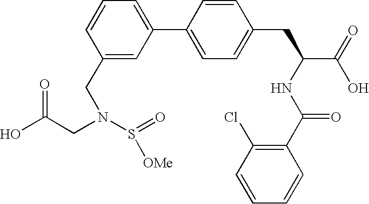 | 097 |
| 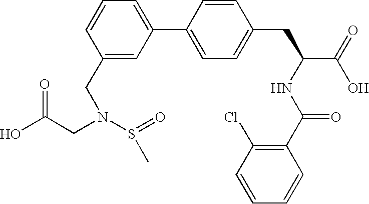 | 098 |
| 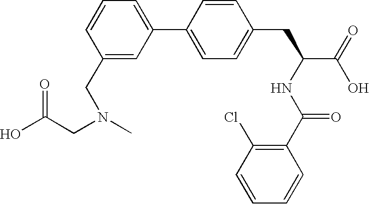 | 099 |
| 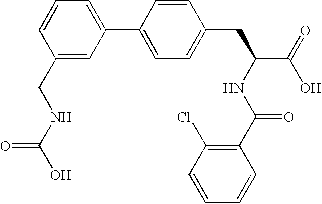 | 100 |
| 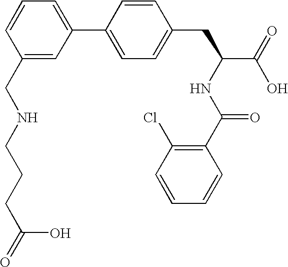 | 101 |
| 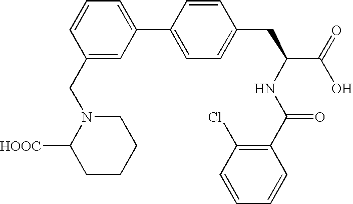 | 102 |
| 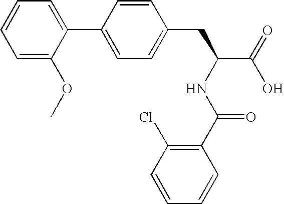 | 103 |
| 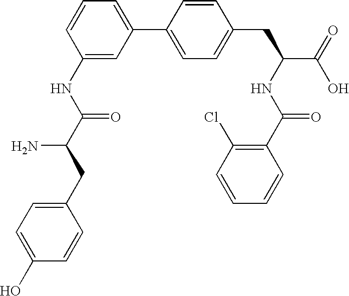 | 104 |
| 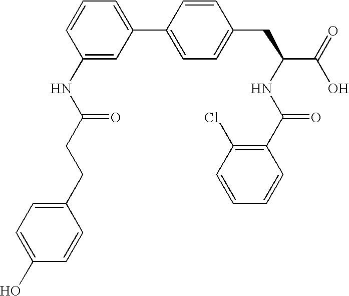 | 105 |
| 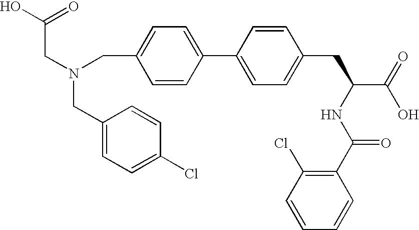 | 106 |
| 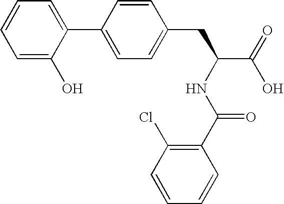 | 107 |
| 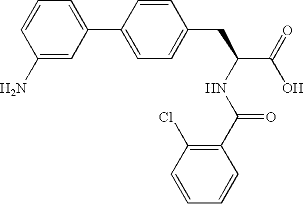 | 108 |
| 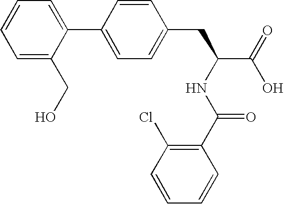 | 109 |
| 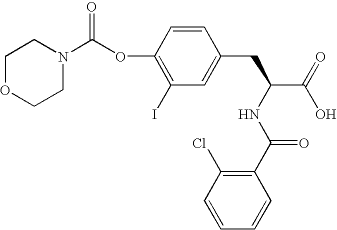 | 111 |
| 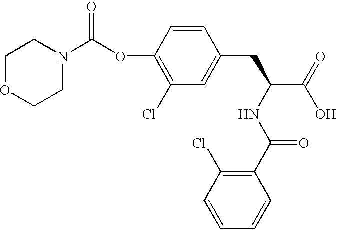 | 112 |
| 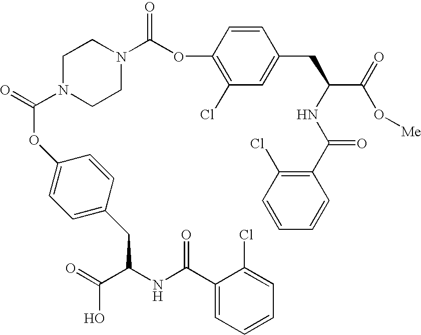 | 113 |
| 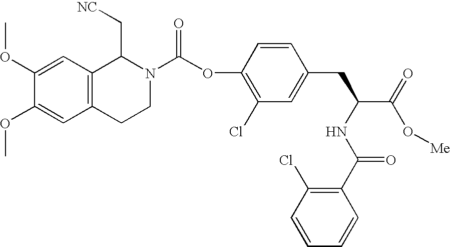 | 114 |
| 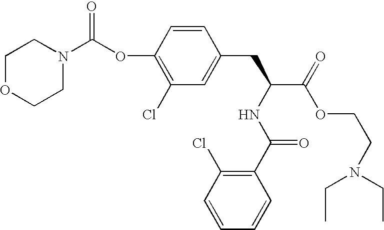 | 115 |
| 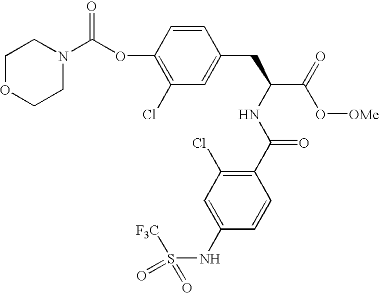 | 116 |
| 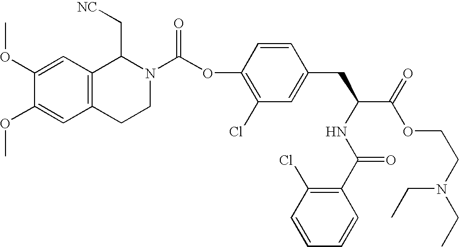 | 117 |
| 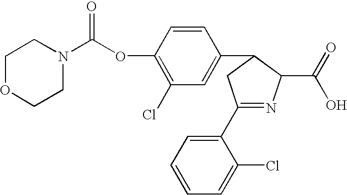 | 118 |
| 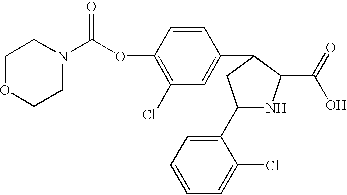 | 119 |
| 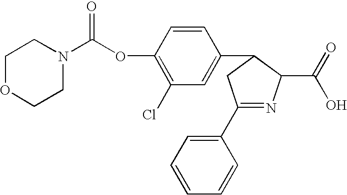 | 120 |
| 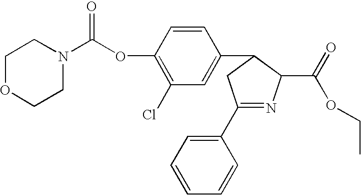 | 121 |
| 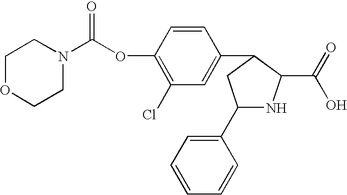 | 122 |
| 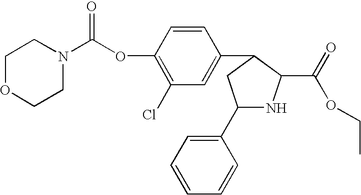 | 123 |
| 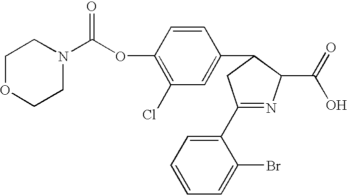 | 124 |
| 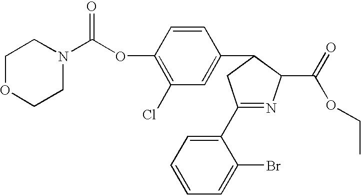 | 125 |
| 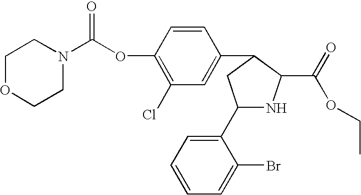 | 126 |
| 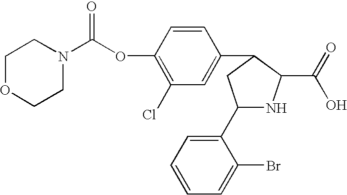 | 127 |
| 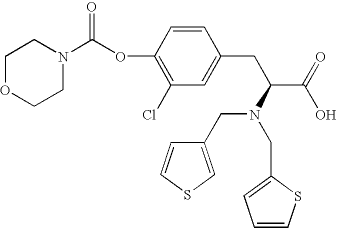 | 128 |

D. AlphaL Integrin

The term “alphaL integrin,” when used herein, refers to a heterodimer comprising an alphaL subunit and a beta subunit. One example of an alphaL integrin contains alphaLbeta2 subunits (LFA-1 or LFA-1 integrin). Examples of the biological activities of an alphaL integrin include any one or combination of the following activities: (1) binding to a ligand of LFA-I (e.g., any one of CD54 (ICAM-1), CD102 (ICAM-2), CD50 (ICAM-3), CD242 (ICAM-4), and ICAM-5 (telencephalin), and (2) promoting attachment of B lymphocytes to an organ or to an immobilized spleen, or to lymph node cells.

1. Ligands of AlphaL Integrin

According to one embodiment, the ligand of alphaLbeta2 (LFA-1) is ICAM-1 (CD-54). An example of a human ICAM-1 (CD-54) polypeptide sequence is shown below (SWISSPROT Accession No. P05362):

| [SEQ ID NO: 22] | |
|---|---|
| 1 | mapssprpal pallvllgal fpgpgnaqts vspskvilpr ggsvlvtcst scdqpkllgi |
| 61 | etplpkkell lpgnnrkvye lsnvqedsqp mcysncpdgq staktfltvy wtpervelap |
| 121 | lpswqpvgkn ltlrcqvegg apranltvvl lrgekelkre pavgepaevt ttvlvrrdhh |
| 181 | ganfscrtel dlrpqglelf entsapyqlq tfvlpatppq lvsprvlevd tqgtvvcsld |
| 241 | glfpvseaqv hlalgdqrln ptvtygndsf sakasvsvta edegtqrltc avilgnqsqe |
| 301 | tlqtvtiysf papnviltkp evsegtevtv kceahprakv tlngvpaqpl gpraqlllka |
| 361 | tpedngrsfs csatlevagq lihknqtrel rvlygprlde rdcpgnwtwp ensqqtpmcq |
| 421 | awgnplpelk clkdgtfplp igesvtvtrd legtylcrar stqgevtrev tvnvlsprye |
| 481 | iviitvvaaa vimgtaglst ylynrqrkik kyrlqqaqkg tpmkpntqat pp |

Residues 1 to 27 comprise a signal sequence, residues 28 to 480 comprise an extracellular domain, residues 481 to 503 comprise a transmembrane domain, and residues 504 to 542 comprise a cytoplasmic domain.

According to one embodiment, the ligand of alphaL integrin, for example, alphaLbeta2 (LFA-1) is ICAM-2 (CD-102). An example of a human ICAM-2 (CD-102) polypeptide sequence is shown below (Genbank Accession No. CAG46633, EMBL Accession No. CR541834.1):

| [SEQ ID NO: 23] | |
|---|---|
| 1 | mssfgyrtlt valftliccp gsdekvfevh vrpkklavep kgslevncst tcnqpevggl |
| 61 | etsldkilld eqaqwkhylv snishdtvlq chftcsgkqe smnsnvsvyq pprqviltlq |
| 121 | ptlvavgksf tiecrvptve pldsltlflf rgnetlhyet fgkaapapqe atatfnstad |
| 181 | redghrnfsc lavldlmsrg gnifhkhsap kmleiyepvs dsqmviivtv vsvllslfvt |
| 241 | svllcfifgq hlrqqrmgty gvraawrrlp qafrp |

Residues 1 to 21 comprise a signal sequence, residues 22 to 224 comprise an extracellular domain, residues 224 to 248 comprise a transmembrane domain, and residues 249 to 275 comprise a cytoplasmic domain.

According to one embodiment, the ligand of alphaL integrin, for example alphaLbeta2 (LFA-1) is ICAM-3 (CD-50). An example of a human ICAM-3 (CD-50) polypeptide sequence is shown below (SWISSPROT Accession No. P32942):

| [SEQ ID NO: 24] | |
|---|---|
| 1 | matmvpsvlw pracwtllvc clltpgvqgq efllrvepqn pvlsaggslf vncstdcpss |
| 61 | ekialetsls kelvasgmgw aafnlsnvtg nsrilcsvyc ngsqitgssn itvyglperv |
| 121 | elaplppwqp vgqnftlrcq veggsprtsl tvvllrweee lsrqpaveep aevtatvlas |
| 181 | rddhgapfsc rteldmqpqg lglfvntsap rqlrtfvlpv tpprlvaprf levetswpvd |
| 241 | ctldglfpas eaqvylalgd qmlnatvmnh gdtltatata taradqegar eivcnvtlgg |
| 301 | errearenlt vfsflgpivn lseptahegs tvtvscmaga rvqvtldgvp aaapgqpaql |
| 361 | qlnatesddg rsffcsatle vdgeflhrns svqlrvlygp kidratcpqh lkwkdktrhv |
| 421 | lqcqargnpy pelrclkegs srevpvgipf fvnvthngty qcqasssrgk ytlvvvmdie |
| 481 | agsshfvpvf vavlltlgvv tivlalmyvf rehqrsgsyh vreestylpl tsmqpteamg |
| 541 | eepsrae |

Residues 1 to 29 comprise a signal sequence, residues 30 to 485 comprise an extracellular domain, residues 486 to 510 comprise a transmembrane domain, and residues 511 to 547 comprise a cytoplasmic domain.

According to one embodiment, the ligand of alphaL integrin, for example alphaLbeta2 (LFA-1) is ICAM-4. An example of a human ICAM-4 polypeptide sequence is shown below (SWISSPROT Accession No. Q14773):

| [SEQ ID NO: 25] | |
|---|---|
| 1 | mgslfplsll fflaaaypgv gsalgrrtkr aqspkgspla psgtsvpfwv rmspefvavq |
| 61 | pgksvqlncs nscpqpqnss lrtplrqgkt lrgpgwvsyq lldvrawssl ahclvtcagk |
| 121 | trwatsrita ykpphsvile ppvlkgrkyt lrchvtqvfp vgylvvtlrh gsrviysesl |
| 181 | erftgldlan vtltyefaag prdfwqpvic harlnldglv vrnssapitl mlawspapta |
| 241 | lasgsiaalv gilltvgaay lckclamksq a |

Residues 1 to 22 comprise a signal sequence, residues 23 to 240 comprise an extracellular domain, residues 241 to 261 comprise a transmembrane domain, and residues 262 to 271 comprise a cytoplasmic domain.

According to one embodiment, the ligand of alphaL integrin, for example alphaLbeta2 (LFA-1) is ICAM-5. An example of a human ICAM-5 polypeptide sequence is shown below (SWISSPROT Accession No. Q9UMF0):

| [SEQ ID NO: 26] | |
|---|---|
| 1 | mpgpspglrr allglwaalg lglfglsavs qepfwadlqp rvafverggs lwlncstncp |
| 61 | rpergglets lrrngtqrgl rwlarqlvdi repetqpvcf frcarrtlqa rglirtfqrp |
| 121 | drvelmplpp wqpvgenftl scrvpgagpr asltltllrg aqelirrsfa gepprargav |
| 181 | ltatvlarre dhganfscra eldlrphglg lfenssapre lrtfslspda prlaaprlle |
| 241 | vgserpvsct ldglfpasea rvylalgdqn lspdvtlegd afvatatata saeqegarql |
| 301 | vcnvtlggen retrenvtiy sfpaplltls epsvsegqmv tvtcaagaqa lvtlegvpaa |
| 361 | vpgqpaqlql natenddrrs ffcdatldvd getliknrsa elrvlyaprl ddsdcprswt |
| 421 | wpegpeqtlr ceargnpeps vhcarsdgga vlalgllgpv tralsgtyrc kaandqgeav |
| 481 | kdvtltveya paldsvgcpe ritwlegtea slscvahgvp ppdvicvrsg elgaviegll |
| 541 | rvarehagty rceatnprgs aaknvavtve ygprfeepsc psnwtwvegs grlfscevdg |
| 601 | kpqpsvkcvg sggttegvll plappdpspr apriprvlap giyvcnatnr hgsvaktvvv |
| 661 | saesppemde stcpshqtwl egaeasalac aargrpspgv rcsregipwp eqqrvsreda |
| 721 | gtyhcvatna hgtdsrtvtv gveyrpvvae laasppggvr pggnftltcr aeawppaqis |
| 781 | wrappralni glssnnstls vagamgshgg eyecartnah grharritvr vagpwlwvav |
| 841 | ggaaggaall aagaglafyv qstackkgey nvqeaessge avclngaggg aggaagaegg |
| 901 | peaaggaaes paegevfaiq ltsa |

Residues 1 to 31 comprise a signal sequence, residues 32 to 835 comprise an extracellular domain, residues 836 to 856 comprise a transmembrane domain, and residues 857 to 924 comprise a cytoplasmic domain.
2. AlphaL Integrin Antagonist

The term “alphaL integrin antagonist,” as used herein, is used in the broadest sense, and includes any molecule that partially or fully blocks a biological activity of an alphaL integrin. According to one embodiment, an alphaL integrin antagonist partially or fully blocks the interaction between an alphaL integrin and its ligand and any one or combination of the following events: (1) promotes the circulation of B lymphocytes in mammals and (2) partially or fully blocks, inhibits, or neutralizes native sequence alphaL integrin signaling. According to one embodiment, the alphaL integrin antagonist inhibits B cell attachment to the spleen or to lymph nodes. In a more specific embodiment, the alphaL integrin antagonist inhibits B cell attachment to the marginal zone and/or germinal center of the spleen and lymph nodes.

Antagonists of αL integrin and α4 integrin can be used alone, or used together, simultaneously or sequentially, to promote the circulation of B lymphocytes in mammals. In one embodiment, multiple different antagonists of αL integrin and α4 integrin can be used alone, or used together, simultaneously or sequentially, to promote the circulation of B lymphocytes in mammals. The antagonist can bind to the alphaL integrin, to the alphaL subunit, or to a ligand of the alphaL integrin.

Suitable alphaL integrin antagonists include any compound that inhibits the interaction of alpha1 integrin and a ligand, such as ICAM-1 (CD-54). The alphaL integrin antagonist may be a small molecule, peptide, protein, immunoadhesin, an anti-alphaL antibody, or a fragment thereof, for example, and may be, for example, an alphaLbeta2 (LFA-1) antagonist. These terms refer to antagonists directed against either the alphaL subunit (CD11a), or the beta subunit, for example, beta2 (CD18), or both. Preferably, the antagonist is directed to or binds to the alpha L (CD11a) subunit or the alphaL integrin as a unit.

3. Antibody Antagonists of AlphaL Integrin

The alphaL antagonist can be an antibody that binds the alphaL integrin, the alphaL subunit, or binds a ligand of the alpha L integrin, for example. Antibodies that bind the alphaL subunit (CD11a) include, for example, the antibody MHM24 (Hildreth et al., 1983, Eur. J. Immunol. 13:202-208), the IgG1 antibody R3.1 (Boehringer Ingelheim Pharmaceuticals, Inc., Ridgefield, Conn.), 25-3 (or 25.3), an IgG1 available from Immunotech, France, as cited in Olive et al., 1986, In: Feldmann, ed., Human T cell Clones. A new Approach to Immune Regulation, Clifton, N.J., Humana, p. 173), KBA (IgG2a) (Nishimura et al., 1987, Cell. Immunol. 107:32; Nishimura et al., 1985, ibid 94:122), M7/15 (IgG2b) (Springer et al., 1982, Immunol. Rev. 68:171), IOT16 (Vermot Desroches et al., 1991, Scand. J. Immunol. 33:277-286), SPVL7 (Vermot Desroches et al., supra), and M17/4 (IgG2a), available from ATCC with hybridoma Accession #TIB-217. A preferred anti-CD11a antibody is the humanized antibody efalizumab, (Raptivam; Genentech, CA). Other preferred anti-CD11a antibodies include the humanized antibodies described in U.S. Pat. No. 6,037,454. It is also generally preferred that the anti-CD11a antibodies are not T-cell depleting antibodies, that is, that the administration of the anti-CD11a antibody does not reduce the level of circulating T-cells.

In one embodiment, the humanized anti-CD11a antibody is one that comprises the

VL sequence of

(SEQ ID NO. 49)

DIQMTQSPSSLSASVGDRVTITCRASKTISKYLAWYQQKPGKAPKLLIYS
GSTLQSGVPSRFSGSGSGTDFTLTISSLQPEDFATYYCQQHNEYPLTFGQ
GTKVEIK,
and
the VH sequence of

(SEQ ID NO. 50)

EVQLVESGGGLVQPGGSLRLSCAASGYSFTGHWMNWVRQAPGKGLEWVGM
IHPSDSETRYNQKFKDRFTISVDKSKNTLYLQMNSLRAEDTAVYYCARGI
YFYGTTYFDYWGQGTLVTVSS;
or
In another embodiment, the anti-CD 11 a antibody
is one that comprises the MHM24 VL sequence

(SEQ ID NO. 51)

DVQITQSPSYLAASPGETISINCRASKTISKYLAWYQEKPGKTNKLLIYS
GSTLQSGIPSRFSGSGSGTDFTLTISSLEPEDFAMYYCQQHNEYPLTFGT
GTKLELK,
and
MHM24 VH sequence

(SEQ ID NO. 52)

EVQLQQPGAELMRPGASVKLSCKASGYSFTGHWMNWVRQRPGQGLEWIGM
IHPSDSETRLNQKFKDKATLTVDKSSSSAYMQLSSPTSEDSAVYYCARGI
YFYGTTYFDYWGQGTTLTVSS

Examples of antibodies that bind the beta subunit include anti-CD 18 antibodies such as MHM23 (Hildreth et al., supra), M18/2 (IgG2a) (Sanches-Madrid et al., 1983, J. Exp. Med. 158:586), H52 (Fekete et al., 1990, J. Clin. Lab Immunol. 31:145-149), Mas 191c (Vermot Desroches et al., supra), IOT18 (Vermot Desroches et al., supra), 60.3 (Taylor et al., 1988, Clin. Exp. Immunol. 71:324-328), and 60.1 (Campana et al., 1986, Eur. J. Immunol. 16:537-542). See also U.S. Pat. No. 5,997,867.

Other examples of suitable alphaLbeta2 (LFA-1) binding molecules, including antibodies, are described, for example, in Hutchings et al., supra, WO 98/51343, WO 91/18011, WO 91/16928, WO 91/16927, Can. Pat. Appln. 2,008,368, WO 90/15076, WO 90/10652, WO 90/13281, WO 93/06864, WO 93/21953, EP 387,668, EP 379,904, EP 346,078, U.S. Pat. No. 5,932,448, U.S. Pat. No. 5,622,700, U.S. Pat. No. 5,597,567, U.S. Pat. No. 5,071,964, U.S. Pat. No. 5,002,869, U.S. Pat. No. 5,730,983, Australian Pat. Appln. 8815518, FR 2700471A, EP 289,949, EP 362526, and EP 303,692.

AlphaLbeta2 (LFA-1) antagonists also include antibodies that inhibit the interaction of alphaLbeta2 (LFA-1) and its receptor, including, for example, antibodies against one or more of ICAM-1, ICAM-2, ICAM-3, ICAM4, and ICAM-5. Such antibodies are commercially available, for example, the anti-ICAM-1 antibodies enlimomab (BIRR-1) and 1A6, available from Boehringer Ingelheim Pharmaceuticals (Ridgefield, Conn.) and Perlan Therapeutics Inc., (San Diego, Calif.), respectively; and the anti-ICAM-3 antibody ICM3, available from ICOS Corp. (Bothell, Wash.).

4. Immunoadhesin Antagonists of AlphaL Integrin

According to yet another embodiment, the integrin antagonist is an immunoadhesin. An example of such an immunoadhesin is one that comprises a soluble portion of a ligand of alphaL integrin that binds to alphaL, for example, the ligand binding domain or the extracellular domain of a ligand of the alphaL integrin, such as ICAM-1, ICAM-2, ICAM-3, ICAM-4, and ICAM 5, for example.

The binding domains of ICAM ligands are known. ICAM-1 binds to LFA-1 (CD11a) within Domain 1 (residues 41-103 according to the Universal Protein Resource catalog (UniProt)). See, for example, Bella et al., 1998, Proc. Natl. Acad. Sci. USA, 95: 4140-4145. ICAM-2 binds to LFA-1 (CD11a) and MAC-1 (CD11b) within Domain I (residues 41-98 according to UniProt). See, for example, Bella et al., 1998, supra; and Hermand et al., 2000, J. Biol. Chem., 275: 26002-26010. ICAM-3 binds to LFA-1 (CD11a) within Domain 1 (residues 46-103 according to UniProt) and does not bind to MAC-1 (CD11b). See, for example, Bella et al., 1998, supra; and Hermand et al., 2000, supra). ICAM-4 binds to LFA-1 (CD11a) within Domain 1 (residues 62-124 according to UniProt) (Hermand et al., 2000, supra). ICAM-5 binds to LFA-1 (CD11a) within Domain 1 (residues 48-130 according to UniProt). See, for example, Tian et al., 2000, Eur. J. Immunol., 30: 810-818.

The integrin or integrin subunit antagonists of the invention specifically include proteins, in particular, antibodies and functional fragments thereof, peptides, immunoadhesins and small molecules. The antibodies can be humanized, human, or chimeric forms, or a fragment of these.

5. Small Molecule Antagonists of AlphaL Integrin

According to one embodiment, the alphaL integrin antagonist is a small molecule. Examples of small molecules that are alphaL integrin antagonists include those disclosed in published PCT applications WO 99/49856, and WO 02/059114. According to one embodiment, the antagonist is any one of the small molecules recited in WO 02/059114 having the Formula (IX) as described in detail below. According to another embodiment, the antagonist is any one of the small molecules recited in WO 02/059114 and shown in Table 4 (i.e., compounds numbered 4, 5, 35, 17, 10, 12, 13, 14, 41, 44, 6, 15, 36, 37, 38, 40, 42, 9, 3 and 51).

a. Formula XI

B cell mobilizing agents also include alphaL integrin antagonists including the alphaL integrin antagonist compounds of formula XI: 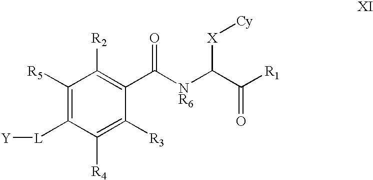
where
   Cy is a non-aromatic carbocycle or heterocycle optionally substituted with hydroxyl (—OH), mercapto (—SH), thioalkyl, halogen (e.g. F, Cl, Br, l), oxo (═O), thio (═S), amino, aminoalkyl, amidine (—C(NH)—NH₂), guanidine (—NH₂—C(NH)—NH₂), nitro, alkyl, alkoxy or acyl;
   X is a divalent hydrocarbon chain optionally substituted with hydroxyl, mercapto, halogen, amino, aminoalkyl, nitro, oxo or thio and optionally interrupted with N, O, S, SO or SO₂;
   Y is a carbocycle or heterocycle optionally substituted with hydroxyl, mercapto, halogen, oxo, thio, a hydrocarbon, a halo-substituted hydrocarbon, amino, amidine, guanidine, cyano, nitro, alkoxy or acyl;
   L is a bond or a divalent hydrocarbon optionally having one or more carbon atoms replaced with N, O, S, SO or SO₂, optionally substituted with hydroxyl, halogen oxo or thio; or three carbon atoms of the hydrocarbon are replaced with an amino acid residue;
   R₁ is H, OH, amino, O-carbocycle or alkoxy optionally substituted with amino, a carbocycle or a heterocycle;
   R₂₋₅ are independently H, hydroxyl, mercapto, halogen, cyano, amino, amidine, guanidine, nitro or alkoxy; or R₃ and R₄ together form a fused carbocycle or heterocycle optionally substituted with hydroxyl, halogen, oxo, thio, amino, amidine, guanidine or alkoxy;
   R₆ is H or a hydrocarbon chain optionally substituted with a carbocycle or a heterocycle; and
   salts, solvates and hydrates thereof;
      with the proviso that when Y is phenyl, R₂, R₄ and R₅ are H, R₃ is Cl and R₁ is OH then X is other than cyclohexyl;
      or a pharmaceutically acceptable salt thereof.

A, Z, Y, X₁, X₂, X₃ and X₄ are as defined above, both generally and preferably.

Cy can be a 3-5 member ring. In another embodiment, Cy can be a 5- or 6-member non-aromatic heterocycle optionally substituted with hydroxyl, mercapto, halogen (preferably F or Cl), oxo (═O), thio (═S), amino, amidine, guanidine, nitro, alkyl, or alkoxy. Cy can be a 5-member non-aromatic heterocycle optionally substituted with hydroxyl, oxo, thio, C₁, C₁₄ alkyl (preferably methyl), or C₁₋₄ alkanoyl (preferably acetyl, propanoyl or butanoyl). The non-aromatic heterocycle can comprise one or heteroatoms (N, O, or S) and is optionally substituted with hydroxyl, oxo, mercapto, thio, methyl, acetyl, propanoyl or butyl. In particular embodiments the non-aromatic heterocycle comprises at least one nitrogen atom that is optionally substituted with methyl or acetyl. In a particularly preferred embodiment, the non-aromatic heterocycle is selected from the group consisting of piperidine, piperazine, morpholine, tetrahydrofuran, tetrahydrothiophene, oxazolidine, thiazolidine optionally substituted with hydroxy, oxo, mercapto, thio, alkyl or alkanoyl. In a most preferred embodiment Cy is a non-aromatic heterocycle selected from the group consisting of tetrahydrofuran-2-yl, thiazolidin-5-yl, thiazolidin-2-one-5-yl, and thiazolidin-2-thione-5-yl and cyclopropapyrrolidine. In another preferred embodiment Cy is a 3-6 member carbocycle optionally substituted with hydroxyl, mercapto, halogen, oxo, thio, amino, amidine, guanidine, alkyl, alkoxy or acyl. In a particular embodiment the carbocycle is saturated or partially unsaturated. In particular embodiments Cy is a carbocycle selected from the group consisting of cyclopropyl, cyclopropenyl, cyclobutyl, cyclobutenyl, cyclopentyl, cyclopentenyl, cyclohexyl and cyclohexenyl.

X is a C₁₋₅ divalent hydrocarbon linker optionally having one or more carbon atoms replaced with N, O, S, SO or SO₂ and optionally being substituted with hydroxyl, mercapto, halogen, amino, aminoalkyl, nitro, oxo or thio. In a preferred embodiment X will have at least one carbon atom. Replacements and substitutions may form an amide moiety (—NRC(O)— or —C(O)NR—) within the hydrocarbon chain or at either or both ends. Other moieties include sulfonamide (—NRSO₂— or —SO₂NR), acyl, ether, thioether and amine. In a particularly preferred embodiment X is the group —CH₂—NR₆—C(O)— wherein the carbonyl —C(O)— portion thereof is adjacent (i.e. covalently bound) to Cy and R₆ is alkyl i.e. methyl and more preferably H.

Y is a carbocycle or heterocycle optionally substituted with hydroxyl, mercapto, halogen, oxo, thio, a hydrocarbon, a halo-substituted hydrocarbon, amino, amidine, guanidine, cyano, nitro, alkoxy or acyl. In particular embodiment, Y is aryl or heteroaryl optionally substituted with halogen or hydroxyl. In a particularly preferred embodiment, Y is phenyl, furan-2-yl, thiophene-2-yl, phenyl substituted with a halogen (preferably Cl) or hydroxyl, preferably at the meta position.

L is a divalent hydrocarbon optionally having one or more carbon atoms replaced with N, O, S, SO or SO₂ and optionally being substituted with hydroxyl, halogen oxo, or thio; or three carbon atoms of the hydrocarbon are replaced with an amino acid residue. Preferably L is less than 10 atoms in length and more preferably 5 or less and most preferably 5 or 3 atoms in length. In particular embodiments, L is selected from the group consisting of —CH═CH—C(O)—NR₆—CH₂—, —CH₂—NR₆—C(O)—, —C(O)—NR₆—CH₂—, —CH(OH)—(CH₂)₂—, —(CH₂)₂—CH(OH)—, —(CH₂)₃—, —C(O)—NR₆—CH(R₇)—C(O)—N R—, —NR₆—C(O)—C H(R₇)—NR₆—C(O)—, —CH(OH)—CH₂—O— and —CH(OH)—CF₂—CH₂— wherein each R₆ is independently H or alkyl and R₇ is an amino acid side chain. Preferred amino acid side chains include non-naturally occurring side chains such as phenyl or naturally occurring side chains. Preferred side chains are those from Phe, Tyr, Ala, Gin and Asn. In a preferred embodiments L is —CH═CH—C(O)—NR₆—CH₂— wherein the —CH═CH— moiety thereof is adjacent (i.e. covalently bound) to Y. In another preferred embodiment, L is —CH₂—NR₆—C(O)— wherein the methylene moiety (—CH₂—) thereof is adjacent to Y.

R₁ is H, OH, amino, O-carbocycle or alkoxy optionally substituted with amino, a carbocycle or a heterocycle. In a preferred embodiment, R₁ is H, phenyl or C₁₋₄ alkoxy optionally substituted with a carbocycle such as phenyl. In a particular embodiment R₁ is H. In another particular embodiment R₁ is methoxy, ethoxy, propyloxy, butyloxy, isobutyloxy, s-butyloxy, t-butyloxy, phenoxy or benzyloxy. In yet another particular embodiment R₁ is NH₂. In a particularly preferred embodiment R₁ is ethoxy. In another particularly preferred embodiment R₁ is isobutyloxy. In another particularly preferred embodiment R₁ is alkoxy substituted with amino, for example 2-aminoethoxy, N-morpholinoethoxy, N,N-dialkyaminoethoxy, quaternary ammonium hydroxy alkoxy (e.g. trimethylammoniumhydroxyethoxy).

R₂₋₅ are independently H, hydroxyl, mercapto, halogen, cyano, amino, amidine, guanidine, nitro or alkoxy; or R₃ and R₄ together form a fused carbocycle or heterocycle optionally substituted with hydroxyl, halogen, oxo, thio, amino, amidine, guanidine or alkoxy. In a particular embodiment R₂ and R₃ are independently H, F, Cl, Br or I. In another particular embodiment, R₄ and R₅ are both H. In another particular embodiment, one of R₂ and R₃ is a halogen while the other is hydrogen or a halogen. In a particularly preferred embodiment, R₃ is Cl while R₂, R₄ and R₅ are each H. In another particularly preferred embodiment, R₂ and R₃ are both Cl while R₄ and R₅ are both H.

R₅ is H or a hydrocarbon chain optionally substituted with a carbocycle or a heterocycle. In a preferred embodiment, R₆ is H or alkyl i.e. methyl, ethyl, propyl, butyl, i-butyl, s-butyl or t-butyl. In a particular embodiment R₆ is H.

b. Preferred Formulas XIa-f

In a preferred embodiment, compounds of the invention have the general formula (XIa)-(XIf) 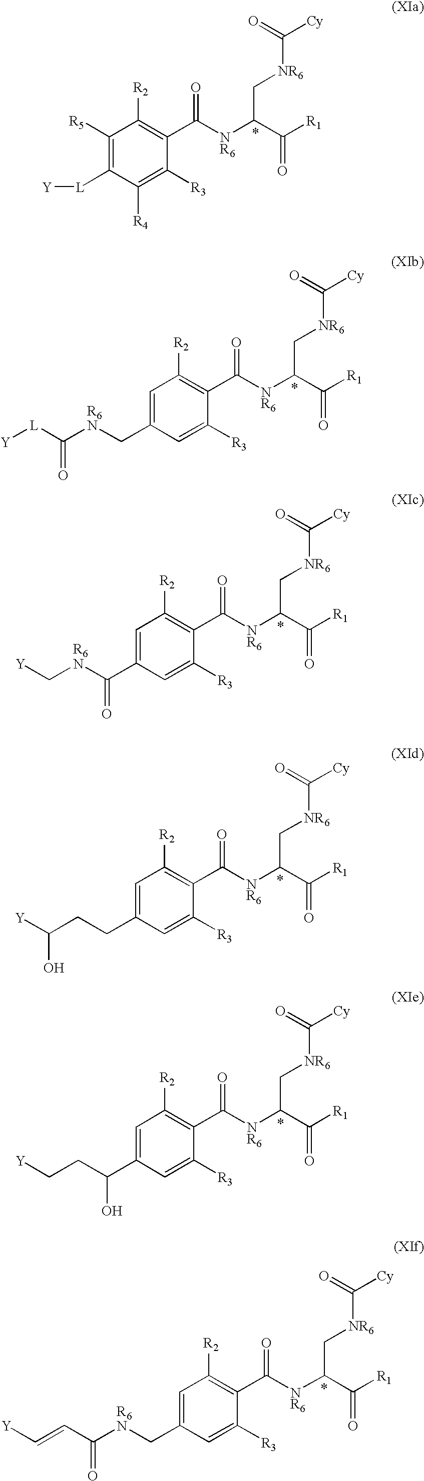
wherein Cy, Y, L and R₁₋₆ are as previously defined. In a particularly preferred embodiment, the carbon atom marked with an asterisk (*) in compounds of formula (IXa)-(IXf) is chiral. In a particular embodiment, the carbon atom has an R-configuration. In another particular embodiment, the carbon atom has an S-configuration.

c. Specific AlphaL Small Molecule Antagonists

Specific alphaL antagonist small molecules include those shown in Table 4 below.

TABLE 4

| | |
|---|---|
| 1 | 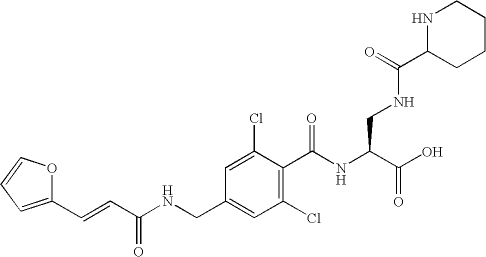 |
| 2 | 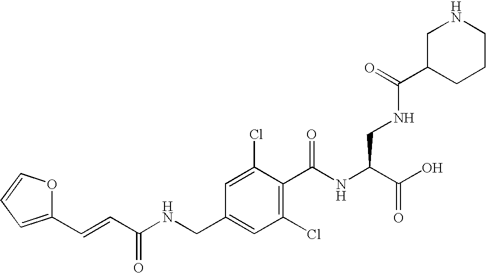 |
| 3 | 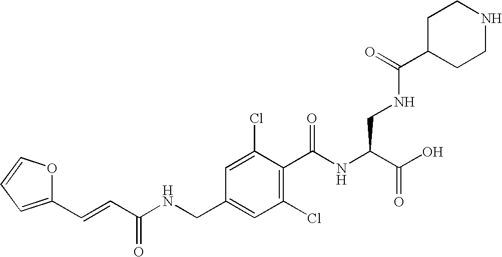 |
| 4 | 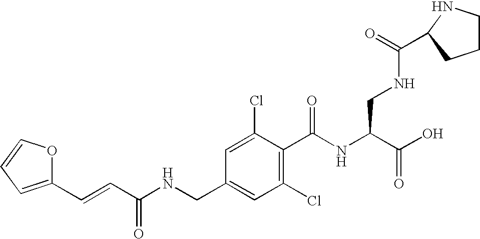 |
| 5 | 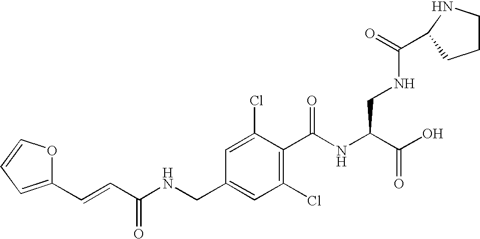 |
| 6 | 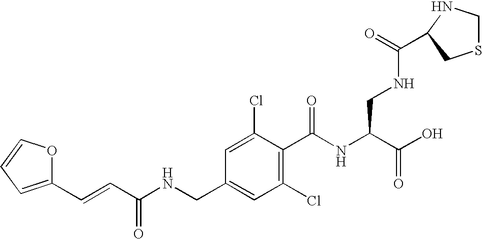 |
| 7 | 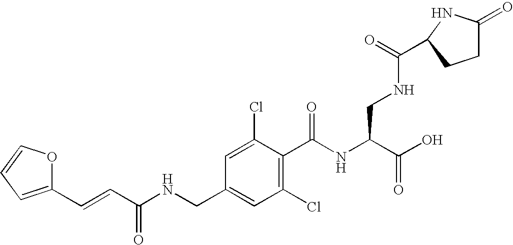 |
| 8 | 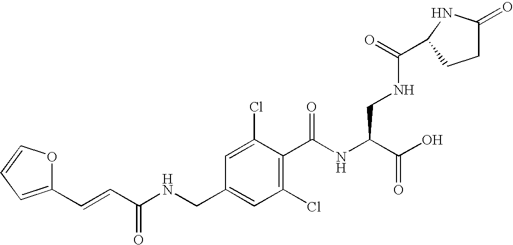 |
| 9 | 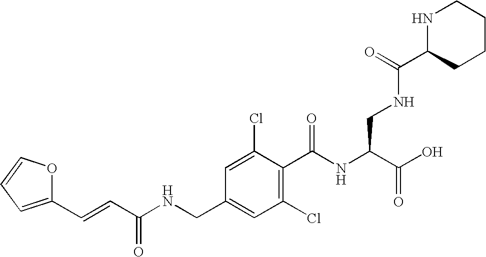 |
| 10 | 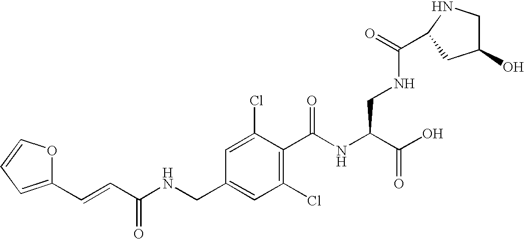 |
| 11 | 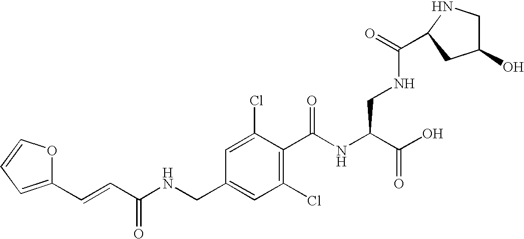 |
| 12 | 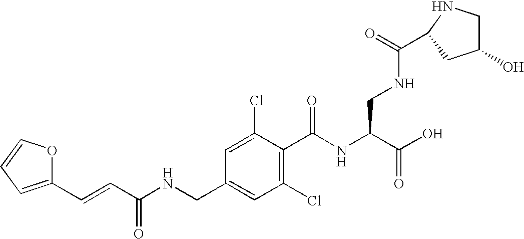 |
| 13 | 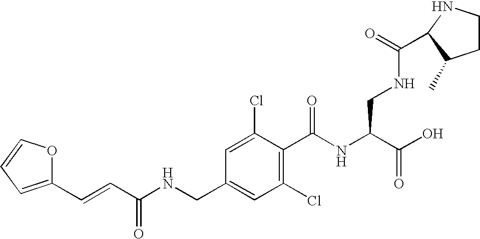 |
| 14 | 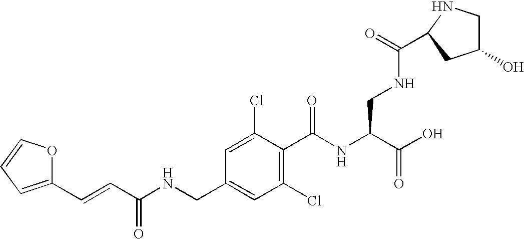 |
| 15 | 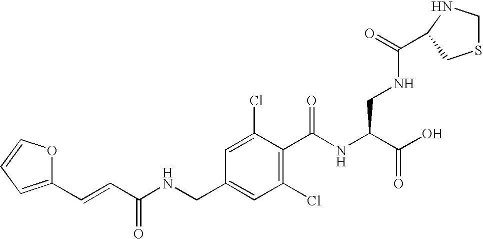 |
| 16 | 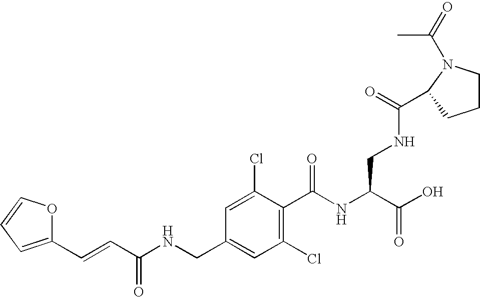 |
| 17 | 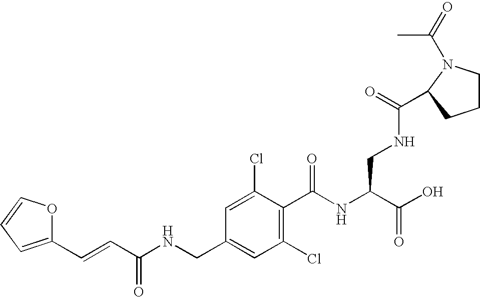 |
| 18 | 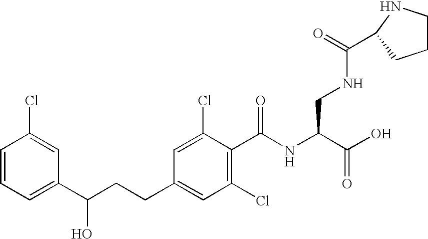 |
| 19 | 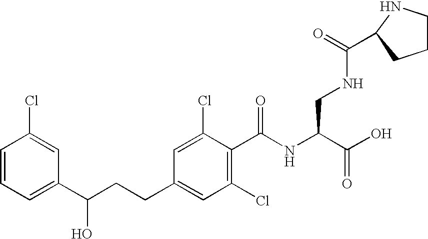 |
| 20 | 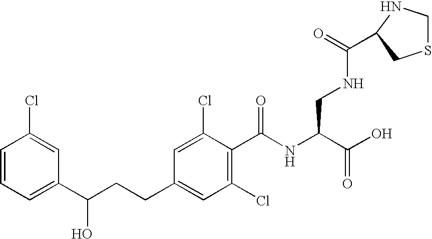 |
| 21 | 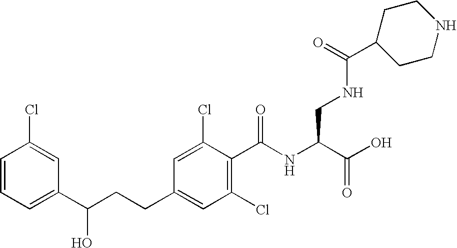 |
| 22 | 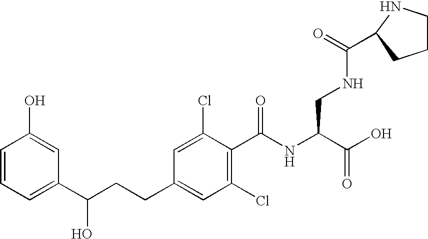 |
| 23 | 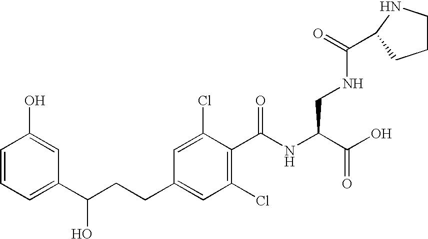 |
| 24 | 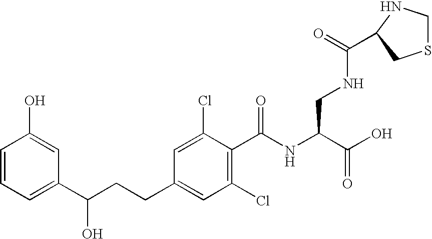 |
| 25 | 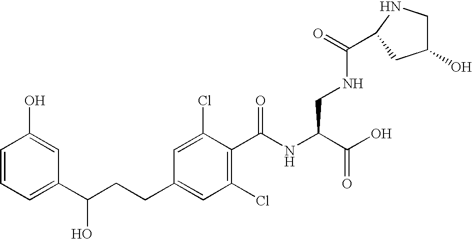 |
| 26 | 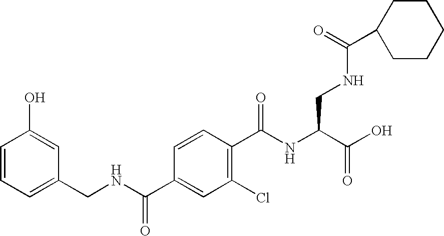 |
| 27 | 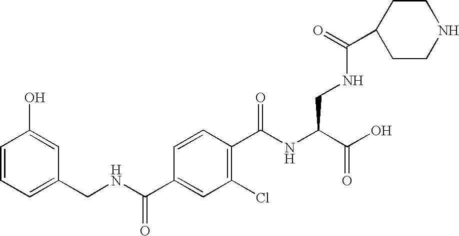 |
| 28 | 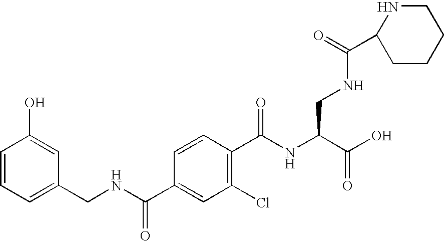 |
| 29 | 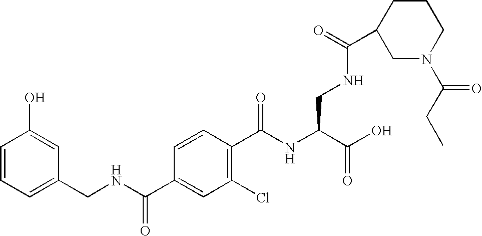 |
| 30 | 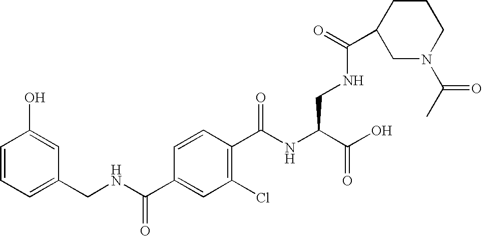 |
| 31 | 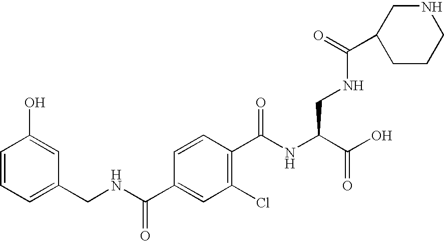 |
| 32 | 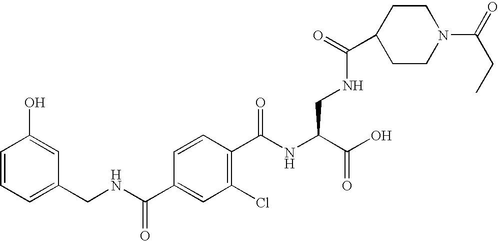 |
| 33 | 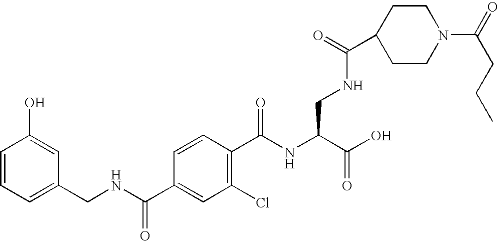 |
| 34 | 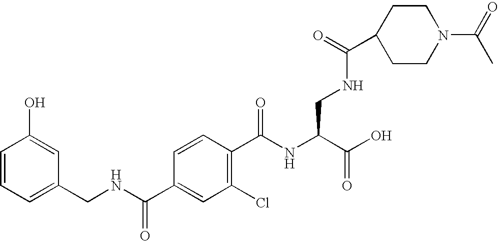 |
| 35 | 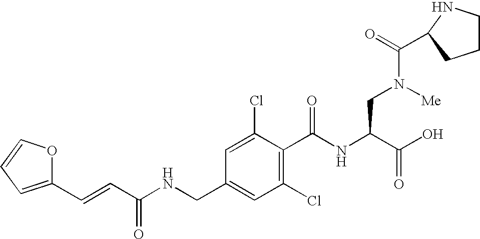 |
| 36 | 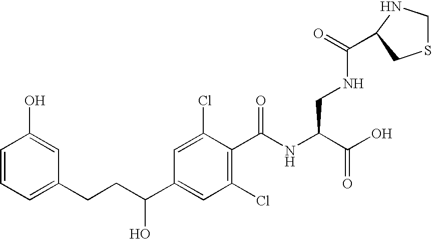 |
| 37 | 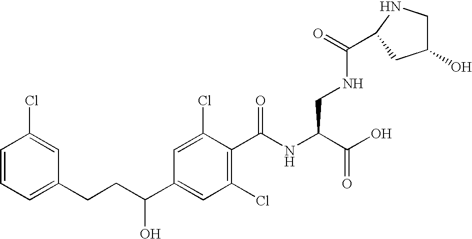 |
| 38 | 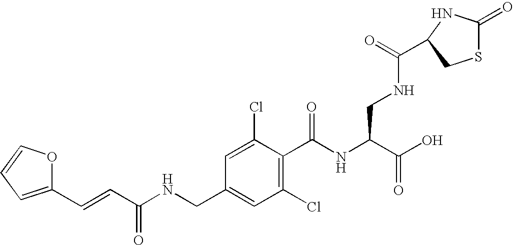 |
| 39 | 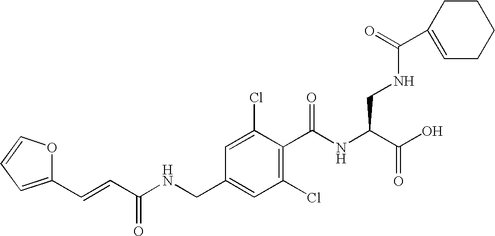 |
| 40 | 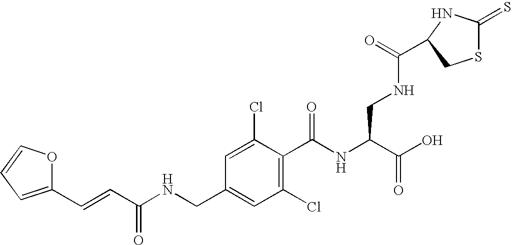 |
| 41 | 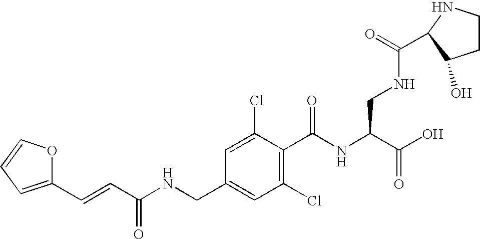 |
| 42 | 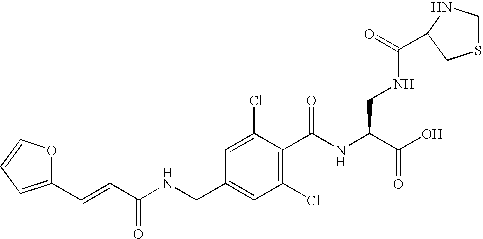 |
| 43 | 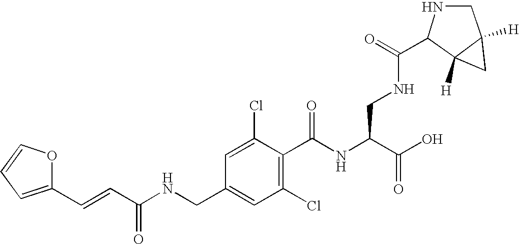 |
| 44 | 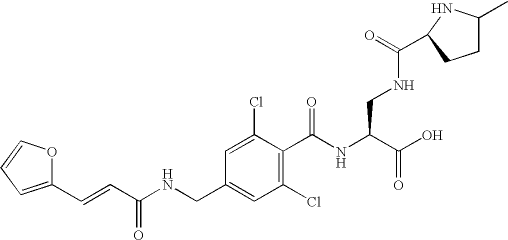 |
| 45 | 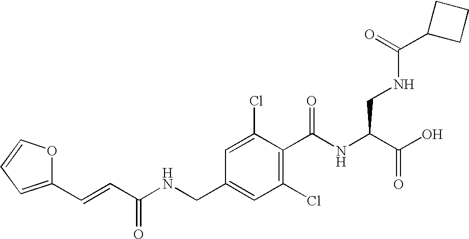 |
| 46 | 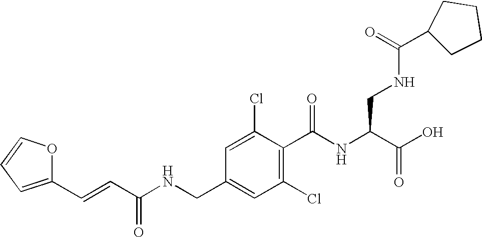 |
| 47 | 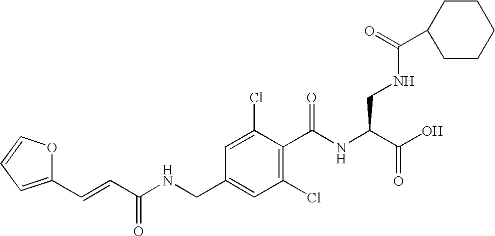 |
| 48 | 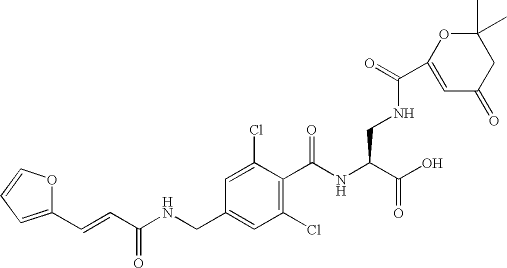 |
| 49 | 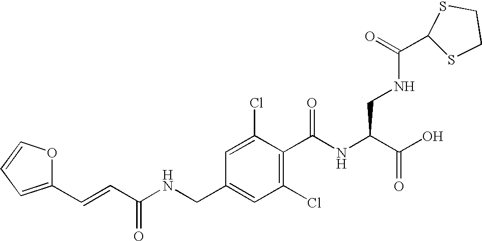 |
| 50 | 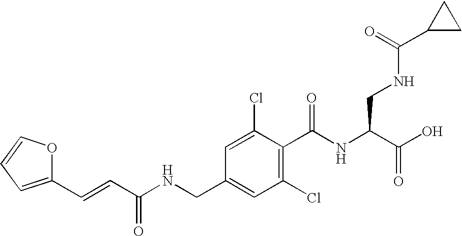 |
| 51 | 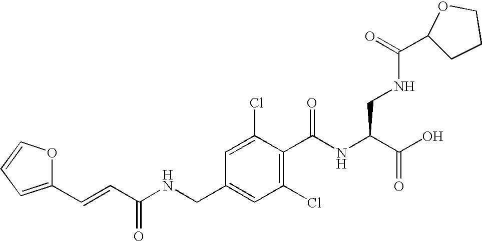 |

E. B cell Depleting Agents

B-cell depleting agents as defined above, are antagonist molecules that target B cells via surface markers, or antigens resulting in the death of the B cells directly or indirectly. Such B cell depletion agents generally bind a B cell surface marker or antigen. B cell depleting agents can be anti-B cell surface antigen antibodies, for example. Examples of such B cell depleting agents include anti-CD20, anti-CD22, and anti-CD52 antibodies, such as the anti-CD20 antibody, natiluzamab.

1. B Cell Surface Markers and Antigens

A “B cell surface marker” or “B cell surface antigen,” as used herein, is an antigen expressed on the surface of a B cell that can be targeted with an antagonist that binds thereto. Exemplary B cell surface markers include CD 10, CD19, CD20, CD21, CD22, CD23, CD24, CD37, CD40, CD53, CD72, CD73, CD74, CDw75, CDw76, CD77, CDw78, CD79a, CD79b, CD80, CD81, CD82, CD83, CDw84, CD85, and CD86 leukocyte surface markers, described for example, in The Leukocyte Antigen Facts Book, 2nd Edition. 1997, Barclay et al., Editors, Academic Press, Harcourt Brace & Co., New York. Other B cell surface markers include CD180 (RP105), FcRH2 (IRTA4), CD79A (Iga), C79B (Igp), B cellCR2, CD196 (CCR6), CD72 (Lyb-2), P2×5, HLA-DOB, CD185 (CXCR5), CD23 (FccRII), BR3, Btig, NAG14, SLGC16270, FcRH1 (IRTA5), CD307 (IRTA2), ATWD578, FcRH3, FcRH1 (IRTA1), FcRH6, CD269 (BCMA).

One particular B cell surface antigen is the “CD20” antigen, a 35 kDa, non-glycosylated phosphoprotein found on the surface of greater than 90% of B cells from peripheral blood or lymphoid organs. CD20 is expressed during early pre-B cell development and remains until plasma cell differentiation. CD20 is present on normal B cells as well as malignant B cells. Other names for CD20 in the literature include “B-lymphocyte-restricted antigen,” “B1,” and “Bp35”. The CD20 antigen is described in Clark et al., 1985, PNAS (USA) 82:1766, for example. The amino acid sequence of human CD20 is shown in The Leukocyte Antigen Facts Book, Barclay et al. supra, page 182, and also EMBL Genbank accession no. X12530 and Swissprot P11836.

Another particular B cell surface antigen is the “CD22” antigen, also known as BL-CAM or Lyb8. CD22 is a type I integral membrane glycoprotein with molecular weight of about 130 (reduced) to 140 kD (unreduced). It is expressed in both the cytoplasm and cell membrane of B-lymphocytes. CD22 antigen appears early in B-cell lymphocyte differentiation at approximately the same stage as the CD19 antigen. Unlike other B-cell markers, CD22 membrane expression is limited to late differentiation stages, for example, between mature B cells (CD22+) and plasma cells (CD22-). The CD22 antigen is described, for example, in Wilson et al., 1991, J. Exp. Med. 173:137 and Wilson et al., 1993, J. Immunol. 150:5013.

Another particular B cell surface antigen is BR3 (also known as BLyS (BAFF) receptor 3 or BAFF-R). The TNF family member BAFF is a ligand for BR3 (Patel et al, 2004, J. Biol. Chem., 279: 16727-16735; Thompson et al., 2001, Science, 293, Issue 5537, 2108-2111).

“Functional fragments” of the B cell surface antigen binding antibodies, for example, anti-CD20 antibodies described herein, are those fragments that retain binding to the antigen, for example, CD20, with substantially the same affinity as the intact full length molecule from which they are derived and demonstrate biological activity such as depleting B cells, as measured by in vitro or in vivo assays.

2. B Cell Depleting Antibodies

Biological activity of B cell depleting antibodies such as anti-CD20 and humanized anti-CD20 binding antibodies, and the like include at least binding of the antibody to a human B cell marker, such as human CD20, more preferably binding to human and other primate B cell markers such as CD20 (including as cynomolgus monkey, rhesus monkey, chimpanzees). Useful antibodies bind the B cell antigen with a K_{d} value no higher than 1×10⁻⁸, preferably a K_{d} value no higher than about 1×10⁻⁹ Useful antibodies are able to kill or deplete B cells in vivo, preferably by at least 20% when compared to the appropriate negative control which is not treated with such an antibody. B cell depletion can be a result of one or more of ADCC, CDC, or other mechanism.

In some embodiments of disease treatment herein, specific effector functions or mechanisms may be desired over others and certain variants of B cell depleting antibody such as an anti-CD20 antibody (for example, the humanized anti-CD20 antibody, 2H7 and the chimeric anti-CD20 antibody, Rituximab) are preferred to achieve those biological functions, for example, ADCC.

The terms “rituximab” or “RITUXAN®” herein refer to the genetically engineered chimeric murine/human monoclonal antibody directed against the CD20 antigen and designated “C2B8” in U.S. Pat. No. 5,736,137, including fragments thereof that retain the ability to bind CD20.

a. Anti-CD20 Antibodies

Examples of CD20 antibodies include: “C2B8,” which is now called “rituximab” (“RITUXAN®”) (U.S. Pat. No. 5,736,137); the yttrium-[90]-labelled 2B8 murine antibody designated “Y2B8” or “Ibritumomab Tiuxetan” (ZEVALIN®) commercially available from IDEC Pharmaceuticals, Inc. (U.S. Pat. No. 5,736,137; 2B8 deposited with ATCC under accession no. HB11388 on Jun. 22, 1993); murine IgG2a “B1,” also called “Tositumomab,” optionally labelled with ¹³¹I to generate the “1311-B1” or “iodine 1131 tositumomab” antibody (BEXXAR™) commercially available from Corixa (see, also, U.S. Pat. No. 5,595,721); murine monoclonal antibody “1F5” (Press et al. Blood 69(2):584-591 (1987) and variants thereof including “framework patched” or humanized 1F5 (WO 2003/002607, Leung, S.; ATCC deposit HB-96450); murine 2H7 and chimeric 2H7 antibody (U.S. Pat. No. 5,677,180); a humanized 2H7 (WO 2004/056312 (Lowman et al.) and as set forth below); HUMAX-CD20™ fully human, high-affinity antibody targeted at the CD20 molecule in the cell membrane of B-cells (Genmab, Denmark; see, for example, Glennie and van de Winkel, Drug Discovery Today 8: 503-510 (2003) and Cragg et al., Blood 101: 1045-1052 (2003)); the human monoclonal antibodies set forth in WO 2004/035607 (Teeling et al.); the antibodies having complex N-glycoside-linked sugar chains bound to the Fc region described in U.S. 2004/0093621 (Shitara et al.); CD20 binding molecules such as the AME series of antibodies, e.g., AME-33™ antibodies as set forth in WO 2004/103404 (Watkins et al., Applied Molecular Evolution); A20 antibody or variants thereof such as chimeric or humanized A20 antibody (cA20, hA20, respectively) (U.S. 2003/0219433, Immunomedics); and monoclonal antibodies L27, G28-2, 93-1 B3, B-C1 or NU-B2 available from the International Leukocyte Typing Workshop (Valentine et al., In: Leukocyte Typing III (McMichael, Ed., p. 440, Oxford University Press (1987)). The preferred CD20 antibodies herein are chimeric, humanized, or human CD20 antibodies, more preferably rituximab, a humanized 2H7, chimeric or humanized A20 antibody (Immunomedics), and HUMAX-CD20™ human CD20 antibody (Genmab).

In each of these antibodies, the C-terminal lysine (residue 447 according to the EU numbering system) of the Fc region may be removed, for example, during purification of the polypeptide or by recombinant engineering the nucleic acid encoding the polypeptide. Accordingly, a composition comprising a polypeptide such as an antibody or an immunoadhesin having an Fc region herein can comprise polypeptides with K447, with all K447 removed, or a mixture of polypeptides with and without the K447 residue. Thus, though the full length H chain sequences provided below include K447, it is intended that compositions of the antibodies below comprise antibodies lacking K447 in the H chain.

The murine anti-human CD20 antibody, m2H7 has the VH sequence:

| (SEQ ID NO: 27) | |
|---|---|
| 1 | QAYLQQSGAE LVRPGASVKM SCKASGYTFT SYNMHWVKQT PRQGLEWIGA IYPGNGDTSY |
| 61 | NQKFKGKATL TVDKSSSTAY MQLSSLTSED SAVYFCARVV YYSNSYWYFD VWGTGTTVTV |
| 121 | S |

And VL sequence:

| (SEQ ID NO: 28) | |
|---|---|
| 1 | QIVLSQSPAI LSASPGEKVT MTCRASSSVS YMHWYQQKPG SSPKPWIYAP SNLASGVPAR |
| 61 | FSGSGSGTSY SLTISRVEAE DAATYYCQQW SFNPPTFGAG TKLELK |

Purely for the purposes herein, “humanized 2H7v.16” refers to an intact antibody or antibody fragment comprising the variable light sequence:

| (SEQ ID NO: 29) | |
|---|---|
| 1 | DIQMTQSPSS LSASVGDRVT ITCRASSSVS YMNWYQQKPG KAPKPLIYAP SNLASGVPSR |
| 61 | FSGSGSGTDF TLTISSLQPE DFATYYCQQW SFNPPTFGQG TKVEIKR; |
| and | |

variable heavy sequence:

| (SEQ ID NO: 30) | |
|---|---|
| 1 | EVQLVESGGG LVQPGGSLRL SCAASGYTFT SYNMHWVRQA PGKGLEWVGA IYPGNGDTSY |
| 61 | NQKFKGRFTI SVDKSKNTLY LQMNSLRAED TAVYYCARVV YYSNSYWYFD VWGQGTLVTV |
| 121 | SS |

Where the humanized 2H7v. 16 antibody is an intact antibody, preferably it comprises the v16 light chain amino acid sequence:

| (SEQ ID NO: 31) | |
|---|---|
| 1 | DIQMTQSPSS LSASVGDRVT ITCRASSSVS YMHWYQQKPG KAPKPLIYAP SNLASGVPSR |
| 61 | FSGSGSGTDF TLTISSLQPE DFATYYCQQW SFNPPTFGQG TKVEIKRTVA APSVFIFPPS |
| 121 | DEQLKSGTAS VVCLLNNFYP REAKVQWKVD NALQSGNSQE SVTEQDSKDS TYSLSSTLTL |
| 181 | SKADYEKHKV YACEVTHQGL SSPVTKSFNR GEC; |
| and | |

v16 heavy chain amino acid sequence

| (SEQ ID NO: 32) | |
|---|---|
| 1 | EVQLVESGGG LVQPGGSLRL SCAASGYTFT SYNMHWVRQA PGKGLEWVGA IYPGNGDTSY |
| 61 | NQKFKGRFTI SVDKSKNTLY LQMNSLRAED TAVYYCARVV YYSNSYWYFD VWGQGTLVTV |
| 121 | SSASTKGPSV FPLAPSSKST SGGTAALGCL VKDYFPEPVT VSWNSGALTS GVHTFPAVLQ |
| 181 | SSGLYSLSSV VTVPSSSLGT QTYICNVNHK PSNTKVDKKV EPKSCDKTHT CPPCPAPELL |
| 241 | GGPSVFLFPP KPKDTLMISR TPEVTCVVVD VSHEDPEVKF NWYVDGVEVH NAKTKPREEQ |
| 301 | YNSTYRVVSV LTVLHQDWLN GKEYKCKVSN KALPAPIEKT ISKAKGQPRE PQVYTLPPSR |
| 361 | EEMTKNQVSL TCLVKGFYPS DIAVEWESNG QPENNYKTTP PVLDSDGSFF LYSKLTVDKS |
| 421 | RWQQGNVFSC SVNHEALHNH YTQKSLSLSP GK |

The V region of all other variants based on version 16 have the amino acid sequences of v16 except at the positions of amino acid substitutions that are indicated in the table below. Unless otherwise indicated, the 2H7 variants have the same L chain as that of v16.

| 2H7 | Heavy chain | Light chain | |
|---|---|---|---|
| version | (V_{H}) changes | (V_{L}) changes | Fc changes |
| 16 | | | — |
| 31 | — | — | S298A, E333A, K334A |
| 73 | N100A | M32L | |
| 75 | N100A | M32L | S298A, E333A, K334A |
| 96 | D56A, N100A | S92A | |
| 114 | D56A, N100A | M32L, S92A | S298A, E333A, K334A |
| 115 | D56A, N100A | M32L, S92A | S298A, E333A, K334A, |
| | | | E356D, M358L |
| 116 | D56A, N100A | M32L, S92A | S298A, K334A, K322A |
| 138 | D56A, N100A | M32L, S92A | S298A, E333A, K334A, |
| | | | K326A |
| 477 | D56A, N100A | M32L, S92A | S298A, E333A, K334A, |
| | | | K326A, N434W |
| 375 | — | — | K334L |
| 511 | D56A, N100Y, | M32L, S92A | S298A, E333A, K334A, |
| | S100aR | | K326A |
| 588 | — | — | S298A, E333A, K334A, |
| | | | K326A |

The sequences of some of the variants of the preceding humanized 2H7v.16 mAb are as follows:

2H7v.31 having the same L chain sequence as SEQ ID NO: 31 above, with the H chain amino acid sequence:

| (SEQ ID NO: 33) | |
|---|---|
| 1 | EVQLVESGGG LVQPGGSLRL SCAASGYTFT SYNMHWVRQA PGKGLEWVGA IYPGNGDTSY |
| 61 | NQKFKGRFTI SVDKSKNTLY LQMNSLRAED TAVYYCARVV YYSNSYWYFD VWGQGTLVTV |
| 121 | SSASTKGPSV FPLAPSSKST SGGTAALGCL VKDYFPEPVT VSWNSGALTS GVHTFPAVLQ |
| 181 | SSGLYSLSSV VTVPSSSLGT QTYICNVNHK PSNTKVDKKV EPKSCDKTHT CPPCPAPELL |
| 241 | GGPSVFLFPP KPKDTLMISR TPEVTCVVVD VSHEDPEVKF NWYVDGVEVH NAKTKPREEQ |
| 301 | YNATYRVVSV LTVLHQDWLN GKEYKCKVSN KALPAPIAAT ISKAKGQPRE PQVYTLPPSR |
| 361 | EEMTKNQVSL TCLVKGFYPS DIAVEWESNG QPENNYKTTP PVLDSDGSFF LYSKLTVDKS |
| 421 | RWQQGNVFSC SVMHEALHNH YTQKSLSLSP GK; |

2H7v.138 having the H chain amino acid sequence:

(SEQ ID NO:43)
EVQLVESGGGLVQPGGSLRLSCAASG
YTFTSYNMHWVRQAPGKGLEWVGAIYPGNGATSYNQKFKGRFTISVDKSKNTLYLQMNSL
RAEDTAVYYCARVVYYSASYWYFDVWGQGTLVTVSSASTKGPSVFPLAPSSKSTSGGTAA
LGCLVKDYFPEPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNATYRVVSVLTVLHQDWLNGKEYKC
KVSNAALPAPIAATISKAKGQPREPQVYTLPPSREEMTKNQVSLTCLVKGFYPSDIAVEW
ESNGQPENNYKTTPPVLDSDGSFFLYSKLTVDKSRWQQGNVFSCSVMHEALHNHYTQKSL
SLSPGK

and v138 L chain amino acid sequence:

(SEQ ID NO:44)
DIQMTQSPSSLSASVGDRVTITCRASSSVSYLHWYQQKPGKAPKPLIYAP
SNLASGVPSRFSGSGSGTDFTLTISSLQPEDFATYYCQQWAFNPPTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC;

2H7v.114 having the same L chain sequence as that of v.138, SEQ ID NO: 44 above, with the H chain amino acid sequence:

(SEQ ID NO:45)
EVQLVESGGGLVQPGGSLRLSCAASG
YTFTSYNMHWVRQAPGKGLEWVGAIYPGNGATSYNQKFKGRFTISVDKSKNTLYLQMNSL
RAEDTAVYYCARVVYYSASYWYFDVWGQGTLVTVSSASTKGPSVFPLAPSSKSTSGGTAA
LGCLVKDYFPEPVTVSWNSGALTSGVHTFPAVLQSSGLYSLSSVVTVPSSSLGTQTYION
VNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNATYRVVSVLTVLHQDWLNGKEYKC
KVSNKALPAPIAATISKAKGQPREPQVYTLPPSREEMTKNQVSLTCLVKGFYPSDIAVEW
ESNGQPENNYKTTPPVLDSDGSFFLYSKLTVDKSRWQQGNVFSCSVMHEALHNHYTQKSL
SLSPGK;

2H7v.477 having the L chain sequence of 2H7v.138 (SEQ ID NO:44), and the H chain amino acid sequence:

(SEQ ID NO:46)
EVQLVESGGGLVQPGGSLRLSCAASG
YTFTSYNMHWVRQAPGKGLEWVGAIYPGNGATSYNQKFKGRFTISVDKSKNTLYLQMNSL
RAEDTAVYYCARVVYYSASYWYFDVWGQGTLVTVSSASTKGPSVFPLAPSSKSTSGGTAA
LGCLVKDYFPEPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNATYRVVSVLTVLHQDWLNGKEYKC
KVSNAALPAPIAATTSKAKGQPREPQVYTLPPSREEMTKNQVSLTCLVKGFYPSDIAVEW
ESNGQPENNYKTTPPVLDSDGSFFLYSKLTVDKSRWQQGNVFSCSVMHEALHWHYTQKSL
SLSPGK;

2H7v.511 having the L chain sequence of 2H7v.138 (SEQ ID NO:44), and the H chain amino acid sequence:

(SEQ ID NO: 47)
EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSYRYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK.

Each of versions 114, 115, 116, 138, 477, 511 comprise the VL sequence:

(SEQ ID NO: 48)

DIQMTQSPSSLSASVGDRVTITCRASSSVSYLHWYQQKPGKAPKPLIYAP
SNLASGVPSRFSGSGSGTDFTLTISSLQPEDFATYYCQQWAFNPPTFGQG
TKVEIKR.

b. Anti-CD22 Antibodies, B Cell Depleting Antibodies, and the Like

B cell depleting antibodies also include antibodies and binding ligands that antagonize CD20, CD22, CD23, BR3, and CD80. Examples include the anti-CD22 antibody LyphoCide™, also known as epratuzumab (Immunomedics, Inc., Morris Plains, N.J.); the BAFF-R (CT) BR3 Blocking Peptide (QED Bioscience, Inc., San Diego, Calif.); the anti-CD23 antibody, IDEC-152, a primatised antibody (Biogen IDEC, Cambridge, Mass.), the anti-CD80 antibody, DEC-114, a primatised antibody (Biogen IDEC, Cambridge, Mass.); and the like.

Chimeric and Humanized A20 Antibodies have the following sequences as disclosed in U.S. Provisional Application 2003/0219433. The cA20 anti-CD20 antibody has the VL sequence:

| (SEQ ID NO: 34) | |
|---|---|
| 1 | DIQLTQSPAI LSASPGEKVT MTCRASSSVS YIHWFQQKPG SSPKPWIYAT SNLASGVPVR |
| 61 | FSGSGSGTSY SLTISRVEAE DAATYYCQQW TSNPPTFGGG TKLEIK |

And VH sequence:

| (SEQ ID NO: 35) | |
|---|---|
| 1 | QVQLQQPGAE LVKPGASVKM SCKASGYTFT SYNMHWVKQT PGRGLEWIGA IYPGNGDTSY |
| 61 | NQKFKGKATL TADKSSSTAY MQLSSLTSED SAVYYCARST YYGGDWYFDV WGQGTTVTVS |
| 121 | S |

One hA20 anti-CD20 antibody has the VL sequence:

| (SEQ ID NO: 36) | |
|---|---|
| 1 | DIQLTQSPSS LSASVGDRVT MTCRASSSVS YIHWFQQKPG KAPKPWIYAT SNLASGVPVR |
| 61 | FSGSGSGTDY TFTISSLQPE DIATYYCQQW TSNPPTFGGG TKLEIK |

And VH1 sequence:

| (SEQ ID NO: 37) | |
|---|---|
| 1 | QVQLQQSGAE VKKPGSSVKV SCKASGYTFT SYNMHWVKQA PGQGLEWIGA IYPGNGDTSY |
| 61 | NQKFKGKATL TADESTNTAY MELSSLRSED TAFYYCARST YYGGDWYFDV WGQGTTVTVS |
| 121 | S |

An alternate hA20VH1 has the sequence:

| (SEQ ID NO: 38) | |
|---|---|
| 1 | QVQLQQSGAE VKKPGSSVKV SCKASGYTFS SYNMHWVRQA PGQGLEWMGA IYPGNGDTSY |
| 61 | NQKFKGRATI TADESTNTAY MELSSLRSED TAFYFCARST YYGGDWYFDV WGQGTTVTVS |
| 121 | S |

Humanized (FR-patched) 1F5 antibodies have the sequences disclosed in U.S. Provisional Application 2003/0040606.

One hu1F5 anti-CD20 antibody has the VL sequence:

| (SEQ ID NO: 39) | |
|---|---|
| 1 | QVQLVASGAE VNKPGASVKV SCKASGYTFT SYNMHWVRQP PGRGLEWIGA IYPGNGDTSY |
| 61 | NQKFKGKATL TADKSSSTAY MQLSSLTSED SAVYYCARSH YGSNYVDYFD YWGQGTTVTV |
| 121 | SS |

And the VH sequence:

| (SEQ ID NO: 40) | |
|---|---|
| 1 | DIQLTQSPSS LSASVGDRvT ITCRASSSLS FMHWYQQKPG SSPKPWIYAT SNLASGVPSR |
| 61 | FSGSGSGTEF TLTISSLQPE DFATYFCHQW SSNPLTFGAG TKLTVLR |

An alternate hu1F5 anti-CD20 antibody has the VL sequence:

| (SEQ ID NO: 41) | |
|---|---|
| 1 | QVQLVASGAE VNKPGASVKV SCKASGYTFT SYNMHWVRQPP GRGLEWIGA IYPGNGDTSY |
| 61 | NQKFKGRVTI TADKSTSTAY MELSSLRSED TAVYYCARSHY GSNYVDYFD YWGQGTTVTV |
| 121 | SS |

And the VH sequence:

| (SEQ ID NO: 42) | |
|---|---|
| 1 | DIQLTQSPSS LSASVGDRVT ITCRASSSLS FMHWYQQKPG QAPVPVIYAT SNLASGVPSR |
| 61 | FSGSGSGTEF TLTISSLQPE DFATYFCHQW SSNPLTFGAG TKLTVLR |

F. Methods of Treatment

The methods of the invention are useful to treat a number of malignant and non-malignant diseases including autoimmune diseases and related conditions, and cancers including B cell lymphomas and leukemias. For example, stem cells (B-cell progenitors) in bone marrow lack the CD20 antigen, allowing healthy B-cells to regenerate after treatment with CD20 antagonists and return to normal levels within several months.

1. Autoimmune Disorders and Related Conditions

Autoimmune diseases or autoimmune related conditions include arthritis (rheumatoid arthritis such as acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), pyoderma gangrenosum, erythema nodosum, primary sclerosing cholangitis, episcleritis), respiratory distress syndrome, including adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, allergic conditions and responses, allergic reaction, eczema including allergic or atopic eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus, alopecia lupus, systemic lupus erythematosus (SLE) such as cutaneous SLE or subacute cutaneous SLE, systemic lupus erythematodes, neonatal lupus syndrome (NLE), and lupus erythematosus disseminatus, juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis including lymphomatoid granulomatosis, Wegener's granulomatosis, agranulocytosis, vasculitides, including vasculitis (including large vessel vasculitis (including polymyalgia rheumatica and giant cell (Takayasu's) arteritis), medium vessel vasculitis (including Kawasaki's disease and polyarteritis nodosa/periarteritis nodosa), microscopic polyarteritis, CNS vasculitis, necrotizing, cutaneous, or hypersensitivity vasculitis, systemic necrotizing vasculitis, and ANCA-associated vasculitis, such as Churg-Strauss vasculitis or syndrome (CSS)), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, immune complex nephritis, antibody-mediated nephritis, polyneuropathies, chronic neuropathy such as IgM polyneuropathies or IgM-mediated neuropathy, thrombocytopenia (as developed by myocardial infarction patients, for example), including thrombotic thrombocytopenic purpura (TTP), post-transfusion purpura (PTP), heparin-induced thrombocytopenia, and autoimmune or immune-mediated thrombocytopenia such as idiopathic thrombocytopenic purpura (ITP) including chronic or acute ITP, autoimmune disease of the testis and ovary including autoimune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, diabetic nephropathy, Dressler's syndrome, alopecia greata, CREST syndrome (calcinosis, Raynaud's phenomenon, esophageal dysmotility, sclerodactyly, and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetal is, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, infertility due to antispermatozoan antobodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, lymphadenitis, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

2. Cancers, CD20⁺ Cancers

A B cell neoplasm or malignancy is characterized by expression of a B cell antigen or surfacemarker such as CD20. For example, CD20 positive cancers are those comprising abnormal proliferation of cells that express CD20 on the cell surface. The CD20 positive B cell neoplasms include CD20-positive Hodgkin's disease including lymphocyte predominant Hodgkin's disease (LPHD); non-Hodgkin's lymphoma (NHL); follicular center cell (FCC) lymphomas; acute lymphocytic leukemia (ALL); chronic lymphocytic leukemia (CLL); Hairy cell leukemia. The non-Hodgkins lymphoma include low grade/follicular non-Hodgkin's lymphoma (NHL), small lymphocytic lymphoma (SLL), intermediate grade/follicular NHL, intermediate grade diffuse NHL, high grade immunoblastic NHL, high grade lymphoblastic NHL, high grade small non-cleaved cell NHL, bulky disease NHL, plasmacytoid lymphocytic lymphoma, mantle cell lymphoma, AIDS-related lymphoma, and Waldenstrom's macroglobulinemia. Treatment of relapses of these cancers are also contemplated. LPHD is a type of Hodgkin's disease that tends to relapse frequently despite radiation or chemotherapy treatment and is characterized by CD20-positive malignant cells. CLL is one of four major types of leukemia. A cancer of mature B-cells called lymphocytes, CLL is manifested by progressive accumulation of cells in blood, bone marrow and lymphatic tissues. Indolent lymphoma is a slow-growing, incurable disease in which the average patient survives between six and 10 years following numerous periods of remission and relapse.

In specific embodiments, the methods of treatment and of augmenting B cell depletion described herein are useful to treat B cell neoplasms or malignancies, such as non-Hodgkin's lymphoma (NHL), lymphocyte predominant Hodgkin's disease (LPHD), small lymphocytic lymphoma (SLL), chronic lymphocytic leukemia, rheumatoid arthritis and juvenile rheumatoid arthritis, systemic lupus erythematosus (SLE) including lupus nephritis, Wegener's disease, inflammatory bowel disease, idiopathic thrombocytopenic purpura (ITP), thrombotic throbocytopenic purpura (TTP), autoimmune thrombocytopenia, multiple sclerosis, psoriasis, IgA nephropathy, IgM polyneuropathies, myasthenia gravis, vasculitis, diabetes mellitus, Reynaud's syndrome, Sjorgen's syndrome, and glomerulonephritis.

The desired level of B cell depletion will depend on the disease. For example, in the treatment of a CD20 positive cancer it may be desirable to maximize depletion of B cells. Thus, for the treatment of a CD20 (or other B cell surface antigen or marker) positive B cell neoplasm, it is desirable that the B cell depletion be sufficient to at least prevent progression of the disease, which can be assessed by the physician of skill in the art, e.g., by monitoring tumor growth (size), proliferation of the cancerous cell type, metastasis, and/or other signs and symptoms of the particular cancer. Preferably, B cell depletion is sufficient to prevent progression of disease for at least 2 months, more preferably 3 months, even more preferably 4 months, more preferably 5 months, even more preferably 6 or more months. In even more preferred embodiments, B cell depletion is sufficient to increase the time in remission by at least 6 months, more preferably 9 months, more preferably one year, more preferably 2 years, more preferably 3 years, even more preferably 5 or more years. In a most preferred embodiment, the B cell depletion is sufficient to cure the disease. In preferred embodiments, the B cell depletion in a cancer patient is at least about 75% and more preferably, 80%, 85%, 90%, 95%, 99% and even 100% of the baseline level before treatment.

3. Autoimmune Disorders

For treatment of an autoimmune disease, it may be desirable to modulate the extent of B cell depletion depending on the disease and/or the severity of the condition in the individual patient, by adjusting the dosage of the B cell depleting agent, for example, CD20 binding antibody. B cell depletion can be complete or partial. Total B cell depletion may be desired during initial treatment, but in subsequent treatments, the dosage may be adjusted to achieve only partial depletion. In one embodiment, the B cell depletion is at least 20%, i.e., 80% or less of targeted, for example, CD20 positive, B cells remain as compared to the baseline level before treatment. In other embodiments, B cell depletion is 25%, 30%, 40%, 50%, 60%, 70% or greater. Preferably, the B cell depletion is sufficient to halt progression of disease, more preferably to alleviate the signs and symptoms of the particular disease under treatment, even more preferably to cure the disease.

The parameters for assessing efficacy or success of treatment of the neoplasm will be known to the physician of skill in the appropriate disease. Generally, the physician of skill will look for reduction in the signs and symptoms of the specific disease. Parameters can include median time to disease progression, time in remission and stable disease.

The following references describe lymphomas and CLL, their diagnoses, treatment and standard medical procedures for measuring treatment efficacy. Canellos G P, Lister, T A, Sklar J L: The Lymphomas. W. B. Saunders Company, Philadelphia, 1998; van Besien K and Cabanillas, F: Clinical Manifestations, Staging and Treatment of Non-Hodgkin's Lymphoma, Chap. 70, pp 1293-1338, in: Hematology, Basic Principles and Practice, 3rd ed. Hoffman et al. (editors). Churchill Livingstone, Philadelphia, 2000; and Rai, K and Patel, D:Chronic Lymphocytic Leukemia, Chap. 72, pp 1350-1362, in: Hematology, Basic Principles and Practice, 3rd ed. Hoffman et al. (editors). Churchill Livingstone, Philadelphia, 2000.

The parameters for assessing efficacy or success of treatment of an autoimmune or autoimmune related disease will be known to the physician of skill in the appropriate disease. Generally, the physician of skill will look for reduction in the signs and symptoms of the specific disease. The following are by way of examples.

In one embodiment, the methods and compositions of the invention are useful to treat rheumatoid arthritis (RA). RA is characterized by inflammation of multiple joints, cartilage loss and bone erosion that leads to joint destruction and ultimately reduced joint function. Additionally, since RA is a systemic disease, it can have effects in other tissues such as the lungs, eyes and bone marrow.

B cell depleting agents such as B cell antigen binding antibodies, for example, CD20 binding antibodies together with B cell mobilizing agents such as integrin antibodies can be used as first-line therapy in patients with early RA (i.e., methotrexate (MTX) naive), or in combination with, e.g., MTX or cyclophosphamide. In another embodiment this combination of B cell depleting agents, for example anti-CD20 antibodies, together with B cell mobilizing agents such as anti-alpha4 and/or anti-alphaL antagonists, including antibodies, can be used in treatment as second-line therapy for patients who were disease-modifying anti-rheumatic drugs and/or methotrexate refractory, in combination with, e.g., methotrexate. These agents, for example, humanized CD20 binding antibodies and integrin antibodies, are useful to prevent and control joint damage, delay structural damage, decrease pain associated with inflammation in rheumatoid arthritis, and generally reduce the signs and symptoms in moderate to severe rheumatoid arthritis. The rheumatoid arthritis patient can be treated with the B cell depleting agent, for example, humanized anti-CD20 antibody, and B cell mobilizing agent, for example, anti-integrin antibody, prior to, after or together with treatment with other drugs used in treating RA (see combination therapy below). In one embodiment, patients who had previously failed disease-modifying antirheumatic drugs and/or had an inadequate response to methotrexate alone are treated with a B cell depleting agent such as an anti-CD20 binding antibody. In another embodiment, in addition to the anti-integrin antibodies, patients are administered humanized anti-CD20 binding antibody, anti-CD20 binding antibody plus cyclophosphamide, or anti-CD20 binding antibody plus methotrexate.

One method of evaluating treatment efficacy in rheumatoid arthritis is based on American College of Rheumatology (ACR) criteria, which measures the percentage of improvement in tender and swollen joints, among other things. The rheumatoid arthritis patient can be scored at for example, ACR 20 (20 percent improvement) compared with no antibody treatment (e.g., baseline before treatment) or treatment with placebo. Other ways of evaluating the efficacy of antibody treatment include X-ray scoring such as the Sharp X-ray score used to score structural damage such as bone erosion and joint space narrowing. Patients can also be evaluated for the prevention of or improvement in disability based on Health Assessment Questionnaire [HAQ] score, AIMS score, SF-36 at time periods during or after treatment. The ACR 20 criteria may include 20% improvement in both tender (painful) joint count and swollen joint count plus a 20% improvement in at least 3 of 5 additional measures:
1. patient's pain assessment by visual analog scale (VAS),
2. patient's global assessment of disease activity (VAS),
3. physician's global assessment of disease activity (VAS),
4. patient's self-assessed disability measured by the Health Assessment Questionnaire, and
5. acute phase reactants, CRP or ESR.

The ACR 50 and 70 are defined analogously. Preferably, the patient is administered an amount of a B cell depleting agent such as an anti-CD20 binding antibody of the invention effective to achieve at least a score of ACR 20, preferably at least ACR 30, more preferably at least ACR50, even more preferably at least ACR70, most preferably at least ACR 75 and higher.

Psoriatic arthritis has unique and distinct radiographic features. For psoriatic arthritis, joint erosion and joint space narrowing can be evaluated by the Sharp score as well. The B cell depleting agents, such as humanized anti-CD20 binding antibodies disclosed herein can be used to prevent the joint damage as well as reduce disease signs and symptoms of the disorder.

Yet another aspect of the invention is a method of treating Lupus or SLE by administering to the patient suffering from SLE, a therapeutically effective amount of a B cell depleting agent such as a humanized anti-CD20 binding antibody. SLEDAI scores provide a numerical quantitation of disease activity. The SLEDAI is a weighted index of 24 clinical and laboratory parameters known to correlate with disease activity, with a numerical range of 0-103. See, for example, Gescuk et al., 2002, Current Opinion in Rheumatology 14:515-521. Antibodies to double-stranded DNA are believed to cause renal flares and other manifestations of lupus. Patients undergoing antibody treatment can be monitored for time to renal flare, which is defined as a significant, reproducible increase in serum creatinine, urine protein or blood in the urine. Alternatively or in addition, patients can be monitored for levels of antinuclear antibodies and antibodies to double-stranded DNA. Treatments for SLE include high-dose corticosteroids and/or cyclophosphamide (HDCC).

Spondyloarthropathies are a group of disorders of the joints, including ankylosing spondylitis, psoriatic arthritis and Crohn's disease. Treatment success can be determined by validated patient and physician global assessment measuring tools.

Various medications are used to treat psoriasis; treatment differs directly in relation to disease severity. Patients with a more mild form of psoriasis typically utilize topical treatments, such as topical steroids, anthralin, calcipotriene, clobetasol, and tazarotene, to manage the disease while patients with moderate and severe psoriasis are more likely to employ systemic (methotrexate, retinoids, cyclosporine, PUVA and UVB) therapies. Tars are also used. These therapies have a combination of safety concerns, time consuming regimens, or inconvenient processes of treatment. Furthermore, some require expensive equipment and dedicated space in the office setting. Systemic medications can produce serious side effects, including hypertension, hyperlipidemia, bone marrow suppression, liver disease, kidney disease and gastrointestinal upset. Also, the use of phototherapy can increase the incidence of skin cancers. In addition to the inconvenience and discomfort associated with the use of topical therapies, phototherapy and systemic treatments require cycling patients on and off therapy and monitoring lifetime exposure due to their side effects.

Treatment efficacy for psoriasis is assessed by monitoring changes in clinical signs and symptoms of the disease including Physician's Global Assessment (PGA) changes and Psoriasis Area and Severity Index (PASI) scores, Psoriasis Symptom Assessment (PSA), compared with the baseline condition. The patient can be measured periodically throughout treatment on the Visual analog scale used to indicate the degree of itching experienced at specific time points.

4. Dosage

Depending on the indication to be treated and factors relevant to the dosing that a physician of skill in the field would be familiar with, the B cell depleting agents and B cell mobilizing agents of the invention will be administered at a dosage that is efficacious for the treatment of that indication while minimizing toxicity and side effects.

For the treatment of a CD20 positive B cell neoplasm, it is desirable that the B cell depletion be sufficient to at least prevent progression of the disease which can be assessed by the physician of skill in the art, e.g., by monitoring tumor growth (size), proliferation of the cancerous cell type, metastasis, other signs and symptoms of the particular cancer. Preferably, the B cell depletion is sufficient to prevent progression of disease for at least 2 months, more preferably 3 months, even more preferably 4 months, more preferably 5 months, even more preferably 6 or more months. In even more preferred embodiments, the B cell depletion is sufficient to increase the time in remission by at least 6 months, more preferably 9 months, more preferably one year, more preferably 2 years, more preferably 3 years, even more preferably 5 or more years. In a most preferred embodiment, the B cell depletion is sufficient to cure the disease. In preferred embodiments, the B cell depletion in a cancer patient is at least about 75% and more preferably, 80%, 85%, 90%, 95%, 99% and even 100% of the baseline level before treatment.

For the treatment of a CD20 positive cancer or an autoimmune disease, the therapeutically effective dosage can be in the range of about 250 mg/m² to about 400 mg/m² or 500 mg/m², preferably about 250-375 mg/m². In one embodiment, the dosage range is 275-375 mg/m². In one embodiment of the treatment of a CD20 positive B cell neoplasm, the antibody is administered at a range of 300-375 mg/m². For the treatment of patients suffering from B-cell lymphoma such as non-Hodgkins lymphoma, in a specific embodiment, the anti-CD20 antibodies and humanized anti-CD20 antibodies of the invention will be administered to a human patient at a dosage of 10 mg/kg or 375 mg/m². In one embodiment, Rituximab can be administered at a dosage range of 7-15 mg/kg. For treating NHL, one dosing regimen would be to administer one dose of the antibody composition a dosage of 10 mg/kg in the first week of treatment, followed by a 2 week interval, then a second dose of the same amount of antibody is administered. Generally, NHL patients receive such treatment once during a year but upon recurrence of the lymphoma, such treatment can be repeated. In another dosing regimen, patients treated with low-grade NHL receive four weeks of a version of humanized 2H7, preferably v16 (375 mg/m2 weekly) followed at week five by three additional courses of the antibody plus standard CHOP (cyclophosphamide, doxorubicin, vincristine and prednisone) or CVP (cyclophosphamide, vincristine, prednisone) chemotherapy, which was given every three weeks for three cycles.

For treating rheumatoid arthritis, in one embodiment, the dosage range for the humanized anti-CD20 antibody is 125 mg/m² (equivalent to about 200 mg/dose) to 600 mg/m², given in two doses, e.g., the first dose of 200 mg is administered on day one followed by a second dose of 200 mg on day 15. In different embodiments, the dosage is 250 mg/dose, 275 mg, 300 mg, 325 mg, 350 mg, 375 mg, 400 mg, 425 mg, 450 mg, 475 mg, 500 mg, 525 mg, 550 mg, 575 mg, 600 mg.

The Genentech and Biogen Idec clinical investigations have evaluated the therapeutic effectiveness of treatment of autoimmune diseases using doses of anti-CD20 (hu2H7.v16 and Rituximab) ranging from as low as 10 mg up to a dose of 1 g (see under Background section for Rituximab studies; and WO 04/056312, Example 16). In general, the antibodies were administered in these clinical investigations in two doses, spaced about two weeks apart. Examples of regimens studied in the clinical investigations include, for humanized CD20 antibody 2H7.v16 in rheumatoid arthritis at 2×10 mg (means 2 doses at 10 mg per dose; total dose of ˜10.1 mg/m² for a 70 kg, 67 inch tall patient), 2×50 mg (total dose of 55 mg/m² for a 70 kg, 67 in tall patient), 2×200 mg (total dose of 220 mg/m² for a 70 kg, 67 in tall patient), 2×500 mg (total dose of −550 mg/m2 for a 70 kg, 67 in tall patient) and 2×1000 mg (total dose of ˜1100 mg/m2 for a 70 kg, 67 in tall patient); and for Rituxan, 2×500 mg (total dose of ˜550 mg/m2 for a 70 kg, 67 in tall patient), 2×1000 mg (total dose of ˜1100 mg/m2 for a 70 kg, 67 in tall patient). At each of these doses, substantial depletion of circulating B-lymphocytes was observed following the administration of the first dose of the antibody.

In the present methods of treating autoimmune diseases and of depleting B cells in a patient having an autoimmune disease, in one embodiment, a humanized 2H7 antibody is administered at a flat dose in the range of 0.1 mg to 1000 mg. We have found that at flat doses of less than 300 mg, even at 10 mg, substantial B cell depletion is achieved. Thus, in the present B cell depletion and treatment methods in different embodiments, hu2H7.v511 antibody is administered at dosages of 0.1, 0.5, 1, 5, 10, 15, 20 25, 30, 40, 50, 75, 100, 125, 150, 200, or 250 mg. Lower doses e.g., at 20 mg, 10 mg or lower can be used if partial or short term B cell depletion is the objective.

Depending on the disease, the anti-integrin antibodies such as the α4 and αL antibodies can be administered to the patient in a dosage range of about 1 mg/kg to 20 mg/kg. In different embodiments, the dosage range is 1-15 mg/kg, 1-10 mg/kg, 2-10 mg/kg, 3-10 mg/kg. In a specific embodiment, each of the α4 and αL antibodies is administered at about 5 mg/kg.

As a general proposition, the initial pharmaceutically effective amount of the small molecule antagonists of alpha4 or alphaL integrins when administered parenterally per dose will be in the range of about 0.01-100 mg/kg, preferably about 0.1 to 20 mg/kg of patient body weight per day, with the typical initial range of compound used being 0.3 to 15 mg/kg/day. Oral unit dosage forms, such as tablets and capsules, preferably contain from about 25 to about 1000 mg of the compound of the invention.

In treating disease, the B cell mobilizing and depleting agents of the invention can be administered to the patient chronically or intermittently, as determined by the physician of skill in the disease.

A patient administered a drug by intravenous infusion or subcutaneously may experience adverse events such as fever, chills, burning sensation, asthenia and headache. To alleviate or minimize such adverse events, the patient may receive an initial conditioning dose(s) of the antibody followed by a therapeutic dose. The conditioning dose(s) will be lower than the therapeutic dose to condition the patient to tolerate higher dosages.

5. Route of Administration

The antagonists and antibodies used in the methods of the invention are administered to a human patient in accord with methods known to medical practitioners, such as by intravenous administration, e.g., as a bolus or by continuous infusion over a period of time, by subcutaneous, intramuscular, intra-arterial, intraperitoneal, intrapulmonary, intracerobrospinal, intra-articular, intrasynovial, intrathecal, intralesional, or inhalation routes (e.g., intranasal), generally by intravenous or subcutaneous administration.

In on embodiment, the humanized 2H7 antibody and/or humanized anti-alpha4beta I antibody, natalizumab, is administered by intravenous infusion with 0.9% sodium chloride solution as an infusion vehicle.

6. Combination Therapy

In treating the B cell neoplasms described above, the patient can be treated with the B cell mobilizing agents and B cell depleting agents in particular, CD20 binding antibodies, of the present invention in conjunction with one or more therapeutic agents such as a chemotherapeutic agent in a multidrug regimen. The B cell mobilizing agent and B cell depleting agent, for example, CD20 binding antibody, can be administered concurrently, sequentially, or alternating with the chemotherapeutic agent, or after non-responsiveness with other therapy. Standard chemotherapy for lymphoma treatment may include cyclophosphamide, cytarabine, melphalan and mitoxantrone plus melphalan. CHOP is one of the most common chemotherapy regimens for treating Non-Hodgkin's lymphoma. The following are the drugs used in the CHOP regimen: cyclophosphamide (brand names cytoxan, neosar); adriamycin (doxorubicin/hydroxydoxorubicin); vincristine (Oncovin); and prednisolone (sometimes called Deltasone or Orasone). In particular embodiments, the B cell depleting agent such as CD20 binding antibody and B cell mobilizing agent, such as alpha4 or alphaL integrin antagonist is administered to a patient in need thereof in combination with one or more of the following chemotherapeutic agents of doxorubicin, cyclophosphamide, vincristine and prednisolone. In a specific embodiment, a patient suffering from a lymphoma (such as a non-Hodgkin's lymphoma) is treated with an anti-CD20 antibody and an anti-alpha4beta I antibody of the present invention in conjunction with CHOP (cyclophosphamide, doxorubicin, vincristine and prednisone) therapy. In another embodiment, the cancer patient can be treated with a humanized CD20 binding antibody and a small molecule alpha4 and/or alphaL integrin antagonist of the invention in combination with CVP (cyclophosphamide, vincristine, and prednisone) chemotherapy. In a specific embodiment, the patient suffering from CD20-positive NHL is treated with humanized 2H7.v16 and natalizumab in conjunction with CVP. In a specific embodiment of the treatment of CLL, a CD20 binding antibody and integrin antagonist is administered in conjunction with chemotherapy with one or both of fludarabine and cytoxan.

In treating the autoimmune diseases or autoimmune related conditions described above, the patient can be treated with the B cell depleting agent such as a CD20 binding antibody and an alpha4 and/or alphaL integrin antagonist in conjunction with a second therapeutic agent, such as an immunosuppressive agent, such as in a multi drug regimen. The B cell depleting agent can be administered concurrently, sequentially, or alternating with the B cell mobilizing agent, and concurrently, sequentially, alternating with the immunosuppressive agent or upon non-responsiveness with other therapy. The immunosuppressive agent can be administered at the same or lesser dosages than as set forth in the art. The preferred adjunct immunosuppressive agent will depend on many factors, including the type of disorder being treated as well as the patient's history.

“Immunosuppressive agent” as used herein for adjunct therapy refers to substances that act to suppress or mask the immune system of a patient. Such agents would include substances that suppress cytokine production, down regulate or suppress self-antigen expression, or mask the MHC antigens. Examples of such agents include steroids such as glucocorticosteroids, e.g., prednisone, methylprednisolone, and dexamethasone; 2-amino-6-aryl-5-substituted pyrimidines (see U.S. Pat. No. 4,665,077), azathioprine (or cyclophosphamide, if there is an adverse reaction to azathioprine); bromocryptine; glutaraldehyde (which masks the MHC antigens, as described in U.S. Pat. No. 4,120,649); anti-idiotypic antibodies for MHC antigens and MHC fragments; cyclosporin A; cytokine or cytokine receptor antagonists including anti-interferon-γ, -β, or -α antibodies; anti-tumor necrosis factor-α antibodies; anti-tumor necrosis factor-β antibodies; anti-interleukin-2 antibodies and anti-IL-2 receptor antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published Jul. 26, 1990); streptokinase; TGF-β; streptodomase; RNA or DNA from the host; FK506; RS-61443; deoxyspergualin; rapamycin; T-cell receptor (U.S. Pat. No. 5,114,721); T-cell receptor fragments (Offner et al., Science 251:430-432 (1991); WO 90/11294; and WO 91/01133); and T cell receptor antibodies (EP 340,109) such as T10B9.

For the treatment of rheumatoid arthritis, the patient can be treated with a B cell depleting agent such as an anti-CD20 antibody and a B cell mobilizing agent such as an alpha4 and/or alphaL integrin antagonist, in conjunction with any one or more of the following drugs: disease-modifying anti-rheumatic drugs (DMARD) (e.g., methotrexate), NSAI or NSAID (non-steroidal anti-inflammatory drugs), HUMIRA™ (adalimumab; Abbott Laboratories), ARAVA® (leflunomide), REMICADE® (infliximab; Centocor Inc., of Malvern, Pa.), ENBREL (etanercept; Immunex, WA), COX-2 inhibitors. DMARDs commonly used in RA are hydroxycloroquine, sulfasalazine, methotrexate, leflunomide, etanercept, infliximab, azathioprine, D-penicillamine, Gold (oral), Gold (intramuscular), minocycline, cyclosporine, Staphylococcal protein A immunoadsorption. Adalimumab is a human monoclonal antibody that binds to TNFα. Infliximab is a chimeric monoclonal antibody that binds to TNFα. Etanercept is an “immunoadhesin” fusion protein consisting of the extracellular ligand binding portion of the human 75 kD (p75) tumor necrosis factor receptor (TNFR) linked to the Fc portion of a human IgG1. For conventional treatment of RA, see, e.g., “Guidelines for the management of rheumatoid arthritis” Arthritis & Rheumatism 46(2): 328-346 (February, 2002). In a specific embodiment, the RA patient is treated with a CD20 antibody of the invention in conjunction with methotrexate (MTX). An exemplary dosage of MTX is about 7.5-25 mg/kg/wk. MTX can be administered orally and subcutaneously.

For the treatment of ankylosing spondylitis, psoriatic arthritis and Crohn's disease, the patient can be treated with a B cell depleting agent such as a CD20 binding antibody and a B cell mobilizing agent such as an alpha4 and/or alphaL integrin antagonist in conjunction with, for example, Remicade® (infliximab; from Centocor Inc., of Malvern, Pa.), ENBREL (etanercept; Immunex, WA).

Treatments for SLE include high-dose corticosteroids and/or cyclophosphamide (HDCC).

For the treatment of psoriasis, patients can be administered a B cell depleting agent such as an anti-CD20 binding antibody and a B cell mobilizing agent such as an alpha4 and/or alphaL integrin antagonist, in conjunction with topical treatments, such as topical steroids, anthralin, calcipotriene, clobetasol, and tazarotene, or with methotrexate, retinoids, cyclosporine, PUVA and UVB therapies. In one embodiment, the psoriasis patient is treated with the CD20 binding antibody sequentially or concurrently with cyclosporine.

7. Pharmaceutical Formulations

Therapeutic formulations of the B cell depletion agents, such as CD20-binding antibodies and B cell mobilizing agents, such as alpha4 and/or alphaL integrin antagonist used in accordance with the present invention are prepared for storage by mixing the agent or small molecule antagonist, for example an antibody having the desired degree of purity with optional pharmaceutically acceptable carriers, excipients, or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Exemplary anti-CD20 antibody formulations are described in WO98/56418, expressly incorporated herein by reference. Another formulation is a liquid multidose formulation comprising the anti-CD20 antibody at 40 mg/mL, 25 mM acetate, 150 mM trehalose, 0.9% benzyl alcohol, 0.02% polysorbate 20 at pH 5.0 that has a minimum shelf life of two years storage at 2-8° C. Another anti-CD20 formulation of interest comprises 10 mg/mL antibody in 9.0 mg/mL sodium chloride, 7.35 mg/mL sodium citrate dihydrate, 0.7 mg/mL polysorbate 80, and Sterile Water for Injection, pH 6.5. Yet another aqueous pharmaceutical formulation comprises 10-30 mM sodium acetate from about pH 4.8 to about pH 5.5, preferably at pH5.5, polysorbate as a surfactant in a an amount of about 0.01-0.1% v/v, trehalose at an amount of about 2-10% w/v, and benzyl alcohol as a preservative (U.S. Pat. No. 6,171,586). Lyophilized formulations adapted for subcutaneous administration are described in WO97/04801. Such lyophilized formulations may be reconstituted with a suitable diluent to a high protein concentration and the reconstituted formulation may be administered subcutaneously to the mammal to be treated herein.

One antibody formulation for the humanized 2H7 variants comprises antibody at 12-14 mg/mL in 10 mM histidine, 6% sucrose, 0.02% polysorbate 20, pH 5.8. In a specific embodiment, 2H7 variants and in particular 2H7.v16 is formulated at 20 mg/mL antibody in 10 mM histidine sulfate, 60 mg/ml sucrose., 0.2 mg/ml polysorbate 20, and Sterile Water for Injection, at pH5.8.

Exemplary formulations of small molecule integrin antagonists are disclosed, for example, in WO02/059114.

The formulation herein may also contain more than one active compound as necessary for the particular indication being treated, preferably those with complementary activities that do not adversely affect each other. For example, it may be desirable to further provide a cytotoxic agent, chemotherapeutic agent, cytokine or immunosuppressive agent (e.g. one which acts on T cells, such as cyclosporin or an antibody that binds T cells, e.g. one which binds LFA-1). The effective amount of such other agents depends on the amount of antibody present in the formulation, the type of disease or disorder or treatment, and other factors discussed above. These are generally used in the same dosages and with administration routes as described herein or about from 1 to 99% of the heretofore employed dosages.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi-permeable matrices of solid hydrophobic polymers containing the antagonist, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

G. Articles of Manufacture and Kits

Another embodiment of the invention is an article of manufacture containing materials useful for the treatment of an autoimmune disease or a cancer such as CLL. The article of manufacture comprises at least one container and a label or package insert on or associated with the container. Suitable containers include, for example, bottles, vials, syringes, etc. The containers may be formed from a variety of materials such as glass or plastic. At least one container holds a composition which is effective for treating the condition and may have a sterile access port (for example the container may be an intravenous solution bag or a vial having a stopper pierceable by a hypodermic injection needle). Two therapeutic compositions may be provided in the article of manufacture. At least one active agent in the first composition is a B cell depleting agent, such as a CD20 binding antibody. The second or second and third compositions containing at least one B cell mobilizing agent, such as an alpha4 or alphaL integrin antagonist, for example antibodies to the αL and α4 integrins, may be held in one or more separate containers. Alternatively, the integrin antagonist composition(s) may be packaged in a separate article of manufacture. The label or package insert indicates that the composition is used for treating the particular condition. The label or package insert will further comprise instructions for administering the compositions to the patient. Package insert refers to instructions customarily included in commercial packages of therapeutic products, that contain information about the indications, usage, dosage, administration, contraindications and/or warnings concerning the use of such therapeutic products. Additionally, the article of manufacture may further comprise a container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

H. Antibody Production

1. Monoclonal Antibodies

Monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol. 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The binding affinity of the monoclonal antibody can, for example, be determined by the Scatchard analysis described in Munson et al., Anal. Biochem. 107:220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal e.g, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., 1993, Curr. Opinion in Immunol. 5:256-262 and Plückthun, 1992, Immunol. Revs. 130:151-188 (1992).

In a further embodiment, monoclonal antibodies or antibody fragments can be isolated from antibody phage libraries generated using the techniques described in McCafferty et al., 1990, Nature, 348:552-554. Clackson et al., Nature, 1991, 352:624-628 and Marks et al., 1991, J. Mol. Biol. 222:581-597 describe the isolation of murine and human antibodies, respectively, using phage libraries. Subsequent publications describe the production of high affinity (nM range) human antibodies by chain shuffling), as well as combinatorial infection and in vivo recombination as a strategy for constructing very large phage libraries (Waterhouse et al., 1993, Nuc. Acids. Res. 21:2265-2266). Thus, these techniques are viable alternatives to traditional monoclonal antibody hybridoma techniques for isolation of monoclonal antibodies.

The DNA that encodes the antibody may be modified to produce chimeric or fusion antibody polypeptides, for example, by substituting human heavy chain and light chain constant domain (C_{H} and C_{L}) sequences for the homologous murine sequences (U.S. Pat. No. 4,816,567; and Morrison, et al., 1984, Proc. Natl. Acad. Sci. USA 81:6851), or by fusing the immunoglobulin coding sequence with all or part of the coding sequence for a non-immunoglobulin polypeptide (heterologous polypeptide). The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

2. Humanized Antibodies

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al. 1986, Nature 321:522-525; Reichmann et al., 1988, Nature, 332:323-327; Verhoeyen et al. 1988, Science 239:1534-1536), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity and HAMA response (human anti-mouse antibody) when the antibody is intended for human therapeutic use. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al., 1993, J. Immunol. 151:2296; Chothia et al., 1987, J. Mol. Biol., 196:901). Another method uses a particular framework region derived from the consensus sequence of all human antibodies of a particular subgroup of light or heavy chains. The same framework may be used for several different humanized antibodies (Carter et al., 1992, Proc. Natl. Acad. Sci. USA 89:4285; Presta et al., 1993, J. Immunol. 151:2623).

It is further important that antibodies be humanized with retention of high binding affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

The humanized antibody may be an antibody fragment, such as a Fab, which is optionally conjugated with one or more cytotoxic agent(s) in order to generate an immunoconjugate. Alternatively, the humanized antibody may be an full length antibody, such as an full length IgG1 antibody.

3. Human Antibodies and Phage Display Methodology

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., 1993, Proc. Natl. Acad. Sci. USA 90:2551; Jakobovits et al., 1993 Nature 362:255-258; Bruggemann et al., 1993, Year in Immuno. 7:33; U.S. Pat. Nos. 5,545,806, 5,569,825, 5,591,669 (all of GenPharm); 5,545,807; and WO 97/17852.

Alternatively, phage display technology (McCafferty et al., 1990, Nature 348:552-553) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson et al., 1993, Current Opinion in Structural Biology 3:564-571. Several sources of V-gene segments can be used for phage display. Clackson et al., 1991, Nature 352:624-628 isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., 1991, J. Mol. Biol. 222:581-597, or Griffith et al., 1993, EMBO J. 12:725-734. See, also, U.S. Pat. Nos. 5,565,332 and 5,573,905.

As discussed above, human antibodies may also be generated by in vitro activated B cells (see U.S. Pat. Nos. 5,567,610 and 5,229,275).

4. Antibody Fragments

In certain circumstances there are advantages of using antibody fragments, rather than whole antibodies. The smaller size of the fragments allows for rapid clearance, and may lead to improved access to solid tumors.

Various techniques have been developed for the production of antibody fragments. Traditionally, these fragments were derived via proteolytic digestion of intact antibodies (see, e.g., Morimoto et al., 1992, Journal of Biochemical and Biophysical Methods 24:107-117; and Brennan et al., 1985, Science, 229:81). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., 1992, Bio/Technology 10:163-167). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

5. Bispecific Antibodies

Bispecific antibodies are antibodies that have binding specificities for at least two different epitopes. Exemplary bispecific antibodies may bind to two different epitopes of the CD20 protein. Other such antibodies may combine a CD20 binding site with a binding site for another protein. Alternatively, an anti-CD20 arm may be combined with an arm which binds to a triggering molecule on a leukocyte such as a T-cell receptor molecule (e.g. CD3), or Fc receptors for IgG (FcγR), such as FcγRI (CD64), FcγRII (CD32) and FcγRIII (CD16), or NKG2D or other NK cell activating ligand, so as to focus and localize cellular defense mechanisms to the CD20-expressing cell. Bispecific antibodies may also be used to localize cytotoxic agents to cells which express CD20. These antibodies possess a CD20-binding arm and an arm which binds the cytotoxic agent (e.g. saporin, anti-interferon-α, vinca alkaloid, ricin A chain, methotrexate or radioactive isotope hapten). Bispecific antibodies can be prepared as full length antibodies or antibody fragments (e.g. F(ab′)₂ bispecific antibodies).

WO 96/16673 describes a bispecific anti-ErbB2/anti-FcγRIII antibody and U.S. Pat. No. 5,837,234 discloses a bispecific anti-ErbB2/anti-FcγRI antibody. A bispecific anti-ErbB2/Fcα antibody is shown in WO98/02463. U.S. Pat. No. 5,821,337 teaches a bispecific anti-ErbB2/anti-CD3 antibody.

Methods for making bispecific antibodies are known in the art. Traditional production of full length bispecific antibodies is based on the co-expression of two immunoglobulin heavy chain-light chain pairs, where the two chains have different specificities (Millstein et al. 1983, Nature, 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., 1991, EMBO J., 10:3655-3659.

According to a different approach, antibody variable domains with the desired binding specificities (antibody-antigen combining sites) are fused to immunoglobulin constant domain sequences. Preferably, the fusion is with an Ig heavy chain constant domain, comprising at least part of the hinge, C_{H}2, and C_{H}3 regions. It is preferred to have the first heavy-chain constant region (CHI) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant affect on the yield of the desired chain combination.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., 1986, Methods in Enzymology 121:210.

According to another approach described in U.S. Pat. No. 5,731,168, the interface between a pair of antibody molecules can be engineered to maximize the percentage of heterodimers which are recovered from recombinant cell culture. The preferred interface comprises at least a part of the C_{H}3 domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al.,, 1985, Science 229:81 describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., 1992, J. Exp. Med., 175: 217-225 describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., 1992, J. Immunol. 148:1547-1553. The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., 1993, Proc. Natl. Acad. Sci. USA 90:6444-6448 has provided an alternative mechanism for making bispecific antibody fragments. The fragments comprise a V_{H} connected to a V_{L} by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., 1994, J. Immunol. 152:5368.

Antibodies with more than two valencies are contemplated. For example, trispecific antibodies can be prepared. Tutt et al., 1991, J. Immunol. 147:60.

6. Multivalent Antibodies

A multivalent antibody may be internalized (and/or catabolized) faster than a bivalent antibody by a cell expressing an antigen to which the antibodies bind. The antibodies of the present invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

7. Selection and Transformation of Host Cells

Suitable host cells for cloning or expressing the recombinant mAbs, immunoadhesins and other polypeptide antagonists described herein are prokaryote, yeast, or higher eukaryote cells. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

Full length antibody, antibody fragments, and antibody fusion proteins can be produced in bacteria, in particular when glycosylation and Fc effector function are not needed, such as when the therapeutic antibody is conjugated to a cytotoxic agent (e.g., a toxin) and the immunoconjugate by itself shows effectiveness in tumor cell destruction. Full length antibodies have greater half life in circulation. Production in E. coli is faster and more cost efficient. For expression of antibody fragments and polypeptides in bacteria, see, e.g., U.S. Pat. No. 5,648,237 (Carter et. al.), U.S. Pat. No. 5,789,199 (Joly et al.), and U.S. Pat. No. 5,840,523 (Simmons et al.) which describes translation initiation region (TIR) and signal sequences for optimizing expression and secretion, these patents incorporated herein by reference. After expression, the antibody is isolated from the E. coli cell paste in a soluble fraction and can be purified through, e.g., a protein A or G column depending on the isotype. Final purification can be carried out similar to the process for purifying antibody expressed e.g., in CHO cells.

In addition to prokaryotes, eukaryotic microbes such as filamentous fungi or yeast are suitable cloning or expression hosts for antibody-encoding, such as CD20 antibody-encoding, vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g. Neurospora, Penicillium, Tolypocladium, and Aspergillus hosts such as A. nidulans and A. niger.

Suitable host cells for the expression of, e.g., glycosylated CD20 binding antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., 1977, J. Gen Virol. 36:59); baby hamster kidney cells (BHK, ATCC CCL 10); Chinese hamster ovary cells/-DHFR (CHO, Urlaub et al., 1980, Proc. Natl. Acad. Sci. USA 77:4216); mouse sertoli cells (TM4, Mather, 1980, Biol. Reprod. 23:243-251; monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., 1982, Annals N.Y. Acad. Sci. 383:44-68); MRC 5 cells; FS4 cells; and a human hepatoma line (Hep G2).

Host cells are transformed with expression or cloning vectors for a B cell depleting antibody such as CD20 binding antibody, or an integrin antagonist antibody production and cultured in conventional nutrient media modified as appropriate for inducing promoters, selecting transformants, or amplifying the genes encoding the desired sequences.

8. Culturing the Host Cells

The host cells used to produce an antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., 1979, Meth. Enz. 58:44, Barnes et al., 1980, Anal. Biochem. 102:255, U.S. Pat. No. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

9. Purification of Antibody

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., 1992, Bio/Technology 10:163-167 describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., 1983, J. Immunol. Meth. 62:1-13). Protein G is recommended for all mouse isotypes and for human γ3 (Guss et al., 1986, EMBO J. 5:15671575). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25 M salt).

10. Antibody Conjugates

The antibody may be conjugated to a cytotoxic agent such as a toxin or a radioactive isotope. In certain embodiments, the toxin is calicheamicin, a maytansinoid, a dolastatin, auristatin E and analogs or derivatives thereof, are preferable.

### EXPERIMENTAL EXAMPLES

These experimental examples are by way of illustration and not intended to be a limitation on the scope of the invention.

### Example 1

### Generation of a Mouse Model of hCD20 Tg Expression

A murine model expressing the human CD20 (hCD20) genomic locus (hCD20Tg⁺⁺ mice) was developed to analyze in vivo mechanisms of function for therapeutic mAbs that eliminate cells by targeting cell surface antigens. Two independent bacterial artificial chromosomes (BACs) were injected into blastocysts derived from FVB mice to generate multiple transgenic founder lines that expressed hCD20. Two founder mice that transmitted hCD20 expression to progeny were subjected to more detailed analysis. Both founder lines demonstrated identical patterns of hCD20 expression and hence data from only one founder line will be presented herein.

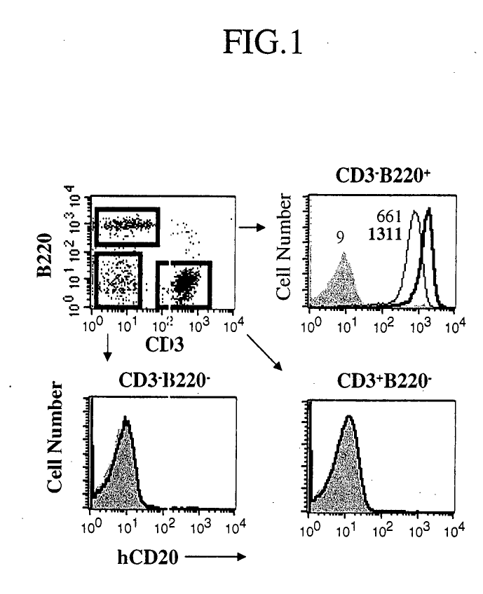

Subpopulations of circulating lymphocytes of the hCD20 transgenic (hCD20 Tg⁺) mice were analyzed by FACS and characterized according to expression of antigens B220 and CD3 in peripheral lymphocytes as shown in FIG. 1 (upper left panel). Each of the populations boxed in the upper left panel was analyzed for hCD20 expression; CD3⁻ B220⁺ (upper right panel), CD3⁺ B220⁻ (lower right panel), and CD3⁻B220⁻ cells (lower left panel).

Analysis of peripheral blood cells revealed that hCD20 was expressed exclusively on circulating B220⁺ B cells (FIG. 1). The expression level in hCD20 Tg^{+/+} mice, as determined by mean fluorescent intensity (MFI), was approximately 50% that of human circulating B cells. No hCD20 expression was detected on peripheral B220⁻ cells. Expression of hCD20 was not detectable on Tg⁻ littermates (FIG. 1, shaded).

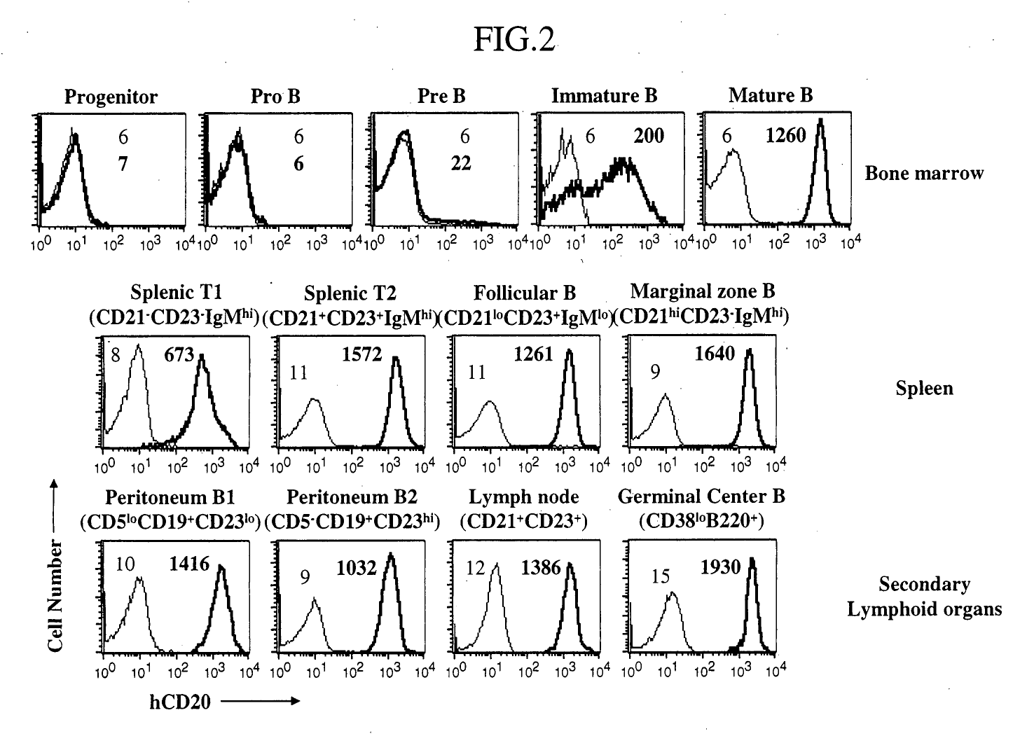

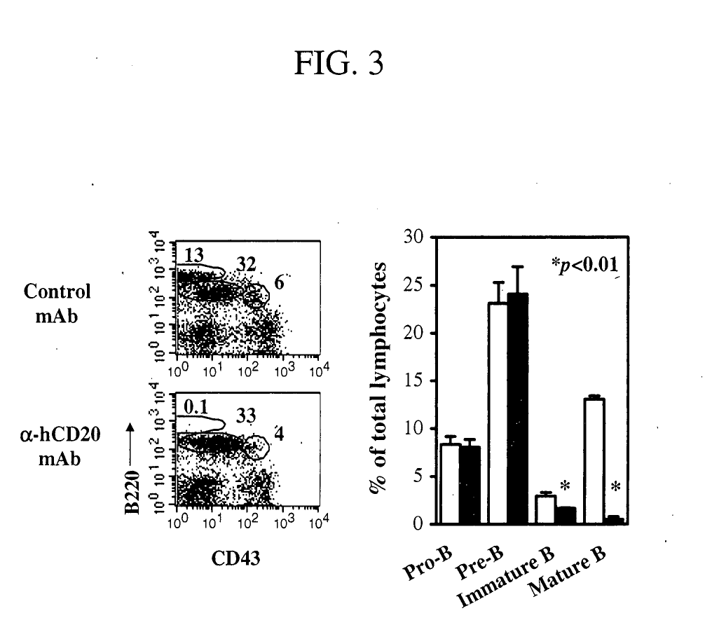

As B cells develop in the bone marrow, hCD20 expression during B cell ontogeny was analyzed. As shown in the top panel of FIG. 2, hCD20 was readily detected on immature B cells, characterized as CD43⁻ B220^{lo}IgM⁺ (see FIG. 3). Further, hCD20 was upregulated in the spleen, with the highest level of hCD20 expression detected on marginal zone (MZ) B cells (FIG. 2, middle). Immunohistochemistry was preformed on Tg⁺ and Tg⁻ mice to analyze hCD20 expression. Spleens from Tg⁺ or Tg⁻ mice were stained for IgM (green), hCD20 (red), or CD3 (blue). Immunohistochemical (1HC) analysis of splenic tissue revealed co-localization of hCD20 staining with IgM amongst the B cell zones (data not shown). hCD20 staining was not co-localized with IgM^{hi} staining plasma cells by IHC analysis, nor on Syndecan 1⁺ plasma cells by FACS analysis. hCD20 was detected on peritoneal B1 and B2 B cells, mature lymph node B cells, and Peyer's Patch germinal center (GC) B cells (FIG. 2—bottom panel). Hence, hCD20 expression in these transgenic mice qualitatively recapitulates the pattern of CD20 expression as described in humans and mice.

### Example 2

### Depletion of B Cells in Vivo by Treatment with Anti-CD20 Antibody

In this study, B cell depletion induced by treatment with anti-hCD20 antibody demonstrated kinetics that differed according to the cellular compartment in which the B cells resided.

1. Anti-hCD20 MAb Treatment

To analyze the biologic consequences of anti-hCD20 mAb treatment, hCD20 Tg⁺ mice were treated intraperitoneally with a single dose of 0.1 mg of control mouse IgG₂ₐ (non-specific antibody) or with a panel anti-hCD20 mAbs that included RITUXAN®, 2H7, B1, and 1F5. RITUXAN®, 2H7, and 1F5 bind comparable epitopes located within the second extracellular domain of CD20; B1 binds a different but overlapping epitope. Incubation of B cells with B1 has been described to not mobilize CD20 into membrane rafts. Since the binding of mouse IgG₂ₐ to mouse Fc receptors (FcRs) best parallels the binding of the human IgG₁ backbone of RITUXAN® to human FcRs, all anti-CD20 mAbs were examined on a murine IgG₂ₐ backbone.

2. Depletion of Circulating B Cells

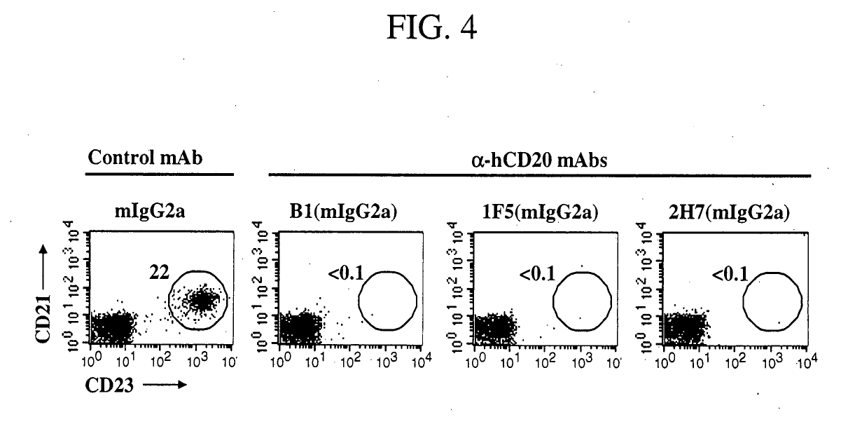

B cells present in the peripheral blood of treated and control mice were analyzed by FACS. Subpopulations of B cells were identified by expression of CD23 and CD21. As shown in FIG. 4, each of the anti-hCD20 mAbs caused depletion of peripheral B cells (circle). Depletion of peripheral B cells was correlated with the circulating serum half-life of the therapeutic mAb (data not shown).

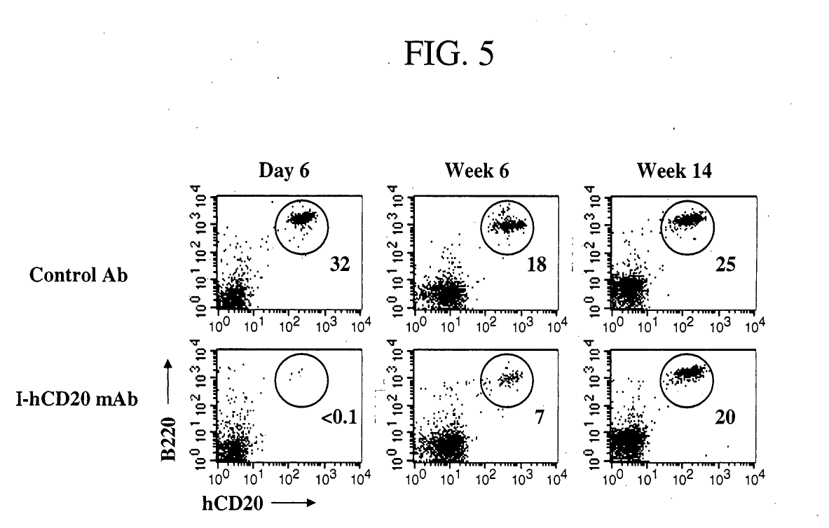

Peripheral blood of hCD20 Tg⁺ mice treated with the anti-hCD20 antibody m2H7 was analyzed on day 6, week 6, and week 14 post-treatment. As shown in FIG. 5, treatment with anti-hCD20 depleted circulating B cells, as shown at day 6 (FIG. 5, left panel). Six weeks post-treatment, when the anti-hCD20 mAb was no longer detectable in the serum (<1 μg/ml), B cells were again detected by FACS analysis within the circulation (FIG. 5, middle panel). Subsequently, circulating B cells normalized to pre-treatment levels, as shown at week 14 (FIG. 5, right panel). Consistent with the lack of expression of hCD20 in the early B cell progenitor population (see FIG. 2), only CD20⁺ immature and mature recirculating B cells in the bone marrow were depleted (circle).

3. Depletion of B Cells in Blood, Lymphnode, Peritoneal Cavity

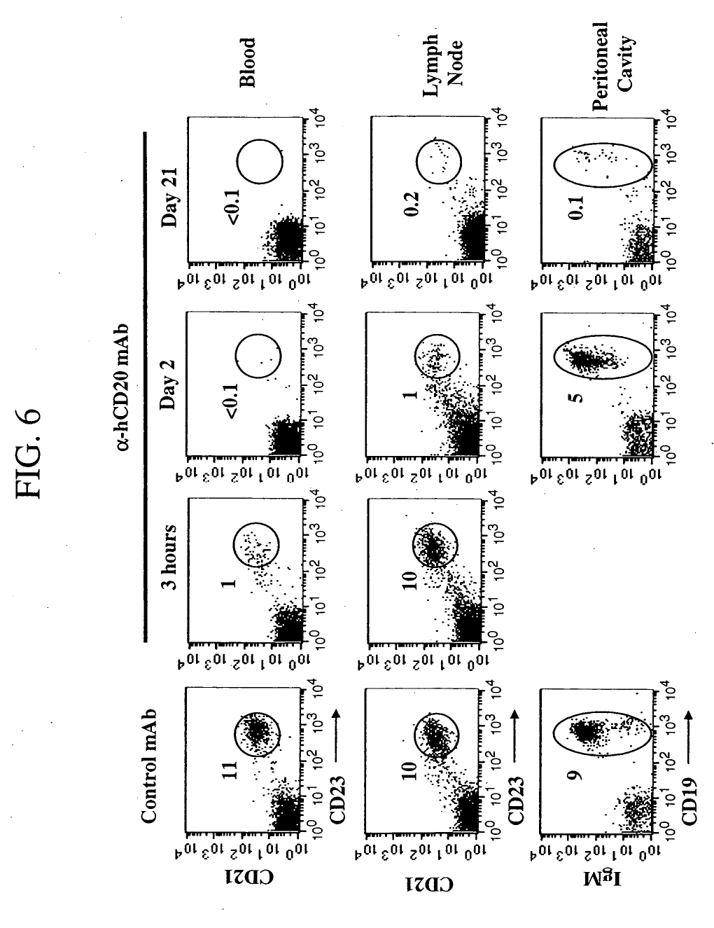

The kinetics of B-cell depletion from blood, lymph node, and peritoneal cavity was analyzed in hCD20 Tg⁺ mice treated with m2H7 anti-hCD20 MAb as described above. Results are shown in FIG. 6. Similar to the depletion of peripheral B cells, analysis of the presence of B cells in blood (top), lymph node (middle), and peritoneal cavity (bottom) at 3 hours, day 2, and day 21 demonstrated that treatment with anti-hCD20 mAb resulted in depletion of B220⁺ cells from lymph nodes and peritoneal cavity of hCD20 Tg⁺ mice (FIG. 6). Interestingly, the kinetics of depletion differed amongst these three compartments. While greater than 90% of circulating B cells were depleted within 3 hours following intravenous (IV) administration of anti-hCD20 mAbs (top panel), lymph node B cells were depleted within 2 days with either IV or intraperitoneal (IP) administration of anti-hCD20 mAbs (middle panel), and peritoneal B cells required about 21 days for greater than 90% depletion, despite IP administration of the anti-hCD20 mAb (lower panel). Since peritoneal B cells re-circulate more slowly than lymph node B cells, the distinct kinetics of depletion parallel the kinetics of lymphocyte circulation.

### Example 3

### Hierarchy of B Cell Subset Susceptibilities

Example 3 demonstrates that B cell subsets show different susceptibilities to B cell depletion upon anti-CD20 antibody treatment.

1. B Cell Depletion in Spleen

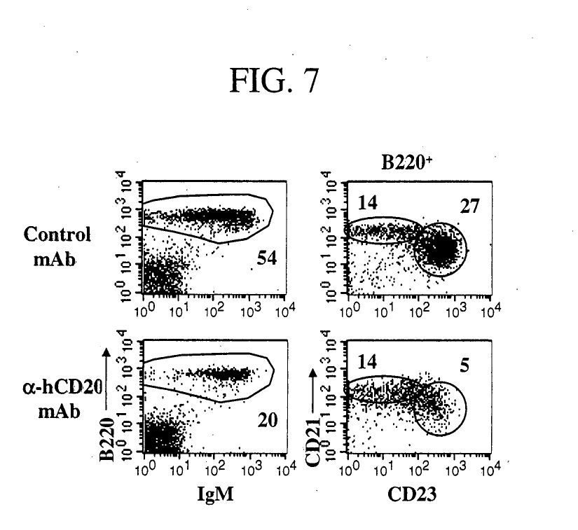

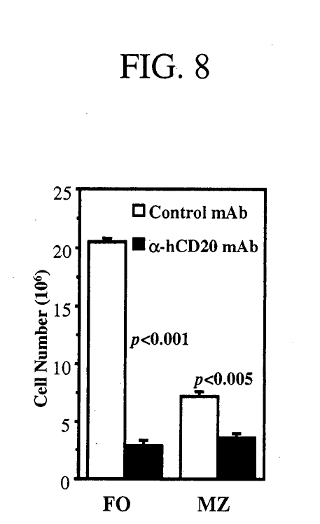

Transgenic mice described above for Example I (hCD20 Tg⁺ mice) were treated with control IgG₂ or anti-hCD20 mAb. Spleens were harvested at day 4 post-treatment and analyzed for B220, IgM, CD21, and CD23 staining, and characterized as CD21^{hi}CD23⁺ follicular (FO) B cells or CD21^{hi}CD23⁻ marginal zone (MZ) B cells (FIG. 7). B cells in each subset were quantified, as shown in FIG. 8.

In contrast to circulating mature B cells that were completely depleted by anti-hCD20 mAb, (see FIGS. 3, 4, 5, and 6), approximately 33% of B220⁺ splenocytes were resistant to anti-hC20 mAb treatment (FIG. 7). Analysis of splenic B cell subsets revealed that follicular (FO) B cells were significantly depleted (greater than 90% depletion), while CD21^{hi}CD23⁻ MZ B cells exhibited greater resistance to anti-hCD20 mAb treatment. Approximately 50% of the MZ B cells remained following anti-hCD20 mAb therapy (FIG. 8).

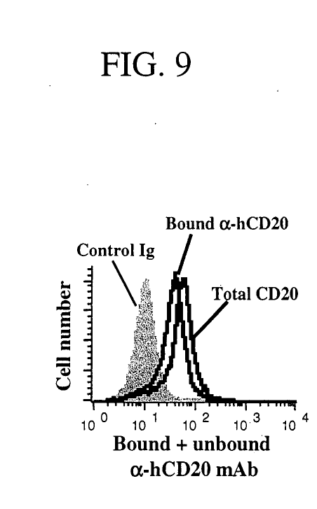

B220⁺ splenocytes isolated from the anti-hCD20 mAb treated mice were analyzed ex vivo with either a FITC-anti-mouse IgG₂ₐ mAb (to detect bound anti-hCD20 mAb) or with additional anti-hCD20 mAb followed by FITC-anti-mouse IgG₂ₐ mAb (to detect the total amount of CD20 expressed) on resistant splenic B cells. The results demonstrated that resistance was not due to the lack of hCD20 expression in MZ B cells, as hCD20 was expressed at a higher level in MZ as compared to FO B cells (FIG. 8), nor was resistance due to the lack of accessibility of the therapeutic mAb, as CD20 on resistant splenic B cells was nearly saturated with the in vivo administered anti-hCD20 mAb (FIG. 9).

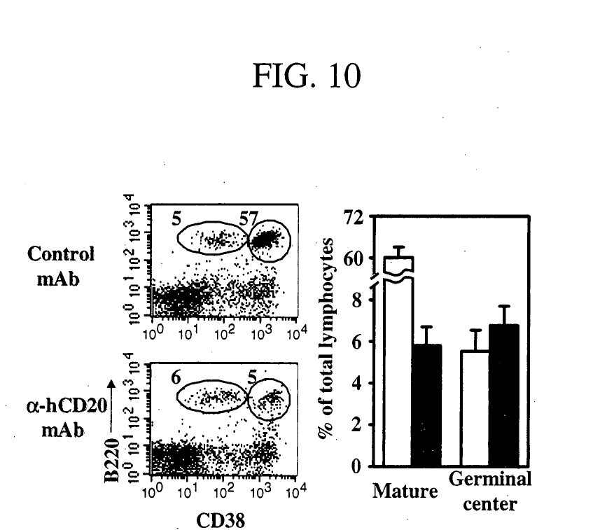

Even more dramatic than resistant splenic marginal zone B cells, germinal center (GC) B cells resident within Peyer's Patches demonstrated greatest resistance to anti-hCD20 mAb treatment. While mature B220⁺CD38^{hi} B cells were readily depleted, the B220⁺CD38^{lo} GC B cells were resistant to anti-hCD20 mAb therapy, as shown in FIG. 10.

To extend these observations on Peyer's Patch resident GC B cells, splenic GC B cells generated through immunization with sheep red blood cells (SRBCs), were tested for resistance. Mice were immunized with SRBCs to induce GC formation. As GCs are maximally formed by day 8 following immunization, mice were treated on day 8 with 0.2 mg control IgG₂ₐ or anti-hCD20 mAb. Splenic GC B cells were characterized and quantified by B220 and PNA (peanut agglutinin) staining. Peanut agglutinin stains for GC B cells.

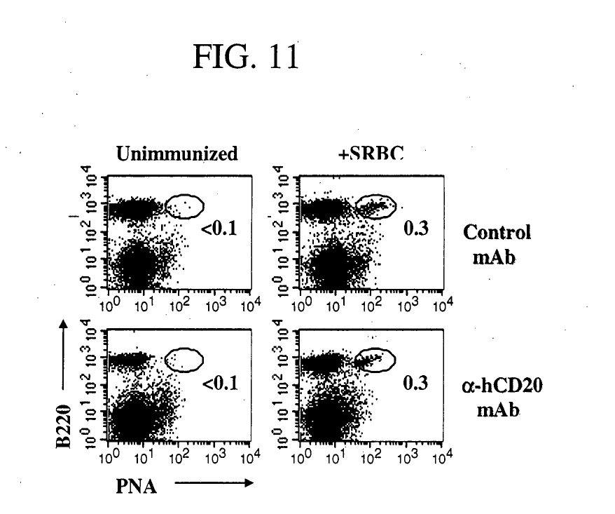

As shown in FIG. 11, non-immunized mice did not develop B220⁺PNA⁺ GC B cells (left panel, circle). SRBC immunized mice did develop PNA⁺ GC B cells (right panel, circle) that were resistant to anti-hCD20 mAb killing (FIG. 11, bottom). Resistance was independent of hCD20 expression, as both Peyer's Patch resident or splenic GC B cells expressed higher levels of hCD20 than the sensitive mature circulating B cells (FIG. 2); independent of mAb binding to GC cells, as in vivo recovered GC B cells were saturated with the administered mnAb; and independent of treatment dose or duration of treatment (data not shown). Hence, the data herein suggests a hierarchy of sensitivity to anti-hCD20 mAb treatment exists in the spleen: follicular (most sensitive)>marginal zone>germinal center (most resistant) B cells.

Further testing the resistance of MZ B cells, transgenic mice were treated with control of anti-hCD20 antibodies for 15 weeks (IP, 0.1 mg every two weeks) of long-term depletion. Splenic B cells (B220+) were characterized by surface expression of CD21 and CD23, and the number of FO and MZ B cells was quantified (n=3). In addition, high doses of anti-hCD20 mAb were administered to transgenic mice. Splenic B cells were analyzed 2 weeks post-treatment (n=4).

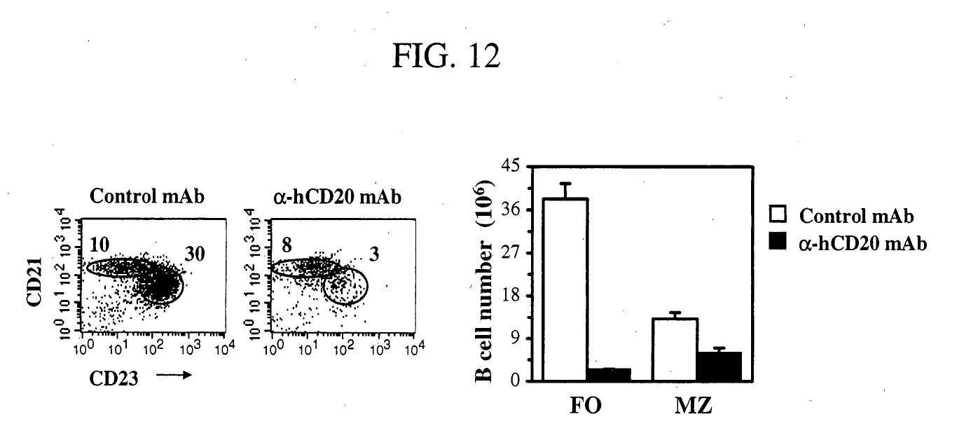

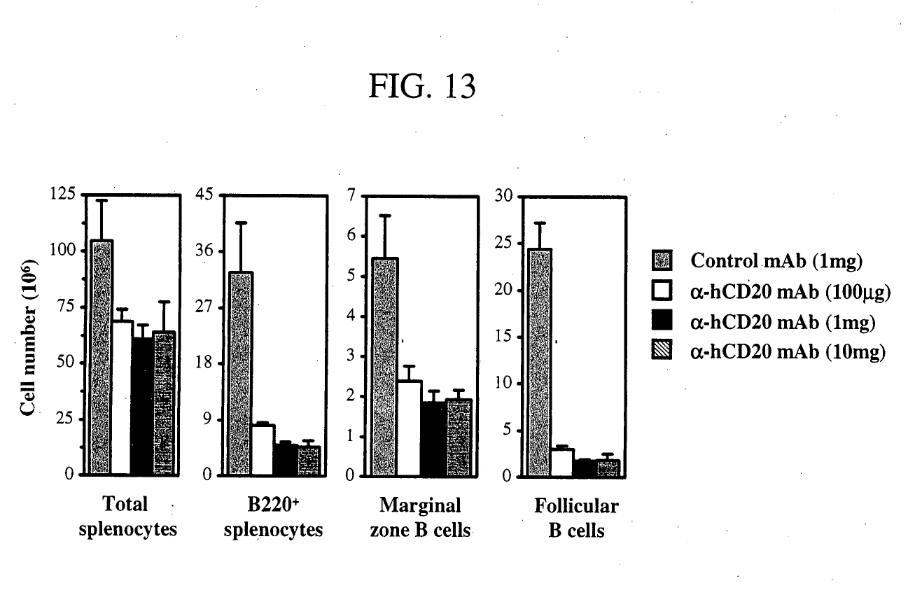

Neither administration of anti-hCD20 mAb up to 10 mg/mouse (equivalent to 15-fold greater than a cumulative four week course of RITUXAN® for NHL patients) (FIG. 13) nor continued treatment of mice with 0.1 mg every other week for 4 months with anti-hCD20 MAb resulted in any greater depletion of MZ B cells (FIG. 12).

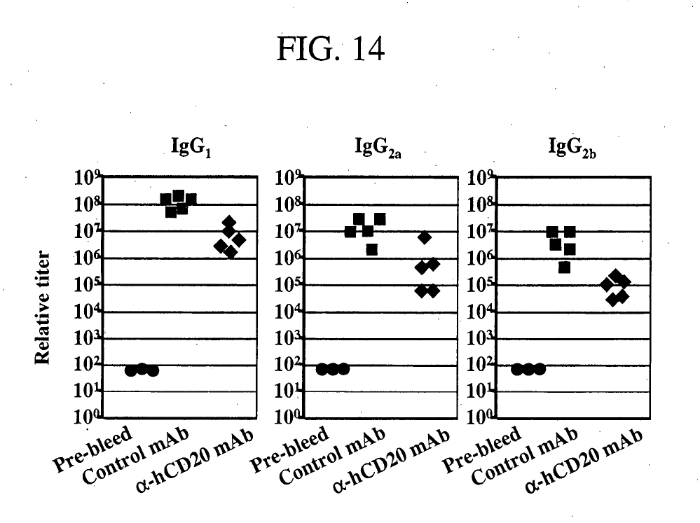

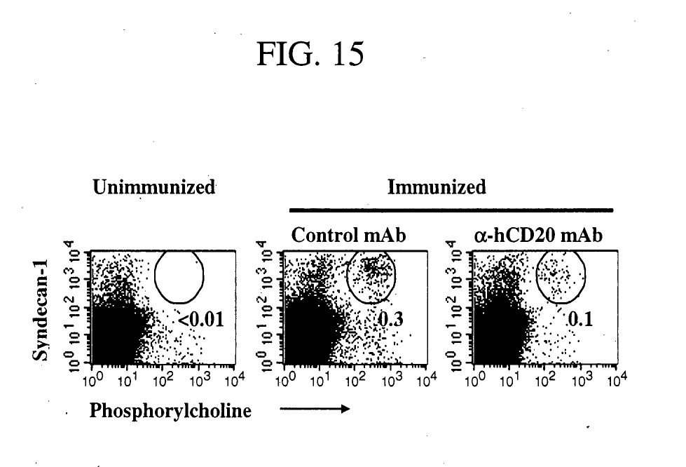

The residual resistant B cells in treated transgenic mice were functional, as anti-hCD20 mAb treated mice were capable of mounting substantial, albeit reduced, immune responses to immunogens and bacteria (FIG. 14 and FIG. 15). Transgenic animals were treated with two doses of control or anti-hCD20 mAb (0.2 mg/dose, IP) at weeks 7 and 10. Mice were immunized (SC) with (4-hydroxy-3-nitrophenyl)acetyl conjugated to keyhole limpet hemocyanin (NP-KLH) at week I and challenged again at week 11. NP-specific Ig levels were assayed at week 12 by ELISA. Data are shown in FIG. 14, where pre-bleed refers to sample taken before immunization with NP-KLH.

FIG. 15 shows T-independent immune response to a bacterial antigen. Complete depletion of peripheral and peritoneal B1 cells was achieved 3 weeks after treatment of two IP doses (0.2 mg/mouse) of control or (α-hCD20 mAbs as shown in FIG. 6. T-independent responses were assessed by FACS analysis (left panel) on antigen (Ag)-specific plasmablasts isolated 4 days following administration of heat-inactivated Streptococcus Pneumoniae. The number of Ag-specific plasmablasts were quantified (right) as mean±standard error (n=4). Syndecan-1 stains for plasmablasts and plasma cells.

### Example 4

### Intravascular Access Enhances B Cell Depletion

This Example shows mobilization of marginal zone B cells enhances the sensitivity of these cells to anti-hCD20 mAb depletion.

The hierarchy of sensitivity to anti-hCD20 mAb treatment might reflect an intrinsic resistance of cells due to the expression of negative regulatory cell surface proteins or intracellular anti-apoptotic factors, survival factors provided by the MZ and GC microenvironment, and/or access to required effector mechanisms. To evaluate the contribution of the microenvironment to the greater resistance of MZ B cells, MZ B cells were mobilized into the vasculature by co-administration of anti-αL and anti-α4 integrin mAbs.

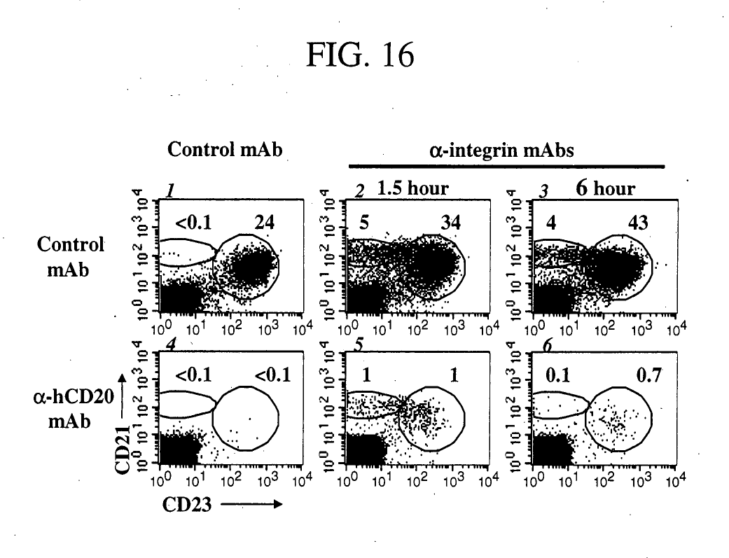

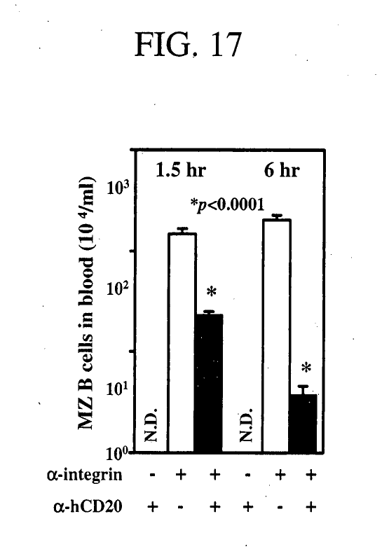

Mice (hCD20 Tg⁺) were pre-treated with control IgG₂a three days prior to the initiation of the study (day-3) to minimize non-specific effects of IgG on cellular trafficking. At day 0, mice were treated with 0.2 mg control IgG₂a or anti-hCD20 mAb. Mice were injected intravenously on day 2 with 0.1 mg each of anti-CD11a (M17) and anti-α4 integrin (PS/2) mAbs. Blood samples were analyzed 1.5 and 6 hours following the administration of the anti-integrin mAbs. As shown in FIG. 16, MZ B cells (CD21^{hi}CD23^{low}) were mobilized by the anti-alpha integrin mAb and depleted by anti-hCD20 mAb. Absolute numbers of MZ B cells (CD21^{hi}CD23^{lo}) in the blood were quantified, and are shown in FIG. 17. Mobilization of CD21^{hi}CD23^{lo} MZ B cells rendered these cells more sensitive to anti-CD20 mAb mediated depletion. See, for example FIG. 16 panels 2 and 5, panels 3 and 6; and FIG. 17.

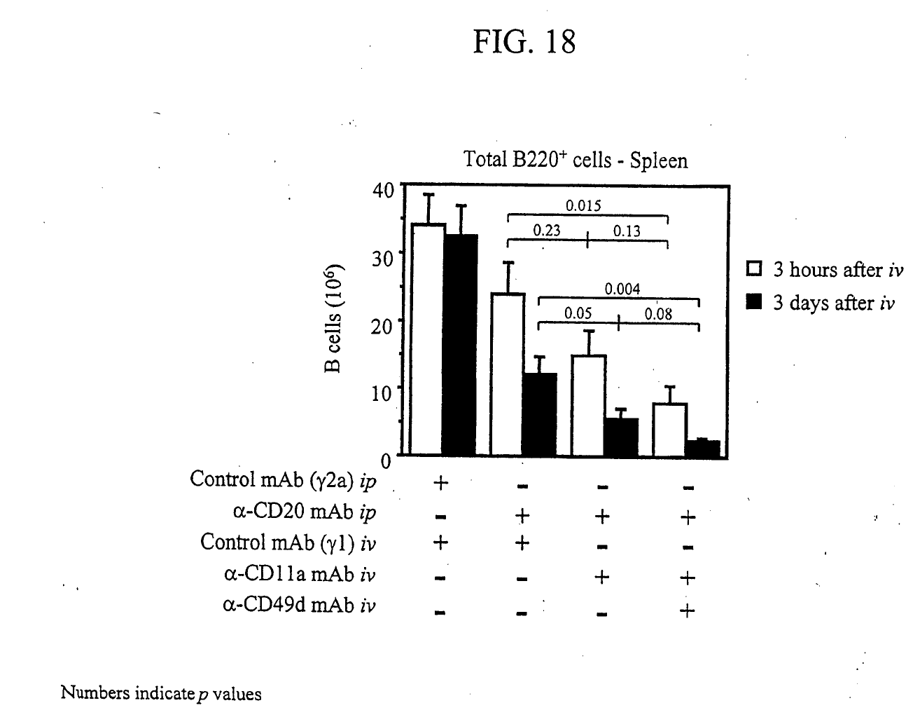

FACS analysis of splenic B cells of the treated mice revealed a concomitant decrease in MZ B cells (data not shown). Quantitation of the total B220⁺ cells in the spleen showed that the combination of an alphaL antagonist (anti-CD11a mAb) plus anti-CD20 mAb resulted in better B cell depletion than anti-CD20 mAb alone (FIG. 18), but the extent of depletion achieved is even greater using the combination of both alphaL and alpha4 mAbs with CD20 mAb (FIG. 18). The alphaL and alpha4 antagonists worked synergistically to increase the number of B cells in the circulation. Not to be limited by any one mechanism, this increase in circulating B cells is likely due to both B cell mobilization and inhibition of B cell homing.

Immunohistochemistry analysis of the spleen confirmed the preferential depletion of MZ B cells outside of the MOMA-1 staining marginal sinus with the combined treatment of anti-integrins and anti-hCD20 mAbs as compared to the relative resistance of MZ B cells outside of the marginal sinus with anti-hCD20 mAbs alone (data not shown). MOMA-1 is used to stain the subset of macrophages that separate the Fc from MZ.

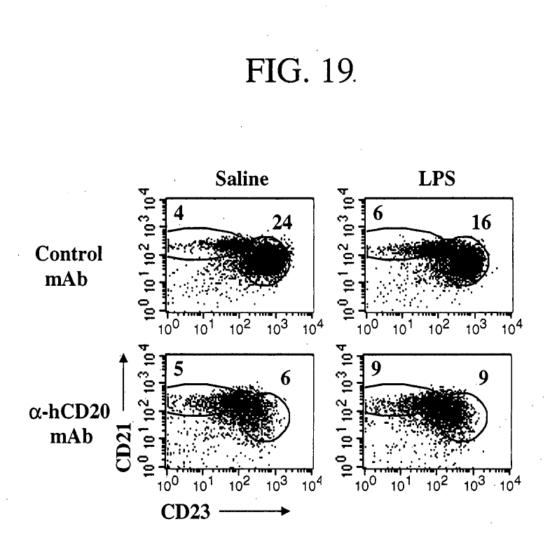

To mobilize B cells into the follicle (FO), mice were treated as described above to mobilize MZ B cells, except that the anti-integrin mAb cocktail was substituted with 25 μg lipopolysaccharide (LPS). FACs analysis of treated and control cells show that LPS treatment results in the mobilization of MZ B cells into the follicle (FIG. 19).

In contrast to the mobilization of MZ B cells into the vasculature shown in FIG. 18, mobilization of cells from the MZ into the follicle with administration of LPS did not result in depletion of the MZ B cells (FIG. 19). Together, these data suggest that MZ B cells are intrinsically susceptible to anti-hCD20 mAb treatment and that trafficking of B cells into the vasculature is essential for efficient B cell depletion.

Immunohistochemistry of splenic tissue from mice treated with control IgG, anti-hCD20 mAb, anti-hCD20, and anti-integrin mAbs, or anti-hCD20 mAb and LPS was compared. With LPS treatment, IgM staining cells were seen inside the metallophilic antigen-1 (MOMA-1) staining border in this enlarged follicle (data not shown).

Compound A, a sphingosine 1-phosphate receptor agonist, was used to prevent mature lymph node B cells from returning to the circulation, to assess if this would interfere with B cell depletion. Mice were treated with vehicle control or a sphingosine 1-phosphate receptor (S1PR) agonist (Compound A) and challenged with anti-hCD20 mAbs.

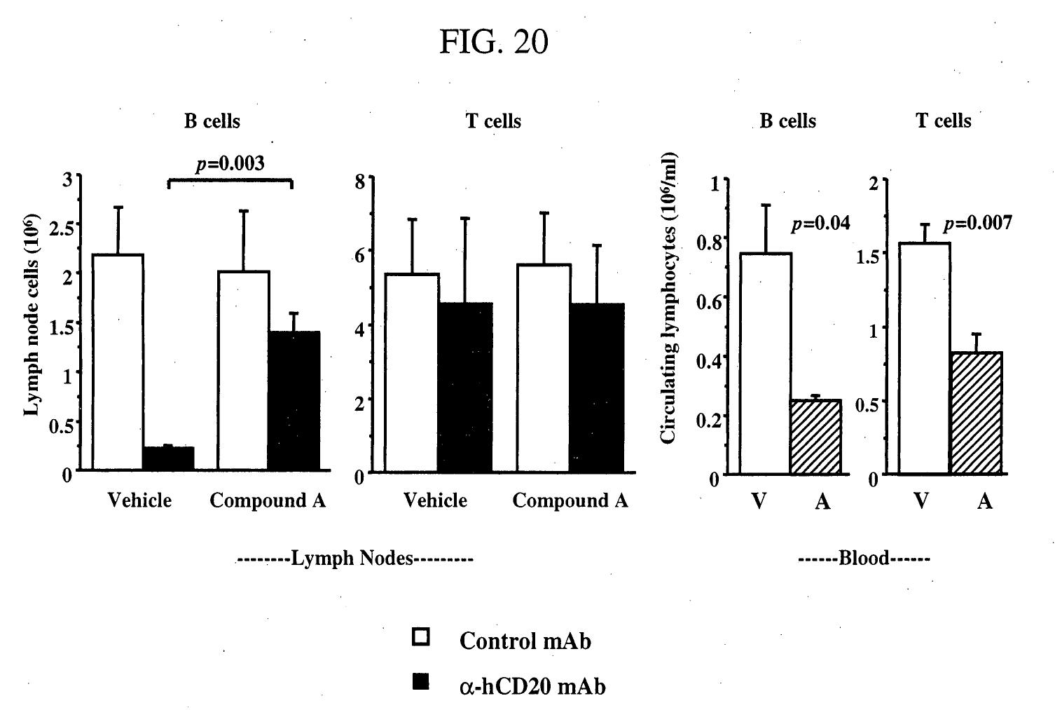

hCD20 Tg⁺ mice were treated by oral gavage with vehicle control or Compound A (10 mg/kg every 6 hours). A single dose of control or anti-hCD20 mAb (0.5 mg IP) was administered two hours after the first dose of Compound A. Lymphocytes, isolated from lymph nodes (FIG. 20, panels 1 and 2) and blood (FIG. 20, panels 3 and 4) at 20 hours, were quantified and expressed as mean±standard error (n=4).

Consistent with the inhibitory effects of S1PR agonists on lymphocyte egress from lymph node to circulation, both B and T cells were significantly decreased in mice treated with compound A (FIG. 20, panels 3 and 4). While lymph node B cells were readily depleted by anti-hCD20 mAbs in vehicle-treated mice, lymph node B cells were not depleted by anti-hCD20 mAbs in the presence of compound A (FIG. 20, panels 1 and 2). Together, this data supports the requirement for B cells to access the circulation for efficient depletion.

### Example 5

### The Role of the Liver and Spleen in B Cell Depletion

Since the reticuloendothelial system (RES) represents a major modality for clearance of apoptotic cells and immune complexes, the contributions of the liver and spleen to B cell depletion were examined. To assess liver contribution, both portal vein and hepatic artery were ligated. Ligation was accomplished by subjecting mice to sham or clamping of the portal vein and hepatic artery followed by immediate IV injection of control or anti-hCD20 (0.2 mg) mAb. Ten minutes following anti-hCD20 mAb administration, peripheral blood was analyzed for B220⁺IgM⁺ B cells, as shown in the FACS plots.

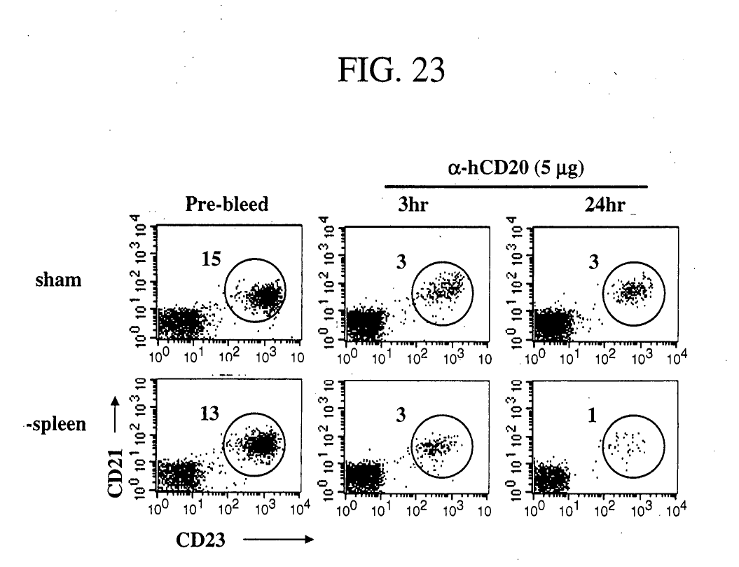

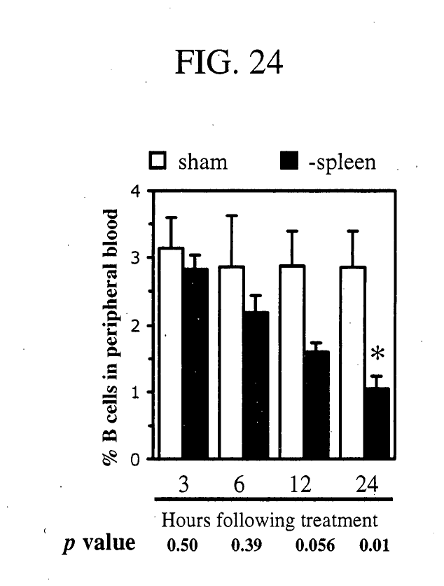

To assess splenic contributions, mice underwent either sham splenectomy (FIG. 23, top row) or splenectomy (FIG. 24, bottom row) and were analyzed for B cell depletion. Blood was analyzed 3 hours and one day following treatment with a suboptimal dose of anti-hCD20 mAb (5 μg). No differences in B cell depletion were detected with higher doses of anti-hCD20 mAb (0.1 mg). Phagocytosis by Kupfer cells of B cells following anti-hCD20 mAb treatment was examined. Mice were treated with 0.1 mg control IgG (top left) or anti-hCD20 mAb. 15 minutes following administration, livers were harvested and analyzed for B220 and F4/80 staining for B cells and macrophages, respectively. Co-localized B220⁺ and F4/80⁺ cells from 4 control and anti-hCD20 mAb treated mice were quantified.

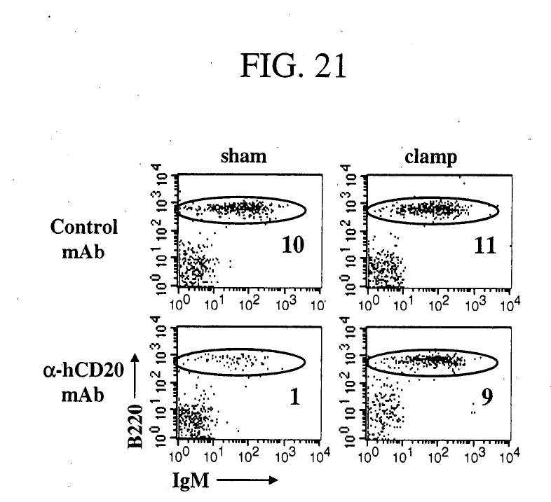

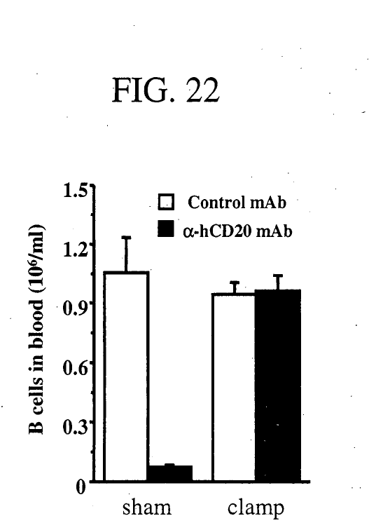

Ligation of the portal vein and hepatic artery resulted in a significant loss in the depleting ability of anti-hCD20 mAbs (FIGS. 21 and 22). In contrast, splenectomized mice demonstrated accelerated B cell depletion (FIGS. 23 and 24), an effect that was likely secondary to reduced B cell numbers in splenectomized mice.

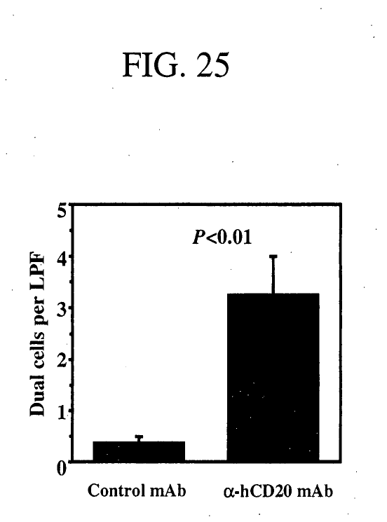

Histologic examination of livers demonstrated co-localization of B220⁺ staining B cells within F4/80⁺ staining macrophages in treated mice (FIG. 25) thus Kupfer cells engulfed B220⁺ B cells. Hence, consistent with the function of the RES, the liver represents the major portal of B cell depletion

### CONCLUSION

These data identify the in vivo mechanisms by which anti-hCD20 mAbs eliminate B cells. Upon administration of anti-hCD20 mAbs, the mAb rapidly binds CD20⁺ B cells and circulating mAb-bound B cells are rapidly cleared through the reticuloendothelial system (RES). It is advantageous for B cells that are coated with anti-hCD20 mAbs, resident in lymphoid tissue to gain access to the vasculature to deliver the targeted B cells to effector cells within the RES. This accounts for the longer periods of time required for depletion of slower recirculating peritoneal and lymph node B cells as compared to the circulating B cells. Similarly, the hierarchy of sensitivities observed for splenic and tissue laden B cell subsets reflect the reduced circulatory capacities of MZ and GC B cells. Moreover, the ability to augment or inhibit B cell depletion as a consequence of lymphocyte mobilization or inhibition of lymphocyte egress, respectively, further support the importance of intravascular access in B cell killing.

The experiments herein demonstrated surprising results in that the combination of treatment with anti-CD20 antibody and one or more integrin antagonists demonstrated great synergy in achieving enhanced depletion of B cells by depleting previously unexposed or undepleted B cell subsets.

### REFERENCES

References cited within this application, including patents, published applications and other publications, are hereby incorporated by reference.

1. A method of augmenting B cell depletion in a mammal suffering from a B cell disorder, comprising administering to the mammal, one or more B cell mobilizing agent and a therapeutically effective amount of one or more B cell depleting agent.

2. The method of claim 1, wherein the mammal is a human.

3. The method of claim 1, wherein the B cell mobilizing agent is an α4 integrin antagonist.

4. The method of claim 3, wherein the α4 integrin antagonist is an antagonist of α4β1.

5. The method of claim 3, wherein the α4 integrin antagonist is an antagonist of α4β7.

6. The method of any one of claims 3, 4, and 5, wherein the α4 integrin antagonist is an antibody, or a biologically active fragment thereof.

7. The method of claim 6, wherein the α4 integrin antagonist is a humanized, human, or chimeric antibody, or a biologically active fragment thereof.

8. The method of claim 6, wherein the antibody or antibody fragment binds the α4 subunit (CD-49d)

9. The method of claim 3, wherein the α4 integrin antagonist is natalizumab.

10. The method of claim 3, wherein the α4 integrin antagonist is the antibody PS/2 produced by the hybridoma ATCC CRL-1911, or a biologically active fragment or a humanized form thereof.

11. The method of claim 3, wherein the α4 integrin antagonist is a small molecule.

12. The method of claim 11, wherein the small molecule antagonist comprises
the formula: 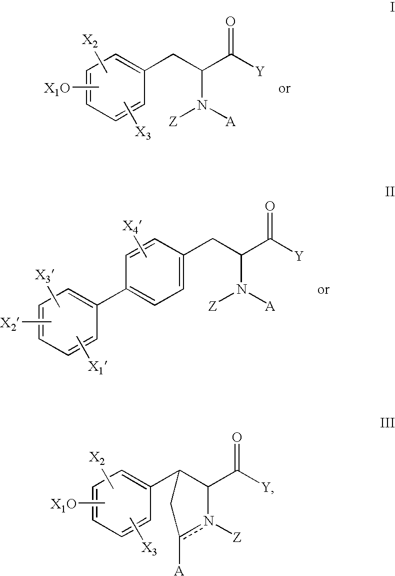
where
Z is H or lower alkyl;
A is: 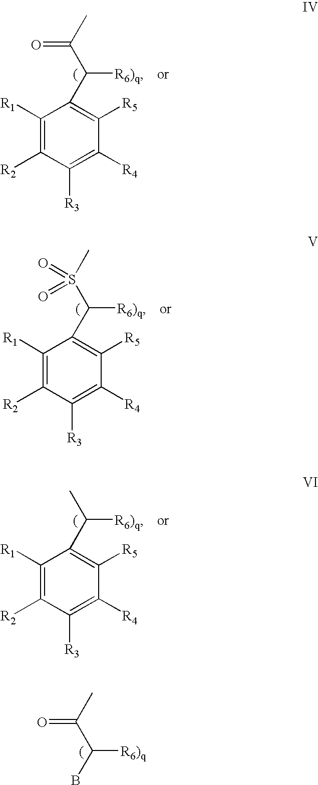
wherein B is cyanoalkyl, a carbocycle or a heterocycle optionally substituted with one or more R₁ substituents; and q is 0-3;
R₁, R₂, R₃, R₄, R₅, and R₆ independently are hydrogen, alkyl, amino, alkylamino, dialkylamino, nitro, urea, cyano, thio, alkylthio, hydroxy, alkoxy, alkoxyalkyl, alkoxycarbonyl, alkoxycarbonylamino, aryloxycarbonylamino, alkylsulfinyl, sulfonyl, alkylsulfonyl, aralkylsulfonyl, arylsulfonyl, heteroarylsulfonyl, alkanoyl, alkanoylamino, cycloalkanoylamino, aryl, arylalkyl, halogen, or alkylphosphonyl, and R₁, R₂, R₃, R₄ and R₅ are substituted with 0-3 substituents selected from the group consisting of hydroxy, carboxyl, lower alkoxycarbonyl, lower alkyl, nitro, oxo, cyano, carbocyclyl, heterocyclyl, heteroaryl, lower alkylthio, lower alkoxy, lower alkylamino, lower alkanoylamino, lower alkylsulfinyl, lower sulfonyl, lower alkylsulfonyl, lower alkanoyl, aryl, aroyl, heterocyclylcarbonyl, halogen and lower alkylphosphonyl; or two of R₁ to R₅ together form a carbocycle or heterocyclic ring;
Y is H, alkoxy, alkoxyalkoxy, aryloxy, alkylaminoalkoxy, dialkylaminoalkoxy, alkylamino, arylamino, heterocyclyl or heteroarylalkyl, where each of the forgoing may be substituted or unsubstituted;
X₁ is H, C(O)OR, C(O)NRaRb, C(O)R, or C(O)SR, wherein R, Ra and Rb, individually, is hydrogen or alkyl, alkoxy, aryl, heterocyclyl, heteroaryl, substituted with 0-4 substituents selected from the group consisting of halogen, hydroxy, amino, carboxyl, nitro, cyano, heterocylyl, heteroaryl, aryl, aroyl, aryloxy, aralkyl, aralkyloxy, aryloxycarbonyl, aralkyloxycarbonyl, alkylenedioxy, lower alkoxycarbonyl, lower alkyl, lower alkenyl, lower alkynyl, lower alkylthio, lower alkoxy, lower alkylamino, lower alkylsulfinyl, lower sulfonyl, lower alkylsulfonyl, lower alkanoyl, lower alkylphosphonyl, aminosulfonyl lower alkyl, hydroxy lower alkyl, alkylsulfinyl lower alkyl, alkylsulfonyl lower alkyl, alkylthio lower alkyl, heteroarylthio lower alkyl, heteroaryloxy lower alkyl, heteroarylamino lower alkyl, halo lower alkyl, and alkoxy lower alkyl; wherein said heterocyclyl, heteroaryl, aryl, aroyl, aryloxy, aralkyl, aralkyloxy, aryloxycarbonyl and aralkyloxycarbonyl is optionally substituted with halogen, hydroxyl, amino, carboxyl, nitro, cyano, alkyl and alkoxy; and wherein Ra and Rb together with the nitrogen to which they are attached may form a heterocyclyl or heteroaryl group substituted with 0-5 substituents R or Rd; wherein Rd has the structure: 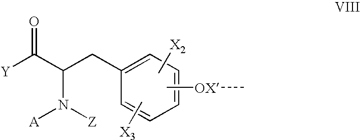
where X′ is a divalent linker selected from the group consisting of C(O)NRa, C(O) or a bond;
X₂ and X₃ are each independently hydrogen, halogen, hydroxy, amino, carboxyl, nitro, cyano, or substituted or unsubstituted alkyl, aryl, heterocylyl, heteroaryl, aryl, aroyl, aryloxy, alkylenedioxy, lower alkyl carbonylamino, lower alkenyl carbonylamino, aryl carbonylamino, arylalkyl carbonylamino, lower alkoxy carbonylamino, lower alkylamino carbonylamino, arylamino carbonylamino, lower alkoxycarbonyl, lower alkyl, lower alkenyl, lower alkynyl, lower alkylthio, lower alkoxy, lower alkylamino, lower alkylsulfinyl, lower sulfonyl, lower alkylsulfonyl, lower alkanoyl, lower alkylphosphonyl, aminosulfonyl lower alkyl, hydroxy lower alkyl, alkylsulfinyl lower alkyl, alkylsulfonyl lower alkyl, alkylthio lower alkyl, heteroarylthio lower alkyl, heteroaryloxy lower alkyl, heteroarylamino lower alkyl, halo lower alkyl, alkoxy lower alkyl; and wherein X₁ and X₂ or X₃ may be bonded together to form a heterocylic or heteroaryl ring(s); or X₃ and Z together form a heterobicyclic ring;
X_{1′}, X_{2′}, X_{3′} and X_{4′} are each independently hydrogen, halogen, hydroxy, amino, carboxyl, nitro, cyano, or substituted or unsubstituted alkyl, alkenyl, alkynyl, arylalkyl, heterocylyl, heteroaryl, aryl, aroyl, aryloxy, alkylenedioxy, lower alkyl carbonylamino, lower alkenyl carbonylamino, aryl carbonylamino, arylalkyl carbonylamino, lower alkoxy carbonylamino, lower alkylamino carbonylamino, arylamino carbonylamino, lower alkoxycarbonyl, lower alkyl, lower alkenyl, lower alkynyl, lower alkylthio, lower alkoxy, lower alkylamino, lower alkylsulfinyl, lower sulfonyl, lower alkylsulfonyl, lower alkanoyl, lower alkylphosphonyl, aminosulfonyl lower alkyl, hydroxy lower alkyl, alkylsulfinyl lower alkyl, alkylsulfonyl lower alkyl, alkylthio lower alkyl, heteroarylthio lower alkyl, heteroaryloxy lower alkyl, heteroarylamino lower alkyl, halo lower alkyl, alkoxy lower alkyl;
or a pharmaceutically acceptable salt thereof.

13. The method of claim 12, wherein the small molecule antagonist comprises a compound of the formula: 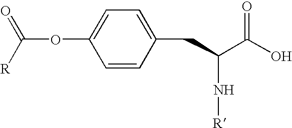
wherein R and R′ comprise any of the designated R and R′ substituents listed in Tables 1 and 2.

14. The method of claim 12, wherein the small molecule antagonist is any one of the compounds listed in Table 3.

15. The method of claim 1, wherein the B cell mobilizing agent is an αL integrin antagonist.

16. The method of claim 15, wherein the B cell mobilizing agent is an αLβ2 integrin antagonist.

17. The method of claim 15, wherein the αL integrin antagonist is an antibody or a biologically active fragment thereof.

18. The method of claim 17, wherein the antibody is a humanized, human, or chimeric antibody, or biologically active fragment thereof.

19. The method of claim 17, wherein the antibody binds the αL subunit (CD11a).

20. The method of claim 17, wherein the CD11a binding antibody comprises the VL and VH sequence of SEQ ID NO. 49 and 50, respectively, or is efalizumab.

21. The method of claim 15, wherein the αL integrin antagonist is a small molecule.

22. The method of claim 21, wherein the small molecule αL antagonist comprises one or more of:
a) a compound of Formula XI: 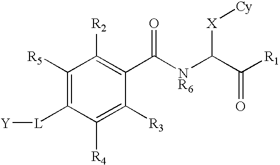
wherein
Cy is a non-aromatic carbocycle or heterocycle optionally substituted with hydroxyl (—OH), mercapto (—SH), thioalkyl, halogen (F, Cl, Br, I), oxo (═O), thio (═S), amino, aminoalkyl, amidine (—C(NH)—NH₂), guanidine (—NH₂—C(NH)—NH₂), nitro, alkyl, alkoxy or acyl;
X is a divalent hydrocarbon chain optionally substituted with hydroxyl, mercapto, halogen, amino, aminoalkyl, nitro, oxo, or thio, and optionally interrupted with N, O, S, SO, or SO₂;
Y is a carbocycle or heterocycle optionally substituted with hydroxyl, mercapto, halogen, oxo, thio, a hydrocarbon, a halo-substituted hydrocarbon, amino, amidine, guanidine, cyano, nitro, alkoxy, or acyl;
L is a bond or a divalent hydrocarbon optionally having one or more carbon atoms replaced with N, O, S, SO, or SO₂, and optionally being substituted with hydroxyl, halogen, oxo, or thio; or three carbon atoms of the hydrocarbon are replaced with an amino acid residue;
R₁ is H, OH, amino, O-carbocycle, or alkoxy optionally substituted with amino, a carbocycle or a heterocycle;
R₂₋₅ are independently H, hydroxyl, mercapto, halogen, cyano, amino, amidine, guanidine, nitro, or alkoxy; or R₃ and R₄ together form a fused carbocycle or heterocycle optionally substituted with hydroxyl, halogen, oxo, thio, amino, amidine, guanidine, or alkoxy;
R₆ is H or a hydrocarbon chain optionally substituted with a carbocycle or a heterocycle;
or salts, solvates, and hydrates thereof;
with the proviso that when Y is phenyl, R₂, R₄, and R₅ are H, R₃, is Cl, and R₁ is OH, then X is other than cyclohexyl;
or a pharmaceutically acceptable salt thereof.

23. The method of claim 21, wherein the small molecule αL antagonist comprises any one of the compounds listed in Table 4.

24. The method of claim 3, wherein the α4 integrin antagonist is an antibody that binds VCAM-1 (CD106).

25. The method of claim 15, wherein the αL integrin antagonist is an antibody that binds ICAM-1 (CD54).

26. The method of claim 3, wherein the antagonist is an immunoadhesin comprising a ligand binding portion of VCAM-1 (CD106) fused to a hinge and Fc of a human IgG.

27. The method of claim 15, wherein the antagonist is an immunoadhesin comprising a ligand binding portion of ICAM-1 (CD54) fused to a hinge and Fc of a human IgG.

28. The method of claim 1, comprising two B cell mobilizing agents, wherein the first mobilizing agent is an αL integrin antagonist and the second mobilizing agent is an α4 integrin antagonist.

29. The method of claim 28, wherein the α4 integrin is α4β1 or α4β7, and the αL is αLβ2.

30. The method of claim 28 or 29, wherein the αL integrin antagonist and the α4 integrin antagonist are both antibodies.

31. The method of claim 28 or 29, wherein the α4 integrin antagonist is natalizumab or a biologically active fragment or humanized for thereof.

32. The method of claim 28 or 29, wherein the αL integrin antagonist is the antibody efalizumab or a biologically active fragment or humanized for thereof.

33. The method of claim 28, wherein the αL integrin antagonist and the α4 integrin antagonist are both small molecules.

34. The method of any one of the preceding claims wherein the B cell depleting agent is an antagonist of a B cell surface marker.

35. The method of claim 34, wherein the B cell surface marker is CD20, CD22, or CD52.

36. The method of claim 35, wherein the B cell surface marker is CD20.

37. The method of claim 36, wherein the B cell depleting agent is an antibody that binds CD20.

38. The method of claim 37, wherein the antibody is rituximab.

39. The method of claim 37, wherein the antibody that binds CD20 is a humanized antibody.

40. The method of claim 39, wherein the humanized antibody is selected from the group of humanized 2H7.v16, v31, v114, v138, v477, v588, v511, and antibody that comprises the amino acid sequence of SEQ ID NO. 29 and SEQ ID NO. 30 as variable light and variable heavy chain, respectively.

41. The method of claim 37, wherein the antibody is a human or chimeric antibody.

42. The method of any of the preceding claims wherein the B cell mobilizing agent and the B cell depleting agent are administered concurrently or sequentially.

43. The method of any one of claims 23 to 29, wherein the first and second B cell mobilizing agents are administered concurrently.

44. A method of enhancing the efficacy of B cell depletion by a CD20 binding antibody, comprising administering to a patient suffering from a B cell disorder, one or more B cell mobilizing agent.

45. The method of claim 44, wherein the CD20 binding antibody is rituximab.

46. The method of claim 44, wherein the CD20 binding antibody is selected from the group consisting of humanized 2H7.v16, v31, v114, v138, v477, v588, v511, and antibody that comprises the amino acid sequence of SEQ ID NO. 29 and SEQ ID NO. 30 as variable light and variable heavy chain, respectively.

47. The method of claim 45 or claim 46, wherein the B cell mobilizing agent is an αL integrin antagonist.

48. The method of claim 47, wherein the αL integrin antagonist is efalizumab or a CD11a binding antibody that comprises the VL and VH sequence of SEQ ID NO. 49 and 50, respectively.

49. The method of any of claims 44-48 comprising two or more B cell mobilizing agents, wherein the first mobilizing agent is an αL integrin antagonist and the second mobilizing agent is an α4 integrin antagonist.

50. The method of claim 49, wherein the α4 integrin antagonist is an antibody that binds α4β1 or α4β7.

51. The method of claim 49 or 50 wherein the αL integrin antagonist is efalizumab or a CD11a binding antibody that comprises the VL and VH sequence of SEQ ID NO. 49 and 50, respectively.

52. The method of claim 50 wherein the αL integrin antagonist and the α4 integrin antagonist act synergistically to enhance B cell depletion.

53. A method of treating a B cell neoplasm or malignancy characterized by B cells expressing CD20, comprising administering to a patient suffering from the neoplasm or malignancy, a therapeutically effective amount of a CD20 binding antibody and at least one B cell mobilizing agent.

54. The method of claim 53, wherein the CD20 binding antibody is rituximab.

55. The method of claim 53, wherein the CD20 binding antibody is selected from the group consisting of humanized 2H7.v16, v31, v114, v138, v477, v588, v511, and antibody that comprises the amino acid sequence of SEQ ID NO. 29 and SEQ ID NO. 30 as variable light and variable heavy chain, respectively.

56. The method of any of claims 53-55, wherein the B cell mobilizing agent is an αL integrin antagonist.

57. The method of claim 56, wherein the αL integrin antagonist is efalizumab or a CD11a binding antibody that comprises the VL and VH sequence of SEQ ID NO. 49 and 50, respectively.

58. The method of claim 57, wherein the α4 integrin antagonist is an antibody or small molecule that binds α4β1.

59. The method of claim 56, wherein the B cell neoplasm is selected from the group consisting of non-Hodgkin's lymphoma (NHL), small lymphocytic (SL) NHL, lymphocyte predominant Hodgkin's disease (LPHD), follicular center cell (FCC) lymphomas, acute lymphocytic leukemia (ALL), chronic lymphocytic leukemia (CLL), and Hairy cell leukemia.

60. A method of alleviating a B-cell regulated autoimmune disorder comprising administering to a patient suffering from the autoimmune disorder, a therapeutically effective amount of a CD20 binding antibody and at least one B cell mobilizing agent.

61. The method of claim 59, wherein the CD20 binding antibody is rituximab.

62. The method of claim 60, wherein the CD20 binding antibody comprising a light chain variable domain sequence of SEQ ID No. 31 and heavy chain variable domain sequence of SEQ ID NO. 32.

63. The method of any of claims 60-62, wherein the B cell mobilizing agent is an αL integrin antagonist.

64. The method of claim 63, wherein the αL integrin antagonist is efalizumab or a CD11a binding antibody that comprises the VL and VH sequence of SEQ ID NO. 49 and 50, respectively.

65. The method of any of claims 60-62, wherein the B cell mobilizing agent is an antibody or small molecule that binds α4β1.

66. The method of any of claims 60-62, wherein the autoimmune disorder is selected from the group consisting of rheumatoid arthritis and juvenile rheumatoid arthritis, systemic lupus erythematosus (SLE) including lupus nephritis, Wegener's disease, inflammatory bowel disease, ulcerative colitis, idiopathic thrombocytopenic purpura (ITP), thrombotic throbocytopenic purpura (TTP), autoimmune thrombocytopenia, multiple sclerosis, psoriasis, IgA nephropathy, IgM polyneuropathies, myasthenia gravis, ANCA associated vasculitis, diabetes mellitus, Reynaud's syndrome, Sjorgen's syndrome, Neuromyelitis Optica (NMO) and glomerulonephritis.

67. The method of claim 64, wherein the autoimmune disorder is multiple sclerosis.

68. A method of depleting marginal zone B cells in the spleen of a patient suffering from a B cell neoplasm or a B-cell regulated autoimmune disorder, comprising administering to the patient, a therapeutically effective amount of a CD20 binding antibody and at least one B cell mobilizing agent.

69. A composition comprising an antibody that binds an αL integrin and an antibody that binds an α4 integrin.

70. The composition of claim 69, wherein the antibody that binds the α4 integrin is efalizumab or a CD11a binding antibody that comprises the VL and VH sequence of SEQ ID NO. 49 and 50, respectively.
